SEQUENCE LISTING (135 sequences)

SEQ ID NO: 1 - protein, 104 aa, Homo sapiens
  Human Axl extracelullar domain 1 (Ig-like domain 1)
ESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEG

SEQ ID NO: 2 - protein, 90 aa, Homo sapiens
  Human Axl extracelullar domain 2 (Ig-like domain 2)
LPYFLEEPEDRTVAANTPFNLSCQAQGPPEPVDLLWLQDAVPLATAPGHGPQRSLHVPGLNKTSSFSCEAHNAKGVTTSRTATITVLPQQ

SEQ ID NO: 3 - protein, 104 aa, Homo sapiens
  Human Axl extracelullar domain 3 (Fnlll-like domain 1)
PRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEG

SEQ ID NO: 4 - protein, 111 aa, Homo sapiens
  Human Axl extracelullar domain 4 (Fnlll-like domain 2)
VPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPS

SEQ ID NO: 5 - protein, 117 aa, Artificial Sequence
  Abcon heavy chain variable region
  misc_feature (11)..(11): Xaa is an amino acid with an aliphatic side chain (for example, Leu or Val)
  misc_feature (13)..(13): Xaa is Lys or Gln
  misc_feature (16)..(16): Xaa is Gly or Arg
  misc_feature (19)..(19): Xaa is an amino acid with a positively charged side chain (for example, Lys or Arg)
  misc_feature (23)..(23): Xaa is Ala or Thr
  misc_feature (49)..(49): Xaa is an amino acid with an aliphatic side chain (for example, Ala or Gly)
  misc_feature (76)..(76): Xaa is Asp, Asn or Glu
  misc_feature (79)..(79): Xaa is an amino acid with a polar side chain (for example, Asn or Ser)
  misc_feature (80)..(80): Xaa is Thr, Ser or Ile
  misc_feature (81)..(81): Xaa is an amino acid with an aliphatic or hydrophobic side chain (for example, Leu or Ala)
  misc_feature (89)..(89): Xaa is an amino acid with a positively charged side chain (for example, Lys or Arg)
  misc_feature (90)..(90): Xaa is Thr or Ala
  misc_feature (112)..(112): Xaa is Leu or Thr
  misc_feature (117)..(117): Xaa is Ser or Ala
EVQLVESGGGXVXPGXSLXLSCXASGFTFSDYWMEWVRQAPGKGLEWVXEIRNKVNNYATYYGKSVKGRFTISRDXSKXXXYLQMNSLXXEDTAVYYCNTLRLFAYWGQGTXVTVSX

SEQ ID NO: 6 - protein, 112 aa, Artificial Sequence
  Ab302/303/304/305/Abcon light chain variable region
DIQMTQSPSSLSASVGDRVTITCRSSKSLLHRQGITYVYWYQQKPGKVPKLLIYRMSTLASGVPSRFSGSGSGTDFTLTISSLQPEDVATYYCGQLLENPYTFGQGTKLEIK

SEQ ID NO: 7 - protein, 117 aa, Artificial Sequence
  Ab302 heavy chain variable region
EVQLVESGGGLVKPGGSLKLSCAASGFTFSDYWMEWVRQAPGKGLEWVAEIRNKVNNYATYYGKSVKGRFTISRDDSKNTLYLQMNSLKTEDTAVYYCNTLRLFAYWGQGTLVTVSS

SEQ ID NO: 8 - protein, 117 aa, Artificial Sequence
  Ab303 heavy chain variable region
EVQLVESGGGVVQPGRSLRLSCAASGFTFSDYWMEWVRQAPGKGLEWVAEIRNKVNNYATYYGKSVKGRFTISRDNSKNTLYLQMNSLRAEDTAVYYCNTLRLFAYWGQGTLVTVSS

SEQ ID NO: 9 - protein, 117 aa, Artificial Sequence
  Ab304 heavy chain variable region
EVQLVESGGGLVQPGGSLRLSCAASGFTFSDYWMEWVRQAPGKGLEWVGEIRNKVNNYATYYGKSVKGRFTISRDESKNSLYLQMNSLKTEDTAVYYCNTLRLFAYWGQGTTVTVSS

SEQ ID NO: 10 - protein, 117 aa, Artificial Sequence
  Ab305 heavy chain variable region
EVQLVESGGGLVQPGRSLRLSCTASGFTFSDYWMEWVRQAPGKGLEWVGEIRNKVNNYATYYGKSVKGRFTISRDDSKSIAYLQMNSLKTEDTAVYYCNTLRLFAYWGQGTLVTVSA

SEQ ID NO: 11 - protein, 117 aa, mouse
  Ab301 heavy chain variable region
EVKLEASGGGLVQPGMSVKLSCTTSGFTFSDYWMEWVRQAPGKGLEWVAEIRNKVNNYATYYGKSVKGRFTISRDDSKSIVYLQMNSIRSEDTGIYYCNTLRLFAYWGQGTLVTVSA

SEQ ID NO: 12 - protein, 112 aa, mouse
  Ab301 light chain variable region
DIVMTQAPLSISVTPGESPSMSCRSSKSLLHRNGITYVYWYLQKPGKSPQLLIYRMSTLASGVPDRFSGSGSETDFTLKINKVEAEDVGIYYCGQLLENPYTFGAGTRLEIK

SEQ ID NO: 13 - DNA, 351 bp, Artificial Sequence
  Ab302 heavy chain variable region
gaggtgcagctggtggaatccggcggaggtttagtgaagcccggtggctctttaaagctgagctgtgccgcctccggcttcaccttctccgactactggatggagtgggtgcggcaagctcccggtaagggtttagagtgggtggccgagatccggaacaaggtgaacaactacgccacctactacggcaagtccgtgaagggccggttcaccatctcccgggacgactccaagaacactttatatttacagatgaactctttaaagaccgaggacaccgccgtgtattactgcaacactttacggctgtttgcctactggggccaaggtactttagtgacagtgtcctcc

SEQ ID NO: 14 - DNA, 336 bp, Artificial Sequence
  Ab302/303/304/305 light chain variable region
gacatccagatgacacagagcccttcctctttatccgcttccgtgggagatcgtgtgaccatcacttgtcgttcctccaagtctttactgcataggcaaggtatcacctacgtgtactggtaccagcagaagcccggcaaggtgcccaagctgctgatctatcgtatgtccactttagcttccggcgtgccttctcgtttttctggctccggctctggcacagacttcactttaaccatctcctctttacagcccgaggacgtggccacatactactgcggccagctgctggagaatccctacaccttcggccaaggtaccaagctggagatcaag

SEQ ID NO: 15 - DNA, 351 bp, Artificial Sequence
  Ab303 heavy chain variable region
gaggtgcagctggtggagtccggaggaggcgtggtgcaacccggtcgttctttaaggctgtcttgtgctgcttccggcttcaccttctccgactactggatggagtgggtgaggcaagctcccggtaaaggactggagtgggtggccgagatccggaacaaggtgaacaactacgccacctactacggcaagagcgtgaagggtcgtttcaccatctctcgtgacaactccaagaacactttatatttacagatgaactccttacgtgccgaggacaccgccgtgtactactgcaacactttacggctgttcgcttactggggacaaggtactttagtgaccgtgtcctcc

SEQ ID NO: 16 - DNA, 351 bp, Artificial Sequence
  Ab304 heavy chain variable region
gaggtgcagctggtggagtccggaggcggactggtgcaacccggtggatctctgaggctgtcttgtgccgcttccggcttcaccttctccgactactggatggagtgggtgaggcaagctcccggcaaaggactggagtgggtcggcgagatccggaacaaggtgaacaactacgccacctactacggcaagtccgtgaagggtcgtttcaccatctctcgtgacgagagcaagaactctttatatttacagatgaactctttaaagaccgaggacaccgccgtgtactactgcaacactttacgtctgttcgcctattggggccaaggtaccacagtgaccgtgtcctcc

SEQ ID NO: 17 - DNA, 351 bp, Artificial Sequence
  Ab305 heavy chain variable region
gaggtgcagctggtggaatccggcggaggactggtgcagcccggtcgttctttaaggctgtcttgtacagcctccggcttcaccttcagcgactactggatggagtgggtgaggcaagctcccggtaagggactggagtgggtgggcgagatccggaacaaggtgaacaactacgccacctactacggcaagagcgtgaagggtcgtttcaccatctctcgtgacgattccaagtccatcgcctatttacagatgaactctttaaagaccgaggacaccgccgtgtactactgcaacactttacggctgttcgcctattggggccaaggtactttagtgaccgtgtccgcc

SEQ ID NO: 18 - DNA, 351 bp, mouse
  Ab301 heavy chain variable region
gaggtgaagctggaagcctccggcggaggactggtgcagcccggtatgagcgtgaagctgagctgcaccaccagcggcttcaccttcagcgactactggatggagtgggtgaggcaagctcccggtaaaggtttagagtgggtggccgaaattcgtaacaaggtgaacaactacgccacctactacggcaagtccgtgaagggtcgttttaccatctctcgtgacgacagcaagagcatcgtgtatttacagatgaacagcattcgtagcgaggacaccggcatctactactgcaacactttaaggctgttcgcctactggggacaaggtactttagtgaccgtgtccgcc

SEQ ID NO: 19 - DNA, 336 bp, Artificial Sequence
  Ab301 light chain variable region
gacattgtgatgacccaagctcctttatccatcagcgtgacacccggtgagagccccagcatgagctgtcgtagcagcaagtctttactgcatcgtaacggcatcacctacgtgtactggtatttacagaagcccggcaagagcccccagctgctgatctatcgtatgagcacactggccagcggcgtgcccgatagattctccggcagcggcagcgagaccgacttcactttaaagatcaacaaggtggaggccgaggacgtgggcatctactactgcggccagctgctggagaatccctacaccttcggcgccggcacaaggctggagatcaag

SEQ ID NO: 20 - protein, 5 aa, Artificial Sequence
  Ab301/Ab302/303/304/305/Abcon VH CDR1 (Kabat)
DYWME

SEQ ID NO: 21 - protein, 19 aa, Artificial Sequence
  Ab301/Ab302/303/304/305/Abcon VH CDR2 (Kabat)
EIRNKVNNYATYYGKSVKG

SEQ ID NO: 22 - protein, 6 aa, Artificial Sequence
  Ab301/Ab302/303/304/305/Abcon VH CDR3 (Kabat, Chothia and AbM)
LRLFAY

SEQ ID NO: 23 - protein, 7 aa, Artificial Sequence
  Ab301/Ab302/303/304/305/Abcon VH CDR1 (Chothia)
GFTFSDY

SEQ ID NO: 24 - protein, 8 aa, Artificial Sequence
  Ab301/Ab302/303/304/305/Abcon VH CDR2 (Chothia)
RNKVNNYA

SEQ ID NO: 25 - protein, 10 aa, Artificial Sequence
  Ab301/Ab302/303/304/305/Abcon VH CDR1 (AbM)
GFTFSDYWME

SEQ ID NO: 26 - protein, 12 aa, Artificial Sequence
  Ab301/Ab302/303/304/305/Abcon VH CDR2 (AbM)
EIRNKVNNYATY

SEQ ID NO: 27 - protein, 6 aa, Artificial Sequence
  Ab301/Ab302/303/304/305/Abcon VH CDR1 (Contact)
SDYWME

SEQ ID NO: 28 - protein, 15 aa, Artificial Sequence
  Abcon VH CDR2 (Contact)
  misc_feature (3)..(3): Xaa is an amino acid with an aliphatic side chain (for example, Ala or Gly)
WVXEIRNKVNNYATY

SEQ ID NO: 29 - protein, 7 aa, Artificial Sequence
  Ab301/Ab302/303/304/305/Abcon VH CDR3 (Contact)
NTLRLFA

SEQ ID NO: 30 - protein, 16 aa, Artificial Sequence
  Ab302/303/304/305/Abcon VL CDR1 (Kabat, Chothia and AbM)
RSSKSLLHRQGITYVY

SEQ ID NO: 31 - protein, 7 aa, Artificial Sequence
  Ab301/Ab302/303/304/305/Abcon VL CDR2 (Kabat, Chothia and AbM)
RMSTLAS

SEQ ID NO: 32 - protein, 9 aa, Artificial Sequence
  Ab301/Ab302/303/304/305/Abcon VL CDR3 (Kabat, Chothia, AbM, IMGT)
GQLLENPYT

SEQ ID NO: 33 - protein, 12 aa, Artificial Sequence
  Ab302/303/304/305/Abcon VL CDR1 (Contact)
LHRQGITYVYWY

SEQ ID NO: 34 - protein, 10 aa, Artificial Sequence
  Ab301/Ab302/303/304/305/Abcon VL CDR2 (Contact)
LLIYRMSTLA

SEQ ID NO: 35 - protein, 8 aa, Artificial Sequence
  Ab301/Ab302/303/304/305/Abcon VL CDR3 (Contact)
GQLLENPY

SEQ ID NO: 36 - protein, 15 aa, Artificial Sequence
  Ab301/Ab302/303 VH CDR2 (Contact)
WVAEIRNKVNNYATY

SEQ ID NO: 37 - protein, 15 aa, Artificial Sequence
  Ab304/305 VH CDR2 (Contact)
WVGEIRNKVNNYATY

SEQ ID NO: 38 - protein, 16 aa, Artificial Sequence
  Ab301 VL CDR1 (Kabat, Chothia, AbM)
RSSKSLLHRNGITYVY

SEQ ID NO: 39 - protein, 12 aa, Artificial Sequence
  Ab301 VL CDR1 (Contact)
LHRNGITYVYWY

SEQ ID NO: 40 - protein, 30 aa, Artificial Sequence
  Abcon VH FR1 (Kabat)
  misc_feature (11)..(11): Xaa is an amino acid with an aliphatic side chain (for example, Leu or Val)
  misc_feature (13)..(13): Xaa is Lys or Gln
  misc_feature (16)..(16): Xaa is Gly or Arg
  misc_feature (19)..(19): Xaa is an amino acid with a positively charged side chain (for example, Lys or Arg)
  misc_feature (23)..(23): Xaa is Ala or Thr
EVQLVESGGGXVXPGXSLXLSCXASGFTFS

SEQ ID NO: 41 - protein, 14 aa, Artificial Sequence
  Abcon VH FR2 (Kabat and AbM)
  misc_feature (14)..(14): Xaa is an amino acid with an aliphatic side chain (for example, Ala or Gly)
WVRQAPGKGLEWVX

SEQ ID NO: 42 - protein, 32 aa, Artificial Sequence
  Abcon VH FR3 (Kabat)
  misc_feature (8)..(8): Xaa is Asp, Asn or Glu
  misc_feature (11)..(11): Xaa is an amino acid with a polar side chain (for example, Asn or Ser)
  misc_feature (12)..(12): Xaa is Thr, Ser or Ile
  misc_feature (13)..(13): Xaa is an amino acid with an aliphatic or hydrophobic side chain (for example, Leu or Ala)
  misc_feature (21)..(21): Xaa is an amino acid with a positively charged side chain (for example, Lys or Arg)
  misc_feature (22)..(22): Xaa is Thr or Ala
RFTISRDXSKXXXYLQMNSLXXEDTAVYYCNT

SEQ ID NO: 43 - protein, 11 aa, Artificial Sequence
  Abcon VH FR4 (Kabat, Chothia, AbM, IMGT)
  misc_feature (6)..(6): Xaa is Leu or Thr
  misc_feature (11)..(11): Xaa is Ser or Ala
WGQGTXVTVSX

SEQ ID NO: 44 - protein, 25 aa, Artificial Sequence
  Abcon VH FR1 (Chothia, AbM, IMGT)
  misc_feature (11)..(11): Xaa is an amino acid with an aliphatic side chain (for example, Leu or Val)
  misc_feature (13)..(13): Xaa is Lys or Gln
  misc_feature (16)..(16): Xaa is Gly or Arg
  misc_feature (19)..(19): Xaa is an amino acid with a positively charged side chain (for example, Lys or Arg)
  misc_feature (23)..(23): Xaa is Ala or Thr
EVQLVESGGGXVXPGXSLXLSCXAS

SEQ ID NO: 45 - protein, 19 aa, Artificial Sequence
  Abcon VH FR2 (Chothia)
  misc_feature (17)..(17): Xaa is an amino acid with an aliphatic side chain (for example, Ala or Gly)
WMEWVRQAPGKGLEWVXEI

SEQ ID NO: 46 - protein, 41 aa, Artificial Sequence
  Abcon VH FR3 (Chothia)
  misc_feature (17)..(17): Xaa is Asp, Asn or Glu
  misc_feature (20)..(20): Xaa is an amino acid with a polar side chain (for example, Asn or Ser)
  misc_feature (21)..(21): Xaa is Thr, Ser or Ile
  misc_feature (22)..(22): Xaa  is an amino acid with an aliphatic or hydrophobic side chain (for example, Leu or Ala)
  misc_feature (30)..(30): Xaa is an amino acid with a positively charged side chain (for example, Lys or Arg)
  misc_feature (31)..(31): Xaa is Thr or Ala
TYYGKSVKGRFTISRDXSKXXXYLQMNSLXXEDTAVYYCNT

SEQ ID NO: 47 - protein, 39 aa, Artificial Sequence
  Abcon VH FR3 (AbM)
  misc_feature (15)..(15): Xaa is Asp, Asn or Glu
  misc_feature (18)..(18): Xaa is an amino acid with a polar side chain (for example, Asn or Ser)
  misc_feature (19)..(19): Xaa is Thr, Ser or Ile
  misc_feature (20)..(20): Xaa  is an amino acid with an aliphatic or hydrophobic side chain (for example, Leu or Ala)
  misc_feature (28)..(28): Xaa is an amino acid with a positively charged side chain (for example, Lys or Arg)
  misc_feature (29)..(29): Xaa is Thr or Ala
YGKSVKGRFTISRDXSKXXXYLQMNSLXXEDTAVYYCNT

SEQ ID NO: 48 - protein, 29 aa, Artificial Sequence
  Abcon VH FR1 (Contact)
  misc_feature (11)..(11): Xaa is an amino acid with an aliphatic side chain (for example, Leu or Val)
  misc_feature (13)..(13): Xaa is Lys or Gln
  misc_feature (16)..(16): Xaa is Gly or Arg
  misc_feature (19)..(19): Xaa is an amino acid with a positively charged side chain (for example, Lys or Arg)
  misc_feature (23)..(23): Xaa is Ala or Thr
EVQLVESGGGXVXPGXSLXLSCXASGFTF

SEQ ID NO: 49 - protein, 11 aa, Artificial Sequence
  Ab301/302/303/304/305/Abcon VH FR2 (Contact)
WVRQAPGKGLE

SEQ ID NO: 50 - protein, 37 aa, Artificial Sequence
  Abcon VH FR3 (Contact)
  misc_feature (15)..(15): Xaa is Asp, Asn or Glu
  misc_feature (18)..(18): Xaa is an amino acid with a polar side chain (for example, Asn or Ser)
  misc_feature (19)..(19): Xaa is Thr, Ser or Ile
  misc_feature (20)..(20): Xaa  is an amino acid with an aliphatic or hydrophobic side chain (for example, Leu or Ala)
  misc_feature (28)..(28): Xaa is an amino acid with a positively charged side chain (for example, Lys or Arg)
  misc_feature (29)..(29): Xaa is Thr or Ala
YGKSVKGRFTISRDXSKXXXYLQMNSLXXEDTAVYYC

SEQ ID NO: 51 - protein, 12 aa, Artificial Sequence
  Abcon VH FR4 (Contact)
  misc_feature (7)..(7): Xaa is Leu or Thr
  misc_feature (12)..(12): Xaa is Ser or Ala
YWGQGTXVTVSX

SEQ ID NO: 52 - protein, 23 aa, Artificial Sequence
  Ab302/303/304/305/Abcon VL FR1 (Kabat, Chothia, AbM)
DIQMTQSPSSLSASVGDRVTITC

SEQ ID NO: 53 - protein, 15 aa, Artificial Sequence
  Ab302/303/304/305/Abcon VL FR2 (Kabat, Chothia, AbM)
WYQQKPGKVPKLLIY

SEQ ID NO: 54 - protein, 32 aa, Artificial Sequence
  Ab302/303/304/305/Abcon VL FR3 (Kabat, Chothia, AbM)
GVPSRFSGSGSGTDFTLTISSLQPEDVATYYC

SEQ ID NO: 55 - protein, 10 aa, Artificial Sequence
  Ab302/303/304/305/Abcon VL FR4 (Kabat, Chothia, AbM)
FGQGTKLEIK

SEQ ID NO: 56 - protein, 29 aa, Artificial Sequence
  Ab302/303/304/305/Abcon VL FR1 (Contact)
DIQMTQSPSSLSASVGDRVTITCRSSKSL

SEQ ID NO: 57 - protein, 9 aa, Artificial Sequence
  Ab302/303/304/305/Abcon VL FR2 (Contact)
QQKPGKVPK

SEQ ID NO: 58 - protein, 33 aa, Artificial Sequence
  Ab302/303/304/305/Abcon VL FR3 (Contact)
SGVPSRFSGSGSGTDFTLTISSLQPEDVATYYC

SEQ ID NO: 59 - protein, 11 aa, Artificial Sequence
  Ab302/303/304/305/Abcon VL FR4 (Contact)
TFGQGTKLEIK

SEQ ID NO: 60 - protein, 30 aa, Artificial Sequence
  Ab302 VH FR1 (Kabat)
EVQLVESGGGLVKPGGSLKLSCAASGFTFS

SEQ ID NO: 61 - protein, 14 aa, Artificial Sequence
  Ab301/302/303 VH FR2 (Kabat)
WVRQAPGKGLEWVA

SEQ ID NO: 62 - protein, 32 aa, Artificial Sequence
  Ab302 VH FR3 (Kabat)
RFTISRDDSKNTLYLQMNSLKTEDTAVYYCNT

SEQ ID NO: 63 - protein, 11 aa, Artificial Sequence
  Ab302/303 VH FR4 (Kabat, Chothia, AbM, IMGT)
WGQGTLVTVSS

SEQ ID NO: 64 - protein, 25 aa, Artificial Sequence
  Ab302 VH FR1 (Chothia, AbM, IMGT)
EVQLVESGGGLVKPGGSLKLSCAAS

SEQ ID NO: 65 - protein, 19 aa, Artificial Sequence
  Ab301/302/303 VH FR2 (Chothia)
WMEWVRQAPGKGLEWVAEI

SEQ ID NO: 66 - protein, 41 aa, Artificial Sequence
  Ab302 VH FR3 (Chothia)
TYYGKSVKGRFTISRDDSKNTLYLQMNSLKTEDTAVYYCNT

SEQ ID NO: 67 - protein, 14 aa, Artificial Sequence
  Ab301/302/303 VH FR2 (AbM)
WVRQAPGKGLEWVA

SEQ ID NO: 68 - protein, 39 aa, Artificial Sequence
  Ab302 VH FR3 (AbM)
YGKSVKGRFTISRDDSKNTLYLQMNSLKTEDTAVYYCNT

SEQ ID NO: 69 - protein, 29 aa, Artificial Sequence
  Ab302 VH FR1 (Contact)
EVQLVESGGGLVKPGGSLKLSCAASGFTF

SEQ ID NO: 70 - protein, 37 aa, Artificial Sequence
  Ab302 VH FR3 (Contact)
YGKSVKGRFTISRDDSKNTLYLQMNSLKTEDTAVYYC

SEQ ID NO: 71 - protein, 30 aa, Artificial Sequence
  Ab303 VH FR1 (Kabat)
EVQLVESGGGVVQPGRSLRLSCAASGFTFS

SEQ ID NO: 72 - protein, 32 aa, Artificial Sequence
  Ab303 VH FR3 (Kabat)
RFTISRDNSKNTLYLQMNSLRAEDTAVYYCNT

SEQ ID NO: 73 - protein, 25 aa, Artificial Sequence
  Ab303 VH FR1 (Chothia, AbM, IMGT)
EVQLVESGGGVVQPGRSLRLSCAAS

SEQ ID NO: 74 - protein, 41 aa, Artificial Sequence
  Ab303 VH FR3 (Chothia)
TYYGKSVKGRFTISRDNSKNTLYLQMNSLRAEDTAVYYCNT

SEQ ID NO: 75 - protein, 39 aa, Artificial Sequence
  Ab303 VH FR3 (AbM)
YGKSVKGRFTISRDNSKNTLYLQMNSLRAEDTAVYYCNT

SEQ ID NO: 76 - protein, 29 aa, Artificial Sequence
  Ab303 VH FR1 (Contact)
EVQLVESGGGVVQPGRSLRLSCAASGFTF

SEQ ID NO: 77 - protein, 37 aa, Artificial Sequence
  Ab303 VH FR3 (Contact)
YGKSVKGRFTISRDNSKNTLYLQMNSLRAEDTAVYYC

SEQ ID NO: 78 - protein, 12 aa, Artificial Sequence
  Ab302/303 VH FR4 (Contact)
YWGQGTLVTVSS

SEQ ID NO: 79 - protein, 30 aa, Artificial Sequence
  Ab304 VH FR1 (Kabat)
EVQLVESGGGLVQPGGSLRLSCAASGFTFS

SEQ ID NO: 80 - protein, 14 aa, Artificial Sequence
  Ab304/305 VH FR2 (Kabat)
WVRQAPGKGLEWVG

SEQ ID NO: 81 - protein, 32 aa, Artificial Sequence
  Ab304 VH FR3 (Kabat)
RFTISRDESKNSLYLQMNSLKTEDTAVYYCNT

SEQ ID NO: 82 - protein, 11 aa, Artificial Sequence
  Ab304 VH FR4 (Kabat, Chothia, AbM INGT)
WGQGTTVTVSS

SEQ ID NO: 83 - protein, 25 aa, Artificial Sequence [sequence omitted]

SEQ ID NO: 84 - protein, 19 aa, Artificial Sequence
  Ab304 VH FR2 (Chothia)
WMEWVRQAPGKGLEWVGEI

SEQ ID NO: 85 - protein, 41 aa, Artificial Sequence
  Ab304 VH FR3 (Chothia)
TYYGKSVKGRFTISRDESKNSLYLQMNSLKTEDTAVYYCNT

SEQ ID NO: 86 - protein, 14 aa, Artificial Sequence
  Ab304/305 VH FR2 (AbM)
WVRQAPGKGLEWVG

SEQ ID NO: 87 - protein, 39 aa, Artificial Sequence
  Ab304 VH FR3 (AbM)
YGKSVKGRFTISRDESKNSLYLQMNSLKTEDTAVYYCNT

SEQ ID NO: 88 - protein, 29 aa, Artificial Sequence
  Ab304 VH FR1 (Contact)
EVQLVESGGGLVQPGGSLRLSCAASGFTF

SEQ ID NO: 89 - protein, 37 aa, Artificial Sequence
  Ab304 VH FR3 (Contact)
YGKSVKGRFTISRDESKNSLYLQMNSLKTEDTAVYYC

SEQ ID NO: 90 - protein, 12 aa, Artificial Sequence
  Ab304 VH FR4 (Contact)
YWGQGTTVTVSS

SEQ ID NO: 91 - protein, 30 aa, Artificial Sequence
  Ab305 VH FR1 (Kabat)
EVQLVESGGGLVQPGRSLRLSCTASGFTFS

SEQ ID NO: 92 - protein, 32 aa, Artificial Sequence
  Ab305 VH FR3 (Kabat)
RFTISRDDSKSIAYLQMNSLKTEDTAVYYCNT

SEQ ID NO: 93 - protein, 11 aa, Artificial Sequence
  Ab301/305 VH FR4 (Kabat, Chothia, AbM, IMGT)
WGQGTLVTVSA

SEQ ID NO: 94 - protein, 25 aa, Artificial Sequence
  Ab305 VH FR1 (Chothia, AbM, INGT)
EVQLVESGGGLVQPGRSLRLSCTAS

SEQ ID NO: 95 - protein, 41 aa, Artificial Sequence
  Ab305 VH FR3 (Chothia)
TYYGKSVKGRFTISRDDSKSIAYLQMNSLKTEDTAVYYCNT

SEQ ID NO: 96 - protein, 39 aa, Artificial Sequence
  Ab305 VH FR3 (AbM)
YGKSVKGRFTISRDDSKSIAYLQMNSLKTEDTAVYYCNT

SEQ ID NO: 97 - protein, 29 aa, Artificial Sequence
  Ab305 VH FR1 (Contact)
EVQLVESGGGLVQPGRSLRLSCTASGFTF

SEQ ID NO: 98 - protein, 37 aa, Artificial Sequence
  Ab305 VH FR3 (Contact)
YGKSVKGRFTISRDDSKSIAYLQMNSLKTEDTAVYYC

SEQ ID NO: 99 - protein, 12 aa, Artificial Sequence
  Ab301/305 VH FR4 (Contact)
YWGQGTLVTVSA

SEQ ID NO: 100 - protein, 30 aa, Artificial Sequence
  Ab301 VH FR1 (Kabat)
EVKLEASGGGLVQPGMSVKLSCTTSGFTFS

SEQ ID NO: 101 - protein, 32 aa, Artificial Sequence
  Ab301 VH FR3 (Kabat)
RFTISRDDSKSIVYLQMNSIRSEDTGIYYCNT

SEQ ID NO: 102 - protein, 25 aa, Artificial Sequence
  Ab301 VH FR1 (Chothia, AbM, IMGT)
EVKLEASGGGLVQPGMSVKLSCTTS

SEQ ID NO: 103 - protein, 41 aa, Artificial Sequence
  Ab301 VH FR3 (Chothia)
TYYGKSVKGRFTISRDDSKSIVYLQMNSIRSEDTGIYYCNT

SEQ ID NO: 104 - protein, 39 aa, Artificial Sequence
  Ab301 VH FR3 (AbM)
YGKSVKGRFTISRDDSKSIVYLQMNSIRSEDTGIYYCNT

SEQ ID NO: 105 - protein, 29 aa, Artificial Sequence
  Ab301 VH FR1 (Contact)
EVKLEASGGGLVQPGMSVKLSCTTSGFTF

SEQ ID NO: 106 - protein, 37 aa, Artificial Sequence
  Ab301 VH FR3 (Contact)
YGKSVKGRFTISRDDSKSIVYLQMNSIRSEDTGIYYC

SEQ ID NO: 107 - protein, 23 aa, Artificial Sequence
  Ab301 VH FR1 (Kabat, Chothia, AbM)
DIVMTQAPLSISVTPGESPSMSC

SEQ ID NO: 108 - protein, 15 aa, Artificial Sequence
  Ab301 VL FR2 (Kabat, Chothia, AbM)
WYLQKPGKSPQLLIY

SEQ ID NO: 109 - protein, 32 aa, Artificial Sequence
  Ab301 VL FR3 (Kabat, Chothia, AbM)
GVPDRFSGSGSETDFTLKINKVEAEDVGIYYC

SEQ ID NO: 110 - protein, 10 aa, Artificial Sequence
  Ab301 VL FR4 (Kabat, Chothia, AbM, IMGT)
FGAGTRLEIK

SEQ ID NO: 111 - protein, 29 aa, Artificial Sequence
  Ab301 VL FR1 (Contact)
DIVMTQAPLSISVTPGESPSMSCRSSKSL

SEQ ID NO: 112 - protein, 9 aa, Artificial Sequence
  Ab301 VL FR2 (Contact)
LQKPGKSPQ

SEQ ID NO: 113 - protein, 33 aa, Artificial Sequence
  Ab301 VL FR3 (Contact)
SGVPDRFSGSGSETDFTLKINKVEAEDVGIYYC

SEQ ID NO: 114 - protein, 11 aa, Artificial Sequence
  Ab301 VL FR4 (Contact)
TFGAGTRLEIK

SEQ ID NO: 115 - protein, 38 aa, Artificial Sequence
  Ab305 VH FR3 (IMGT)
YYGKSVKGRFTISRDDSKSIAYLQMNSLKTEDTAVYYC

SEQ ID NO: 116 - protein, 38 aa, Artificial Sequence
  Ab301 VH FR3 (IMGT)
YYGKSVKGRFTISRDDSKSIVYLQMNSIRSEDTGIYYC

SEQ ID NO: 117 - protein, 26 aa, Artificial Sequence
  Ab301 VL FR1 (IMGT)
DIVMTQAPLSISVTPGESPSMSCRSS

SEQ ID NO: 118 - protein, 17 aa, Artificial Sequence
  Ab301 VL FR2 (IMGT)
VYWYLQKPGKSPQLLIY

SEQ ID NO: 119 - protein, 36 aa, Artificial Sequence
  Ab301 VL FR3 (IMGT)
TLASGVPDRFSGSGSETDFTLKINKVEAEDVGIYYC

SEQ ID NO: 120 - protein, 8 aa, Artificial Sequence
  Ab301/302/303/304/305/Abcon VH CDR1 (IMGT)
GFTFSDYW

SEQ ID NO: 121 - protein, 10 aa, Artificial Sequence
  Ab301/302/303/304/305/Abcon VH CDR2 (IMGT)
IRNKVNNYAT

SEQ ID NO: 122 - protein, 8 aa, Artificial Sequence
  Ab301/302/303/304/305/Abcon VH CDR3 (IMGT)
NTLRLFAY

SEQ ID NO: 123 - protein, 11 aa, Artificial Sequence
  Ab302/303/304/305/Abcon VL CDR1 (IMGT)
KSLLHRQGITY

SEQ ID NO: 124 - protein, 3 aa, Artificial Sequence
  Ab301/302/303/304/305/Abcon VL CDR2 (IMGT)
RMS

SEQ ID NO: 125 - protein, 17 aa, Artificial Sequence
  Abcon VH FR2 (IMGT)
  misc_feature (16)..(16): Xaa is an amino acid with an aliphatic side chain (for example, Ala or Gly)
MEWVRQAPGKGLEWVXE

SEQ ID NO: 126 - protein, 38 aa, Artificial Sequence
  Abcon VH FR3 (IMGT)
  misc_feature (16)..(16): Xaa is Asp, Asn or Glu
  misc_feature (19)..(19): Xaa is an amino acid with a polar side chain (for example, Asn or Ser)
  misc_feature (20)..(20): Xaa is Thr, Ser or Ile
  misc_feature (21)..(21): Xaa is an amino acid with an aliphatic or hydrophobic side chain (for example, Leu or Ala)
  misc_feature (29)..(29): Xaa is an amino acid with a positively charged side chain (for example, Lys or Arg)
  misc_feature (30)..(30): Xaa is Thr or Ala
YYGKSVKGRFTISRDXSKXXXYLQMNSLXXEDTAVYYC

SEQ ID NO: 127 - protein, 26 aa, Artificial Sequence [sequence omitted]

SEQ ID NO: 128 - protein, 17 aa, Artificial Sequence
  Ab302/303/304/305/Abcon VL FR2 (IMGT)
VYWYQQKPGKVPKLLIY

SEQ ID NO: 129 - protein, 36 aa, Artificial Sequence
  Ab302/303/304/305/Abcon VL FR3 (IMGT)
TLASGVPSRFSGSGSGTDFTLTISSLQPEDVATYYC

SEQ ID NO: 130 - protein, 11 aa, Artificial Sequence
  Ab301 VL CDR1 (IMGT)
KSLLHRNGITY

SEQ ID NO: 131 - protein, 17 aa, Artificial Sequence
  Ab301/302/303 VH FR2 (IMGT)
MEWVRQAPGKGLEWVAE

SEQ ID NO: 132 - protein, 38 aa, Artificial Sequence
  Ab302 VH FR3 (IMGT)
YYGKSVKGRFTISRDDSKNTLYLQMNSLKTEDTAVYYC

SEQ ID NO: 133 - protein, 38 aa, Artificial Sequence
  Ab303 VH FR3 (IMGT)
YYGKSVKGRFTISRDNSKNTLYLQMNSLRAEDTAVYYC

SEQ ID NO: 134 - protein, 17 aa, Artificial Sequence
  Ab304/305 VH FR2 (IMGT)
MEWVRQAPGKGLEWVGE

SEQ ID NO: 135 - protein, 38 aa, Artificial Sequence
  Ab304 VH FR3 (IMGT)
YYGKSVKGRFTISRDESKNSLYLQMNSLKTEDTAVYYC

(19) United States
(12) Patent Application Publication
(10) Pub. No.: US 2022/0177593 A1
(43) Pub. Date: Jun. 9, 2022

(54) ANTI-AXL ANTIBODIES AND METHODS OF USE THEREOF

(71) Applicant: Celldex Therapeutics, Inc., Hampton, NJ (US)

(72) Inventors: Tibor Keler, Pipersville, PA (US); Diego Alvarado, Madison, CT (US); Richard W. Gedrich, Guilford, CT (US); Joel Goldstein, Hopewell, NJ (US); Laura Vitale, Doylestown, PA (US); Thomas O'Neill, Washington, NJ (US); Andrea Crocker, New Hope, PA (US); Jenifer Widger, Alpha, NJ (US); Michael B. Murphy, Wallingford, CT (US); Shannon Pankratz, South Windsor, CT (US)

(73) Assignee: Celldex Therapeutics, Inc., Hampton, NJ (US)

(21) Appl. No.: 17/598,005

(22) PCT Filed: Mar. 27, 2020
(86) PCT No.: PCT/US2020/025368

§ 371 Date: Sep. 24, 2021

Related U.S. Application Data
(60) Provisional application No. 62/826,909, filed on Mar. 29, 2019.

(51) Int. Cl. C07K 16/28

(52) U.S. Cl. CPC C07K 16/2863; C07K 2317/24; C07K 2317/92; C07K 2317/732; C07K 2317/76; C07K 2317/565

(57) ABSTRACT

Provided herein are compositions, methods and uses involving antibodies that specifically bind to Axl, a receptor tyrosine kinase. Also provided are uses and methods for managing, treating, or preventing disorders, such as cancer using the anti-Axl antibodies.

### CROSS-REFERENCE TO RELATED APPLICATIONS

This application claims the benefit of U.S. Provisional Application No. 62/826,909, filed Mar. 29, 2019, which is incorporated by reference herein in its entirety.

### REFERENCE TO SEQUENCE LISTING SUBMITTED ELECTRONICALLY

This application incorporates by reference a Sequence Listing submitted with this application as text file entitled 12638-156-228_ST25.txt created on Mar. 23, 2020 and having a size of 62,054 bytes.

### 1. FIELD

Provided herein are compositions, methods and uses involving antibodies that specifically bind to Axl, a receptor tyrosine kinase. Also provided are uses and methods for managing, treating, or preventing disorders, such as cancer using the anti-Axl antibodies.

### 2. BACKGROUND

Axl, a transmembrane receptor, belongs to the TAM (Tyro3, Axl and Mertk) family of receptor tyrosine kinases. The extracellular domain of Axl has two immunoglobulin (Ig) and two fibronectin (FN) type III motifs. Axl is a highly conserved gene across species but has two alternative variants due to splicing site of exon 10 within the trans-membrane domain.

Growth arrest specific gene 6 (Gas6) is a ligand for Axl. Binding of Gas6 to Axl involves initial formation of a high affinity 1:1 Gas6/Axl complex followed by dimerization of two 1:1 Gas6/Axl complexes. Gas6 binding to Axl results in autophosphorylation of tyrosine residues on the intracellular tyrosine kinase domain of Axl and formation of signaling complexes with phosphotyrosine-binding domains. Autophosphorylation sites include Y779, Y821, and Y866 on the intracellular domain of Axl. These residues are involved in binding Axl with subunits of phosphatidylinositol 3-kinase (PI3K), phospholipase C (PLC) and growth factor receptor-bound protein 2 (Grb2). Activation of PI3K and its downstream target, serine/threonine protein kinase Akt (Akt), is an important aspect of Axl-dependent signal transduction. In some cell types, Axl activates the ERK pathway. Protein S is closely related to Gas6 and also has been identified as a ligand of TAM receptors.

Axl heterodimerization with other TAM receptors, Tyro3 or Mertk, has been reported. This process seems to be ligand-independent, and can be the result of receptor overexpression.

Release of a soluble form of Axl (sAxl), an extracellular domain of Axl, represents another aspect of Axl signaling. Formation of the sAxl/Gas6 complexes limits ligand-dependent signaling. In addition, detection of Axl in the range between 100 and 140 kDa in molecular weight indicates that Axl is posttranslationally modified, for example, via glycosylation, phosphorylation and/or ubiquitination.

The Gas6/Axl pathway increases cell survival, promotes proliferation, aggregation and migration and is necessary for angiogenesis and immune cell activation in cancer.

Over-expression and activation of Axl protein has been implicated in oncogenesis, for example, tumor growth in mesothelioma and breast cancer. Also, Axl expression correlates with metastasis and poor prognosis in breast cancer. Axl-dependent signaling also is associated with progression of cardiovascular diseases and autoimmune disorders.

Axl signaling also plays a role in protecting innate immune cells (e.g., macrophages, dendritic and NK cells) from apoptosis and in phagocytosis of apoptotic bodies. For example, Axl signaling can negatively regulate pro-inflammatory signals, such as Twist1, SOCS-1 and SOCS-3, and phagocytosis in innate immune cells.

There is a need for therapies modulating Axl to manage, treat or prevent conditions involving Axl and/or abnormal Axl signaling or abnormal Axl expression.

### 3. SUMMARY

In one aspect, provided herein are antibodies, including antigen-binding fragments, which specifically bind to Axl, for example, the extracellular domain (ECD) of Axl. In one embodiment, antibodies and antigen binding fragments presented herein specifically bind the ECD of human Axl. Also provided herein are polynucleotides and vectors comprising sequences encoding such antibodies, cells comprising such polynucleotides and vectors, and compositions, reagents and kits comprising such antibodies. In another aspect, provided herein are methods for modulating Axl activity, e.g., inhibiting Axl activity, diagnostic methods and uses, and therapeutic methods and uses of such anti-Axl antibodies.

In a particular embodiment, provided herein is an isolated antibody, or an antigen-binding fragment thereof, which specifically binds to human Axl, comprising: (i) a light chain variable region (VL) comprising SEQ ID NO: 12; and/or (ii) a heavy chain variable region (VH) comprising SEQ ID NO: 11.

In another particular embodiment, provided here is an isolated antibody, or an antigen-binding fragment thereof, which specifically binds to human Axl, comprising: (i) a light chain variable region (VL) comprising SEQ ID NO: 12; and (ii) a heavy chain variable region (VH) comprising SEQ ID NO: 11.

In one embodiment, provided herein is antibody or an antigen binding fragment thereof that specifically binds to human Axl, comprising a VL that comprises SEQ ID NO: 12 or sequences having at least 80%, 85%, 90%, 95% or 98% amino acid sequence identity to SEQ ID NO: 12.

In a particular embodiment, provided herein is an isolated antibody, or an antigen-binding fragment thereof, which specifically binds to human Axl, comprising: (i) a light chain variable region (VL) comprising SEQ ID NO: 6; and/or (ii) a heavy chain variable region (VH) comprising SEQ ID NO: 7.

In another particular embodiment, provided here is an isolated antibody, or an antigen-binding fragment thereof, which specifically binds to human Axl, comprising: (i) a light chain variable region (VL) comprising SEQ ID NO: 6; and (ii) a heavy chain variable region (VH) comprising SEQ ID NO: 7.

In one embodiment, provided herein is antibody or an antigen binding fragment thereof that specifically binds to human Axl, comprising a VL that comprises SEQ ID NO: 6 or sequences having at least 80%, 85%, 90%, 95% or 98% amino acid sequence identity to SEQ ID NO: 6.

In a particular embodiment, provided herein is an isolated antibody, or an antigen-binding fragment thereof, which specifically binds to human Axl, comprising: (i) a light chain variable region (VL) comprising SEQ ID NO: 6; and/or (ii) a heavy chain variable region (VH) comprising SEQ ID NO: 8.

In another particular embodiment, provided here is an isolated antibody, or an antigen-binding fragment thereof, which specifically binds to human Axl, comprising: (i) a light chain variable region (VL) comprising SEQ ID NO: 6; and (ii) a heavy chain variable region (VH) comprising SEQ ID NO: 8.

In a particular embodiment, provided herein is an isolated antibody, or an antigen-binding fragment thereof, which specifically binds to human Axl, comprising: (i) a light chain variable region (VL) comprising SEQ ID NO: 6; and/or (ii) a heavy chain variable region (VH) comprising SEQ ID NO: 9.

In another particular embodiment, provided here is an isolated antibody, or an antigen-binding fragment thereof, which specifically binds to human Axl, comprising: (i) a light chain variable region (VL) comprising SEQ ID NO: 6; and (ii) a heavy chain variable region (VH) comprising SEQ ID NO: 9.

In a particular embodiment, provided herein is an isolated antibody, or an antigen-binding fragment thereof, which specifically binds to human Axl, comprising: (i) a light chain variable region (VL) comprising SEQ ID NO: 6; and/or (ii) a heavy chain variable region (VH) comprising SEQ ID NO: 10.

In another particular embodiment, provided here is an isolated antibody, or an antigen-binding fragment thereof, which specifically binds to human Axl, comprising: (i) a light chain variable region (VL) comprising SEQ ID NO: 6; and (ii) a heavy chain variable region (VH) comprising SEQ ID NO: 10.

In particular embodiments, provided herein is an isolated antibody, or an antigen-binding fragment thereof, which specifically binds to human Axl, comprising: (i) a light chain variable region (VL) comprising SEQ ID NO: 6; and/or (ii) a heavy chain variable region (VH) comprising SEQ ID NO: 5.

In another particular embodiment, provided here is an isolated antibody, or an antigen-binding fragment thereof, which specifically binds to human Axl, comprising: (i) a light chain variable region (VL) comprising SEQ ID NO: 6; and (ii) a heavy chain variable region (VH) comprising SEQ ID NO: 5.

In a certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 38, 31, and 32, respectively, or conservative sequence modifications thereof. In a specific embodiment, the VL that comprises VL CDR1, VL CDR2, and VL CDR3 (that comprise the amino acid sequences of SEQ ID NOS: 38, 31, and 32, respectively, or conservative sequence modifications thereof) comprises at least 80%, 85%, 90%. 95%, or 98% identity to SEQ ID NO: 12.

In another certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 38, 31, and 32, respectively. In a specific embodiment, the VL that comprises VL CDR1, VL CDR2, and VL CDR3 (that comprise the amino acid sequences of SEQ ID NOS: 38, 31, and 32, respectively) comprises at least 80%, 85%, 90%. 95%, or 98% identity to SEQ ID NO: 12.

In a certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 30, 31, and 32, respectively, or conservative sequence modifications thereof. In a specific embodiment, the VL that comprises VL CDR1, VL CDR2, and VL CDR3 (that comprise the amino acid sequences of SEQ ID NOS: 30, 31, and 32, respectively, or conservative sequence modifications thereof) comprises at least 80%, 85%, 90%. 95%, or 98% identity to SEQ ID NO: 6.

In another certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 30, 31, and 32, respectively. In a specific embodiment, the VL that comprises VL CDR1, VL CDR2, and VL CDR3 (that comprise the amino acid sequences of SEQ ID NOS: 30, 31, and 32, respectively) comprises at least 80%, 85%, 90%. 95%, or 98% identity to SEQ ID NO: 6.

In a certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 39, 34, and 35, respectively, or conservative sequence modifications thereof. In a specific embodiment, the VL that comprises VL CDR1, VL CDR2, and VL CDR3 (that comprise the amino acid sequences of SEQ ID NOS: 39, 34, and 35, respectively, or conservative sequence modifications thereof) comprises at least 80%, 85%, 90%. 95%, or 98% identity to SEQ ID NO: 12.

In another certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 39, 34, and 35, respectively. In a specific embodiment, the VL that comprises VL CDR1, VL CDR2, and VL CDR3 (that comprise the amino acid sequences of SEQ ID NOS: 39, 34, and 35, respectively) comprises at least 80%, 85%, 90%. 95%, or 98% identity to SEQ ID NO: 12.

In a certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 33, 34, and 35, respectively, or conservative sequence modifications thereof. In a specific embodiment, the VL that comprises VL CDR1, VL CDR2, and VL CDR3 (that comprise the amino acid sequences of SEQ ID NOS: 33, 34, and 35, respectively, or conservative sequence modifications thereof) comprises at least 80%, 85%, 90%. 95%, or 98% identity to SEQ ID NO: 6.

In another certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 33, 34, and 35, respectively. In a specific embodiment, the VL that comprises VL CDR1, VL CDR2, and VL CDR3 (that comprise the amino acid sequences of SEQ ID NOS: 33, 34, and 35, respectively) comprises at least 80%, 85%, 90%. 95%, or 98% identity to SEQ ID NO: 6.

In a certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 130, 124, and 32, respectively, or conservative sequence modifications thereof. In a specific embodiment, the VL that comprises VL CDR1, VL CDR2, and VL CDR3 (that comprise the amino acid sequences of SEQ ID NOS: 130, 124, and 32, respectively, or conservative sequence modifications thereof) comprises at least 80%, 85%, 90%. 95%, or 98% identity to SEQ ID NO: 12.

In another certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 130, 124, and 32, respectively. In a specific embodiment, the VL that comprises VL CDR1, VL CDR2, and VL CDR3 (that comprise the amino acid sequences of SEQ ID NOS: 130, 124, and 32, respectively) comprises at least 80%, 85%, 90%. 95%, or 98% identity to SEQ ID NO: 12.

In a certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 123, 124, and 32, respectively, or conservative sequence modifications thereof. In a specific embodiment, the VL that comprises VL CDR1, VL CDR2, and VL CDR3 (that comprise the amino acid sequences of SEQ ID NOS: 123, 124, and 32, respectively, or conservative sequence modifications thereof) comprises at least 80%, 85%, 90%. 95%, or 98% identity to SEQ ID NO: 6.

In another certain embodiment, provided herein is an antibody or an antigen binding fragment thereof that specifically binds to human Axl, comprising a VL that comprises a VL complementarity determining region 1 (CDR1), VL CDR2, and VL CDR3 that comprise the amino acid sequences of SEQ ID NOS: 123, 124, and 32, respectively. In a specific embodiment, the VL that comprises VL CDR1, VL CDR2, and VL CDR3 (that comprise the amino acid sequences of SEQ ID NOS: 123, 124, and 32, respectively) comprises at least 80%, 85%, 90%. 95%, or 98% identity to SEQ ID NO: 6.

In one embodiment with respect to an antibody described herein or an antigen binding fragment thereof that specifically binds to human Axl, comprising a VH that comprises SEQ ID NO: 11 or sequences having at least 80%, 85%, 90%, 95% or 98% amino acid sequence identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH that comprises VH CDR1 VH CDR2, and VH CDR3 (that comprise the amino acid sequences of SEQ ID NOS: 20, 21, and 22, respectively, or conservative sequence modifications thereof) comprises at least 80%, 85%, 90%. 95%, or 98% identity to SEQ ID NO: 11

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH comprises at least 80% amino acid sequence identity to SEQ ID NO: 11, the VH comprises at least 85% amino acid sequence identity to SEQ ID NO: 11; the VH comprises at least 90% amino acid sequence identity to SEQ ID NO: 11; the VH comprises at least 95% amino acid sequence identity to SEQ ID NO: 11; or at least 98% amino acid sequence identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH that comprises VH CDR1 VH CDR2, and VH CDR3 (that comprise the amino acid sequences of SEQ ID NOS: 23, 24, and 22, respectively, or conservative sequence modifications thereof) comprises at least 80%, 85%, 90%. 95%, or 98% identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH comprises at least 80% amino acid sequence identity to SEQ ID NO: 11, the VH comprises at least 85% amino acid sequence identity to SEQ ID NO: 11; the VH comprises at least 90% amino acid sequence identity to SEQ ID NO: 11; the VH comprises at least 95% amino acid sequence identity to SEQ ID NO: 11; or at least 98% amino acid sequence identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH that comprises VH CDR1 VH CDR2, and VH CDR3 (that comprise the amino acid sequences of SEQ ID NOS: 25, 26, and 22, respectively, or conservative sequence modifications thereof) comprises at least 80%, 85%, 90%. 95%, or 98% identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH comprises at least 80% amino acid sequence identity to SEQ ID NO: 11, the VH comprises at least 85% amino acid sequence identity to SEQ ID NO: 11; the VH comprises at least 90% amino acid sequence identity to SEQ ID NO: 11; the VH comprises at least 95% amino acid sequence identity to SEQ ID NO: 11; or at least 98% amino acid sequence identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 36, and 29, respectively, or conservative sequence modifications thereof. In a specific embodiment, the VH that comprises VH CDR1 VH CDR2, and VH CDR3 (that comprise the amino acid sequences of SEQ ID NOS: 27, 36, and 29, respectively, or conservative sequence modifications thereof) comprises at least 80%, 85%, 90%. 95%, or 98% identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 36, and 29, respectively, or conservative sequence modifications thereof. In a specific embodiment, the VH comprises at least 80% amino acid sequence identity to SEQ ID NO: 11, the VH comprises at least 85% amino acid sequence identity to SEQ ID NO: 11; the VH comprises at least 90% amino acid sequence identity to SEQ ID NO: 11; the VH comprises at least 95% amino acid sequence identity to SEQ ID NO: 11; or at least 98% amino acid sequence identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH that comprises VH CDR1 VH CDR2, and VH CDR3 (that comprise the amino acid sequences of SEQ ID NOS: 120, 121, and 122, respectively, or conservative sequence modifications thereof) comprises at least 80%, 85%, 90%. 95%, or 98% identity to SEQ ID NO: 11.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH comprises at least 80% amino acid sequence identity to SEQ ID NO: 11, the VH comprises at least 85% amino acid sequence identity to SEQ ID NO: 11; the VH comprises at least 90% amino acid sequence identity to SEQ ID NO: 11; the VH comprises at least 95% amino acid sequence identity to SEQ ID NO: 11; or at least 98% amino acid sequence identity to SEQ ID NO: 11.

In one embodiment with respect to an antibody described herein or an antigen binding fragment thereof that specifically binds to human Axl, comprising a VH that comprises SEQ ID NO: 7 or sequences having at least 80%, 85%, 90%, 95% or 98% amino acid sequence identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH that comprises VH CDR1 VH CDR2, and VH CDR3 (that comprise the amino acid sequences of SEQ ID NOS: 20, 21, and 22, respectively, or conservative sequence modifications thereof) comprises at least 80%, 85%, 90%. 95%, or 98% identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH comprises at least 80% amino acid sequence identity to SEQ ID NO: 7, the VH comprises at least 85% amino acid sequence identity to SEQ ID NO: 7; the VH comprises at least 90% amino acid sequence identity to SEQ ID NO: 7; the VH comprises at least 95% amino acid sequence identity to SEQ ID NO: 7; or at least 98% amino acid sequence identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH that comprises VH CDR1 VH CDR2, and VH CDR3 (that comprise the amino acid sequences of SEQ ID NOS: 23, 24, and 22, respectively, or conservative sequence modifications thereof) comprises at least 80%, 85%, 90%. 95%, or 98% identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH comprises at least 80% amino acid sequence identity to SEQ ID NO: 7, the VH comprises at least 85% amino acid sequence identity to SEQ ID NO: 7; the VH comprises at least 90% amino acid sequence identity to SEQ ID NO: 7; the VH comprises at least 95% amino acid sequence identity to SEQ ID NO: 7; or at least 98% amino acid sequence identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH that comprises VH CDR1 VH CDR2, and VH CDR3 (that comprise the amino acid sequences of SEQ ID NOS: 25, 26, and 22, respectively, or conservative sequence modifications thereof) comprises at least 80%, 85%, 90%. 95%, or 98% identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH comprises at least 80% amino acid sequence identity to SEQ ID NO: 7, the VH comprises at least 85% amino acid sequence identity to SEQ ID NO: 7; the VH comprises at least 90% amino acid sequence identity to SEQ ID NO: 7; the VH comprises at least 95% amino acid sequence identity to SEQ ID NO: 7; or at least 98% amino acid sequence identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 36, and 29, respectively, or conservative sequence modifications thereof. In a specific embodiment, the VH that comprises VH CDR1 VH CDR2, and VH CDR3 (that comprise the amino acid sequences of SEQ ID NOS: 27, 36, and 29, respectively, or conservative sequence modifications thereof) comprises at least 80%, 85%, 90%. 95%, or 98% identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 36, and 29, respectively, or conservative sequence modifications thereof. In a specific embodiment, the VH comprises at least 80% amino acid sequence identity to SEQ ID NO: 7, the VH comprises at least 85% amino acid sequence identity to SEQ ID NO: 7; the VH comprises at least 90% amino acid sequence identity to SEQ ID NO: 7; the VH comprises at least 95% amino acid sequence identity to SEQ ID NO: 7; or at least 98% amino acid sequence identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH that comprises VH CDR1 VH CDR2, and VH CDR3 (that comprise the amino acid sequences of SEQ ID NOS: 120, 121, and 122, respectively, or conservative sequence modifications thereof) comprises at least 80%, 85%, 90%. 95%, or 98% identity to SEQ ID NO: 7.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH comprises at least 80% amino acid sequence identity to SEQ ID NO: 7, the VH comprises at least 85% amino acid sequence identity to SEQ ID NO: 7; the VH comprises at least 90% amino acid sequence identity to SEQ ID NO: 7; the VH comprises at least 95% amino acid sequence identity to SEQ ID NO: 7; or at least 98% amino acid sequence identity to SEQ ID NO: 7.

In one embodiment with respect to an antibody described herein or an antigen binding fragment thereof that specifically binds to human Axl, comprising a VH that comprises SEQ ID NO: 8 or sequences having at least 80%, 85%, 90%, 95% or 98% amino acid sequence identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH that comprises VH CDR1 VH CDR2, and VH CDR3 (that comprise the amino acid sequences of SEQ ID NOS: 20, 21, and 22, respectively, or conservative sequence modifications thereof) comprises at least 80%, 85%, 90%. 95%, or 98% identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH comprises at least 80% amino acid sequence identity to SEQ ID NO: 8, the VH comprises at least 85% amino acid sequence identity to SEQ ID NO: 8; the VH comprises at least 90% amino acid sequence identity to SEQ ID NO: 11; the VH comprises at least 95% amino acid sequence identity to SEQ ID NO: 8; or at least 98% amino acid sequence identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH that comprises VH CDR1 VH CDR2, and VH CDR3 (that comprise the amino acid sequences of SEQ ID NOS: 23, 24, and 22, respectively, or conservative sequence modifications thereof) comprises at least 80%, 85%, 90%. 95%, or 98% identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH comprises at least 80% amino acid sequence identity to SEQ ID NO: 8, the VH comprises at least 85% amino acid sequence identity to SEQ ID NO: 8; the VH comprises at least 90% amino acid sequence identity to SEQ ID NO: 8; the VH comprises at least 95% amino acid sequence identity to SEQ ID NO: 8; or at least 98% amino acid sequence identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH that comprises VH CDR1 VH CDR2, and VH CDR3 (that comprise the amino acid sequences of SEQ ID NOS: 25, 26, and 22, respectively, or conservative sequence modifications thereof) comprises at least 80%, 85%, 90%. 95%, or 98% identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH comprises at least 80% amino acid sequence identity to SEQ ID NO: 8, the VH comprises at least 85% amino acid sequence identity to SEQ ID NO: 8; the VH comprises at least 90% amino acid sequence identity to SEQ ID NO: 8; the VH comprises at least 95% amino acid sequence identity to SEQ ID NO: 8; or at least 98% amino acid sequence identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 36, and 29, respectively, or conservative sequence modifications thereof. In a specific embodiment, the VH that comprises VH CDR1 VH CDR2, and VH CDR3 (that comprise the amino acid sequences of SEQ ID NOS: 27, 36, and 29, respectively, or conservative sequence modifications thereof) comprises at least 80%, 85%, 90%. 95%, or 98% identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 36, and 29, respectively, or conservative sequence modifications thereof. In a specific embodiment, the VH comprises at least 80% amino acid sequence identity to SEQ ID NO: 8, the VH comprises at least 85% amino acid sequence identity to SEQ ID NO: 8; the VH comprises at least 90% amino acid sequence identity to SEQ ID NO: 8; the VH comprises at least 95% amino acid sequence identity to SEQ ID NO: 8; or at least 98% amino acid sequence identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH that comprises VH CDR1 VH CDR2, and VH CDR3 (that comprise the amino acid sequences of SEQ ID NOS: 120, 121, and 122, respectively, or conservative sequence modifications thereof) comprises at least 80%, 85%, 90%. 95%, or 98% identity to SEQ ID NO: 8.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH comprises at least 80% amino acid sequence identity to SEQ ID NO: 8, the VH comprises at least 85% amino acid sequence identity to SEQ ID NO: 8; the VH comprises at least 90% amino acid sequence identity to SEQ ID NO: 8; the VH comprises at least 95% amino acid sequence identity to SEQ ID NO: 8; or at least 98% amino acid sequence identity to SEQ ID NO: 8.

In one embodiment with respect to an antibody described herein or an antigen binding fragment thereof that specifically binds to human Axl, comprising a VH that comprises SEQ ID NO: 9 or sequences having at least 80%, 85%, 90%, 95% or 98% amino acid sequence identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH that comprises VH CDR1 VH CDR2, and VH CDR3 (that comprise the amino acid sequences of SEQ ID NOS: 20, 21, and 22, respectively, or conservative sequence modifications thereof) comprises at least 80%, 85%, 90%. 95%, or 98% identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH comprises at least 80% amino acid sequence identity to SEQ ID NO: 9, the VH comprises at least 85% amino acid sequence identity to SEQ ID NO: 9; the VH comprises at least 90% amino acid sequence identity to SEQ ID NO: 9; the VH comprises at least 95% amino acid sequence identity to SEQ ID NO: 9; or at least 98% amino acid sequence identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH that comprises VH CDR1 VH CDR2, and VH CDR3 (that comprise the amino acid sequences of SEQ ID NOS: 23, 24, and 22, respectively, or conservative sequence modifications thereof) comprises at least 80%, 85%, 90%. 95%, or 98% identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH comprises at least 80% amino acid sequence identity to SEQ ID NO: 9, the VH comprises at least 85% amino acid sequence identity to SEQ ID NO: 9; the VH comprises at least 90% amino acid sequence identity to SEQ ID NO: 9; the VH comprises at least 95% amino acid sequence identity to SEQ ID NO: 9; or at least 98% amino acid sequence identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH that comprises VH CDR1 VH CDR2, and VH CDR3 (that comprise the amino acid sequences of SEQ ID NOS: 25, 26, and 22, respectively, or conservative sequence modifications thereof) comprises at least 80%, 85%, 90%. 95%, or 98% identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH comprises at least 80% amino acid sequence identity to SEQ ID NO: 9, the VH comprises at least 85% amino acid sequence identity to SEQ ID NO: 9; the VH comprises at least 90% amino acid sequence identity to SEQ ID NO: 9; the VH comprises at least 95% amino acid sequence identity to SEQ ID NO: 9; or at least 98% amino acid sequence identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 37, and 29, respectively, or conservative sequence modifications thereof. In a specific embodiment, the VH that comprises VH CDR1 VH CDR2, and VH CDR3 (that comprise the amino acid sequences of SEQ ID NOS: 27, 37, and 29, respectively, or conservative sequence modifications thereof) comprises at least 80%, 85%, 90%. 95%, or 98% identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 37, and 29, respectively, or conservative sequence modifications thereof. In a specific embodiment, the VH comprises at least 80% amino acid sequence identity to SEQ ID NO: 9, the VH comprises at least 85% amino acid sequence identity to SEQ ID NO: 9; the VH comprises at least 90% amino acid sequence identity to SEQ ID NO: 9; the VH comprises at least 95% amino acid sequence identity to SEQ ID NO: 9; or at least 98% amino acid sequence identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH that comprises VH CDR1 VH CDR2, and VH CDR3 (that comprise the amino acid sequences of SEQ ID NOS: 120, 121, and 122, respectively, or conservative sequence modifications thereof) comprises at least 80%, 85%, 90%. 95%, or 98% identity to SEQ ID NO: 9.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH comprises at least 80% amino acid sequence identity to SEQ ID NO: 9, the VH comprises at least 85% amino acid sequence identity to SEQ ID NO: 9; the VH comprises at least 90% amino acid sequence identity to SEQ ID NO: 9; the VH comprises at least 95% amino acid sequence identity to SEQ ID NO: 9; or at least 98% amino acid sequence identity to SEQ ID NO: 9.

In one embodiment with respect to an antibody described herein or an antigen binding fragment thereof that specifically binds to human Axl, comprising a VH that comprises SEQ ID NO: 10 or sequences having at least 80%, 85%, 90%, 95% or 98% amino acid sequence identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH that comprises VH CDR1 VH CDR2, and VH CDR3 (that comprise the amino acid sequences of SEQ ID NOS: 20, 21, and 22, respectively, or conservative sequence modifications thereof) comprises at least 80%, 85%, 90%. 95%, or 98% identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH comprises at least 80% amino acid sequence identity to SEQ ID NO: 10, the VH comprises at least 85% amino acid sequence identity to SEQ ID NO: 10; the VH comprises at least 90% amino acid sequence identity to SEQ ID NO: 11; the VH comprises at least 95% amino acid sequence identity to SEQ ID NO: 10; or at least 98% amino acid sequence identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH that comprises VH CDR1 VH CDR2, and VH CDR3 (that comprise the amino acid sequences of SEQ ID NOS: 23, 24, and 22, respectively, or conservative sequence modifications thereof) comprises at least 80%, 85%, 90%. 95%, or 98% identity to SEQ ID NO: 10 comprises.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH comprises at least 80% amino acid sequence identity to SEQ ID NO: 10, the VH comprises at least 85% amino acid sequence identity to SEQ ID NO: 10; the VH comprises at least 90% amino acid sequence identity to SEQ ID NO: 10; the VH comprises at least 95% amino acid sequence identity to SEQ ID NO: 10; or at least 98% amino acid sequence identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH that comprises VH CDR1 VH CDR2, and VH CDR3 (that comprise the amino acid sequences of SEQ ID NOS: 25, 26, and 22, respectively, or conservative sequence modifications thereof) comprises at least 80%, 85%, 90%. 95%, or 98% identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH comprises at least 80% amino acid sequence identity to SEQ ID NO: 10, the VH comprises at least 85% amino acid sequence identity to SEQ ID NO: 10; the VH comprises at least 90% amino acid sequence identity to SEQ ID NO: 10; the VH comprises at least 95% amino acid sequence identity to SEQ ID NO: 10; or at least 98% amino acid sequence identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 37, and 29, respectively, or conservative sequence modifications thereof. In a specific embodiment, the VH that comprises VH CDR1 VH CDR2, and VH CDR3 (that comprise the amino acid sequences of SEQ ID NOS: 27, 37, and 29, respectively, or conservative sequence modifications thereof) comprises at least 80%, 85%, 90%. 95%, or 98% identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 37, and 29, respectively, or conservative sequence modifications thereof. In a specific embodiment, the VH comprises at least 80% amino acid sequence identity to SEQ ID NO: 10, the VH comprises at least 85% amino acid sequence identity to SEQ ID NO: 10; the VH comprises at least 90% amino acid sequence identity to SEQ ID NO: 10; the VH comprises at least 95% amino acid sequence identity to SEQ ID NO: 10; or at least 98% amino acid sequence identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH that comprises VH CDR1 VH CDR2, and VH CDR3 (that comprise the amino acid sequences of SEQ ID NOS: 120, 121, and 122, respectively, or conservative sequence modifications thereof) comprises at least 80%, 85%, 90%. 95%, or 98% identity to SEQ ID NO: 10.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH comprises at least 80% amino acid sequence identity to SEQ ID NO: 10, the VH comprises at least 85% amino acid sequence identity to SEQ ID NO: 10; the VH comprises at least 90% amino acid sequence identity to SEQ ID NO: 10; the VH comprises at least 95% amino acid sequence identity to SEQ ID NO: 10; or at least 98% amino acid sequence identity to SEQ ID NO: 10.

In one embodiment with respect to an antibody described herein or an antigen binding fragment thereof that specifically binds to human Axl, comprising a VH that comprises SEQ ID NO: 5 or sequences having at least 80%, 85%, 90%, 95% or 98% amino acid sequence identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH that comprises VH CDR1 VH CDR2, and VH CDR3 (that comprise the amino acid sequences of SEQ ID NOS: 20, 21, and 22, respectively, or conservative sequence modifications thereof) comprises at least 80%, 85%, 90%. 95%, or 98% identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 20, 21, and 22, respectively, or conservative sequence modifications thereof. In a specific embodiment, the VH comprises at least 80% amino acid sequence identity to SEQ ID NO: 5 the VH comprises at least 85% amino acid sequence identity to SEQ ID NO: 5; the VH comprises at least 90% amino acid sequence identity to SEQ ID NO: 5; the VH comprises at least 95% amino acid sequence identity to SEQ ID NO: 5 or at least 98% amino acid sequence identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH that comprises VH CDR1 VH CDR2, and VH CDR3 (that comprise the amino acid sequences of SEQ ID NOS: 23, 24, and 22, respectively, or conservative sequence modifications thereof) comprises at least 80%, 85%, 90%. 95%, or 98% identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 23, 24, and 22, respectively, or conservative sequence modifications thereof. In a specific embodiment, the VH comprises at least 80% amino acid sequence identity to SEQ ID NO: 5, the VH comprises at least 85% amino acid sequence identity to SEQ ID NO: 5; the VH comprises at least 90% amino acid sequence identity to SEQ ID NO: 5; the VH comprises at least 95% amino acid sequence identity to SEQ ID NO: 5; or at least 98% amino acid sequence identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH that comprises VH CDR1 VH CDR2, and VH CDR3 (that comprise the amino acid sequences of SEQ ID NOS: 25, 26, and 22, respectively, or conservative sequence modifications thereof) comprises at least 80%, 85%, 90%. 95%, or 98% identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 25, 26, and 22, respectively, or conservative sequence modifications thereof. In a specific embodiment, the VH comprises at least 80% amino acid sequence identity to SEQ ID NO: 5, the VH comprises at least 85% amino acid sequence identity to SEQ ID NO: 5; the VH comprises at least 90% amino acid sequence identity to SEQ ID NO: 5; the VH comprises at least 95% amino acid sequence identity to SEQ ID NO: 5; or at least 98% amino acid sequence identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 28, and 29, respectively, or conservative sequence modifications thereof. In a specific embodiment, the VH that comprises VH CDR1 VH CDR2, and VH CDR3 (that comprise the amino acid sequences of SEQ ID NOS: 27, 28, and 29, respectively, or conservative sequence modifications thereof) comprises at least 80%, 85%, 90%. 95%, or 98% identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 27, 28, and 29, respectively, or conservative sequence modifications thereof. In a specific embodiment, the VH comprises at least 80% amino acid sequence identity to SEQ ID NO: 5, the VH comprises at least 85% amino acid sequence identity to SEQ ID NO: 5; the VH comprises at least 90% amino acid sequence identity to SEQ ID NO: 5; the VH comprises at least 95% amino acid sequence identity to SEQ ID NO: 5; or at least 98% amino acid sequence identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH that comprises VH CDR1 VH CDR2, and VH CDR3 (that comprise the amino acid sequences of SEQ ID NOS: 120, 121, and 122, respectively, or conservative sequence modifications thereof) comprises at least 80%, 85%, 90%. 95%, or 98% identity to SEQ ID NO: 5.

In a certain embodiment, provided herein is an antibody or an antigen binding fragment thereof that specifically binds to human Axl, comprising a VH that comprises a VH complementarity determining region 1 (CDR1), VH CDR2, and VH CDR3 that comprise the amino acid sequences of SEQ ID NOS: 120, 121, and 122, respectively, or conservative sequence modifications thereof. In a specific embodiment, the VH comprises at least 80% amino acid sequence identity to SEQ ID NO: 5, the VH comprises at least 85% amino acid sequence identity to SEQ ID NO: 5; the VH comprises at least 90% amino acid sequence identity to SEQ ID NO: 5; the VH comprises at least 95% amino acid sequence identity to SEQ ID NO: 5; or at least 98% amino acid sequence identity to SEQ ID NO: 5.

In another embodiment, provide herein is an antibody or an antigen binding fragment thereof that specifically binds to human Axl, comprising a VL that comprises SEQ ID NO: 12 or sequences having at least 80%, 85%, 90%, 95% or 98% amino acid sequence identity to SEQ ID NO: 12, and a VH that comprises SEQ ID NO: 11 or sequences having at least 80%, 85%, 90%, 95% or 98% amino acid sequence identity to SEQ ID NO: 11.

In another embodiment, provide herein is an antibody or an antigen binding fragment thereof that specifically binds to human Axl, comprising a VL that comprises SEQ ID NO: 6 or sequences having at least 80%, 85%, 90%, 95% or 98% amino acid sequence identity to SEQ ID NO: 6, and a VH that comprises SEQ ID NO: 7 or sequences having at least 80%, 85%, 90%, 95% or 98% amino acid sequence identity to SEQ ID NO: 7.

In another embodiment, provide herein is an antibody or an antigen binding fragment thereof that specifically binds to human Axl, comprising a VL that comprises SEQ ID NO: 6 or sequences having at least 80%, 85%, 90%, 95% or 98% amino acid sequence identity to SEQ ID NO: 6, and a VH that comprises SEQ ID NO: 8 or sequences having at least 80%, 85%, 90%, 95% or 98% amino acid sequence identity to SEQ ID NO: 8.

In another embodiment, provide herein is an antibody or an antigen binding fragment thereof that specifically binds to human Axl, comprising a VL that comprises SEQ ID NO: 6 or sequences having at least 80%, 85%, 90%, 95% or 98% amino acid sequence identity to SEQ ID NO: 6, and a VH that comprises SEQ ID NO: 9 or sequences having at least 80%, 85%, 90%, 95% or 98% amino acid sequence identity to SEQ ID NO: 9.

In another embodiment, provide herein is an antibody or an antigen binding fragment thereof that specifically binds to human Axl, comprising a VL that comprises SEQ ID NO: 6 or sequences having at least 80%, 85%, 90%, 95% or 98% amino acid sequence identity to SEQ ID NO: 6, and a VH that comprises SEQ ID NO: 10 or sequences having at least 80%, 85%, 90%, 95% or 98% amino acid sequence identity to SEQ ID NO: 10.

In another embodiment, provide herein is an antibody or an antigen binding fragment thereof that specifically binds to human Axl, comprising a VL that comprises SEQ ID NO: 6 or sequences having at least 80%, 85%, 90%, 95% or 98% amino acid sequence identity to SEQ ID NO: 6, and a VH that comprises SEQ ID NO: 5 or sequences having at least 80%, 85%, 90%, 95% or 98% amino acid sequence identity to SEQ ID NO: 5.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22 respectively or conservative sequence modifications thereof.

In one embodiment, presented herein is an antibody or an antigen binding fragment thereof that specifically binds to human Axl, the VL CDR1, VL CDR2, and VL CDR3 comprise the amino acid sequences of SEQ ID NOS: 38, 31, and 32, respectively or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 20, 21, and 22, respectively, or sequences having at least 80% amino acid sequence identity thereto.

In a particular embodiment, provided herein is an antibody or an antigen binding fragment thereof that specifically binds to human Axl, the VL CDR1, VL CDR2, and VL CDR3 comprise the amino acid sequences of SEQ ID NOS: 38, 31, and 32, respectively, or sequences having at least 80% amino acid sequence identity thereto, and the VH CDR1, VH CDR2, and VH CDR3 comprise the amino acid sequences of SEQ ID NOS: 20, 21, and 22, respectively, or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 30, 31 and 32, respectively, or sequences having at least 80% amino acid sequence identity thereto.

In a certain embodiment provided herein is an antibody or an antigen binding fragment thereof that specifically binds to human Axl, the VH CDR1, VH CDR2, and VH CDR3 comprise the amino acid sequences of SEQ ID NOS: 20, 21 and 22, respectively or sequences having at least 80% amino acid sequence identity thereto.

In a particular embodiment presented herein is an antibody or an antigen binding fragment thereof that specifically binds to human Axl, comprising a VL that comprises a VL CDR1, VL CDR2, and VL CDR3 comprising the amino acid sequences of SEQ ID NOS: 30, 31 and 32, respectively, or sequences having at least 80% amino acid sequence identity thereto, and comprises a VH that comprises VH CDR1, VH CDR2, and VH CDR3 comprising the amino acid sequences of SEQ ID NOS: 20, 21 and 22, respectively or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 38, 31 and 32, respectively, or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 23, 24 and 22, respectively, or sequences having at least 80% amino acid sequence identity thereto.

In a particular embodiment, provided herein is an antibody or an antigen binding fragment thereof that specifically binds to human Axl, the VL CDR1, VL CDR2, and VL CDR3 comprise the amino acid sequences of SEQ ID NOS: 38, 31, and 32, respectively, or sequences having at least 80% amino acid sequence identity thereto, and the VH CDR1, VH CDR2, and VH CDR3 comprise the amino acid sequences of SEQ ID NOS: 23, 24 and 22, respectively, or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 30, 31 and 32, respectively, or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 23, 24 and 22, respectively, or sequences having at least 80% amino acid sequence identity thereto.

In a particular embodiment presented herein is an antibody or an antigen binding fragment thereof that specifically binds to human Axl, comprising a VL that comprises a VL CDR1, VL CDR2, and VL CDR3 comprising the amino acid sequences of SEQ ID NOS: 30, 31 and 32, respectively, or sequences having at least 80% amino acid sequence identity thereto, and comprises a VH that comprises VH CDR1, VH CDR2, and VH CDR3 comprising the amino acid sequences of SEQ ID NOS: 23, 24 and 22, respectively or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 38, 31 and 32, respectively, or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 23, 24 and 22, respectively, or sequences having at least 80% amino acid sequence identity thereto.

In a particular embodiment, provided herein is an antibody or an antigen binding fragment thereof that specifically binds to human Axl, the VL CDR1, VL CDR2, and VL CDR3 comprise the amino acid sequences of SEQ ID NOS: 38, 31, and 32, respectively, or sequences having at least 80% amino acid sequence identity thereto, and the VH CDR1, VH CDR2, and VH CDR3 comprise the amino acid sequences of SEQ ID NOS: 23, 24 and 22, respectively, or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 30, 31 and 32, respectively, or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 25, 26 and 22, respectively, or sequences having at least 80% amino acid sequence identity thereto.

In a particular embodiment presented herein is an antibody or an antigen binding fragment thereof that specifically binds to human Axl, comprising a VL that comprises a VL CDR1, VL CDR2, and VL CDR3 comprising the amino acid sequences of SEQ ID NOS: 30, 31 and 32, respectively, or sequences having at least 80% amino acid sequence identity thereto, and comprises a VH that comprises VH CDR1, VH CDR2, and VH CDR3 comprising the amino acid sequences of SEQ ID NOS: 25, 26 and 22, respectively or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29 respectively or conservative sequence modifications thereof.

In one embodiment, presented herein is an antibody or an antigen binding fragment thereof that specifically binds to human Axl, the VL CDR1, VL CDR2, and VL CDR3 comprise the amino acid sequences of SEQ ID NOS: 39, 34 and 35, respectively or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 27, 36 and 29, respectively, or sequences having at least 80% amino acid sequence identity thereto.

In a particular embodiment, provided herein is an antibody or an antigen binding fragment thereof that specifically binds to human Axl, the VL CDR1, VL CDR2, and VL CDR3 comprise the amino acid sequences of SEQ ID NOS: 39, 34 and 35, respectively, or sequences having at least 80% amino acid sequence identity thereto, and the VH CDR1, VH CDR2, and VH CDR3 comprise the amino acid sequences of SEQ ID NOS: 27, 36 and 29, respectively, or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 33, 34 and 35, respectively, or sequences having at least 80% amino acid sequence identity thereto.

In a certain embodiment provided herein is an antibody or an antigen binding fragment thereof that specifically binds to human Axl, the VH CDR1, VH CDR2, and VH CDR3 comprise the amino acid sequences of SEQ ID NOS: 27, 36 and 29, respectively or sequences having at least 80% amino acid sequence identity thereto.

In a particular embodiment presented herein is an antibody or an antigen binding fragment thereof that specifically binds to human Axl, comprising a VL that comprises a VL CDR1, VL CDR2, and VL CDR3 comprising the amino acid sequences of SEQ ID NOS: 33, 34 and 35, respectively, or sequences having at least 80% amino acid sequence identity thereto, and comprises a VH that comprises VH CDR1, VH CDR2, and VH CDR3 comprising the amino acid sequences of SEQ ID NOS: 27, 36 and 29, respectively or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 33, 34 and 35, respectively, or sequences having at least 80% amino acid sequence identity thereto.

In a certain embodiment provided herein is an antibody or an antigen binding fragment thereof that specifically binds to human Axl, the VH CDR1, VH CDR2, and VH CDR3 comprise the amino acid sequences of SEQ ID NOS: 27, 37 and 29, respectively or sequences having at least 80% amino acid sequence identity thereto.

In a particular embodiment presented herein is an antibody or an antigen binding fragment thereof that specifically binds to human Axl, comprising a VL that comprises a VL CDR1, VL CDR2, and VL CDR3 comprising the amino acid sequences of SEQ ID NOS: 33, 34 and 35, respectively, or sequences having at least 80% amino acid sequence identity thereto, and comprises a VH that comprises VH CDR1, VH CDR2, and VH CDR3 comprising the amino acid sequences of SEQ ID NOS: 27, 37 and 29, respectively or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 33, 34 and 35, respectively, or sequences having at least 80% amino acid sequence identity thereto.

In a certain embodiment provided herein is an antibody or an antigen binding fragment thereof that specifically binds to human Axl, the VH CDR1, VH CDR2, and VH CDR3 comprise the amino acid sequences of SEQ ID NOS: 27, 28 and 29, respectively or sequences having at least 80% amino acid sequence identity thereto.

In a particular embodiment presented herein is an antibody or an antigen binding fragment thereof that specifically binds to human Axl, comprising a VL that comprises a VL CDR1, VL CDR2, and VL CDR3 comprising the amino acid sequences of SEQ ID NOS: 33, 34 and 35, respectively, or sequences having at least 80% amino acid sequence identity thereto, and comprises a VH that comprises VH CDR1, VH CDR2, and VH CDR3 comprising the amino acid sequences of SEQ ID NOS: 27, 28 and 29, respectively or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 130, 124 and 32, respectively, or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 120, 121 and 122, respectively, or sequences having at least 80% amino acid sequence identity thereto.

In a particular embodiment, provided herein is an antibody or an antigen binding fragment thereof that specifically binds to human Axl, the VL CDR1, VL CDR2, and VL CDR3 comprise the amino acid sequences of SEQ ID NOS: 130, 124, and 32, respectively, or sequences having at least 80% amino acid sequence identity thereto, and the VH CDR1, VH CDR2, and VH CDR3 comprise the amino acid sequences of SEQ ID NOS: 120, 121 and 122, respectively, or sequences having at least 80% amino acid sequence identity thereto.

In a particular embodiment, provided herein is an isolated antibody (e.g., monoclonal antibody), or an antigen-binding fragment thereof, which specifically binds to human Axl, comprising:
(i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122 respectively or conservative sequence modifications thereof.

In one embodiment provided herein is an antibody or an antigen binding fragment thereof that specifically binds to human Axl, comprising a VL that comprises VL CDR1, VL CDR2, and VL CDR3 that comprise the amino acid sequences of SEQ ID NOS: 123, 124 and 32, respectively, or sequences having at least 80% amino acid sequence identity thereto.

In a certain embodiment, provided herein is an antibody or an antigen binding fragment thereof that specifically binds to human Axl, comprising a VH CDR1, VH CDR2, and VH CDR3 that comprises the amino acid sequences of SEQ ID NOS: 120, 121 and 122, respectively, or sequences having at least 80% amino acid sequence identity thereto.

In a particular embodiment presented herein is an antibody or an antigen binding fragment thereof that specifically binds to human Axl, comprising a VL that comprises a VL CDR1, VL CDR2, and VL CDR3 comprising the amino acid sequences of SEQ ID NOS: 123, 124 and 32, respectively, or sequences having at least 80% amino acid sequence identity thereto, and comprises a VH that comprises VH CDR1, VH CDR2, and VH CDR3 comprising the amino acid sequences of SEQ ID NOS: 120, 121 and 122, respectively or sequences having at least 80% amino acid sequence identity thereto.

In one embodiment provided herein is an antibody or an antigen binding fragment thereof that specifically binds to human Axl, comprising a VL that comprises a VL CDR1, VL CDR2, and VL CDR3 comprising the amino acid sequences of SEQ ID NOS: 38, 31, and 32, respectively, or sequences having at least 80% amino acid sequence identity. In another embodiment provided herein is an antibody or an antigen binding fragment thereof that specifically binds to human Axl, comprising a VL that comprises a VL CDR1, VL CDR2, and VL CDR3 comprising the amino acid sequences of SEQ ID NOS: 39, 34, and 35, respectively, or sequences having at least 80% amino acid sequence identity thereto. In another embodiment provided herein is an antibody or an antigen binding fragment thereof that specifically binds to human Axl, comprising a VL that comprises a VL CDR1, VL CDR2, and VL CDR3 comprising the amino acid sequences of SEQ ID NOS: 130, 124, and 32, respectively, or sequences having at least 80% amino acid sequence identity thereto. In another embodiment provided herein is an antibody or an antigen binding fragment thereof that specifically binds to human Axl, comprising a VL that comprises a VL CDR1, VL CDR2, and VL CDR3 comprising the amino acid sequences of SEQ ID NOS: 30, 31, and 32, respectively, or sequences having at least 80% amino acid sequence identity thereto. In another embodiment provided herein is an antibody or an antigen binding fragment thereof that specifically binds to human Axl, comprising a VL that comprises a VL CDR1, VL CDR2, and VL CDR3 comprising the amino acid sequences of SEQ ID NOS: 33, 34, and 35, respectively, or sequences having at least 80% amino acid sequence identity thereto. In another embodiment provided herein is an antibody or an antigen binding fragment thereof that specifically binds to human Axl, comprising a VL that comprises a VL CDR1, VL CDR2, and VL CDR3 comprising the amino acid sequences of SEQ ID NOS: 123, 124, and 32, respectively, or sequences having at least 80% amino acid sequence identity thereto.

In a certain embodiment provided herein is an antibody or an antigen binding fragment thereof that specifically binds to human Axl, comprising a VH that comprises a VH CDR1, VH CDR2, and VH CDR3 comprising the amino acid sequences of SEQ ID NOS: 20, 21, and 22, respectively, or sequences having at least 80% amino acid sequence identity thereto. In another certain embodiment provided herein is an antibody or an antigen binding fragment thereof that specifically binds to human Axl, comprising a VH that comprises a VH CDR1, VH CDR2, and VH CDR3 comprising the amino acid sequences of SEQ ID NOS: 23, 24, and 22, respectively, or sequences having at least 80% amino acid sequence identity thereto. In another certain embodiment provided herein is an antibody or an antigen binding fragment thereof that specifically binds to human Axl, comprising a VH that comprises a VH CDR1, VH CDR2, and VH CDR3 comprising the amino acid sequences of SEQ ID NOS: 25, 26, and 22, respectively, or sequences having at least 80% amino acid sequence identity thereto. In another certain embodiment provided herein is an antibody or an antigen binding fragment thereof that specifically binds to human Axl, comprising a VH that comprises a VH CDR1, VH CDR2, and VH CDR3 comprising the amino acid sequences of SEQ ID NOS: 27, 36, and 29, respectively, or sequences having at least 80% amino acid sequence identity thereto. In another certain embodiment provided herein is an antibody or an antigen binding fragment thereof that specifically binds to human Axl, comprising a VH that comprises a VH CDR1, VH CDR2, and VH CDR3 comprising the amino acid sequences of SEQ ID NOS: 27, 37, and 29, respectively, or sequences having at least 80% amino acid sequence identity thereto. In another certain embodiment provided herein is an antibody or an antigen binding fragment thereof that specifically binds to human Axl, comprising a VH that comprises a VH CDR1, VH CDR2, and VH CDR3 comprising the amino acid sequences of SEQ ID NOS: 27, 28, and 29, respectively, or sequences having at least 80% amino acid sequence identity thereto. In another certain embodiment provided herein is an antibody or an antigen binding fragment thereof that specifically binds to human Axl, comprising a VH that comprises a VH CDR1, VH CDR2, and VH CDR3 comprising the amino acid sequences of SEQ ID NOS: 120, 121, and 122, respectively, or sequences having at least 80% amino acid sequence identity thereto.

In a particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 38, 31, and 32, respectively, or sequences having at least 80% amino acid sequence identity thereto, and comprise a VH that comprises a VH CDR1, VH CDR2, and VH CDR3 comprising the amino acid sequences of SEQ ID NOS: 20, 21, and 22, respectively, or sequences having at least 80% amino acid sequence identity thereto. In another particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 30, 31, and 32, respectively, or sequences having at least 80% amino acid sequence identity thereto, and comprise a VH that comprises a VH CDR1, VH CDR2, and VH CDR3 comprising the amino acid sequences of SEQ ID NOS: 20, 21, and 22, respectively, or sequences having at least 80% amino acid sequence identity thereto.

In a particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 38, 31, and 32, respectively, or sequences having at least 80% amino acid sequence identity thereto, and comprise a VH that comprises a VH CDR1, VH CDR2, and VH CDR3 comprising the amino acid sequences of SEQ ID NOS: 23, 24, and 22, respectively, or sequences having at least 80% amino acid sequence identity thereto. In another particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 30, 31, and 32, respectively, or sequences having at least 80% amino acid sequence identity thereto, and comprise a VH that comprises a VH CDR1, VH CDR2, and VH CDR3 comprising the amino acid sequences of SEQ ID NOS: 23, 24, and 22, respectively, or sequences having at least 80% amino acid sequence identity thereto.

In a particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 38, 31, and 32, respectively, or sequences having at least 80% amino acid sequence identity thereto, and comprise a VH that comprises a VH CDR1, VH CDR2, and VH CDR3 comprising the amino acid sequences of SEQ ID NOS: 25, 26, and 22, respectively, or sequences having at least 80% amino acid sequence identity thereto. In another particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 30, 31, and 32, respectively, or sequences having at least 80% amino acid sequence identity thereto, and comprise a VH that comprises a VH CDR1, VH CDR2, and VH CDR3 comprising the amino acid sequences of SEQ ID NOS: 25, 26, and 22, respectively, or sequences having at least 80% amino acid sequence identity thereto.

In a particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 39, 34, and 35, respectively, or sequences having at least 80% amino acid sequence identity thereto, and comprise a VH that comprises a VH CDR1, VH CDR2, and VH CDR3 comprising the amino acid sequences of SEQ ID NOS: 27, 36, and 29, respectively, or sequences having at least 80% amino acid sequence identity thereto. In another particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 33, 34, and 35, respectively, or sequences having at least 80% amino acid sequence identity thereto, and comprise a VH that comprises a VH CDR1, VH CDR2, and VH CDR3 comprising the amino acid sequences of SEQ ID NOS: 27, 36, and 29, respectively, or sequences having at least 80% amino acid sequence identity thereto. In another particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 33, 34, and 35, respectively, or sequences having at least 80% amino acid sequence identity thereto, and comprise a VH that comprises a VH CDR1, VH CDR2, and VH CDR3 comprising the amino acid sequences of SEQ ID NOS: 27, 37, and 29, respectively, or sequences having at least 80% amino acid sequence identity thereto. In another particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 33, 34, and 35, respectively, or sequences having at least 80% amino acid sequence identity thereto, and comprise a VH that comprises a VH CDR1, VH CDR2, and VH CDR3 comprising the amino acid sequences of SEQ ID NOS: 27, 28, and 29, respectively, or sequences having at least 80% amino acid sequence identity thereto.

In a particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 130, 124, and 32, respectively, or sequences having at least 80% amino acid sequence identity thereto, and comprise a VH that comprises a VH CDR1, VH CDR2, and VH CDR3 comprising the amino acid sequences of SEQ ID NOS: 120, 121, and 122, respectively, or sequences having at least 80% amino acid sequence identity thereto. In another particular embodiment provided herein is an antibody or an antigen binding fragment thereof that specifically binds to human Axl, comprising a VL that comprise a VL CDR1, VL CDR2, and VL CDR3 comprising the amino acid sequences of SEQ ID NOS: 123, 124, and 32, respectively, or sequences having at least 80% amino acid sequence identity thereto, and comprise a VH that comprises a VH CDR1, VH CDR2, and VH CDR3 comprising the amino acid sequences of SEQ ID NOS: 120, 121, and 122, respectively, or sequences having at least 80% amino acid sequence identity thereto. 1001661 In one embodiment, provided herein is an antibody or an antigen binding fragment thereof that specifically binds to human Axl, that comprises a VL CDR1, VL CDR2, and VL CDR3 present in one polypeptide, and a comprises a VH CDR1, VH CDR2, and VH CDR3 on a second polypeptide. In a specific embodiment, an antibody or antigen binding fragment described herein which specifically binds to human Axl comprises a VL and a VH, wherein the VL and VH are present in the same polypeptide.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively. In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively, or conservative sequence modifications thereof.

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In one aspect, provided herein is an isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

In one aspect, provided herein is an isolated antibody, or an antigen-binding fragment thereof, which specifically binds to human Axl, comprising:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively, or conservative sequence modifications thereof.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively.

In a specific embodiment of the antibody or antigen-binding fragment described herein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively.

As a person of ordinary skill in the art would understand, the CDRs and FRs of an antibody or antigen-binding fragment thereof are configured in the following order: FR1-CDR1-FR2-CDR2-FR3-CDR3-FR4.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 5.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 5.

In a specific embodiment of the antibody or antigen-binding fragment described herein: (i) the light chain variable region (VL) comprises an amino acid sequence of SEQ ID NO: 6; and/or (ii) the heavy chain variable region (VH) comprises an amino acid sequence of SEQ ID NO: 5.

In a specific embodiment of the antibody or antigen-binding fragment described herein: (i) the light chain variable region (VL) comprises an amino acid sequence of SEQ ID NO: 6; and (ii) the heavy chain variable region (VH) comprises an amino acid sequence of SEQ ID NO: 5.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 7.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 7.

In a specific embodiment of the antibody or antigen-binding fragment described herein: (i) the light chain variable region (VL) comprises an amino acid sequence of SEQ ID NO: 6; and/or (ii) the heavy chain variable region (VH) comprises an amino acid sequence of SEQ ID NO: 7.

In a specific embodiment of the antibody or antigen-binding fragment described herein: (i) the light chain variable region (VL) comprises an amino acid sequence of SEQ ID NO: 6; and (ii) the heavy chain variable region (VH) comprises an amino acid sequence of SEQ ID NO: 7.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 8.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 8.

In a specific embodiment of the antibody or antigen-binding fragment described herein: (i) the light chain variable region (VL) comprises an amino acid sequence of SEQ ID NO: 6; and/or (ii) the heavy chain variable region (VH) comprises an amino acid sequence of SEQ ID NO: 8.

In a specific embodiment of the antibody or antigen-binding fragment described herein: (i) the light chain variable region (VL) comprises an amino acid sequence of SEQ ID NO: 6; and (ii) the heavy chain variable region (VH) comprises an amino acid sequence of SEQ ID NO: 8.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 9.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 9.

In a specific embodiment of the antibody or antigen-binding fragment described herein: (i) the light chain variable region (VL) comprises an amino acid sequence of SEQ ID NO: 6; and/or (ii) the heavy chain variable region (VH) comprises an amino acid sequence of SEQ ID NO: 9.

In a specific embodiment of the antibody or antigen-binding fragment described herein: (i) the light chain variable region (VL) comprises an amino acid sequence of SEQ ID NO: 6; and (ii) the heavy chain variable region (VH) comprises an amino acid sequence of SEQ ID NO: 9.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 10.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 10.

In a specific embodiment of the antibody or antigen-binding fragment described herein: (i) the light chain variable region (VL) comprises an amino acid sequence of SEQ ID NO: 6; and/or (ii) the heavy chain variable region (VH) comprises an amino acid sequence of SEQ ID NO: 10.

In a specific embodiment of the antibody or antigen-binding fragment described herein: (i) the light chain variable region (VL) comprises an amino acid sequence of SEQ ID NO: 6; and (ii) the heavy chain variable region (VH) comprises an amino acid sequence of SEQ ID NO: 10.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 12; and/or (ii) the heavy chain variable region (VH) comprises an amino acid sequence having at least 80%, at least 85%, at least 90%, at least 95%, at least 98%, or 100% sequence identity to SEQ ID NO: 11.

In a specific embodiment of the antibody or antigen-binding fragment described herein: (i) the light chain variable region (VL) comprises an amino acid sequence having at least 80%, at least 85%, at least 90%, at least 95%, at least 98%, or 100% sequence identity to SEQ ID NO: 12; and (ii) the heavy chain variable region (VH) comprises an amino acid sequence having at least 80%, at least 85%, at least 90%, at least 95%, at least 98%, or 100% sequence identity to SEQ ID NO: 11.

In a specific embodiment of the antibody or antigen-binding fragment described herein: (i) the light chain variable region (VL) comprises an amino acid sequence of SEQ ID NO: 6; and/or (ii) the heavy chain variable region (VH) comprises an amino acid sequence of SEQ ID NO: 12.

In a specific embodiment of the antibody or antigen-binding fragment described herein: (i) the light chain variable region (VL) comprises an amino acid sequence of SEQ ID NO: 6; and (ii) the heavy chain variable region (VH) comprises an amino acid sequence of SEQ ID NO: 11.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 5.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 5.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and/or (ii) the heavy chain variable region (VH) comprises an amino acid sequence of SEQ ID NO: 5.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and (ii) the heavy chain variable region (VH) comprises an amino acid sequence of SEQ ID NO: 5.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 7.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 7.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and/or (ii) the heavy chain variable region (VH) comprises an amino acid sequence of SEQ ID NO: 7.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and (ii) the heavy chain variable region (VH) comprises an amino acid sequence of SEQ ID NO: 7.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 8.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 8.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and/or (ii) the heavy chain variable region (VH) comprises an amino acid sequence of SEQ ID NO: 8.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and (ii) the heavy chain variable region (VH) comprises an amino acid sequence of SEQ ID NO: 8.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 9.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 9.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and/or (ii) the heavy chain variable region (VH) comprises an amino acid sequence of SEQ ID NO: 9.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and (ii) the heavy chain variable region (VH) comprises an amino acid sequence of SEQ ID NO: 9.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and/or (ii) the heavy chain variable region (VH) comprises an amino acid sequence having at least 80%, at least 85%, at least 90%, at least 95%, at least 98%, or 100% sequence identity to SEQ ID NO: 10.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 6; and (ii) the heavy chain variable region (VH) comprises an amino acid sequence having at least 80%, at least 85%, at least 90%, at least 95%, at least 98%, or 100% sequence identity to SEQ ID NO: 10.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and/or (ii) the heavy chain variable region (VH) comprises an amino acid sequence of SEQ ID NO: 10.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and (ii) the heavy chain variable region (VH) comprises an amino acid sequence of SEQ ID NO: 10.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 12; and/or (ii) the heavy chain variable region (VH) comprises an amino acid sequence having at least 80%, at least 85%, at least 90%, at least 95%, at least 98%, or 100% sequence identity to SEQ ID NO: 11.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence having at least 80%, at least 85%, at least 90%, at least 95%, at least 98%, or 100% sequence identity to SEQ ID NO: 12; and (ii) the heavy chain variable region (VH) comprises an amino acid sequence having at least 80%, at least 85%, at least 90%, at least 95%, at least 98%, or 100% sequence identity to SEQ ID NO: 11.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and/or (ii) the heavy chain variable region (VH) comprises an amino acid sequence of SEQ ID NO: 12.

In one aspect, provided herein is an isolated antibody, or an antigen-binding fragment thereof, which specifically binds to human Axl, comprising a light chain variable region (VL) and a heavy chain variable region (VH), wherein: (i) the light chain variable region (VL) comprises an amino acid sequence of SEQ ID NO: 6; and (ii) the heavy chain variable region (VH) comprises an amino acid sequence of SEQ ID NO: 11.

In one embodiment, the antibody or antigen-binding fragment described herein specifically binds to Ig-like domain 1 in the extracellular domain of human Axl.

In one embodiment, the antibody or antigen-binding fragment described herein does not specifically bind to the extracellular domain of MerTK.

In another embodiment, provided herein is an isolated antibody, or an antigen-binding fragment thereof, which specifically binds to Ig-like domain 1 in the extracellular domain of human Axl. In a certain embodiment, provided herein is an isolated antibody, or an antigen-binding fragment thereof, which binds to the same epitope of human Axl as an antibody described herein or antigen-binding fragment thereof. In another embodiment, provided herein is an isolated antibody, or an antigen-binding fragment thereof, which competes for binding to human Axl with an antibody described herein or antigen-binding fragment thereof. In another embodiment, provided herein is an isolated antibody, or an antigen-binding fragment thereof, which binds to Domain Ig-like domain 1 (D1) of the extracellular domain of human Axl and inhibits binding of Gas6 to Axl and/or, inhibits Axl phosphorylation.

In one embodiment, an antibody or antigen binding fragment presented herein exhibits an Axl receptor antagonist activity.

In one embodiment, an antibody or antigen binding fragment described herein, which specifically binds to human Axl, comprises a heavy chain constant region (for example, a human heavy chain constant region). In a specific embodiment, an antibody or antigen-binding fragment described herein, which specifically binds to human Axl, comprises a human heavy chain comprising a VH and a human heavy chain constant region.

In another embodiment, an antibody or antigen binding fragment described herein, which specifically binds to human Axl, comprises a human light chain constant region. In a specific embodiment, an antibody or antigen-binding fragment described herein, which specifically binds to human Axl, comprises a human light chain comprising a VL and a human light chain constant region.

In yet another embodiment, an antibody or antigen binding fragment described herein which specifically binds to human Axl comprises a human heavy chain constant region and a human light chain constant region. In a specific embodiment, such an antibody or antigen-binding fragment comprises a human heavy chain comprising a VH and a human heavy chain constant region and a human light chain comprising a VL and a human light chain constant region.

In a certain embodiment, an antibody or antigen binding fragment described herein, which specifically binds to human Axl, is an IgG antibody or antigen-binding fragment thereof. In a specific embodiment, an antibody or antigen binding fragment described herein, which specifically binds to human Axl, is a human IgG1 antibody or antigen-binding fragment thereof.

In some embodiments, the antibody or antigen-binding fragment comprises a heavy chain constant region or a light chain constant region. In some embodiments, the antibody or antigen-binding fragment comprises a heavy chain constant region and a light chain constant region. In certain embodiments, the antibody or antigen-binding fragment comprises a human heavy chain constant region or a human light chain constant region. In some embodiments, the antibody or antigen-binding fragment comprises a human heavy chain constant region and a human light chain constant region.

In certain embodiments, the antibody or antigen-binding fragment is an IgG1 antibody or antigen-binding fragment. In certain embodiments, the antibody or antigen-binding fragment is an IgG2 antibody or antigen-binding fragment. In some embodiments, the antibody or antigen-binding fragment is a human IgG1 antibody or antigen-binding fragment or a human IgG2 antibody or antigen-binding fragment. In some embodiments, the antibody or antigen-binding fragment comprises a kappa light chain constant region or a lambda light chain constant region. In some embodiments, the antibody or antigen-binding fragment comprises a human kappa light chain constant region or a human lambda light chain constant region.

In a certain embodiment, an antibody or antigen binding fragment described herein which specifically binds to human Axl comprises a human kappa light chain constant region or a human gamma heavy chain constant region.

In a certain embodiment, an isolated antibody described herein, or an antigen-binding fragment thereof, which specifically binds to human Axl, is a monoclonal antibody.

In a certain embodiment, an antibody (e.g., monoclonal antibody) described herein, or an antigen-binding fragment thereof, which specifically binds to human Axl, is a human antibody.

In a certain embodiment, an antibody (e.g., monoclonal antibody) described herein, or an antigen-binding fragment thereof, which specifically binds to human Axl, is a chimeric antibody.

In a certain embodiment, an isolated antibody (e.g., monoclonal antibody) described herein, or an antigen-binding fragment thereof, which specifically binds to human Axl, is a humanized antibody.

In a certain embodiment, an isolated antibody (e.g., monoclonal antibody) described herein, or an antigen-binding fragment thereof, which specifically binds to human Axl, is a single chain antibody.

In a certain embodiment, an isolated antibody (e.g., monoclonal antibody) described herein, or an antigen-binding fragment thereof, which specifically binds to human Axl, is a multispecific, e.g., bispecific, antibody.

In a certain embodiment, an isolated antibody (e.g., monoclonal antibody) described herein, or an antigen-binding fragment thereof, which specifically binds to human Axl, is a monovalent antibody.

In a certain embodiment, an isolated antibody (e.g., monoclonal antibody) described herein, or an antigen-binding fragment thereof, which specifically binds to human Axl, is fused to a heterologous polypeptide.

In a certain embodiment, an isolated antibody (e.g., monoclonal antibody) described herein, or an antigen-binding fragment thereof, which specifically binds to human Axl, is conjugated to an agent. In a particular embodiment, the agent is a toxin. In one embodiment, the toxin is abrin, ricin A, pseudomonas exotoxin, cholera toxin, or diphtheria toxin.

In some embodiments, an antibody, or an antigen-binding fragment thereof, that binds to human Axl has a mutated IgG Fc domain which binds to Fc gamma receptors with a greater affinity than the corresponding native IgG Fc domain. In some embodiments, an antibody, or an antigen-binding fragment thereof, that binds to human Axl has a mutated IgG Fc domain which binds to Fc gamma receptors with a greater affinity than the corresponding native IgG Fc domain.

In a specific embodiment, the antibody or antigen-binding fragment described herein comprises an Fc region or domain which is capable of binding Fc receptors. In a specific embodiment, the antibody or antigen-binding fragment described herein does not comprise a full-length Fc region or domain. In a particular embodiment, the antibody or antigen-binding fragment described herein does not comprise an Fc region or domain. In a specific embodiment, the antibody or antigen-binding fragment described herein comprises an Fc region or domain which is not capable of binding Fc receptors.

In specific embodiments, the antibody or antigen-binding fragment described herein is capable of eliciting an effector function, e.g., antibody-dependent cell-mediated cytotoxicity (ADCC), antibody-dependent cellular phagocytosis (ADCP) and complement-dependent cytotoxicity (CDC). In a specific embodiment, the antibody or antigen-binding fragment described herein is capable of eliciting ADCC.

In a specific embodiment, the antibody described herein is a full-length antibody.

In some embodiments, the antibody or antigen-binding fragment described herein inhibits binding of Gas6 to Axl.

In certain embodiments, provided herein is a human or humanized monoclonal antibody, or an antigen-binding fragment thereof that preferably binds to Ig-like domain 1 (D1) of the extracellular domain of human Axl. As a non-limiting example, in some embodiments, an antibody or antigen-binding fragment thereof that binds to Ig-like domain 1 (D1) of the extracellular domain of human Axl can exhibit one or more of the following properties: i) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; ii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iv) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; vi) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vii) when tested in vitro increases IL-1RA secretion from human dendritic cells by at least 2-fold (or at least 3-fold) as determined by ELISA when compared to isotype control; and viii) when tested in vitro increases IL-2 secretion from a co-culture of CD4⁺ T cells and dendritic cells by at least 40% (e.g., at a concentration of 30 nM or higher) as determined by ELISA when compared to isotype control.

In certain embodiments, provided herein is a human or humanized monoclonal antibody, or an antigen-binding fragment thereof that binds to Ig-like domain 1 (D1) of the extracellular domain of human Axl. As a non-limiting example, in some embodiments, an antibody or antigen-binding fragment thereof that binds to Ig-like domain 1 (D1) of the extracellular domain of human Axl can exhibit one or more of the following properties: i) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; ii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iv) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; vi) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vii) when tested in vitro increases IL-1RA secretion from human dendritic cells by at least 2-fold (or at least 3-fold) as determined by ELISA when compared to isotype control; and viii) when tested in vitro increases IL-2 secretion from a co-culture of CD4⁺ T cells and dendritic cells by at least 40% (e.g., at a concentration of 30 nM or higher) as determined by ELISA when compared to isotype control.

In certain embodiments, provided herein is a human or humanized monoclonal antibody, or an antigen-binding fragment thereof that preferably binds to Ig-like domain 1 (D1) of the extracellular domain of human Axl and which exhibits one or more of the following properties: i) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; ii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iv) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; vi) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vii) when tested in vitro increases IL-1RA secretion from human dendritic cells by at least 2-fold (or at least 3-fold) as determined by ELISA when compared to isotype control; and viii) when tested in vitro increases IL-2 secretion from a co-culture of CD4⁺ T cells and dendritic cells by at least 40% (e.g., at a concentration of 30 nM or higher) as determined by ELISA when compared to isotype control.

In certain embodiments, provided herein is a human or humanized monoclonal antibody, or an antigen-binding fragment thereof that binds to Ig-like domain 1 (D1) of the extracellular domain of human Axl and which exhibits one or more of the following properties: i) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; ii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iv) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; vi) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vii) when tested in vitro increases IL-1RA secretion from human dendritic cells by at least 2-fold (or at least 3-fold) as determined by ELISA when compared to isotype control; and viii) when tested in vitro increases IL-2 secretion from a co-culture of CD4⁺ T cells and dendritic cells by at least 40% (e.g., at a concentration of 30 nM or higher) as determined by ELISA when compared to isotype control.

In certain embodiments, provided herein is a human or humanized monoclonal antibody, or an antigen-binding fragment thereof that exhibits one or more of the following properties: i) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; ii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iv) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; vi) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vii) when tested in vitro increases IL-1RA secretion from human dendritic cells by at least 2-fold (or at least 3-fold) as determined by ELISA when compared to isotype control; and viii) when tested in vitro increases IL-2 secretion from a co-culture of CD4⁺ T cells and dendritic cells by at least 40% (e.g., at a concentration of 30 nM or higher) as determined by ELISA when compared to isotype control.

In a certain embodiment, provided herein is a composition comprising a therapeutically effective amount of an antibody described herein or antigen-binding fragment thereof, which specifically binds to human Axl.

In a certain embodiment, provided herein is a pharmaceutical composition comprising an antibody described herein or antigen-binding fragment thereof, which specifically binds to human Axl and a pharmaceutically acceptable carrier.

In a certain embodiment, provided herein is a polynucleotide comprising one or more nucleotide sequence encoding a VH, a VL, or both a VL and a VH, of an antibody described herein or antigen-binding fragment thereof, which specifically binds to human Axl.

In a certain embodiment, provided herein is a polynucleotide comprising one or more nucleotide sequence encoding a heavy chain, a light chain, or both heavy chain and a light chain of an antibody described herein or antigen-binding fragment thereof, which specifically binds to human Axl.

In a certain embodiment, provided herein is a population of polynucleotides comprising (i) a first polynucleotide comprising a nucleotide sequence encoding a VH or a heavy chain of an antibody described herein or antigen-binding fragment thereof, which specifically binds to human Axl, and (ii) a second polypeptide comprising nucleotide sequences encoding a VL or a light chain of the antibody.

In a certain embodiment, provided herein is a vector comprising a polynucleotide described herein comprising nucleotide sequences encoding a VH, or a VL, or a VH and VL of an antibody described herein or antigen-binding fragment thereof, which specifically binds to human Axl.

In a certain embodiment, provided herein is a vector comprising a polynucleotide described herein comprising nucleotide sequences encoding a heavy chain, a light chain, or both heavy chain and a light chain of an antibody described herein or antigen-binding fragment thereof, which specifically binds to human Axl.

In a certain embodiment, provided herein is a population of vectors comprising (i) a first vector comprising a nucleotide sequence encoding a VH or a heavy chain of an anti-Axl antibody or antigen-binding fragment described herein, and (ii) a second vector comprising a nucleotide sequence encoding a VL or a light chain of an anti-Axl antibody or antigen-binding fragment described herein.

In a certain embodiment, provided herein is an isolated cell comprising a polynucleotide comprising nucleotide sequences encoding a VH, a VL, or both a VH and a VL of an antibody described herein or antigen-binding fragment thereof, which specifically binds to human Axl.

In a certain embodiment, provided herein is an isolated cell comprising a polynucleotide comprising nucleotide sequences encoding a heavy chain, a light chain, or both heavy chain and a light chain of an antibody described herein or antigen-binding fragment thereof, which specifically binds to human Axl.

In a certain embodiment, provided herein is an isolated cell comprising a population of polynucleotides described herein.

In a certain embodiment, provided herein is a population of cells comprising (i) a first host cell comprising a polynucleotide described herein comprising a nucleotide sequence encoding a VH or a heavy chain of an anti-Axl antibody or antigen-binding fragment described herein, and (ii) a second host cell comprising a polynucleotide comprising a nucleotide sequence encoding a VL or a light chain of an anti-Axl antibody or antigen-binding fragment described herein.

In a certain embodiment, provided herein is an isolated cell producing an antibody described herein or antigen-binding fragment thereof, which specifically binds to human Axl.

In a certain embodiment, provided herein is a kit comprising an antibody described herein or antigen-binding fragment thereof, which specifically binds to human Axl.

In a particular aspect, provided herein is a method of managing, protecting against, or treating cancer, for example, breast cancer, prostate cancer, gastric cancer, lung cancer, e.g., non-small cell lung cancer, adenoma, melanoma, lymphoma, or leukemia, or infection, for example, bacterial, e.g., gram-negative or gram-positive bacteria, fungal, viral, or parasitic infection in a subject, comprising administering to a subject in need thereof a therapeutically effective amount of an antibody or antigen binding fragment described herein, for example, Ab301, Ab302, Ab303, Ab304 or Ab305, or antigen-binding fragment thereof, which specifically binds to human Axl.

In a certain embodiment, provided herein is a method of modulating an immune response, e.g., enhancing an immune response, in a subject, comprising administering to a subject in need thereof an effective amount of an antibody described herein, for example, Ab301, Ab302, Ab303, Ab304 or Ab305, or antigen-binding fragment thereof, which specifically binds to human Axl. In a particular embodiment, the subject is a subject suffering from an infection, a subject having cancer, or an immunocompromised subject such as, for example, a subject who is undergoing, or had undergone treatment with, an anti-cancer therapy, is HIV positive, or who has AIDS or SCID, has diabetes, or has had a transplant and is taking an immunosuppressant. In a particular embodiment, the subject has been treated with an immunosuppressant.

In a certain embodiment, provided herein is a method of enhancing a proinflammatory response in a subject, comprising administering to a subject in need thereof an effective amount of an antibody described herein, for example, Ab301, Ab302, Ab303, Ab304 or Ab305, or antigen-binding fragment thereof, which specifically binds to human Axl. In a particular embodiment, such a method of enhancing a proinflammatory response in a subject results in an increase in TNF-α secretion.

In a specific embodiment, provided herein is a method of enhancing an immune response to a vaccine in a subject, comprising administering to a subject in need thereof, who is or has been administered the vaccine, an effective amount of an antibody described herein or antigen-binding fragment thereof, which specifically binds to human Axl. In a certain embodiment, the vaccine is a cancer or tumor vaccine.

In one embodiment, provided herein is a vaccine antigen that can also be targeted, for example, to particular cell types or to particular tissues. In a specific embodiment, the vaccine antigen can be targeted to Antigen Presenting Cells (APCs), for example by use of agents such as antibodies targeted to APC-surface receptors such as DEC-205, for example as discussed in WO 2009/061996 (Celldex Therapeutics, Inc.), or the Mannose Receptor (CD206) for example as discussed in WO 03040169 (Medarex, Inc.).

In one embodiment, provided herein is a method of managing, preventing, protecting against, or treating metastasis in a subject, comprising administering to a subject in need thereof a therapeutically effective amount of an antibody described herein or antigen-binding fragment thereof, which specifically binds to human Axl.

In one embodiment, provided herein is a method of treating sepsis in a subject, comprising administering to a subject in need thereof a therapeutically effective amount of an antibody described herein or antigen-binding fragment thereof, which specifically binds to human Axl.

In a certain embodiment, provided herein is a method for activating or increasing/enhancing an innate immune response in a subject, comprising administering to a subject in need thereof an effective amount of an antibody described herein or antigen-binding fragment thereof, which specifically binds to human Axl. In one embodiment, the subject has cancer, or is being treated for cancer with an anti-cancer therapeutic agent, or the subject has an infection. In a particular embodiment, the subject has cancer and the method is effective in treating or managing the subject's cancer.

In a certain embodiment, provided herein is a method for stimulating cytotoxicity of NK cells in a subject, comprising administering to a subject in need thereof an effective amount of an antibody described herein, for example, Ab301, Ab302, Ab303, Ab304 or Ab305, or antigen-binding fragment thereof, which specifically binds to human Axl. In one embodiment, the subject has cancer, or is being treated for cancer with an anti-cancer therapeutic, or the subject has an infection.

In a specific embodiment, provided herein is a method of making an antibody or an antigen-binding fragment thereof that specifically binds to human Axl, comprising culturing a cell, host cell, or population of cells described herein to express an anti-Axl antibody or antigen-binding fragment thereof.

In a certain embodiment, provided herein is a method of making an antibody or an antigen-binding fragment thereof that specifically binds to human Axl, comprising expressing a polynucleotide described herein or a population of polynucleotides described herein.

In another embodiment, presented herein is a method of increasing proinflammatory cytokine production in a subject in need thereof, comprising administering to the subject an antibody or an antigen-binding fragment thereof that specifically binds to human Axl such that the production of at least one proinflammatory cytokine is increased. For example, the cytokine secretion can be increase by about 5%, about 10%, about 15%, about 20%, about 30%, about 40%, about 50%, about 60%, about 70%, about 80%, about 90%, about 100%, about 2-fold, about 5-fold, about 10-fold, about 15-fold, about 20-fold, about 30-fold, or about 50-fold or more relative to secretion in the absence of the antibody. In a specific embodiment, the at least one proinflammatory cytokine is TNFα (tumor necrosis factor a). In further embodiments, the at least one proinflammatory cytokine is Eotaxin-1, G-CSF, Flt-3L, GM-CSF, Fractalkine, IFNa2, IFNγ, GRO alpha, IL-2, IL-10, MCP-3, IL-12P40, MDC, IL-1RA, IL-1B, IL-4, TNFα, RANTES, MIP-1B, IL-6, IL-8, IP-10, VEGF-A, and/or MIP-1a. In yet another embodiment, the antibody or antigen-binding fragment does not substantially induce phosphorylation of Axl. For example, the antibody or antigen-binding fragment induces phosphorylation of Axl at least 50%, 60%, 70%, 80%, 90% or less than the phosphorylation induced by Gas6 binding to Axl. In a particular embodiment, the antibody or antigen-binding fragment is one that specifically binds to Ig-like domain 1 (D1) of the extracellular domain of human Axl. In yet another embodiment, the antibody or antigen-binding fragment is one of the antibodies or antigen-binding fragments described herein.

In a certain aspect, further provided herein is a method of increasing proinflammatory cytokine production in a subject in need thereof, comprising administering to the subject an antibody or an antigen-binding fragment thereof disclosed herein such that the production of at least one cytokine is increased. In certain embodiments, the at least one proinflammatory cytokine is Eotaxin-1, G-CSF, Flt-3L, GM-CSF, Fractalkine, IFNa2, IFNγ, GRO alpha, IL-2, IL-10, MCP-3, IL-12P40, MDC, IL-1RA, IL-1B, IL-4, TNFα, RANTES, MIP-1B, IL-6, IL-8, IP-10, VEGF-A, and/or MIP-1a. In some embodiments, the production of proinflammatory cytokine GRO alpha is increased. In certain embodiments, the production of proinflammatory cytokine MCP-3 is increased. In certain embodiments, the production of proinflammatory cytokine IL-12P40 is increased. In certain embodiments, the production of proinflammatory cytokine MDC is increased. In certain embodiments, the production of proinflammatory cytokine IL-1RA is increased. In certain embodiments, the production of proinflammatory cytokine IL-1B is increased. In certain embodiments, the production of proinflammatory cytokine TNFα is increased. In certain embodiments, the production of proinflammatory cytokine RANTES is increased. In certain embodiments, the production of proinflammatory cytokine MIP-1B is increased. In certain embodiments, the production of proinflammatory cytokine IL-2 is increased. In certain embodiments, the production of proinflammatory cytokine IL-6 is increased. In certain embodiments, the production of proinflammatory cytokine IL-8 is increased. In certain embodiments, the production of proinflammatory cytokine MIP-1a is increased. In certain embodiments, the production of proinflammatory cytokine Eotaxin-1 is increased. In certain embodiments, the production of proinflammatory cytokine G-CSF is increased. In certain embodiments, the production of proinflammatory cytokine Flt-3L is increased. In certain embodiments, the production of proinflammatory cytokine GM-CSF is increased. In certain embodiments, the production of proinflammatory cytokine Fractalkine is increased. In certain embodiments, the production of proinflammatory cytokine IFNa2 is increased. In certain embodiments, the production of proinflammatory cytokine IFNγ is increased. In certain embodiments, the production of proinflammatory cytokine IL-10 is increased. In certain embodiments, the production of proinflammatory cytokine IP-10 is increased. In certain embodiments, the production of proinflammatory cytokine VEGF-A is increased.

In a certain embodiment, provided herein is a method of increasing proinflammatory secretory factor production and/or secretion in a subject in need thereof, comprising administering to the subject an antibody or an antigen-binding fragment thereof disclosed herein such that the production and/or secretion of about 1 or more, about 2 or more, about 3 or more, about 4 or more, about 5 or more, about 10 or more, about 15 or more, or about 20 or more proinflammatory secretory factors is increased. In a certain embodiment, the production and/or secretion of one or more proinflammatory factors can be increase by about 5%, about 10%, about 15%, about 20%, about 30%, about 40%, about 50%, about 60%, about 70%, about 80%, about 90%, about 100%, about 2-fold, about 5-fold, about 10-fold, about 15-fold, about 20-fold, about 30-fold, or about 50-fold or more relative to secretion in the absence of the antibody.

In a specific embodiment, the one or more proinflammatory secretory factor cytokine can be any 1, 2, 3, 4 or 5 of TNFα, IL-1RA, fibroblast growth factor 2 (FGF-2), eotaxin-1 (CCL11), transforming growth factor alpha (TGF-a) granulocyte-colony stimulating factor (G-CSF), Fms-related tyrosine kinase 3 ligand (Flt-3L), granulocyte macrophage-colony stimulating factor (GM-CSF), fractalkine (CX3CL1), interferon alpha-2 (IFN-a2), interferon-gamma (IFN-γ), growth-regulated oncogene alpha (GRO alpha), interleukin-2 (IL-2), interleukin-10 (IL-10), monocyte chemotactic protein 3 (MCP-3), interleukin-12 p40 (IL-12P40), macrophage-derived chemokine (MDC), platelet-derived growth factor AA homodimer (PDGF-AA), interleukin-13 (IL-13), platelet-derived growth factor BB homodimer (PDGF-BB), soluble CD40 ligand (sCD40L), interleukin-1B (IL-1B), interleukin-4 (IL-4), interleukin-6 (IL-6), interleukin-8 (IL-8), interferon γ-induced protein 10 (IP-10), macrophage inflammatory protein (MIP)-1α, MIP-1β, Regulated on Activation Normal T cell Expressed and Secreted (RANTES), vascular endothelial growth factor A (VEGF-A) and IL-18.

In some embodiments, provided herein is a method of increasing proinflammatory secretory factor production in a subject in need thereof, comprising administering to the subject an antibody or an antigen-binding fragment thereof disclosed herein such that the production of one or more secretory factor is increased. In certain embodiments, provided herein is a method wherein the production of proinflammatory secretory factor TNFα, IL-1RA, fibroblast growth factor 2 (FGF-2), eotaxin-1 (CCL11), transforming growth factor alpha (TGF-a) granulocyte-colony stimulating factor (G-CSF), Fms-related tyrosine kinase 3 ligand (Flt-3L), granulocyte macrophage-colony stimulating factor (GM-CSF), fractalkine (CX3CL1), interferon alpha-2 (IFN-a2), interferon-gamma (IFN-γ), growth-regulated oncogene alpha (GRO alpha), interleukin-2 (IL-2), interleukin-10 (IL-10), monocyte chemotactic protein 3 (MCP-3), interleukin-12 p40 (IL-12P40), macrophage-derived chemokine (MDC), platelet-derived growth factor AA homodimer (PDGF-AA), interleukin-13 (IL-13), platelet-derived growth factor BB homodimer (PDGF-BB), soluble CD40 ligand (sCD40L), interleukin-1B (IL-1B), interleukin-4 (IL-4), interleukin-6 (IL-6), interleukin-8 (IL-8), interferon γ-induced protein 10 (IP-10), macrophage inflammatory protein (MIP)-1α, MIP-1β, Regulated on Activation Normal T cell EXPRESSED and Secreted (RANTES), vascular endothelial growth factor A (VEGF-A) and/or IL-18 is/are increased.

In one aspect, provided herein is a method of increasing proinflammatory secretory factor production in a subject in need thereof, comprising administering to the subject an antibody or an antigen-binding fragment thereof disclosed herein such that the production of any 1, 2, 3, 4, 5, 6, 7, 8, 9, 10, 15, 20, or more proinflammatory secretory factors is increased. As a non-limiting example, in certain embodiments, provided herein are methods wherein the production of one or more of proinflammatory secretory factors TNFα, IL-1RA, fibroblast growth factor 2 (FGF-2), eotaxin-1 (CCL11), transforming growth factor alpha (TGF-a) granulocyte-colony stimulating factor (G-CSF), Fms-related tyrosine kinase 3 ligand (Flt-3L), granulocyte macrophage-colony stimulating factor (GM-CSF), fractalkine (CX3CL1), interferon alpha-2 (IFN-a2), interferon-gamma (IFN-γ), growth-regulated oncogene alpha (GRO alpha), interleukin-2 (IL-2), interleukin-10 (IL-10), monocyte chemotactic protein 3 (MCP-3), interleukin-12 p40 (IL-12P40), macrophage-derived chemokine (MDC), platelet-derived growth factor AA homodimer (PDGF-AA), interleukin-13 (IL-13), platelet-derived growth factor BB homodimer (PDGF-BB), soluble CD40 ligand (sCD40L), interleukin-1B (IL-1B), interleukin-4 (IL-4), interleukin-6 (IL-6), interleukin-8 (IL-8), interferon γ-induced protein 10 (IP-10), macrophage inflammatory protein (MIP)-1α, MIP-1β, Regulated on Activation Normal T cell Expressed and Secreted (RANTES), vascular endothelial growth factor A (VEGF-A) and IL-18 is/are increased.

In one aspect, the method described herein further comprises administering to the subject a second therapeutic agent. In certain embodiments, the second therapeutic agent is an immune checkpoint blockade. In a specific embodiment, the immune checkpoint blockade inhibits the activity of PD-1, PD-L1, PD-L2, CTLA-4, TIM-3, or LAG-3. In one embodiment, the immune checkpoint blockade inhibits the activity of PD-1. In another embodiment, the immune checkpoint blockade inhibits the activity of PD-L1. In various embodiments, the second therapeutic agent is a monoclonal antibody.

### 3.1 Illustrative Embodiments

1. An isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

2. The antibody or antigen-binding fragment of embodiment 1, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

3. The antibody or antigen-binding fragment of embodiment 1, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

4. The antibody or antigen-binding fragment of embodiment 1, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

5. The antibody or antigen-binding fragment of any one of embodiments 1-4, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively, or conservative sequence modifications thereof.

6. The antibody or antigen-binding fragment of any one of embodiments 1-4, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively, or conservative sequence modifications thereof.

7. The antibody or antigen-binding fragment of any one of embodiments 1-4, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively.

8. The antibody or antigen-binding fragment of any one of embodiments 1-4, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively.

9. An isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

10. The antibody or antigen-binding fragment of embodiment 9, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

11. The antibody or antigen-binding fragment of embodiment 9, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

12. The antibody or antigen-binding fragment of embodiment 9, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

13. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively, or conservative sequence modifications thereof.

14. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively, or conservative sequence modifications thereof.

15. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively.

16. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively.

17. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively, or conservative sequence modifications thereof.

18. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively, or conservative sequence modifications thereof.

19. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively.

20. The antibody or antigen-binding fragment of any one of embodiments 9-12, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively.

21. An isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

22. The antibody or antigen-binding fragment of embodiment 21, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

23. The antibody or antigen-binding fragment of embodiment 21, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

24. The antibody or antigen-binding fragment of embodiment 21, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

25. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively, or conservative sequence modifications thereof.

26. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively, or conservative sequence modifications thereof.

27. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively.

28. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively.

29. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively, or conservative sequence modifications thereof.

30. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively, or conservative sequence modifications thereof.

31. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively.

32. The antibody or antigen-binding fragment of any one of embodiments 21-24, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively.

33. An isolated antibody, or an antigen-binding fragment thereof, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

34. The antibody or antigen-binding fragment of embodiment 33, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

35. The antibody or antigen-binding fragment of embodiment 33, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

36. The antibody or antigen-binding fragment of embodiment 33, which specifically binds to human Axl, comprising:

(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;

(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;

(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;

(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or

(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

37. The antibody or antigen-binding fragment of any one of embodiments 33-36, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively, or conservative sequence modifications thereof.

38. The antibody or antigen-binding fragment of any one of embodiments 33-36, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively, or conservative sequence modifications thereof;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively, or conservative sequence modifications thereof;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively, or conservative sequence modifications thereof;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively, or conservative sequence modifications thereof; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively, or conservative sequence modifications thereof.

39. The antibody or antigen-binding fragment of any one of embodiments 33-36, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively.

40. The antibody or antigen-binding fragment of any one of embodiments 33-36, wherein:

(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively;

(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively;

(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively;

(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively; or

(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively.

41. The antibody or antigen-binding fragment of any one of embodiments 1-36, wherein:
(i) the light chain variable region (VL) comprises an amino acid sequence having at least 80%, at least 85%, at least 90%, at least 95%, or at least 98% sequence identity to SEQ ID NO: 6; and/or
(ii) the heavy chain variable region (VH) comprises an amino acid sequence having at least 80%, at least 85%, at least 90%, at least 95%, or at least 98% sequence identity to SEQ ID NO: 5.

42. The antibody or an antigen-binding fragment of embodiment 41, wherein:
(i) the light chain variable region (VL) comprises an amino acid sequence having at least 80%, at least 85%, at least 90%, at least 95%, or at least 98% sequence identity to SEQ ID NO: 6; and
(ii) the heavy chain variable region (VH) comprises an amino acid sequence having at least 80%, at least 85%, at least 90%, at least 95%, or at least 98% sequence identity to SEQ ID NO: 5.

43. The antibody or antigen-binding fragment of any one of embodiments 1-8, wherein:
(i) the light chain variable region (VL) comprises an amino acid sequence of SEQ ID NO: 6; and/or
(ii) the heavy chain variable region (VH) comprises an amino acid sequence of SEQ ID NO: 5.

44. The antibody or an antigen-binding fragment of embodiment 43, wherein:
(i) the light chain variable region (VL) comprises an amino acid sequence of SEQ ID NO: 6; and
(ii) the heavy chain variable region (VH) comprises an amino acid sequence of SEQ ID NO: 5.

45. The antibody or antigen-binding fragment of any one of embodiments 9-16, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 7.

46. The antibody or an antigen-binding fragment of embodiment 45, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 7.

47. The antibody or antigen-binding fragment of any one of embodiments 9-16, wherein:
(i) the light chain variable region (VL) comprises an amino acid sequence of SEQ ID NO: 6; and/or
(ii) the heavy chain variable region (VH) comprises an amino acid sequence of SEQ ID NO: 7.

48. The antibody or an antigen-binding fragment of embodiment 47, wherein:
(i) the light chain variable region (VL) comprises an amino acid sequence of SEQ ID NO: 6; and
(ii) the heavy chain variable region (VH) comprises an amino acid sequence of SEQ ID NO: 7.

49. The antibody or antigen-binding fragment of any one of embodiments 9-12 and 17-20, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 8.

50. The antibody or an antigen-binding fragment of embodiment 49, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 8.

51. The antibody or antigen-binding fragment of any one of embodiments 9-12 and 17-20, wherein:
(i) the light chain variable region (VL) comprises an amino acid sequence of SEQ ID NO: 6; and/or
(ii) the heavy chain variable region (VH) comprises an amino acid sequence of SEQ ID NO: 8.

52. The antibody or an antigen-binding fragment of embodiment 51, wherein:
(i) the light chain variable region (VL) comprises an amino acid sequence of SEQ ID NO: 6; and
(ii) the heavy chain variable region (VH) comprises an amino acid sequence of SEQ ID NO: 8.

53. The antibody or antigen-binding fragment of any one of embodiments 21-28, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 9.

54. The antibody or an antigen-binding fragment of embodiment 53, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 9.

55. The antibody or antigen-binding fragment of any one of embodiments 21-28, wherein:
(i) the light chain variable region (VL) comprises an amino acid sequence of SEQ ID NO: 6; and/or
(ii) the heavy chain variable region (VH) comprises an amino acid sequence of SEQ ID NO: 9.

56. The antibody or an antigen-binding fragment of embodiment 55, wherein:
(i) the light chain variable region (VL) comprises an amino acid sequence of SEQ ID NO: 6; and
(ii) the heavy chain variable region (VH) comprises an amino acid sequence of SEQ ID NO: 9.

57. The antibody or antigen-binding fragment of any one of embodiments 21-24 and 29-32, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 10.

58. The antibody or an antigen-binding fragment of embodiment 49, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 10.

59. The antibody or antigen-binding fragment of any one of embodiments 21-24 and 29-32, wherein:
(i) the light chain variable region (VL) comprises an amino acid sequence of SEQ ID NO: 6; and/or
(ii) the heavy chain variable region (VH) comprises an amino acid sequence of SEQ ID NO: 10.

60. The antibody or an antigen-binding fragment of embodiment 51, wherein:
(i) the light chain variable region (VL) comprises an amino acid sequence of SEQ ID NO: 6; and
(ii) the heavy chain variable region (VH) comprises an amino acid sequence of SEQ ID NO: 10.

61. The antibody or antigen-binding fragment of any one of embodiments 33-40, wherein:
(i) the light chain variable region (VL) comprises an amino acid sequence having at least 80%, at least 85%, at least 90%, at least 95%, at least 98%, or 100% sequence identity to SEQ ID NO: 12; and/or
(ii) the heavy chain variable region (VH) comprises an amino acid sequence having at least 80%, at least 85%, at least 90%, at least 95%, at least 98%, or 100% sequence identity to SEQ ID NO: 11.

62. The antibody or an antigen-binding fragment of embodiment 61, wherein:
(i) the light chain variable region (VL) comprises an amino acid sequence having at least 80%, at least 85%, at least 90%, at least 95%, at least 98%, or 100% sequence identity to SEQ ID NO: 12; and
(ii) the heavy chain variable region (VH) comprises an amino acid sequence having at least 80%, at least 85%, at least 90%, at least 95%, at least 98%, or 100% sequence identity to SEQ ID NO: 11.

63. The antibody or antigen-binding fragment of any one of embodiments 33-40, wherein:
(i) the light chain variable region (VL) comprises an amino acid sequence of SEQ ID NO: 12; and/or
(ii) the heavy chain variable region (VH) comprises an amino acid sequence of SEQ ID NO: 11.

64. The antibody or an antigen-binding fragment of embodiment 63, wherein:
(i) the light chain variable region (VL) comprises an amino acid sequence of SEQ ID NO: 12; and
(ii) the heavy chain variable region (VH) comprises an amino acid sequence of SEQ ID NO: 11.

65. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 5.

66. The antibody or an antigen-binding fragment of embodiment 65, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 5.

67. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 5.

68. The antibody or an antigen-binding fragment of embodiment 67, wherein:
(i) the light chain variable region (VL) comprises an amino acid sequence of SEQ ID NO: 6; and
(ii) the heavy chain variable region (VH) comprises an amino acid sequence of SEQ ID NO: 5.

69. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 7.

70. The antibody or an antigen-binding fragment of embodiment 69, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 7.

71. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 7.

72. The antibody or an antigen-binding fragment of embodiment 71, wherein:
(i) the light chain variable region (VL) comprises an amino acid sequence of SEQ

ID NO: 6; and
(ii) the heavy chain variable region (VH) comprises an amino acid sequence of SEQ ID NO: 7.

73. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 8.

74. The antibody or an antigen-binding fragment of embodiment 73, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 8.

75. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 8.

76. The antibody or an antigen-binding fragment of embodiment 75, wherein:
(i) the light chain variable region (VL) comprises an amino acid sequence of SEQ ID NO: 6; and
(ii) the heavy chain variable region (VH) comprises an amino acid sequence of SEQ ID NO: 8.

77. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 9.

78. The antibody or an antigen-binding fragment of embodiment 77, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 9.

79. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 9.

80. The antibody or an antigen-binding fragment of embodiment 79, wherein:
(i) the light chain variable region (VL) comprises an amino acid sequence of SEQ ID NO: 6; and
(ii) the heavy chain variable region (VH) comprises an amino acid sequence of SEQ ID NO: 9.

81. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 10.

82. The antibody or an antigen-binding fragment of embodiment 81, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 10.

83. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 10.

84. The antibody or an antigen-binding fragment of embodiment 83, wherein:
(i) the light chain variable region (VL) comprises an amino acid sequence of SEQ ID NO: 6; and
(ii) the heavy chain variable region (VH) comprises an amino acid sequence of SEQ ID NO: 10.

85. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 12; and/or
(ii) the heavy chain variable region (VH) comprises an amino acid sequence having at least 80%, at least 85%, at least 90%, at least 95%, at least 98%, or 100% sequence identity to SEQ ID NO: 11.

86. The antibody or an antigen-binding fragment of embodiment 85, wherein:
(i) the light chain variable region (VL) comprises an amino acid sequence having at least 80%, at least 85%, at least 90%, at least 95%, at least 98%, or 100% sequence identity to SEQ ID NO: 12; and
(ii) the heavy chain variable region (VH) comprises an amino acid sequence having at least 80%, at least 85%, at least 90%, at least 95%, at least 98%, or 100% sequence identity to SEQ ID NO: 11.

87. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 12; and/or
(ii) the heavy chain variable region (VH) comprises an amino acid sequence of SEQ ID NO: 11.

88. The antibody or an antigen-binding fragment of embodiment 87, wherein:
(i) the light chain variable region (VL) comprises an amino acid sequence of SEQ ID NO: 12; and
(ii) the heavy chain variable region (VH) comprises an amino acid sequence of SEQ ID NO: 11.

89. The antibody or antigen-binding fragment of any one of embodiments 1 to 63 and 65 to 87, which is a humanized antibody.

90. The antibody or antigen-binding fragment of any one of embodiments 1-89, which specifically binds to Ig-like domain 1 in the extracellular domain of human Axl.

91. The antibody or antigen-binding fragment of any one of embodiments 1-90, which does not specifically bind to the extracellular domain of MerTK.

92. An isolated antibody, or an antigen-binding fragment thereof, which specifically binds to Ig-like domain 1 in the extracellular domain of human Axl.

93. The antibody or antigen-binding fragment of embodiment 92, which does not specifically bind to the extracellular domain of MerTK.

94. An isolated antibody, or an antigen-binding fragment thereof, which binds to the same epitope of human Axl as the antibody of any of embodiments 1 to 93.

95. An isolated antibody, or an antigen-binding fragment thereof, which competes for binding to human Axl with the antibody of any of embodiments 1 to 93.

96. An isolated antibody, or an antigen-binding fragment thereof, which binds to Ig-like domain 1 in the extracellular domain of human Axl and inhibits binding of Gas6 to Axl and/or, inhibits Axl phosphorylation.

97. The antibody or antigen-binding fragment of embodiment 96, which does not specifically bind to the extracellular domain of MerTK.

98. The antibody or antigen-binding fragment of any one of embodiments 1 to 97, wherein the antibody comprises a heavy chain constant region or a light chain constant region.

99. The antibody or antigen-binding fragment of embodiment 98, wherein the antibody comprises a heavy chain constant region and a light chain constant region.

100. The antibody or antigen-binding fragment of embodiment 98, wherein the antibody comprises a human heavy chain constant region or a human light chain constant region.

101. The antibody or antigen-binding fragment of embodiment 100 wherein the antibody comprises a human heavy chain constant region and a human light chain constant region.

102. The antibody or antigen-binding fragment of any one of embodiments 98 to 101, wherein the antibody or antigen-binding fragment is an IgG antibody or antigen-binding fragment thereof.

103. The antibody or antigen-binding fragment of embodiment 102, wherein the antibody or antigen-binding fragment is an IgG1 antibody or antigen-binding fragment thereof.

104. The antibody or antigen-binding fragment of any one of embodiments 98 to 103, wherein the antibody comprises a kappa light chain constant region or a lambda light chain constant region.

105. The antibody or antigen-binding fragment of any one of embodiments 98 to 103, wherein the antibody comprises a human kappa light chain constant region or a human lambda light chain constant region.

106. The antibody or antigen-binding fragment of any one of embodiments 1 to 105, which is a monoclonal antibody.

107. The antibody or antigen-binding fragment of any one of embodiments 1 to 106, which is a bispecific antibody.

108. The antibody or antigen-binding fragment of any one of embodiments 1 to 107, which is fused to a heterologous polypeptide.

109. The antibody or antigen-binding fragment of any one of embodiments 1 to 108, which is conjugated to an agent.

110. The antibody or antigen-binding fragment of embodiment 109, wherein the agent is a toxin.

111. The antibody or antigen-binding fragment of any one of embodiments 1-110, which inhibits binding of Gas6 to Axl.

112. The antibody or antigen-binding fragment of any one of embodiments 1-111, which comprises an Fc region or domain that is capable of binding Fc receptors.

113. The antibody or antigen-binding fragment of any one of embodiments 1-112, which is capable of eliciting an effector function.

114. The antibody or antigen-binding fragment of any one of embodiments 1-113, which is capable of eliciting antibody-dependent cell-mediated cytotoxicity.

115. A human or humanized monoclonal antibody, or an antigen-binding fragment thereof, that preferably binds to Ig-like domain 1 in the extracellular domain of human Axl and which exhibits one or more of the following properties:
i) binds to human Axl with an affinity constant (equilibrium dissociation constant) KD of 10 nm or less, preferably 5 nM or less or preferably 1 nM or less as determined by bio-layer interferometry;
ii) binds to human Axl in cells with an EC50 of 1 μg/mL or less, or preferably 0.1 μg/mL or less, as determined by flow cytometry;
iii) inhibits Gas6 binding to human Axl;
iv) inhibits Gas6-dependent phosphorylation of Axl, AKT and ERK;
v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% as determined by a Luminex assay when compared to isotype control;
vi) when tested in vitro increases IL-1RA secretion from human PBMCs by at least 5-fold as determined by ELISA when compared to isotype control;
vii) when tested in vitro increases IL-1RA secretion from human dendritic cells by at least 2-fold as determined by ELISA when compared to isotype control; and
viii) when tested in vitro increases IL-2 secretion from a co-culture of CD4⁺ T cells and dendritic cells by at least 40% as determined by ELISA when compared to isotype control.

116. A composition comprising a therapeutically effective amount of the antibody or antigen-binding fragment of any one of embodiments 1 to 115.

117. A pharmaceutical composition comprising the antibody or antigen-binding fragment of any one of embodiments 1 to 115 and a pharmaceutically acceptable carrier.

118. A polynucleotide comprising one or more nucleotide sequences encoding a VH chain region, a VL chain region, or both a VL chain region and a VH chain region, of an antibody or antigen-binding fragment of any one of embodiments 1 to 115.

119. A polynucleotide comprising one or more nucleotide sequences encoding a heavy chain, a light chain, or both heavy chain and a light chain of an antibody or antigen-binding fragment of any one of embodiments 1 to 115.

120. A population of polynucleotides comprising (i) a first polynucleotide comprising a nucleotide sequence encoding a VH or a heavy chain of the antibody or antigen-binding fragment of any one of embodiments 1 to 115 and (ii) a second polypeptide comprising a nucleotide sequence encoding a VL or a light chain of the antibody or antigen-binding fragment.

121. A vector comprising the polynucleotide of embodiment 118 or 119.

122. A population of vectors comprising (i) a first vector comprising a nucleotide sequence encoding a VH or a heavy chain of the antibody or antigen-binding fragment of any one of embodiments 1 to 115, and (ii) a second vector comprising a nucleotide sequence encoding a VL or a light chain of the antibody or antigen-binding fragment.

123. An isolated cell comprising the polynucleotide of embodiment 118 or 119.

124. An isolated cell comprising the population of polynucleotides of embodiment 120.

125. A population of cells comprising (i) a first host cell comprising a polynucleotide comprising a nucleotide sequence encoding a VH or a heavy chain of the antibody or antigen-binding fragment of any one of embodiments 1 to 115, and (ii) a second host cell comprising a polynucleotide comprising a nucleotide sequence encoding a VL or a light chain of the antibody or antigen-binding fragment.

126. An isolated cell producing the antibody or antigen-binding fragment of any one of embodiments 1 to 115.

127. A kit comprising the antibody or antigen-binding fragment of any one of embodiments 1 to 115.

128. A method of making an antibody or an antigen-binding fragment thereof that specifically binds to human Axl, comprising culturing the cell or population of cells of any one of embodiments 123 to 126 to express the antibody or antigen-binding fragment.

129. A method of making an antibody or an antigen-binding fragment thereof that specifically binds to human Axl, comprising expressing the polynucleotide or population of polynucleotides of any one of embodiments 118 to 120.

130. A method of managing, protecting against, or treating cancer in a subject, comprising administering to a subject in need thereof a therapeutically effective amount of the antibody or antigen-binding fragment of any one of embodiments 1 to 115.

131. A method of enhancing an immune response in a subject comprising administering to a subject in need thereof an effective amount of the antibody or antigen-binding fragment of any one of embodiments 1 to 115.

132. The method of embodiment 131, wherein the subject is an immunocompromised subject.

133. The method of embodiment 132, wherein the immunocompromised subject is suffering from an infection, has cancer, is undergoing, or has had undergone treatment with, an anti-cancer therapy, is HIV positive, or has AIDS or SCID, or diabetes, or has had a transplant and is taking immunosuppressants.

134. The method of embodiment 133, wherein the subject has been treated with an immunosuppressant.

135. A method of enhancing an immune response to a vaccine in a subject, comprising administering to a subject in need thereof, who is or has been administered the vaccine, an effective amount of the antibody or antigen-binding fragment of any one of embodiments 1 to 115.

136. The method of embodiment 135, wherein the vaccine is a cancer or tumor vaccine.

137. A method of managing, preventing, protecting against, or treating metastasis in a subject, comprising administering to a subject in need thereof a therapeutically effective amount of the antibody or antigen-binding fragment of any one of embodiments 1 to 115.

138. A method of managing or treating sepsis in a subject, comprising administering to a subject in need thereof a therapeutically effective amount of the antibody or antigen-binding fragment of any one of embodiments 1 to 115.

139. A method for activating or enhancing an innate immune response in a subject, comprising administering to a subject in need thereof an effective amount of the antibody or antigen-binding fragment of any one of embodiments 1 to 115.

140. The method of embodiment 139, wherein the subject has cancer, or is being treated for cancer with an anti-cancer therapeutic agent, or the subject has an infection.

141. A method of increasing proinflammatory cytokine production in a subject in need thereof, comprising administering to the subject an antibody or antigen-binding fragment thereof that specifically binds to human Axl such that the production of at least one proinflammatory cytokine is increased.

142. The method of embodiment 141, wherein the at least one proinflammatory cytokine is Eotaxin-1, G-CSF, Flt-3L, GM-CSF, Fractalkine, IFNa2, IFNγ, GRO alpha, IL-2, IL-10, MCP-3, IL-12P40, MDC, IL-1RA, IL-1B, IL-4, TNFα, RANTES, MIP-1B, IL-6, IL-8, IP-10, VEGF-A, and/or MIP-1a.

143. A method of increasing proinflammatory cytokine production in a subject in need thereof, comprising administering to the subject an antibody or an antigen-binding fragment thereof of any one of embodiments 1 to 115 such that the production of at least one proinflammatory cytokine is increased.

144. The method of embodiment 143, wherein the at least one proinflammatory cytokine is Eotaxin-1, G-CSF, Flt-3L, GM-CSF, Fractalkine, IFNa2, IFNγ, GRO alpha, IL-2, IL-10, MCP-3, IL-12P40, MDC, IL-1RA, IL-1B, IL-4, TNFα, RANTES, MIP-1B, IL-6, IL-8, IP-10, VEGF-A, and/or MIP-1a.

145. The method of embodiment 143, wherein the at least one proinflammatory cytokine is TNFα.

146. The method of embodiment 143, wherein the at least one proinflammatory cytokine is RANTES.

147. The method of embodiment 143, wherein the at least one proinflammatory cytokine is MIP-1B.

148. The method of embodiment 143, wherein the at least one proinflammatory cytokine is IL-8

149. The method of embodiment 143, wherein the at least one proinflammatory cytokine is MIP-1 a.

150. The method of embodiment 143, wherein the at least one proinflammatory cytokine is IP-10.

151. The method of embodiment 143, wherein the at least one proinflammatory cytokine is IL-1RA.

152. The method of embodiment 143, wherein the at least one proinflammatory cytokine is IL-2.

153. A method of increasing proinflammatory secretory factor production in a subject in need thereof, comprising administering to the subject an antibody or an antigen-binding fragment thereof of any one of embodiments 1 to 115 such that the production of at least one proinflammatory secretory factor is increased.

154. The method of embodiment 153, wherein the production of at least one proinflammatory secretory factor is TNFα, IL-1RA, fibroblast growth factor 2 (FGF-2), eotaxin-1 (CCL11), transforming growth factor alpha (TGF-a) granulocyte-colony stimulating factor (G-CSF), Fms-related tyrosine kinase 3 ligand (Flt-3L), granulocyte macrophage-colony stimulating factor (GM-CSF), fractalkine (CX3CL1), interferon alpha-2 (IFN-a2), interferon-gamma (IFN-γ), growth-regulated oncogene alpha (GRO alpha), interleukin-2 (IL-2), interleukin-10 (IL-10), monocyte chemotactic protein 3 (MCP-3), interleukin-12 p40 (IL-12P40), macrophage-derived chemokine (MDC), platelet-derived growth factor AA homodimer (PDGF-AA), interleukin-13 (IL-13), platelet-derived growth factor BB homodimer (PDGF-BB), soluble CD40 ligand (sCD40L), interleukin-1B (IL-1B), interleukin-4 (IL-4), interleukin-6 (IL-6), interleukin-8 (IL-8), interferon γ-induced protein 10 (IP-10), macrophage inflammatory protein (MIP)-1a, MIP-1β, Regulated on Activation Normal T cell Expressed and Secreted (RANTES), vascular endothelial growth factor A (VEGF-A) and/or IL-18.

155. The method of any one of embodiments 130 to 154, wherein the antibody or antigen-binding fragment thereof does not substantially induce phosphorylation of Axl.

156. The method of any one of embodiments 130 to 155, further comprising administering to the subject a second therapeutic agent.

157. The method of embodiment 156, wherein the second therapeutic agent is an immune checkpoint blockade.

158. The method of embodiment 157, wherein the immune checkpoint blockade inhibits the activity of PD-1, PD-L1, PD-L2, CTLA-4, TIM-3, or LAG-3.

159. The method of embodiment 157, wherein the immune checkpoint blockade inhibits the activity of PD-1.

160. The method of embodiment 157, wherein the immune checkpoint blockade inhibits the activity of PD-L1.

161. The method of any one of embodiments 156 to 160, wherein the second therapeutic agent is a monoclonal antibody.

### 4. BRIEF DESCRIPTION OF THE FIGURES

FIG. 1A depicts the binding curve of Ab301, Ab302, Ab303, Ab304 and Ab305, and isotype controls to human Axl (huAxl) as determined by ELISA. FIG. 1B shows binding of Ab301 to human Axl-ECD and its lack of binding to human MerTK-ECD.

FIG. 2A depicts the binding of Ab301, Ab302, Ab303, Ab304 and Ab305 and isotype control to human Axl (huAxl) expressed on the surface of H1299 cells. FIG. 2B depicts binding of Ab301 to human Axl expressed on the surface of L cells.

FIG. 3 depicts the binding affinity and kinetics parameters of anti-Axl antibody Ab301, Ab302, Ab303, Ab304 and Ab305.

FIG. 4 shows that Ab301 binds to Ig-like domain 1 of the Axl extracellular domain.

FIG. 5A depicts the inhibition of binding of Gas6 binding to human Axl. FIG. 5B shows the inhibition of Gas6 binding of human Axl expressed on the surface of H1299 cells. MFI=mean fluorescence intensity.

FIG. 6 shows that treatment with Ab301 inhibits Gas6-dependent phosphorylation of Axl, AKT and ERK in H1299 cells, as measured by Western Blot.

FIG. 7A depicts analysis of the production of 23 different cytokines, chemokines and growth factors in response to Ab301 in human dendritic cells. Fold increase in protein production in response to Ab301 over isotype control (solid line) is plotted. FIG. 7B depicts analysis of the production of IL-1RA in response to Ab301, Ab302, Ab303, Ab304 or Ab305 in human dendritic cells.

FIG. 8 shows IL-IRA secretion from human peripheral blood mononucleocytes (PBMCs) in response to Ab301.

FIG. 9A shows the effect of Ab302 on Gas6 binding to Axl-expressing cells. FIG. 9B shows the displacement of Axl-bound Gas6 by Ab302.

FIG. 10A shows the induction of p38α phosphorylation by Ab302. FIG. 10B shows the induction of p42/44 phosphorylation by Ab302. FIG. 10C shows the induction of p65 phosphorylation by Ab302. FIG. 10D shows the induction of AKT phosphorylation by Ab302. PBS, phosphate-buffered saline; LPS, lipopolysaccharide. Experiments performed using primary human macrophages.

FIG. 11A shows the induction of antibody-dependent cell-mediated cytotoxicity (ADCC) in Axl-expressing SKOV3 cells in response to Ab302 treatment, as measured by fluorescence-activated cell sorting (FACS).

FIG. 11B shows the induction of antibody-dependent cell-mediated cytotoxicity (ADCC) in Axl-expressing SKOV3 cells in response to Ab302 treatment, as measured by the ADCC Reporter Bioassay (Promega).

FIG. 12 shows the induction of interleukin 2 (IL-2) production by a co-culture of CD4⁺ T cells and dendritic cells in response to Ab302 as measured by ELISA.

### 5. DETAILED DESCRIPTION

Axl is a receptor tyrosine kinase of the TAM group (which includes Tyro-3, Axl, and MerTK) (Lemke and Lu, 2003, Curr Opin Immunol, 2003, 15:31-36; Linger et al., Adv Cancer Res. 2008; 100:35-83; Smart et al., Cancers 2018, 10: 474). In a specific embodiment, Axl is human Axl. UniProtKB P30530 provides an exemplary human Axl amino acid sequence. Native Axl comprises an extracellular domain consisting of two fibronectin type 3-like repeats and two immunoglobulin-like repeats, and an intracellular tyrosine kinase domain (Gay et al., BJC (2017) 116, 415-423).

### 5.1 Antibodies

In a specific aspect, provided herein are antibodies and antigen-binding fragments thereof that specifically bind to human Axl, for example, an extracellular domain (ECD) of human Axl, and modulate Axl expression and/or Axl activity.

In certain embodiments, an antibody or antigen-binding fragment disclosed herein can bind to one or more domains of the extracellular domain of human Axl. In certain embodiments, an antibody or antigen-binding fragment disclosed herein can bind to one or more purified domains of the extracellular domain of human Axl. As a non-limiting example, in certain embodiments, an antibody or antigen-binding fragment disclosed herein can bind to Ig-like domain 1 of the extracellular domain of human Axl. In some embodiments, an antibody or antigen-binding fragment disclosed herein can have diminished binding to one or more purified domains of the extracellular domain of human Axl (e.g., Ig-like domain 2, FnIII-like domain 1 and/or FnIII-like domain 2). In some embodiments, an antibody or antigen-binding fragment disclosed herein can bind to a purified Ig-like domain D 1 of the extracellular domain of human Axl but does not bind to, or exhibits reduced binding to, a purified Ig-like domain 2, FnIII-like domain 1, and/or FnIII-like domain 2 of the extracellular domain of human Axl.

An antibody or antigen-binding fragment (for example, a human or humanized antibody or antigen-binding fragment) presented herein that specifically binds to Axl (e.g., human Axl) can, for example, exhibit one or more of the following properties: i) binds to Ig-like domain 1 of the extracellular domain of human Axl; ii) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; iii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iv) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; v) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; vi) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; vii) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; viii) when tested in vitro increases IL-1RA secretion from human dendritic cells by at least 2-fold (or at least 3-fold) as determined by ELISA when compared to isotype control; ix) when tested in vitro increases IL-2 secretion from a co-culture of CD4⁺ T cells and dendritic cells by at least 40% (e.g., at a concentration of 30 nM or higher) as determined by ELISA when compared to isotype control; and x) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, preferably bind to Ig-like domain 1 of the extracellular domain of human Axl and exhibit one or more of the following properties: i) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; ii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iv) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex assay when compared to isotype control; vi) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vii) when tested in vitro increases IL-1RA secretion from human dendritic cells by at least 2-fold (or at least 3-fold) as determined by ELISA when compared to isotype control; viii) when tested in vitro increases IL-2 secretion from a co-culture of CD4⁺ T cells and dendritic cells by at least 40% (e.g., at a concentration of 30 nM or higher) as determined by ELISA when compared to isotype control; and ix) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, bind to Ig-like domain 1 of the extracellular domain of human Axl and exhibit one or more of the following properties: i) binds to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry; ii) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; iii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iv) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex ® assay when compared to isotype control; vi) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vii) when tested in vitro increases IL-1RA secretion from human dendritic cells by at least 2-fold (or at least 3-fold) as determined by ELISA when compared to isotype control; viii) when tested in vitro increases IL-2 secretion from a co-culture of CD4⁺ T cells and dendritic cells by at least 40% (e.g., at a concentration of 30 nM or higher) as determined by ELISA when compared to isotype control; and ix) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, preferably bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, and exhibit one or more of the following properties: i) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; ii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iii) inhibits Gas6-dependent phosphorylation of Axl, AKT and ERK (for example, in a H1299 lung cancer cell line) (e.g., by 50% or more, 60% or more, 70% or more, 80% or more, 90% or more, 95% or more, or 98% or more), for example, as determined by western blot; iv) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; v) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vi) when tested in vitro increases IL-1RA secretion from human dendritic cells by at least 2-fold (or at least 3-fold) as determined by ELISA when compared to isotype control; vii) when tested in vitro increases IL-2 secretion from a co-culture of CD4⁺ T cells and dendritic cells by at least 40% (e.g., at a concentration of 30 nM or higher) as determined by ELISA when compared to isotype control; and viii) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, and exhibit one or more of the following properties: i) binds to human Axl on cells with an EC50 of 1 μg/mL or less (for example, 0.5 μg/mL or less), or preferably 0.1 μg/mL or less (for example, 0.05 μg/mL, or less) as determined by flow cytometry; ii) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; iii) inhibits Gas6-dependent phosphorylation of Axl, AKT and ERK (for example, in a H1299 lung cancer cell line) (e.g., by 50% or more, 60% or more, 70% or more, 80% or more, 90% or more, 95% or more, or 98% or more), for example, as determined by western blot; iv) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; v) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; vi) when tested in vitro increases IL-1RA secretion from human dendritic cells by at least 2-fold (or at least 3-fold) as determined by ELISA when compared to isotype control; vii) when tested in vitro increases IL-2 secretion from a co-culture of CD4⁺ T cells and dendritic cells by at least 40% (e.g., at a concentration of 30 nM or higher) as determined by ELISA when compared to isotype control; and viii) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, preferably bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, bind to human Axl on cells with an EC50 of 1 μg/mL or less (for example, 0.5 μg/mL or less), or preferably 0.1 μg/mL or less (for example, 0.05 μg/mL, or less) as determined by flow cytometry, and exhibit one or more of the following properties: i) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; ii) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; iii) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex ® assay when compared to isotype control; iv) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; v) when tested in vitro increases IL-1RA secretion from human dendritic cells by at least 2-fold (or at least 3-fold) as determined by ELISA when compared to isotype control; vi) when tested in vitro increases IL-2 secretion from a co-culture of CD4⁺ T cells and dendritic cells by at least 40% (e.g., at a concentration of 30 nM or higher) as determined by ELISA when compared to isotype control; and vii) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, bind to human Axl on cells with an EC50 of 1 μg/mL or less (for example, 0.5 μg/mL or less), or preferably 0.1 μg/mL or less (for example, 0.05 μg/mL, or less) as determined by flow cytometry, and exhibit one or more of the following properties: i) inhibits Gas6 binding to human Axl (e.g., by 50% or more, 60% or more, 70% or more, 80% or more, 90% or more, 95% or more, or 98% or more), for example, as determined by an ELISA assay or a flow cytometry assay; ii) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; iii) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; iv) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; v) when tested in vitro increases IL-1RA secretion from human dendritic cells by at least 2-fold (or at least 3-fold) as determined by ELISA when compared to isotype control; vi) when tested in vitro increases IL-2 secretion from a co-culture of CD4⁺ T cells and dendritic cells by at least 40% (e.g., at a concentration of 30 nM or higher) as determined by ELISA when compared to isotype control; and vii) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, preferably bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, bind to human Axl on cells with an EC50 of 1 μg/mL or less (for example, 0.5 μg/mL or less), or preferably 0.1 μg/mL or less (for example, 0.05 μg/mL, or less) as determined by flow cytometry, inhibit Gas6 binding to human Axl (e.g., by 50% or more, 60% or more, 70% or more, 80% or more, 90% or more, 95% or more, or 98% or more), for example, as determined by an ELISA assay or a flow cytometry assay, and exhibit one or more of the following properties: i) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; ii) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; iii) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; iv) when tested in vitro increases IL-1RA secretion from human dendritic cells by at least 2-fold (or at least 3-fold) as determined by ELISA when compared to isotype control; v) when tested in vitro increases IL-2 secretion from a co-culture of CD4⁺ T cells and dendritic cells by at least 40% (e.g., at a concentration of 30 nM or higher) as determined by ELISA when compared to isotype control; and vi) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, bind to human Axl on cells with an EC50 of 1 μg/mL or less (for example, 0.5 μg/mL or less), or preferably 0.1 μg/mL or less (for example, 0.05 μg/mL, or less) as determined by flow cytometry, inhibit Gas6 binding to human Axl (e.g., by 50% or more, 60% or more, 70% or more, 80% or more, 90% or more, 95% or more, or 98% or more), for example, as determined by an ELISA assay or a flow cytometry assay, and exhibit one or more of the following properties: i) inhibits Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot; ii) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; iii) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; iv) when tested in vitro increases IL-1RA secretion from human dendritic cells by at least 2-fold (or at least 3-fold) as determined by ELISA when compared to isotype control; v) when tested in vitro increases IL-2 secretion from a co-culture of CD4⁺ T cells and dendritic cells by at least 40% (e.g., at a concentration of 30 nM or higher) as determined by ELISA when compared to isotype control; and vi) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, preferably bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, bind to human Axl on cells with an EC50 of 1 μg/mL or less (for example, 0.5 μg/mL or less), or preferably 0.1 μg/mL or less (for example, 0.05 μg/mL, or less) as determined by flow cytometry, inhibit Gas6 binding to human Axl (e.g., by 50% or more, 60% or more, 70% or more, 80% or more, 90% or more, 95% or more, or 98% or more), for example, as determined by an ELISA assay or a flow cytometry assay, inhibit Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot, and exhibit one or more of the following properties: i) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; ii) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; iii) when tested in vitro increases IL-1RA secretion from human dendritic cells by at least 2-fold (or at least 3-fold) as determined by ELISA when compared to isotype control; iv) when tested in vitro increases IL-2 secretion from a co-culture of CD4⁺ T cells and dendritic cells by at least 40% (e.g., at a concentration of 30 nM or higher) as determined by ELISA when compared to isotype control; and v) when tested in vitro increases IL-2 secretion from a co-culture of CD4⁺ T cells and dendritic cells by at least 80% (e.g., at a concentration of 100 nM or higher) as determined by ELISA when compared to isotype control.

An antibody or antigen-binding fragment (for example, a human or humanized antibody or antigen-binding fragment) presented herein that specifically binds to Axl (e.g., human Axl) can, for example, bind to Ig-like domain 1 of the extracellular domain of human Axl, bind to human Axl with an affinity constant (equilibrium dissociation constant) KD of 10 nM or less (for example 5 nM or less, or 2 nM or less), or preferably 1 nM or less (for example, 0.5 nM or less) as determined by bio-layer interferometry, bind to human Axl on cells with an EC50 of 1 μg/mL or less (for example, 0.5 μg/mL or less), or preferably 0.1 μg/mL or less (for example, 0.05 μg/mL, or less) as determined by flow cytometry, inhibit Gas6 binding to human Axl (e.g., by 50% or more, 60% or more, 70% or more, 80% or more, 90% or more, 95% or more, or 98% or more), for example, as determined by an ELISA assay or a flow cytometry assay, inhibit Gas6-dependent phosphorylation of Axl, AKT and ERK(for example, in a H1299 lung cancer cell line) (e.g., by 50% or more, 60% or more, 70% or more, 80% or more, 90% or more, 95% or more, or 98% or more), for example, as determined by western blot, and exhibit one or more of the following properties: i) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% (or at least 2-fold, at least 3-fold, at least 4-fold, at least 5-fold, at least 10-fold, at least 20-fold, at least 30-fold, or at least 40-fold) as determined by a Luminex® assay when compared to isotype control; ii) when tested in vitro increases IL-1RA secretion from human PBCMs by at least 5-fold (or at least 2-fold, at least 3-fold, at least 4-fold, at least 6-fold, at least 7-fold, at least 8-fold) as determined by ELISA when compared to isotype control; iii) when tested in vitro increases IL-1RA secretion from human dendritic cells by at least 2-fold (or at least 3-fold) as determined by ELISA when compared to isotype control; iv) when tested in vitro increases IL-2 secretion from a co-culture of CD4⁺ T cells and dendritic cells by at least 40% (e.g., at a concentration of 30 nM or higher) as determined by ELISA when compared to isotype control; and v) when tested in vitro increases IL-2 secretion from a co-culture of CD4⁺ T cells and dendritic cells by at least 80% (e.g., at a concentration of 100 nM or higher) as determined by ELISA when compared to isotype control.

As used herein, “Luminex® assay” and “Luminex assay” are interchangeable terms and refer to an immunoassay performed using the well known Luminex® technology.

An antibody or antigen-binding fragment presented herein can, for example, be antibody Ab301 or an antigen-binding fragment thereof, Ab302 or an antigen-binding fragment thereof, Ab303 or an antigen-binding fragment thereof, Ab304 or an antigen-binding fragment thereof, or Ab305 or an antigen-binding fragment thereof. An antibody or antigen-binding fragment presented herein can comprise CDRs of any of Table 1, Table 4, Table 6, Table 8, Table 10, or Table 12. An antibody or an antibody or antigen-binding fragment presented herein can comprise a VH and/or VL of Table 3. An antibody or antigen-binding fragment thereof presented herein can comprise Framework Regions (FRs) of any one of Table 2, Table 5, Table 7, Table 9, Table 11 or Table 13.

As used herein, the terms “antibody” and “immunoglobulin” and “Ig” are terms of art and can be used interchangeably herein, and refer to a molecule with an antigen binding site that specifically binds an antigen.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)₂ fragments, disulfide-linked Fvs (sdFv), anti-idiotypic (anti-Id) antibodies (including, e.g., anti-anti-Id antibodies), and epitope-binding fragments of any of the above. In certain embodiments, antibodies described herein refer to polyclonal antibody populations.

In a certain embodiment, an isolated antibody (e.g., monoclonal antibody) described herein, or an antigen-binding fragment thereof, which specifically binds to human Axl, is fused to a heterologous polypeptide. In a certain embodiment, an isolated antibody (e.g., monoclonal antibody) described herein, or an antigen-binding fragment thereof, which specifically binds to human Axl, is conjugated to an agent. In a particular embodiment, the agent is a toxin. In one embodiment, the toxin is abrin, ricin A, pseudomonas exotoxin, cholera toxin, or diphtheria toxin.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.

As used herein, an “antigen” is a moiety or molecule that contains an epitope to which an antibody can specifically bind. As such, an antigen is also is specifically bound by an antibody. In a specific embodiment, the antigen, to which an antibody described herein binds is Axl (e.g., human Axl), or a fragment thereof, for example, an extracellular domain of Axl (e.g., human Axl).

As used herein, an “epitope” is a term known in the art and refers to a localized region of an antigen to which an antibody can specifically bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

As used herein, the terms “specifically binds,” “specifically recognizes,” “immunospecifically binds,” “immunospecifically recognizes” and “immunospecific” are analogous terms in the context of antibodies and refer to molecules that bind to an antigen (e.g., epitope) as such binding is understood by one skilled in the art. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, Id.), or other assays known in the art. In a specific embodiment, molecules that specifically bind to an antigen bind to the antigen with a Kₐ that is at least 2 logs, 2.5 logs, 3 logs, 4 logs or greater than the Kₐ when the molecules bind to another antigen. In another specific embodiment, molecules that specifically bind to an antigen do not cross react with other proteins. In another specific embodiment, molecules that specifically bind to an antigen do not cross react with other non-Axl proteins.

As used herein, the term “constant region” or “constant domain” is a well-known antibody term of art (sometimes referred to as “Fc”), and refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

As used herein, the term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, an “isolated” or “purified” antibody or antigen binding fragment is substantially free of cellular material or other contaminating proteins from the cell or tissue source from which the antibody or antigen binding fragment is derived, or substantially free of chemical precursors or other chemicals when the antibody or antigen binding fragment is chemically synthesized.

As used herein, the term “light chain” when used in reference to an antibody can refer to any distinct types, e.g., kappa (κ) of lambda (λ) based on the amino acid sequence of the constant domains. Light chain amino acid sequences are well known in the art. In specific embodiment is a human light chain.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to a Axl epitope (e.g., an epitope of the extracellular domain of human Axl) as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

As used herein, the term “polyclonal antibodies” refers to an antibody population that includes a variety of different antibodies that specifically bind to the same and/or to different epitopes within an antigen or antigens.

As used herein, the terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, M.-P., 1999, The Immunologist, 7:132-136 and Lefranc, M.-P. et al., 1999, Nucleic Acids Res., 27:209-212 (“IMGT CDRs”); or (iv) the AbM numbering system, which will be referred to herein as the “AbM CDRs”, for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., (1996) J Mol Biol 5:732-745)).

In certain embodiments, an Axl-binding antibody, or antigen-binding fragment thereof, described herein comprises CDRs, or CDRs and FRs, of antibody Ab301, Ab302, Ab303, Ab304, Ab305, or Abcon, as determined by the Kabat numbering system.

In certain embodiments, provided herein is an antibody or an antigen-binding fragment thereof that specifically binds to human Axl, which comprises VL and/or VH CDRs as set forth in Table 1, which utilizes standard one letter amino acid abbreviations.

TABLE 1
VL and VH CDR Amino Acid Sequences (Kabat)

| | | CDR1 | CDR2 | CDR3 |
|---|---|---|---|---|
| Ab301 | VH | DYWME | EIRNKVNNYA | LRLFAY |
| | | (SEQ ID | TYYGKSVKG | (SEQ ID |
| | | NO: 20) | (SEQ ID | NO: 22) |
| | | | NO: 21) | |
| | VL | RSSKSLLH | RMSTLAS | GQLLENPYT |
| | | RNGITYVY | (SEQ ID | (SEQ ID |
| | | (SEQ ID | NO: 31) | NO: 32) |
| | | NO: 38) | | |
| Ab302 | VH | DYWME | EIRNKVNNYA | LRLFAY |
| | | (SEQ ID | TYYGKSVKG | (SEQ ID |
| | | NO: 20) | (SEQ ID | NO: 22) |
| | | | NO: 21) | |
| | VL | RSSKSLLH | RMSTLAS | GQLLENPYT |
| | | RQGITYVY | (SEQ ID | (SEQ ID |
| | | (SEQ ID | NO: 31) | NO: 32) |
| | | NO: 30) | | |
| Ab303 | VH | DYWME | EIRNKVNNYA | LRLFAY |
| | | (SEQ ID | TYYGKSVKG | (SEQ ID |
| | | NO: 20) | (SEQ ID | NO: 22) |
| | | | NO: 21) | |
| | VL | RSSKSLLH | RMSTLAS | GQLLENPYT |
| | | RQGITYVY | (SEQ ID | (SEQ ID |
| | | (SEQ ID | NO: 31) | NO: 32) |
| | | NO: 30) | | |
| Ab304 | VH | DYWME | EIRNKVNNYA | LRLFAY |
| | | (SEQ ID | TYYGKSVKG | (SEQ ID |
| | | NO: 20) | (SEQ ID | NO: 22) |
| | | | NO: 21) | |
| | VL | RSSKSLLH | RMSTLAS | GQLLENPYT |
| | | RQGITYVY | (SEQ ID | (SEQ ID |
| | | (SEQ ID | NO: 31) | NO: 32) |
| | | NO: 30) | | |
| Ab305 | VH | DYWME | EIRNKVNNYA | LRLFAY |
| | | (SEQ ID | TYYGKSVKG | (SEQ ID |
| | | NO: 20) | (SEQ ID | NO: 22) |
| | | | NO: 21) | |
| | VL | RSSKSLLH | RMSTLAS | GQLLENPYT |
| | | RQGITYVY | (SEQ ID | (SEQ ID |
| | | (SEQ ID | NO: 31) | NO: 32) |
| | | NO: 30) | | |
| Abcon | VH | DYWME | EIRNKVNNYA | LRLFAY |
| | | (SEQ ID | TYYGKSVKG | (SEQ ID |
| | | NO: 20) | (SEQ ID | NO: 22) |
| | | | NO: 21) | |
| | VL | RSSKSLLH | RMSTLAS | GQLLENPYT |
| | | RQGITYVY | (SEQ ID | (SEQ ID |
| | | (SEQ ID | NO: 31) | NO: 32) |
| | | NO: 30) | | |

In certain embodiments, the antibody or an antigen-binding fragment thereof that specifically binds to human Axl further comprises VL and/or VH framework regions (FRs) as set forth in Table 2, which utilizes standard one letter amino acid abbreviations. It will be clear to one of ordinary skill in the art, that the VL and VH of the antibody or antigen-binding fragment thereof provided herein comprise the CDRs and FRs in the order of FR1-CDR1-FR2-CDR2-FR3-CDR3-FR4.

TABLE 2
VL and VH Framework Amino Acid Sequences (Kabat)

| | | FR1 | FR2 | FR3 | FR4 |
|---|---|---|---|---|---|
| Ab301 | VH | EVKLEASGGGLVQPGMS | WVRQAPGKGLEWVA | RFTISRDDSKSIVYLQMNS | WGQGTLVTVSA |
| | | VKLSCTTSGFTFS | (SEQ ID NO: 61) | IRSEDTGIYYCNT | (SEQ ID NO: 93) |
| | | (SEQ ID NO: 100) | | (SEQ ID NO: 101) | |
| | VL | DIVMTQAPLSISVTPGES | WYLQKPGKSPQLLIY | GVPDRFSGSGSETDFTLKI | FGAGTRLEIK |
| | | PSMSC | (SEQ ID NO: 108) | NKVEAEDVGIYYC | (SEQ ID NO: 110) |
| | | (SEQ ID NO: 107) | | (SEQ ID NO: 109) | |
| Ab302 | VH | EVQLVESGGGLVKPGGS | WVRQAPGKGLEWVA | RFTISRDDSKNTLYLQMN | WGQGTLVTVSS |
| | | LKLSCAASGFTFS | (SEQ ID NO: 61) | SLKTEDTAVYYCNT | (SEQ ID NO: 63) |
| | | (SEQ ID NO: 60) | | (SEQ ID NO: 62) | |
| | VL | DIQMTQSPSSLSASVGDR | WYQQKPGKVPKLLIY | GVPSRFSGSGSGTDFTLTI | FGQGTKLEIK |
| | | VTITC | (SEQ ID NO: 53) | SSLQPEDVATYYC | (SEQ ID NO: 55) |
| | | (SEQ ID NO: 52) | | (SEQ ID NO: 54) | |
| Ab303 | VH | EVQLVESGGGVVQPGRS | WVRQAPGKGLEWVA | RFTISRDNSKNTLYLQMN | WGQGTLVTVSS |
| | | LRLSCAASGFTFS | (SEQ ID NO: 61) | SLRAEDTAVYYCNT | (SEQ ID NO: 63) |
| | | (SEQ ID NO: 71) | | (SEQ ID NO: 72) | |
| | VL | DIQMTQSPSSLSASVGDR | WYQQKPGKVPKLLIY | GVPSRFSGSGSGTDFTLTI | FGQGTKLEIK |
| | | VTITC | (SEQ ID NO: 53) | SSLQPEDVATYYC | (SEQ ID NO: 55) |
| | | (SEQ ID NO: 52) | | (SEQ ID NO: 54) | |
| Ab304 | VH | EVQLVESGGGLVQPGGS | WVRQAPGKGLEWVG | RFTISRDESKNSLYLQMN | WGQGTTVTVSS |
| | | LRLSCAASGFTFS | (SEQ ID NO: 80) | SLKTEDTAVYYCNT | (SEQ ID NO: 82) |
| | | (SEQ ID NO: 79) | | (SEQ ID NO: 81) | |
| | VL | DIQMTQSPSSLSASVGDR | WYQQKPGKVPKLLIY | GVPSRFSGSGSGTDFTLTI | FGQGTKLEIK |
| | | VTITC | (SEQ ID NO: 53) | SSLQPEDVATYYC | (SEQ ID NO: 55) |
| | | (SEQ ID NO: 52) | | (SEQ ID NO: 54) | |
| Ab305 | VH | EVQLVESGGGLVQPGRS | WVRQAPGKGLEWVG | RFTISRDDSKSIAYLQMNS | WGQGTLVTVSA |
| | | LRLSCTASGFTFS | (SEQ ID NO: 80) | LKTEDTAVYYCNT | (SEQ ID NO: 93) |
| | | (SEQ ID NO: 91) | | (SEQ ID NO: 92) | |
| | VL | DIQMTQSPSSLSASVGDR | WYQQKPGKVPKLLIY | GVPSRFSGSGSGTDFTLTI | FGQGTKLEIK |
| | | VTITC | (SEQ ID NO: 53) | SSLQPEDVATYYC | (SEQ ID NO: 55) |
| | | (SEQ ID NO: 52) | | (SEQ ID NO: 54) | |
| Abcon | VH | EVQLVESGGGX₁VX₂PGX₃ | WVRQAPGKGLEWVX₆ | RFTISRDX₇SKX₈X₉X₁₀YLQ | WGQGTX₁₃VTVSX₁₄ |
| | | SLX₄LSCX₅ASGFTFS | (X₆ is an amino acid | MNSLX₁₁X₁₂EDTAVYYCNT | (X₁₃ is L or T, |
| | | (X₁ is an amino acid with an | with an aliphatic | (X₇ is D, N or E, X₈ is an | and X₁₄ is |
| | | aliphatic side chain (for | side chain (for | amino acid with a polar side | S or A) |
| | | example, L or V), X₂ is K or | example, A or G)) | chain (for example, N or S), | (SEQ ID NO: 43) |
| | | Q, X₃ is G or R, X₄ is an | (SEQ ID NO: 41) | X₉ is T, S or I, X₁₀ is an | |
| | | amino acid with a positively | | amino acid with an aliphatic | |
| | | charged side chain (for | | or hydrophobic side chain | |
| | | example, K or R), X₅ is A or T) | | (for example, L or A), X₁₁ | |
| | | (SEQ ID NO: 40) | | is an amino acid with a | |
| | | | | positively charged side | |
| | | | | chain (for example, K or | |
| | | | | R), X₁₂ is T or A) | |
| | | | | (SEQ ID NO: 42) | |
| | VL | DIQMTQSPSSLSASVGDR | WYQQKPGKVPKLLIY | GVPSRFSGSGSGTDFTLTI | FGQGTKLEIK |
| | | VTITC | (SEQ ID NO: 53) | SSLQPEDVATYYC | (SEQ ID NO: 55) |
| | | (SEQ ID NO: 52) | | (SEQ ID NO: 54) | |

In certain embodiments, provided herein is an antibody or an antigen-binding fragment thereof that specifically binds to human Axl and comprises a VL and/or a VH as set forth in Table 3, which utilizes standard one letter amino acid abbreviations.

In a particular embodiment, provided herein is an antibody or an antigen-binding fragment thereof that specifically binds to human Axl, and comprises VL (SEQ ID NO: 12) and VH (SEQ ID NO: 11). For ease of description, an antibody that comprises the VL and VH amino acid sequences of SEQ ID NOS: 12 and 11, respectively, is referred to herein as “Ab301.” In one embodiment, such an antibody or antigen-binding fragment comprises a separate light chain comprising the VL amino acid sequence and a separate heavy chain comprising the VH amino acid sequence. In another embodiment, such an antibody or antigen-binding fragment comprises a single chain comprising the VL amino acid sequence and the VH amino acid sequence.

In another particular embodiment, provided herein is an antibody or an antigen-binding fragment thereof that specifically binds to human Axl, and comprises VL (SEQ ID NO: 6) and VH (SEQ ID NO: 7). For ease of description, an antibody that comprises the VL and VH amino acid sequences of SEQ ID NOS: 6 and 7, respectively, is referred to herein as “Ab302.” In one embodiment, such an antibody or antigen-binding fragment comprises a separate light chain comprising the VL amino acid sequence and a separate heavy chain comprising the VH amino acid sequence. In another embodiment, such an antibody or antigen-binding fragment comprises a single chain comprising the VL amino acid sequence and the VH amino acid sequence.

In another particular embodiment, provided herein is an antibody or an antigen-binding fragment thereof that specifically binds to human Axl, and comprises VL (SEQ ID NO: 6) and VH (SEQ ID NO: 8). For ease of description, an antibody that comprises the VL and VH amino acid sequences of SEQ ID NOS: 6 and 8, respectively, is referred to herein as “Ab303.” In one embodiment, such an antibody or antigen-binding fragment comprises a separate light chain comprising the VL amino acid sequence and a separate heavy chain comprising the VH amino acid sequence. In another embodiment, such an antibody or antigen-binding fragment comprises a single chain comprising the VL amino acid sequence and the VH amino acid sequence.

In another particular embodiment, provided herein is an antibody or an antigen-binding fragment thereof that specifically binds to human Axl, and comprises VL (SEQ ID NO: 6) and VH (SEQ ID NO: 9). For ease of description, an antibody that comprises the VL and VH amino acid sequences of SEQ ID NOS: 6 and 9, respectively, is referred to herein as “Ab304.” In one embodiment, such an antibody or antigen-binding fragment comprises a separate light chain comprising the VL amino acid sequence and a separate heavy chain comprising the VH amino acid sequence. In another embodiment, such an antibody or antigen-binding fragment comprises a single chain comprising the VL amino acid sequence and the VH amino acid sequence.

In another particular embodiment, provided herein is an antibody or an antigen-binding fragment thereof that specifically binds to human Axl, and comprises VL (SEQ ID NO: 6) and VH (SEQ ID NO: 10). For ease of description, an antibody that comprises the VL and VH amino acid sequences of SEQ ID NOS: 6 and 10, respectively, is referred to herein as “Ab305.” In one embodiment, such an antibody or antigen-binding fragment comprises a separate light chain comprising the VL amino acid sequence and a separate heavy chain comprising the VH amino acid sequence. In another embodiment, such an antibody or antigen-binding fragment comprises a single chain comprising the VL amino acid sequence and the VH amino acid sequence.

In another particular embodiment, provided herein is an antibody or an antigen-binding fragment thereof that specifically binds to human Axl, and comprises VL (SEQ ID NO: 6) and VH (SEQ ID NO: 5). For ease of description, an antibody that comprises the VL and VH amino acid sequences of SEQ ID NOS: 6 and 5, respectively, is referred to herein as “Abcon” (a person of ordinary skill in the art would understand that an Abcon antibody can also be, in certain embodiments, an Ab302, Ab303, Ab304, or Ab305 antibody. In one embodiment, such an antibody or antigen-binding fragment comprises a separate light chain comprising the VL amino acid sequence and a separate heavy chain comprising the VH amino acid sequence. In another embodiment, such an antibody or antigen-binding fragment comprises a single chain comprising the VL amino acid sequence and the VH amino acid sequence.

In a specific embodiment, provided herein is an antibody or an antigen-binding fragment thereof that specifically binds to human Axl and comprises the VL of Ab301 as set forth in Table 3. In another specific embodiment, provided herein is an antibody or an antigen-binding fragment thereof that specifically binds to human Axl and comprises the VL of Ab302-Ab305 and Abcon as set forth in Table 3.

In a specific embodiment, provided herein is an antibody or an antigen-binding fragment thereof that specifically binds to human Axl and comprises the VH of Ab301 as set forth in Table 3. In another specific embodiment, provided herein is an antibody or an antigen-binding fragment thereof that specifically binds to human Axl and comprises the VH of Ab302 as set forth in Table 3. In another specific embodiment, provided herein is an antibody or an antigen-binding fragment thereof that specifically binds to human Axl and comprises the VH of Ab303 as set forth in Table 3. In another specific embodiment, provided herein is an antibody or an antigen-binding fragment thereof that specifically binds to human Axl and comprises the VH of Ab304 as set forth in Table 3. In another specific embodiment, provided herein is an antibody or an antigen-binding fragment thereof that specifically binds to human Axl and comprises the VH of Ab305 as set forth in Table 3. In another specific embodiment, provided herein is an antibody or an antigen-binding fragment thereof that specifically binds to human Axl and comprises the VH of Abcon as set forth in Table 3.

TABLE 3
Ab301-Ab305 and Abcon VL and
VH Amino Acid Sequences

| | | Amino Acid Sequence |
|---|---|---|
| Ab301 | VH | EVKLEASGGGLVQPGMSVKLSCTTSGFTFSDYWMEWVR |
| | | QAPGKGLEWVAEIRNKVNNYATYYGKSVKGRFTISRDD |
| | | SKSIVYLQMNSIRSEDTGIYYCNTLRLFAYWGQGTLVT |
| | | VSA (SEQ ID NO: 11) |
| | VL | DIVMTQAPLSISVTPGESPSMSCRSSKSLLHRNGITYV |
| | | YWYLQKPGKSPQLLIYRMSTLASGVPDRFSGSGSETDF |
| | | TLKINKVEAEDVGIYYCGQLLENPYTFGAGTRLEIK |
| | | (SEQ ID NO: 12) |
| Ab302 | VH | EVQLVESGGGLVKPGGSLKLSCAASGFTFSDYWMEWVR |
| | | QAPGKGLEWVAEIRNKVNNYATYYGKSVKGRFTISRDD |
| | | SKNTLYLQMNSLKTEDTAVYYCNTLRLFAYWGQGTLVT |
| | | VSS (SEQ ID NO: 7) |
| | VL | DIQMTQSPSSLSASVGDRVTITCRSSKSLLHRQGITYV |
| | | YWYQQKPGKVPKLLIYRMSTLASGVPSRFSGSGSGTDF |
| | | TLTISSLQPEDVATYYCGQLLENPYTFGQGTKLEIK |
| | | (SEQ ID NO: 6) |
| Ab303 | VH | EVQLVESGGGVVQPGRSLRLSCAASGFTFSDYWMEWVR |
| | | QAPGKGLEWVAEIRNKVNNYATYYGKSVKGRFTISRDN |
| | | SKNTLYLQMNSLRAEDTAVYYCNTLRLFAYWGQGTLVT |
| | | VSS (SEQ ID NO: 8) |
| | VL | DIQMTQSPSSLSASVGDRVTITCRSSKSLLHRQGITYV |
| | | YWYQQKPGKVPKLLIYRMSTLASGVPSRFSGSGSGTDF |
| | | TLTISSLQPEDVATYYCGQLLENPYTFGQGTKLEIK |
| | | (SEQ ID NO: 6) |
| Ab304 | VH | EVQLVESGGGLVQPGGSLRLSCAASGFTFSDYWMEWVR |
| | | QAPGKGLEWVGEIRNKVNNYATYYGKSVKGRFTISRDE |
| | | SKNSLYLQMNSLKTEDTAVYYCNTLRLFAYWGQGTTVT |
| | | VSS (SEQ ID NO: 9) |
| | VL | DIQMTQSPSSLSASVGDRVTITCRSSKSLLHRQGITYV |
| | | YWYQQKPGKVPKLLIYRMSTLASGVPSRFSGSGSGTDF |
| | | TLTISSLQPEDVATYYCGQLLENPYTFGQGTKLEIK |
| | | (SEQ ID NO: 6) |
| Ab305 | VH | EVQLVESGGGLVQPGRSLRLSCTASGFTFSDYWMEWVR |
| | | QAPGKGLEWVGEIRNKVNNYATYYGKSVKGRFTISRDD |
| | | SKSIAYLQMNSLKTEDTAVYYCNTLRLFAYWGQGTLVT |
| | | VSA (SEQ ID NO: 10) |
| | VL | DIQMTQSPSSLSASVGDRVTITCRSSKSLLHRQGITYV |
| | | YWYQQKPGKVPKLLIYRMSTLASGVPSRFSGSGSGTDF |
| | | TLTISSLQPEDVATYYCGQLLENPYTFGQGTKLEIK |
| | | (SEQ ID NO: 6) |
| Abcon | VH | EVQLVESGGGX₁VX₂PGX₃SLX₄LSCX₅ASGFTFSDYWME |
| | | WVRQAPGKGLEWVX₆EIRNKVNNYATYYGKSVKGRFTI |
| | | SRDX₇SKX₈X₉X₁₀YLQMNSLX₁₁X₁₂EDTAVYYCNTLRLF |
| | | AYWGQGTX₁₃VTVSX₁₄ |
| | | (X₁ is an amino acid with an aliphatic |
| | | side chain (for example, L or V), X₂ |
| | | is K or Q, X₃ is G or R, X₄ is an amino |
| | | acid with a positively charged side |
| | | chain (for example, K or R), X₅ is A or |
| | | T, X₆ is an amino acid with an aliphatic |
| | | side chain (for example, A or G), X₇ is |
| | | D, N or E, X₈ is an amino acid with a |
| | | polar side chain (for example, N or S), |
| | | X₉ is T, S or I, X₁₀ is an amino acid with |
| | | an aliphatic or hydrophobic side chain |
| | | (for example, L or A), X₁₁ is an amino |
| | | acid with a positively charged side chain |
| | | (for example, K or R), X₁₂ is T or A, X₁₃ |
| | | is L or T, and X₁₄ is S or A) |
| | | (SEQ ID NO: 5) |
| | VL | DIQMTQSPSSLSASVGDRVTITCRSSKSLLHRQGITYVYWY |
| | | QQKPGKVPKLLIYRMSTLASGVPSRFSGSGSGTDFTLTISS |
| | | LQPEDVATYYCGQLLENPYTFGQGTKLEIK |
| | | (SEQ ID NO: 6) |

In certain embodiments, an Axl-binding antibody, or antigen-binding fragment thereof, described herein comprises CDRs, or CDRs and FRs, of antibody Ab301, Ab302, Ab303, Ab304, Ab305, or Abcon, as determined by the IMGT (Immunogenetics) numbering system.

In certain embodiments, provided herein is an antibody or an antigen-binding fragment thereof that specifically binds to human Axl, which comprises VL and/or VH CDR as set forth in Table 4, which utilizes standard one letter amino acid abbreviations.

TABLE 4
VL and VH CDR Amino Acid Sequences (IMGT)

| | | CDR1 | CDR2 | CDR3 |
|---|---|---|---|---|
| Ab301 | VH | GFTFSDYW | IRNKVNNYAT | NTLRLFAY |
| | | (SEQ ID NO: 120) | (SEQ ID NO: 121) | (SEQ ID NO: 122) |
| | VL | KSLLHRNGITY | RMS | GQLLENPYT |
| | | (SEQ ID NO: 130) | (SEQ ID NO: 124) | (SEQ ID NO: 32) |
| Ab302 | VH | GFTFSDYW | IRNKVNNYAT | NTLRLFAY |
| | | (SEQ ID NO: 120) | (SEQ ID NO: 121) | (SEQ ID NO: 122) |
| | VL | KSLLHRQGITY | RMS | GQLLENPYT |
| | | (SEQ ID NO: 123) | (SEQ ID NO: 124) | (SEQ ID NO: 32) |
| Ab303 | VH | GFTFSDYW | IRNKVNNYAT | NTLRLFAY |
| | | (SEQ ID NO: 120) | (SEQ ID NO: 121) | (SEQ ID NO: 122) |
| | VL | KSLLHRQGITY | RMS | GQLLENPYT |
| | | (SEQ ID NO: 123) | (SEQ ID NO: 124) | (SEQ ID NO: 32) |
| Ab304 | VH | GFTFSDYW | IRNKVNNYAT | NTLRLFAY |
| | | (SEQ ID NO: 120) | (SEQ ID NO: 121) | (SEQ ID NO: 122) |
| | VL | KSLLHRQGITY | RMS | GQLLENPYT |
| | | (SEQ ID NO: 123) | (SEQ ID NO: 124) | (SEQ ID NO: 32) |
| Ab305 | VH | GFTFSDYW | IRNKVNNYAT | NTLRLFAY |
| | | (SEQ ID NO: 120) | (SEQ ID NO: 121) | (SEQ ID NO: 122) |
| | VL | KSLLHRQGITY | RMS | GQLLENPYT |
| | | (SEQ ID NO: 123) | (SEQ ID NO: 124) | (SEQ ID NO: 32) |
| Abcon | VH | GFTFSDYW | IRNKVNNYAT | NTLRLFAY |
| | | (SEQ ID NO: 120) | (SEQ ID NO: 121) | (SEQ ID NO: 122) |
| | VL | KSLLHRQGITY | RMS | GQLLENPYT |
| | | (SEQ ID NO: 123) | (SEQ ID NO: 124) | (SEQ ID NO: 32) |

In certain embodiments, the antibody or an antigen-binding fragment thereof that specifically binds to human Axl further comprises VL and/or VH framework regions (FRs) as set forth in Table 5, which utilizes standard one letter amino acid abbreviations. It will be clear to one of ordinary skill in the art, that the VL and VH of the antibody or antigen-binding fragment thereof provided herein comprise the CDRs and FRs in the order of FR1-CDR1-FR2-CDR2-FR3-CDR3-FR4.

TABLE 5
VL and VH Framework Amino Acid Sequences (IMGT)

| | | FR1 | FR2 | FR3 | FR4 |
|---|---|---|---|---|---|
| Ab301 | VH | EVKLEASGGGLVQPGMS | MEWVRQAPGKGL | YYGKSVKGRFTISRDDSKSIV | WGQGTLVTVSA |
| | | VKLSCTTS | EWVAE | YLQMNSIRSEDTGIYYC | (SEQ ID NO: 93) |
| | | (SEQ ID NO: 102) | (SEQ ID NO: 131) | (SEQ ID NO: 116) | |
| | VL | DIVMTQAPLSISVTPGES | VYWYLQKPGKSP | TLASGVPDRFSGSGSETDFTL | FGAGTRLEIK |
| | | PSMSCRSS | QLLIY | KINKVEAEDVGIYYC | (SEQ ID NO: 110) |
| | | (SEQ ID NO: 117) | (SEQ ID NO: 118) | (SEQ ID NO: 119) | |
| Ab302 | VH | EVQLVESGGGLVKPGGS | MEWVRQAPGKGL | YYGKSVKGRFTISRDDSKNT | WGQGTLVTVSS |
| | | LKLSCAAS | EWVAE | LYLQMNSLKTEDTAVYYC | (SEQ ID NO: 63) |
| | | (SEQ ID NO: 64) | (SEQ ID NO: 131) | (SEQ ID NO: 132) | |
| | VL | DIQMTQSPSSLSASVGDR | VYWYQQKPGKVP | TLASGVPSRFSGSGSGTDFTL | FGQGTKLEIK |
| | | VTITCRSS | KLLIY | TISSLQPEDVATYYC | (SEQ ID NO: 55) |
| | | (SEQ ID NO: 127) | (SEQ ID NO: 128) | (SEQ ID NO: 129) | |
| Ab303 | VH | EVQLVESGGGVVQPGRS | MEWVRQAPGKGL | YYGKSVKGRFTISRDNSKNT | WGQGTLVTVSS |
| | | LRLSCAAS | EWVAE | LYLQMNSLRAEDTAVYYC | (SEQ ID NO: 63) |
| | | (SEQ ID NO: 73) | (SEQ ID NO: 131) | (SEQ ID NO: 133) | |
| | VL | DIQMTQSPSSLSASVGDR | VYWYQQKPGKVP | TLASGVPSRFSGSGSGTDFTL | FGQGTKLEIK |
| | | VTITCRSS | KLLIY | TISSLQPEDVATYYC | (SEQ ID NO: 55) |
| | | (SEQ ID NO: 127) | (SEQ ID NO: 128) | (SEQ ID NO: 129) | |
| Ab304 | VH | EVQLVESGGGLVQPGGS | MEWVRQAPGKGL | YYGKSVKGRFTISRDESKNS | WGQGTTVTVSS |
| | | LRLSCAAS | EWVGE | LYLQMNSLKTEDTAVYYC | (SEQ ID NO: 82) |
| | | (SEQ ID NO: 83) | (SEQ ID NO: 134) | (SEQ ID NO: 135) | |
| | VL | DIQMTQSPSSLSASVGDR | VYWYQQKPGKVP | TLASGVPSRFSGSGSGTDFTL | FGQGTKLEIK |
| | | VTITCRSS | KLLIY | TISSLQPEDVATYYC | (SEQ ID NO: 55) |
| | | (SEQ ID NO: 127) | (SEQ ID NO: 128) | (SEQ ID NO: 129) | |
| Ab305 | VH | EVQLVESGGGLVQPGRS | MEWVRQAPGKGL | YYGKSVKGRFTISRDDSKSIA | WGQGTLVTVSA |
| | | LRLSCTAS | EWVGE | YLQMNSLKTEDTAVYYC | (SEQ ID NO: 93) |
| | | (SEQ ID NO: 94) | (SEQ ID NO: 134) | (SEQ ID NO: 115) | |
| | VL | DIQMTQSPSSLSASVGDR | VYWYQQKPGKVP | TLASGVPSRFSGSGSGTDFTL | FGQGTKLEIK |
| | | VTITCRSS | KLLIY | TISSLQPEDVATYYC | (SEQ ID NO: 55) |
| | | (SEQ ID NO: 127) | (SEQ ID NO: 128) | (SEQ ID NO: 129) | |
| Abcon | VH | EVQLVESGGGX₁VX₂PG | MEWVRQAPGKGL | YYGKSVKGRFTISRDX₇SKX₈ | WGQGTX₁₃VTVSX₁₄ |
| | | X₃SLX₄LSCX₅AS | EWVX₆E | X₉X₁₀YLQMNSLX₁₁X₁₂EDTA | (X₁₃ is L or T, and |
| | | (X₁ is an amino acid | (X₆ is an amino acid | VYYC | X₁₄ is S or A) |
| | | with an aliphatic | with an aliphatic | (X₇ is D, N or E, X₈ is | (SEQ ID NO: 43) |
| | | side chain (for | side chain (for | an amino acid with a | |
| | | example, L or V), X₂ | example, A or G)) | polar side chain (for | |
| | | is K or Q, X₃ is G | (SEQ ID NO: 125) | example, N or S), X₉ is | |
| | | or R, X₄ is an amino | | T, S or I, X₁₀ is an | |
| | | acid with a | | amino acid with an | |
| | | positively charged | | aliphatic or hydrophobic | |
| | | side chain (for | | side chain (for | |
| | | example, K or R), | | example, L or A), X₁₁ | |
| | | X₅ is A or T) | | is an amino acid with a | |
| | | (SEQ ID NO: 44) | | positively charged side | |
| | | | | chain (for example, K or | |
| | | | | R), X₁₂ is T or A) | |
| | | | | (SEQ ID NO: 126) | |
| | VL | DIQMTQSPSSLSASVGDR | VYWYQQKPGKVP | TLASGVPSRFSGSGSGTDFTL | FGQGTKLEIK |
| | | VTITCRSS | KLLIY | TISSLQPEDVATYYC | (SEQ ID NO: 55) |
| | | (SEQ ID NO: 127) | (SEQ ID NO: 128) | (SEQ ID NO: 129) | |

In certain embodiments, an Axl-binding antibody, or antigen-binding fragment thereof, described herein comprises CDRs, or CDRs and FRs, of antibody Ab301, Ab302, Ab303, Ab304, Ab305, or Abcon, as determined by the Chothia numbering system.

In certain embodiments, provided herein is an antibody or an antigen-binding fragment thereof that specifically binds to human Axl, which comprises VL and/or VH CDRs as set forth in Table 6, which utilizes standard one letter amino acid abbreviations.

TABLE 6
VL and VH CDR Amino Acid Sequences (Chothia)

| | | CDR1 | CDR2 | CDR3 |
|---|---|---|---|---|
| Ab301 | VH | GFTFSDY | RNKVNNYA | LRLFAY |
| | | (SEQ ID NO: 23) | (SEQ ID NO: 24) | (SEQ ID NO: 22) |
| | VL | RSSKSLLHRNGITYVY | RMSTLAS | GQLLENPYT |
| | | (SEQ ID NO: 38) | (SEQ ID NO: 31) | (SEQ ID NO: 32) |
| Ab302 | VH | GFTFSDY | RNKVNNYA | LRLFAY |
| | | (SEQ ID NO: 23) | (SEQ ID NO: 24) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab303 | VH | GFTFSDY | RNKVNNYA | LRLFAY |
| | | (SEQ ID NO: 23) | (SEQ ID NO: 24) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab304 | VH | GFTFSDY | RNKVNNYA | LRLFAY |
| | | (SEQ ID NO: 23) | (SEQ ID NO: 24) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab305 | VH | GFTFSDY | RNKVNNYA | LRLFAY |
| | | (SEQ ID NO: 23) | (SEQ ID NO: 24) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Abcon | VH | GFTFSDY | RNKVNNYA | LRLFAY |
| | | (SEQ ID NO: 23) | (SEQ ID NO: 24) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |

In certain embodiments, the antibody or an antigen-binding fragment thereof that specifically binds to human Axl further comprises VL and/or VH framework regions (FRs) as set forth in Table 7, which utilizes standard one letter amino acid abbreviations. It will be clear to one of ordinary skill in the art, that the VL and VH of the antibody or antigen-binding fragment thereof provided herein comprise the CDRs and FRs in the order of FR1-CDR1-FR2-CDR2-FR3-CDR3-FR4.

TABLE 7
VL and VH Framework Amino Acid Sequences (Clothia)

| | | FR1 | FR2 | FR3 | FR4 |
|---|---|---|---|---|---|
| Ab301 | VH | EVKLEASGGGLVQPG | WMEWVRQAPGKGLE | TYYGKSVKGRFTISRD | WGQGTLVTVSA |
| | | MSVKLSCTTS | WVAEI | DSKSIVYLQMNSIRSE | (SEQ ID NO: 93) |
| | | (SEQ ID NO: 102) | (SEQ ID NO: 65) | DTGIYYCNT | |
| | | | | (SEQ ID NO: 103) | |
| | VL | DIVMTQAPLSISVTPGE | WYLQKPGKSPQLLIY | GVPDRFSGSGSETDFT | FGAGTRLEIK |
| | | SPSMSC | (SEQ ID NO: 108) | LKINKVEAEDVGIYYC | (SEQ ID NO: 110) |
| | | (SEQ ID NO: 107) | | (SEQ ID NO: 109) | |
| Ab302 | VH | EVQLVESGGGLVKPG | WMEWVRQAPGKGLE | TYYGKSVKGRFTISRD | WGQGTLVTVSS |
| | | GSLKLSCAAS | WVAEI | DSKNTLYLQMNSLKT | (SEQ ID NO: 63) |
| | | (SEQ ID NO: 64) | (SEQ ID NO: 65) | EDTAVYYCNT | |
| | | | | (SEQ ID NO: 66) | |
| | VL | DIQMTQSPSSLSASVG | WYQQKPGKVPKLLIY | GVPSRFSGSGSGTDFT | FGQGTKLEIK |
| | | DRVTITC | (SEQ ID NO: 53) | LTISSLQPEDVATYYC | (SEQ ID NO: 55) |
| | | (SEQ ID NO: 52) | | (SEQ ID NO: 54) | |
| Ab303 | VH | EVQLVESGGGVVQPG | WMEWVRQAPGKGLE | TYYGKSVKGRFTISRD | WGQGTLVTVSS |
| | | RSLRLSCAAS | WVAEI | NSKNTLYLQMNSLRA | (SEQ ID NO: 63) |
| | | (SEQ ID NO: 73) | (SEQ ID NO: 65) | EDTAVYYCNT | |
| | | | | (SEQ ID NO: 74) | |
| | VL | DIQMTQSPSSLSASVG | WYQQKPGKVPKLLIY | GVPSRFSGSGSGTDFT | FGQGTKLEIK |
| | | DRVTITC | (SEQ ID NO: 53) | LTISSLQPEDVATYYC | (SEQ ID NO: 55) |
| | | (SEQ ID NO: 52) | | (SEQ ID NO: 54) | |
| Ab304 | VH | EVQLVESGGGLVQPG | WMEWVRQAPGKGLE | TYYGKSVKGRFTISRD | WGQGTTVTVSS |
| | | GSLRLSCAAS | WVGEI | ESKNSLYLQMNSLKTE | (SEQ ID NO: 82) |
| | | (SEQ ID NO: 83) | (SEQ ID NO: 84) | DTAVYYCNT | |
| | | | | (SEQ ID NO: 85) | |
| | VL | DIQMTQSPSSLSASVG | WYQQKPGKVPKLLIY | GVPSRFSGSGSGTDFT | FGQGTKLEIK |
| | | DRVTITC | (SEQ ID NO: 53) | LTISSLQPEDVATYYC | (SEQ ID NO: 55) |
| | | (SEQ ID NO: 52) | | (SEQ ID NO: 54) | |
| Ab305 | VH | EVQLVESGGGLVQPG | WMEWVRQAPGKGLE | TYYGKSVKGRFTISRD | WGQGTLVTVSA |
| | | RSLRLSCTAS | WVGEI | DSKSIAYLQMNSLKTE | (SEQ ID NO: 93) |
| | | (SEQ ID NO: 94) | (SEQ ID NO: 84) | DTAVYYCNT | |
| | | | | (SEQ ID NO: 95) | |
| | VL | DIQMTQSPSSLSASVG | WYQQKPGKVPKLLIY | GVPSRFSGSGSGTDFT | FGQGTKLEIK |
| | | DRVTITC | (SEQ ID NO: 53) | LTISSLQPEDVATYYC | (SEQ ID NO: 55) |
| | | (SEQ ID NO: 52) | | (SEQ ID | NO: 54) |
| Abcon | VH | EVQLVESGGGX₁VX₂P | WMEWVRQAPGKGLE | TYYGKSVKGRFTISRD | WGQGTX₁₃VTVSX₁₄ |
| | | GX₃SLX₄LSCX₅AS | WVX₆EI | X₇SKX₈X₉X₁₀YLQMNSL | (X₁₃ is L or T, |
| | | (X₁ is an amino acid | (X₆ is an amino acid | X₁₁X₁₂EDTAVYYCNT | and X₁₄ is S or A) |
| | | with an aliphatic | with an aliphatic | (X₇ is D, N or E, | (SEQ ID NO: 43) |
| | | side chain (for | side chain (for | X₈ is an amino acid | |
| | | example, L or V), X₂ | example, A or G)) | with a polar side | |
| | | is K or Q, X₃ is G | (SEQ ID NO: 45) | chain (for example, | |
| | | or R, X₄ is an | | N or S), X₉ is T, S | |
| | | mino acid with a | | or I, X₁₀ is an amino | |
| | | positively charged | | acid with an | |
| | | side chain (for | | aliphatic or | |
| | | example, K or R), | | hydrophobic side | |
| | | X₅ is A or T) | | chain (for example, L | |
| | | (SEQ ID NO: 44) | | or A), X₁₁ is an amino | |
| | | | | acid with a | |
| | | | | positively charged | |
| | | | | side chain (for | |
| | | | | example, K or R), X₁₂ | |
| | | | | is T or A) | |
| | | | | (SEQ ID NO: 46) | |
| | VL | DIQMTQSPSSLSASVG | WYQQKPGKVPKLLIY | GVPSRFSGSGSGTDFT | FGQGTKLEIK |
| | | DRVTITC | (SEQ ID NO: 53) | LTISSLQPEDVATYYC | (SEQ ID NO: 55) |
| | | (SEQ ID NO: 52) | | (SEQ ID NO: 54) | |

In certain embodiments, an Axl-binding antibody, or antigen-binding fragment thereof, described herein comprises CDRs, or CDRs and FRs, of antibody Ab301, Ab302, Ab303, Ab304, Ab305, or Abcon, as determined by the AbM numbering system.

In certain embodiments, provided herein is an antibody or an antigen-binding fragment thereof that specifically binds to human Axl, which comprises VL and/or VH CDRs as set forth in Table 8, which utilizes standard one letter amino acid abbreviations.

TABLE 8
VL and VH CDR Amino Acid Sequences (AbM)

| | | CDR1 | CDR2 | CDR3 |
|---|---|---|---|---|
| Ab301 | VH | GFTFSDYWME | EIRNKVNNYATY | LRLFAY |
| | | (SEQ ID NO: 25) | (SEQ ID NO: 26) | (SEQ ID NO: 22) |
| | VL | RSSKSLLHRNGITYVY | RMSTLAS | GQLLENPYT |
| | | (SEQ ID NO: 38) | (SEQ ID NO: 31) | (SEQ ID NO: 32) |
| Ab302 | VH | GFTFSDYWME | EIRNKVNNYATY | LRLFAY |
| | | (SEQ ID NO: 25) | (SEQ ID NO: 26) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab303 | VH | GFTFSDYWME | EIRNKVNNYATY | LRLFAY |
| | | (SEQ ID NO: 25) | (SEQ ID NO: 26) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab304 | VH | GFTFSDYWME | EIRNKVNNYATY | LRLFAY |
| | | (SEQ ID NO: 25) | (SEQ ID NO: 26) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab305 | VH | GFTFSDYWME | EIRNKVNNYATY | LRLFAY |
| | | (SEQ ID NO: 25) | (SEQ ID NO: 26) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Abcon | VH | GFTFSDYWME | EIRNKVNNYATY | LRLFAY |
| | | (SEQ ID NO: 25) | (SEQ ID NO: 26) | (SEQ ID NO: 22) |
| | VL | RSSKSLLHRQGITYVY | RMSTLAS | GQLLENPYT |
| | | (SEQ ID NO: 30) | (SEQ ID NO: 31) | (SEQ ID NO: 32) |

In certain embodiments, the antibody or an antigen-binding fragment thereof that specifically binds to human Axl further comprises VL and/or VH framework regions (FRs) as set forth in Table 9, which utilizes standard one letter amino acid abbreviations. It will be clear to one of ordinary skill in the art, that the VL and VH of the antibody or antigen-binding fragment thereof provided herein comprise the CDRs and FRs in the order of FR1-CDR1-FR2-CDR2-FR3-CDR3-FR4.

TABLE 9
VL and VH Framework Amino Acid Sequences (AbM)

| | | FR1 | FR2 | FR3 | FR4 |
|---|---|---|---|---|---|
| Ab301 | VH | EVKLEASGGGLVQPG | WVRQAPGKGLEWVA | YGKSVKGRFTISRDDSKSIV | WGQGTLVTVSA |
| | | MSVKLSCTTS | (SEQ ID NO: 67) | YLQMNSIRSEDTGIYYCNT | (SEQ ID NO: 93) |
| | | (SEQ ID NO: 102) | | (SEQ ID NO: 104) | |
| | VL | DIVMTQAPLSISVTPGE | WYLQKPGKSPQLLIY | GVPDRFSGSGSETDFTLKIN | FGAGTRLEIK |
| | | SPSMSC | (SEQ ID NO: 108) | KVEAEDVGIYYC | (SEQ ID NO: 110) |
| | | (SEQ ID NO: 107) | | (SEQ ID NO: 109) | |
| Ab302 | VH | EVQLVESGGGLVKPGG | WVRQAPGKGLEWVA | YGKSVKGRFTISRDDSKNTL | WGQGTLVTVSS |
| | | SLKLSCAAS | (SEQ ID NO: 67) | YLQMNSLKTEDTAVYYCNT | (SEQ ID NO: 63) |
| | | (SEQ ID NO: 64) | | (SEQ ID NO: 68) | |
| | VL | DIQMTQSPSSLSASVGD | WYQQKPGKVPKLLIY | GVPSRFSGSGSGTDFTLTISS | FGQGTKLEIK |
| | | RVTITC | (SEQ ID NO: 53) | LQPEDVATYYC | (SEQ ID NO: 55) |
| | | (SEQ ID NO: 52) | | (SEQ ID NO: 54) | |
| Ab303 | VH | EVQLVESGGGVVQPGR | WVRQAPGKGLEWVA | YGKSVKGRFTISRDNSKNTL | WGQGTLVTVSS |
| | | SLRLSCAAS | (SEQ ID NO: 67) | YLQMNSLRAEDTAVYYCNT | (SEQ ID NO: 63) |
| | | (SEQ ID NO: 73) | | (SEQ ID NO: 75) | |
| | VL | DIQMTQSPSSLSASVGD | WYQQKPGKVPKLLIY | GVPSRFSGSGSGTDFTLTISS | FGQGTKLEIK |
| | | RVTITC | (SEQ ID NO: 53) | LQPEDVATYYC | (SEQ ID NO: 55) |
| | | (SEQ ID NO: 52) | | (SEQ ID NO: 54) | |
| Ab304 | VH | EVQLVESGGGLVQPGG | WVRQAPGKGLEWVG | YGKSVKGRFTISRDESKNSL | WGQGTTVTVSS |
| | | SLRLSCAAS | (SEQ ID NO: 86) | YLQMNSLKTEDTAVYYCNT | (SEQ ID NO: 82) |
| | | (SEQ ID NO: 83) | | (SEQ ID NO: 87) | |
| | VL | DIQMTQSPSSLSASVGD | WYQQKPGKVPKLLIY | GVPSRFSGSGSGTDFTLTISS | FGQGTKLEIK |
| | | RVTITC | (SEQ ID NO: 53) | LQPEDVATYYC | (SEQ ID NO: 55) |
| | | (SEQ ID NO: 52) | | (SEQ ID NO: 54) | |
| Ab305 | VH | EVQLVESGGGLVQPGR | WVRQAPGKGLEWVG | YGKSVKGRFTISRDDSKSIA | WGQGTLVTVSA |
| | | SLRLSCTAS | (SEQ ID NO: 86) | YLQMNSLKTEDTAVYYCNT | (SEQ ID NO: 93) |
| | | (SEQ ID NO: 94) | | (SEQ ID NO: 96) | |
| | VL | DIQMTQSPSSLSASVGD | WYQQKPGKVPKLLIY | GVPSRFSGSGSGTDFTLTISS | FGQGTKLEIK |
| | | RVTITC | (SEQ ID NO: 53) | LQPEDVATYYC | (SEQ ID NO: 55) |
| | | (SEQ ID NO: 52) | | (SEQ ID NO: 54) | |
| Abcon | VH | EVQLVESGGGX₁VX₂PG | WVRQAPGKGLEWVX₆ | YGKSVKGRFTISRDX₇SKX₈ | WGQGTX₁₃VTVSX₁₄ |
| | | X₃SLX₄LSCX₅AS | (X₆ is an amino | X₉X₁₀YLQMNSLX₁₁X₁₂EDTA | (X₁₃ is L or T, |
| | | (X₁ is an amino acid | acid with an | VYYCNT | and X₁₄ is S or |
| | | with an aliphatic | aliphatic side | (X₇ is D, N or E, X₈ is | A) |
| | | side chain (for | chain (for | an amino acid with a | (SEQ ID NO: 43) |
| | | example, L or V), X₂ | example, A or G)) | polar side chain (for | |
| | | is K or Q, X₃ is G or | (SEQ ID NO: 41) | example, N or S), X₉ is | |
| | | R, X₄ is an amino | | T, S or I, X₁₀ is an | |
| | | acid with a | | amino acid with an | |
| | | positively charged | | aliphatic or hydrophobic | |
| | | side chain (for | | side chain (for example, | |
| | | example, K or | | L or A), X₁₁ is an amino | |
| | | R), X₅ is A or T) | | acid with a positively | |
| | | (SEQ ID NO: 44) | | charged side chain | |
| | | | | (for example, K or R), | |
| | | | | X₁₂ is T or A) | |
| | | | | (SEQ ID NO: 47) | |
| | VL | DIQMTQSPSSLSASVGD | WYQQKPGKVPKLLIY | GVPSRFSGSGSGTDFTLTISS | FGQGTKLEIK |
| | | RVTITC | (SEQ ID NO: 53) | LQPEDVATYYC | (SEQ ID NO: 55) |
| | | (SEQ ID NO: 52) | | (SEQ ID NO: 54) | |

In certain embodiments, an Axl-binding antibody, or antigen-binding fragment thereof, described herein comprises CDRs, or CDRs and FRs, of antibody Ab301, Ab302, Ab303, Ab304, Ab305, or Abcon, as determined by the Contact numbering system.

In certain embodiments, provided herein is an antibody or an antigen-binding fragment thereof that specifically binds to human Axl, which comprises VL and/or VH CDRs as set forth in Table 10, which utilizes standard one letter amino acid abbreviations.

TABLE 10
VL and VH CDR Amino Acid Sequences (Contact)

| | | CDR1 | CDR2 | CDR3 |
|---|---|---|---|---|
| Ab301 | VH | SDYWME | WVAEIRNKVNNYATY | NTLRLFA |
| | | (SEQ ID NO: 27) | (SEQ ID NO: 36) | (SEQ ID NO: 29) |
| | VL | LHRNGITYVYWY | LLIYRMSTLA | GQLLENPY |
| | | (SEQ ID NO: 39) | (SEQ ID NO: 34) | (SEQ ID NO: 35) |
| Ab302 | VH | SDYWME | WVAEIRNKVNNYATY | NTLRLFA |
| | | (SEQ ID NO: 27) | (SEQ ID NO: 36) | (SEQ ID NO: 29) |
| | VL | LHRQGITYVYWY | LLIYRMSTLA | GQLLENPY |
| | | (SEQ ID NO: 33) | (SEQ ID NO: 34) | (SEQ ID NO: 35) |
| Ab303 | VH | SDYWME | WVAEIRNKVNNYATY | NTLRLFA |
| | | (SEQ ID NO: 27) | (SEQ ID NO: 36) | (SEQ ID NO: 29) |
| | VL | LHRQGITYVYWY | LLIYRMSTLA | GQLLENPY |
| | | (SEQ ID NO: 33) | (SEQ ID NO: 34) | (SEQ ID NO: 35) |
| Ab304 | VH | SDYWME | WVGEIRNKVNNYATY | NTLRLFA |
| | | (SEQ ID NO: 27) | (SEQ ID NO: 37) | (SEQ ID NO: 29) |
| | VL | LHRQGITYVYWY | LLIYRMSTLA | GQLLENPY |
| | | (SEQ ID NO: 33) | (SEQ ID NO: 34) | (SEQ ID NO: 35) |
| Ab305 | VH | SDYWME | WVGEIRNKVNNYATY | NTLRLFA |
| | | (SEQ ID NO: 27) | (SEQ ID NO: 37) | (SEQ ID NO: 29) |
| | VL | LHRQGITYVYWY | LLIYRMSTLA | GQLLENPY |
| | | (SEQ ID NO: 33) | (SEQ ID NO: 34) | (SEQ ID NO: 35) |
| Abcon | VH | SDYWME | WVX₆EIRNKVNNYATY | NTLRLFA |
| | | (SEQ ID NO: 27) | (X₆ is an amino acid | (SEQ ID NO: 29) |
| | | | with an aliphatic | |
| | | | side chain (for | |
| | | | example, A or G)) | |
| | | | (SEQ ID NO: 28) | |
| | VL | LHRQGITYVYWY | LLIYRMSTLA | GQLLENPY |
| | | (SEQ ID NO: 33) | (SEQ ID NO: 34) | (SEQ ID NO: 35) |

In certain embodiments, the antibody or an antigen-binding fragment thereof that specifically binds to human Axl further comprises VL and/or VH framework regions (FRs) as set forth in Table 11, which utilizes standard one letter amino acid abbreviations. It will be clear to one of ordinary skill in the art, that the VL and VH of the antibody or antigen-binding fragment thereof provided herein comprise the CDRs and FRs in the order of FR1-CDR1-FR2-CDR2-FR3-CDR3-FR4.

TABLE 11
VL and VH Framework Amino Acid Sequences (Contact)

| | | FR1 | FR2 | FR3 | FR4 |
|---|---|---|---|---|---|
| Ab301 | VH | EVKLEASGGGLVQPG | WVRQAPGKGLE | YGKSVKGRFTISRDDS | YWGQGTLVTVSA |
| | | MSVKLSCTTSGFTF | (SEQ ID NO: 49) | KSIVYLQMNSIRSEDTG | (SEQ ID NO: 99) |
| | | (SEQ ID NO: 105) | | IYYC | |
| | | | | (SEQ ID NO: 106) | |
| | VL | DIVMTQAPLSISVTPG | LQKPGKSPQ | SGVPDRFSGSGSETDFT | TFGAGTRLEIK |
| | | ESPSMSCRSSKSL | (SEQ ID NO: 112) | LKINKVEAEDVGIYYC | (SEQ ID NO: 114) |
| | | (SEQ ID NO: 111) | | (SEQ ID NO: 113) | |
| Ab302 | VH | EVQLVESGGGLVKPG | WVRQAPGKGLE | YGKSVKGRFTISRDDS | YWGQGTLVTVSS |
| | | GSLKLSCAASGFTF | (SEQ ID NO: 49) | KNTLYLQMNSLKTEDT | (SEQ ID NO: 78) |
| | | (SEQ ID NO: 69) | | AVYYC | |
| | | | | (SEQ ID NO: 70) | |
| | VL | DIQMTQSPSSLSASVG | QQKPGKVPK | SGVPSRFSGSGSGTDFT | TFGQGTKLEIK |
| | | DRVTITCRSSKSL | (SEQ ID NO: 57) | LTISSLQPEDVATYYC | (SEQ ID NO: 59) |
| | | (SEQ ID NO: 56) | | (SEQ ID NO: 58) | |
| Ab303 | VH | EVQLVESGGGVVQPG | WVRQAPGKGLE | YGKSVKGRFTISRDNS | YWGQGTLVTVSS |
| | | RSLRLSCAASGFTF | (SEQ ID NO: 49) | KNTLYLQMNSLRAED | (SEQ ID NO: 78) |
| | | (SEQ ID NO: 76) | | TAVYYC | |
| | | | | (SEQ ID NO: 77) | |
| | VL | DIQMTQSPSSLSASVG | QQKPGKVPK | SGVPSRFSGSGSGTDFT | TFGQGTKLEIK |
| | | DRVTITCRSSKSL | (SEQ ID NO: 57) | LTISSLQPEDVATYYC | (SEQ ID NO: 59) |
| | | (SEQ ID NO: 56) | | (SEQ ID NO: 58) | |
| Ab304 | VH | EVQLVESGGGLVQPG | WVRQAPGKGLE | YGKSVKGRFTISRDES | YWGQGTTVTVSS |
| | | GSLRLSCAASGFTF | (SEQ ID NO: 49) | KNSLYLQMNSLKTEDT | (SEQ ID NO: 90) |
| | | (SEQ ID NO: 88) | | AVYYC | |
| | | | | (SEQ ID NO: 89) | |
| | VL | DIQMTQSPSSLSASVG | QQKPGKVPK | SGVPSRFSGSGSGTDFT | TFGQGTKLEIK |
| | | DRVTITCRSSKSL | (SEQ ID NO: 57) | LTISSLQPEDVATYYC | (SEQ ID NO: 59) |
| | | (SEQ ID NO: 56) | | (SEQ ID NO: 58) | |
| Ab305 | VH | EVQLVESGGGLVQPG | WVRQAPGKGLE | YGKSVKGRFTISRDDS | YWGQGTLVTVSA |
| | | RSLRLSCTASGFTF | (SEQ ID NO: 49) | KSIAYLQMNSLKTEDT | (SEQ ID NO: 99) |
| | | (SEQ ID NO: 97) | | AVYYC | |
| | | | | (SEQ ID NO: 98) | |
| | VL | DIQMTQSPSSLSASVG | QQKPGKVPK | SGVPSRFSGSGSGTDFT | TFGQGTKLEIK |
| | | DRVTITCRSSKSL | (SEQ ID NO: 57) | LTISSLQPEDVATYYC | (SEQ ID NO: 59) |
| | | (SEQ ID NO: 56) | | (SEQ ID NO: 58) | |
| Abcon | VH | EVQLVESGGGX₁VX₂P | WVRQAPGKGLE | YGKSVKGRFTISRDX₇S | YWGQGTX₁₃VTVSX₁₄ |
| | | GX₃SLX₄LSCX₅ASGFT | (SEQ ID NO: 49) | KX₈X₉X₁₀YLQMNSLX₁₁ | (X₁₃ is L or T, and |
| | | F | | X₁₂EDTAVYYC | X₁₄ is S or A) |
| | | (X₁ is an amino | | (X₇ is D, N or E, X₈ | (SEQ ID NO: 51) |
| | | acid with an | | is an amino acid with | |
| | | aliphatic side | | a polar side chain | |
| | | chain | | (for example, N or S), | |
| | | (for example, L or | | X₉ is T, S or I, X₁₀ is | |
| | | V), X₂ is K or Q, | | an amino acid with an | |
| | | X₃ is G or R, X₄ is | | aliphatic or | |
| | | an amino acid with | | hydrophobic side | |
| | | a positively | | chain (for example, L | |
| | | charged side | | or A), X₁₁ is an amino | |
| | | chain (for | | acid with a | |
| | | example, K or | | positively charged | |
| | | R), X₅ is A or T) | | side chain (for | |
| | | (SEQ ID NO: 48) | | example, K | |
| | | | | or R), X₁₂ is T or A) | |
| | | | | (SEQ ID NO: 50) | |
| | VL | DIQMTQSPSSLSASVG | QQKPGKVPK | SGVPSRFSGSGSGTDFT | TFGQGTKLEIK |
| | | DRVTITCRSSKSL | (SEQ ID NO: 57) | LTISSLQPEDVATYYC | (SEQ ID NO: 59) |
| | | (SEQ ID NO: 56) | | (SEQ ID NO: 58) | |

In certain embodiments, an Axl-binding antibody, or antigen-binding fragment thereof, described herein comprises CDRs of antibody Ab301, Ab302, Ab303, Ab304, Ab305, or Abcon, as determined by combining the respective sequences of the CDRs determined by the Kabat numbering system and by the IMGT numbering system, or such CDRs and the corresponding FRs.

In certain embodiments, provided herein is an antibody or an antigen-binding fragment thereof that specifically binds to human Axl, which comprises VL and/or VH CDRs as set forth in Table 12, which utilizes standard one letter amino acid abbreviations.

TABLE 12
CDRs by Kabat and IMGT combined

| | | CDR1 | CDR2 | CDR3 |
|---|---|---|---|---|
| Ab301 | VH | GFTFSDYWME | EIRNKVNNYATYYGKSVKG | NTLRLFAY |
| | | (SEQ ID NO: 136) | (SEQ ID NO: 21) | (SEQ ID NO: 122) |
| | VL | RSSKSLLHRNGITYVY | RMSTLAS | GQLLENPYT |
| | | (SEQ ID NO: 38) | (SEQ ID NO: 31) | (SEQ ID NO: 32) |
| Ab302 | VH | GFTFSDYWME | EIRNKVNNYATYYGKSVKG | NTLRLFAY |
| | | (SEQ ID NO: 136) | (SEQ ID NO: 21) | (SEQ ID NO: 122) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab303 | VH | GFTFSDYWME | EIRNKVNNYATYYGKSVKG | NTLRLFAY |
| | | (SEQ ID NO: 136) | (SEQ ID NO: 21) | (SEQ ID NO: 122) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab304 | VH | GFTFSDYWME | EIRNKVNNYATYYGKSVKG | NTLRLFAY |
| | | (SEQ ID NO: 136) | (SEQ ID NO: 21) | (SEQ ID NO: 122) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Ab305 | VH | GFTFSDYWME | EIRNKVNNYATYYGKSVKG | NTLRLFAY |
| | | (SEQ ID NO: 136) | (SEQ ID NO: 21) | (SEQ ID NO: 122) |
| | VL | RSSKSLLHRQGITYVY | RMSTLAS | GQLLENPYT |
| | | (SEQ ID NO: 30) | (SEQ ID NO: 31) | (SEQ ID NO: 32) |
| Abcon | VH | GFTFSDYWME | EIRNKVNNYATYYGKSVKG | NTLRLFAY |
| | | (SEQ ID NO: 136) | (SEQ ID NO: 21) | (SEQ ID NO: 122) |
| | VL | RSSKSLLHRQGITYVY | RMSTLAS | GQLLENPYT |
| | | (SEQ ID NO: 30) | (SEQ ID NO: 31) | (SEQ ID NO: 32) |

In certain embodiments, the antibody or an antigen-binding fragment thereof that specifically binds to human Axl further comprises VL and/or VH framework regions (FRs) as set forth in Table 13, which utilizes standard one letter amino acid abbreviations. It will be clear to one of ordinary skill in the art, that the VL and VH of the antibody or antigen-binding fragment thereof provided herein comprise the CDRs and FRs in the order of FR1-CDR1-FR2-CDR2-FR3-CDR3-FR4.

TABLE 13
FRs (when CDRs are determined by combining Kabat and IMGT CDR sequences)

| | | FR1 | FR2 | FR3 | FR4 |
|---|---|---|---|---|---|
| Ab301 | VH | EVKLEASGGGLVQPG | WVRQAPGKGLEWVA | RFTISRDDSKSIVYLQ | WGQGTLVTVSA |
| | | MSVKLSCTTS | (SEQ ID NO: 61) | MNSIRSEDTGIYYC | (SEQ ID NO: 93) |
| | | (SEQ ID NO: 137) | | (SEQ ID NO: 138) | |
| | VL | DIVMTQAPLSISVTPGE | WYLQKPGKSPQLLIY | GVPDRFSGSGSETDF | FGAGTRLEIK |
| | | SPSMSC | (SEQ ID NO: 108) | TLKINKVEAEDVGIY | (SEQ ID NO: 110) |
| | | (SEQ ID NO: 107) | | YC | |
| | | | | (SEQ ID NO: 109) | |
| Ab302 | VH | EVQLVESGGGLVKPG | WVRQAPGKGLEWVA | RFTISRDDSKNTLYL | WGQGTLVTVSS |
| | | GSLKLSCAAS | (SEQ ID NO: 61) | QMNSLKTEDTAVYY | (SEQ ID NO: 63) |
| | | (SEQ ID NO: 139) | | C | |
| | | | | (SEQ ID NO: 140) | |
| | VL | DIQMTQSPSSLSASVG | WYQQKPGKVPKLLIY | GVPSRFSGSGSGTDF | FGQGTKLEIK |
| | | DRVTITC | (SEQ ID NO: 53) | TLTISSLQPEDVATY | (SEQ ID NO: 55) |
| | | (SEQ ID NO: 52) | | YC | |
| | | | | (SEQ ID NO: 54) | |
| Ab303 | VH | EVQLVESGGGVVQPG | WVRQAPGKGLEWVA | RFTISRDNSKNTLYL | WGQGTLVTVSS |
| | | RSLRLSCAAS | (SEQ ID NO: 61) | QMNSLRAEDTAVYY | (SEQ ID NO: 63) |
| | | (SEQ ID NO: 141) | | C | |
| | | | | (SEQ ID NO: 142) | |
| | VL | DIQMTQSPSSLSASVG | WYQQKPGKVPKLLIY | GVPSRFSGSGSGTDF | FGQGTKLEIK |
| | | DRVTITC | (SEQ ID NO: 53) | TLTISSLQPEDVATY | (SEQ ID NO: 55) |
| | | (SEQ ID NO: 52) | | YC | |
| | | | | (SEQ ID NO: 54) | |
| Ab304 | VH | EVQLVESGGGLVQPG | WVRQAPGKGLEWVG | RFTISRDESKNSLYL | WGQGTTVTVSS |
| | | GSLRLSCAAS | (SEQ ID NO: 80) | QMNSLKTEDTAVYY | (SEQ ID NO: 82) |
| | | (SEQ ID NO: 143) | | C | |
| | | | | (SEQ ID NO: 144) | |
| | VL | DIQMTQSPSSLSASVG | WYQQKPGKVPKLLIY | GVPSRFSGSGSGTDF | FGQGTKLEIK |
| | | DRVTITC | (SEQ ID NO: 53) | TLTISSLQPEDVATY | (SEQ ID NO: 55) |
| | | (SEQ ID NO: 52) | | YC | |
| | | | | (SEQ ID NO: 54) | |
| Ab305 | VH | EVQLVESGGGLVQPG | WVRQAPGKGLEWVG | RFTISRDDSKSIAYLQ | WGQGTLVTVSA |
| | | RSLRLSCTAS | (SEQ ID NO: 80) | MNSLKTEDTAVYYC | (SEQ ID NO: 93) |
| | | (SEQ ID NO: 145) | | (SEQ ID NO: 146) | |
| | VL | DIQMTQSPSSLSASVG | WYQQKPGKVPKLLIY | GVPSRFSGSGSGTDF | FGQGTKLEIK |
| | | DRVTITC | (SEQ ID NO: 53) | TLTISSLQPEDVATY | (SEQ ID NO: 55) |
| | | (SEQ ID NO: 52) | | YC | |
| | | | | (SEQ ID NO: 54) | |
| Abcon | VH | EVQLVESGGGX₁VX₂P | WVRQAPGKGLEWVX₆ | RFTISRDX₇SKX₈X₉X₁₀ | WGQGTX₁₃VTVS |
| | | GX₃SLX₄LSCX₅AS | (X₆ is an amino acid | YLQMNSLX₁₁X₁₂EDT | X₁₄ |
| | | (X₁ is an amino acid | with an aliphatic | AVYYC | (X₁₃ is L or T, and |
| | | with an aliphatic | side chai | (X₇ is D, N or E, X₈ | |
| | | side chain (for | (for example, A or | is an amino acid with | X₁₄ is S or A) |
| | | example, L or V), X₂ | G)) | a polar side chain | (SEQ ID NO: 43) |
| | | is K or Q, X₃ is G | (SEQ ID NO: 41) | (for example, N or S), | |
| | | or R, X₄ is | | X₉ is T, S or I, X₁₀ is | |
| | | an amino acid with a | | an amino acid with | |
| | | positively charged | | an aliphatic or | |
| | | side chain (for | | hydrophobic | |
| | | example, K or R), | | side chain (for | |
| | | X₅ is A or T) | | example, L or A), X₁₁ | |
| | | (SEQ ID NO: 44) | | is an amino acid with | |
| | | | | a positively charged | |
| | | | | side chain (for | |
| | | | | example, K or R), X₁₂ | |
| | | | | is T or A) | |
| | | | | (SEQ ID NO: 147) | |
| | VL | DIQMTQSPSSLSASVG | WYQQKPGKVPKLLIY | GVPSRFSGSGSGTDF | FGQGTKLEIK |
| | | DRVTITC | (SEQ ID NO: 53) | TLTISSLQPEDVATY | (SEQ ID NO: 55) |
| | | (SEQ ID NO: 52) | | YC | |
| | | | | (SEQ ID NO: 54) | |

In a particular embodiment, an antibody or an antigen-binding fragment described herein, comprises the VL CDR1, VL CDR2, and VL CDR3 of Ab301. In a specific embodiment, the VL CDR1, VL CDR2, and VL CDR3 are as set forth in Table 1. In another specific embodiment, the VL CDR1, VL CDR2, and VL CDR3 are as set forth in Table 4. In another specific embodiment, the VL CDR1, VL CDR2, and VL CDR3 are as set forth in Table 6. In another specific embodiment, the VL CDR1, VL CDR2, and VL CDR3 are as set forth in Table 8. In another specific embodiment, the VL CDR1, VL CDR2, and VL CDR3 are as set forth in Table 10. In another specific embodiment, the VL CDR1, VL CDR2, and VL CDR3 are as set forth in Table 12. In one embodiment, the antibody or antigen-binding fragment further comprises a VH as set forth in Table 3.

In a particular embodiment, an antibody described herein, or an antigen-binding fragment thereof, comprises the VH CDR1, VH CDR2, and VH CDR3 of Ab301. In a specific embodiment, the VH CDR1, VH CDR2, and VH CDR3 are as set forth in Table 1. In another specific embodiment, the VH CDR1, VH CDR2, and VH CDR3 are as set forth in Table 4. In another specific embodiment, the VH CDR1, VH CDR2, and VH CDR3 are as set forth in Table 6. In another specific embodiment, the VH CDR1, VH CDR2, and VH CDR3 are as set forth in Table 8. In another specific embodiment, the VH CDR1, VH CDR2, and VH CDR3 are as set forth in Table 10. In another specific embodiment, the VH CDR1, VH CDR2, and VH CDR3 are as set forth in Table 12. In one embodiment, the antibody or antigen-binding fragment further comprises a VL as set forth in Table 3.

In a particular embodiment, an antibody or an antigen-binding fragment described herein, which specifically binds to human Axl, comprises a VL as set forth in Table 3. In one embodiment, the antibody or antigen-binding fragment further comprises the VH CDR1, VH CDR2 and/or VH CDR3 of Ab301. In a specific embodiment, the VH CDR1, VH CDR2 and/or VH CDR3 of Ab301 are as set forth in Table 1. In another specific embodiment, the VH CDR1, VH CDR2 and/or VH CDR3 of Ab301 are as set forth in Table 4. In another specific embodiment, the VH CDR1, VH CDR2 and/or VH CDR3 of Ab301 are as set forth in Table 6. In another specific embodiment, the VH CDR1, VH CDR2 and/or VH CDR3 of Ab301 are as set forth in Table 8. In another specific embodiment, the VH CDR1, VH CDR2 and/or VH CDR3 of Ab301 are as set forth in Table 10. In another specific embodiment, the VH CDR1, VH CDR2 and/or VH CDR3 of Ab301 are as set forth in Table 12.

In a particular embodiment, an antibody described herein, or an antigen-binding fragment thereof, which specifically binds to human Axl, comprises a VH as set forth in Table 3. In one embodiment, the antibody or antigen-binding fragment further comprises the VL CDR1, VL CDR2 and/or VL CDR3 of Ab301. In a specific embodiment, the VL CDR1, VL CDR2 and/or VL CDR3 of Ab301 are as set forth in Table 1. In another specific embodiment, the VL CDR1, VL CDR2 and/or VL CDR3 of Ab301 are as set forth in Table 4. In another specific embodiment, the VL CDR1, VL CDR2 and/or VL CDR3 of Ab301 are as set forth in Table 6. In another specific embodiment, the VL CDR1, VL CDR2 and/or VL CDR3 of Ab301 are as set forth in Table 8. In another specific embodiment, the VL CDR1, VL CDR2 and/or VL CDR3 of Ab301 are as set forth in Table 10. In another specific embodiment, the VL CDR1, VL CDR2 and/or VL CDR3 of Ab301 are as set forth in Table 12.

In a particular embodiment, an antibody described herein, or an antigen-binding fragment thereof, comprises the VL CDR1, VL CDR2, and VL CDR3 of Ab301, and the VH CDR1, VH CDR2, and VH CDR3 of Ab301. In a specific embodiment, the VL CDR1, VL CDR2, and VL CDR3 are as set forth in Table 1, and the VH CDR1, VH CDR2, and VH CDR3 are as set forth in Table 1. In another specific embodiment, the VL CDR1, VL CDR2, and VL CDR3 are as set forth in Table 4, and the VH CDR1, VH CDR2, and VH CDR3 are as set forth in Table 4. In another specific embodiment, the VL CDR1, VL CDR2, and VL CDR3 are as set forth in Table 6, and the VH CDR1, VH CDR2, and VH CDR3 are as set forth in Table 6. In another specific embodiment, the VL CDR1, VL CDR2, and VL CDR3 are as set forth in Table 8, and the VH CDR1, VH CDR2, and VH CDR3 are as set forth in Table 8. In another specific embodiment, the VL CDR1, VL CDR2, and VL CDR3 are as set forth in Table 10, and the VH CDR1, VH CDR2, and VH CDR3 are as set forth in Table 10. In another specific embodiment, the VL CDR1, VL CDR2, and VL CDR3 are as set forth in Table 12, and the VH CDR1, VH CDR2, and VH CDR3 are as set forth in Table 12.

In a particular embodiment, an antibody described herein, or an antigen-binding fragment thereof, comprises the VL CDR1, VL CDR2, and VL CDR3 of Ab301, and the VL FR1, VL FR2, VL FR3, and VL FR4 of Ab301. In a specific embodiment, the VL CDR1, VL CDR2, and VL CDR3 are as set forth in Table 1, and the VL FR1, VL FR2, VL FR3, and VL FR4 are as set forth in Table 2. In another specific embodiment, the VL CDR1, VL CDR2, and VL CDR3 are as set forth in Table 4, and the VL FR1, VL FR2, VL FR3, and VL FR4 are as set forth in Table 5. In another specific embodiment, the VL CDR1, VL CDR2, and VL CDR3 are as set forth in Table 6, and the VL FR1, VL FR2, VL FR3, and VL FR4 are as set forth in Table 7. In another specific embodiment, the VL CDR1, VL CDR2, and VL CDR3 are as set forth in Table 8, and the VL FR1, VL FR2, VL FR3, and VL FR4 are as set forth in Table 9. In another specific embodiment, the VL CDR1, VL CDR2, and VL CDR3 are as set forth in Table 10, and the VL FR1, VL FR2, VL FR3, and VL FR4 are as set forth in Table 11. In another specific embodiment, the VL CDR1, VL CDR2, and VL CDR3 are as set forth in Table 12, and the VL FR1, VL FR2, VL FR3, and VL FR4 are as set forth in Table 13.

In a particular embodiment, an antibody described herein, or an antigen-binding fragment thereof, comprises the VH CDR1, VH CDR2, and VH CDR3 of Ab301, and the VH FR1, VH FR2, VH FR3, and VH FR4 of Ab301. In a specific embodiment, the VH CDR1, VH CDR2, and VH CDR3 are as set forth in Table 1, and the VH FR1, VH FR2, VH FR3, and VH FR4 are as set forth in Table 2. In another specific embodiment, the VH CDR1, VH CDR2, and VH CDR3 are as set forth in Table 4, and the VH FR1, VH FR2, VH FR3, and VH FR4 are as set forth in Table 5. In another specific embodiment, the VH CDR1, VH CDR2, and VH CDR3 are as set forth in Table 6, and the VH FR1, VH FR2, VH FR3, and VH FR4 are as set forth in Table 7. In another specific embodiment, the VH CDR1, VH CDR2, and VH CDR3 are as set forth in Table 8, and the VH FR1, VH FR2, VH FR3, and VH FR4 are as set forth in Table 9. In another specific embodiment, the VH CDR1, VH CDR2, and VH CDR3 are as set forth in Table 10, and the VH FR1, VH FR2, VH FR3, and VH FR4 are as set forth in Table 11. In another specific embodiment, the VH CDR1, VH CDR2, and VH CDR3 are as set forth in Table 12, and the VH FR1, VH FR2, VH FR3, and VH FR4 are as set forth in Table 13.

In a particular embodiment, an antibody or an antigen-binding fragment described herein, comprises the VL CDR1, VL CDR2, and VL CDR3 of Ab302. In a specific embodiment, the VL CDR1, VL CDR2, and VL CDR3 are as set forth in Table 1. In another specific embodiment, the VL CDR1, VL CDR2, and VL CDR3 are as set forth in Table 4. In another specific embodiment, the VL CDR1, VL CDR2, and VL CDR3 are as set forth in Table 6. In another specific embodiment, the VL CDR1, VL CDR2, and VL CDR3 are as set forth in Table 8. In another specific embodiment, the VL CDR1, VL CDR2, and VL CDR3 are as set forth in Table 10. In another specific embodiment, the VL CDR1, VL CDR2, and VL CDR3 are as set forth in Table 12. In one embodiment, the antibody or antigen-binding fragment further comprises a VH as set forth in Table 3.

In a particular embodiment, an antibody described herein, or an antigen-binding fragment thereof, comprises the VH CDR1, VH CDR2, and VH CDR3 of Ab302. In a specific embodiment, the VH CDR1, VH CDR2, and VH CDR3 are as set forth in Table 1. In another specific embodiment, the VH CDR1, VH CDR2, and VH CDR3 are as set forth in Table 4. In another specific embodiment, the VH CDR1, VH CDR2, and VH CDR3 are as set forth in Table 6. In another specific embodiment, the VH CDR1, VH CDR2, and VH CDR3 are as set forth in Table 8. In another specific embodiment, the VH CDR1, VH CDR2, and VH CDR3 are as set forth in Table 10. In another specific embodiment, the VH CDR1, VH CDR2, and VH CDR3 are as set forth in Table 12. In one embodiment, the antibody or antigen-binding fragment further comprises a VL as set forth in Table 3.

In a particular embodiment, an antibody or an antigen-binding fragment described herein, which specifically binds to human Axl, comprises a VL as set forth in Table 3. In one embodiment, the antibody or antigen-binding fragment further comprises the VH CDR1, VH CDR2 and/or VH CDR3 of Ab302. In a specific embodiment, the VH CDR1, VH CDR2 and/or VH CDR3 of Ab302 are as set forth in Table 1. In another specific embodiment, the VH CDR1, VH CDR2 and/or VH CDR3 of Ab302 are as set forth in Table 4. In another specific embodiment, the VH CDR1, VH CDR2 and/or VH CDR3 of Ab302 are as set forth in Table 6. In another specific embodiment, the VH CDR1, VH CDR2 and/or VH CDR3 of Ab302 are as set forth in Table 8. In another specific embodiment, the VH CDR1, VH CDR2 and/or VH CDR3 of Ab302 are as set forth in Table 10. In another specific embodiment, the VH CDR1, VH CDR2 and/or VH CDR3 of Ab302 are as set forth in Table 12.

In a particular embodiment, an antibody described herein, or an antigen-binding fragment thereof, which specifically binds to human Axl, comprises a VH as set forth in Table 3. In one embodiment, the antibody or antigen-binding fragment further comprises the VL CDR1, VL CDR2 and/or VL CDR3 of Ab302. In a specific embodiment, the VL CDR1, VL CDR2 and/or VL CDR3 of Ab302 are as set forth in Table 1. In another specific embodiment, the VL CDR1, VL CDR2 and/or VL CDR3 of Ab302 are as set forth in Table 4. In another specific embodiment, the VL CDR1, VL CDR2 and/or VL CDR3 of Ab302 are as set forth in Table 6. In another specific embodiment, the VL CDR1, VL CDR2 and/or VL CDR3 of Ab302 are as set forth in Table 8. In another specific embodiment, the VL CDR1, VL CDR2 and/or VL CDR3 of Ab302 are as set forth in Table 10. In another specific embodiment, the VL CDR1, VL CDR2 and/or VL CDR3 of Ab302 are as set forth in Table 12.

In a particular embodiment, an antibody described herein, or an antigen-binding fragment thereof, comprises the VL CDR1, VL CDR2, and VL CDR3 of Ab302, and the VH CDR1, VH CDR2, and VH CDR3 of Ab302. In a specific embodiment, the VL CDR1, VL CDR2, and VL CDR3 are as set forth in Table 1, and the VH CDR1, VH CDR2, and VH CDR3 are as set forth in Table 1. In another specific embodiment, the VL CDR1, VL CDR2, and VL CDR3 are as set forth in Table 4, and the VH CDR1, VH CDR2, and VH CDR3 are as set forth in Table 4. In another specific embodiment, the VL CDR1, VL CDR2, and VL CDR3 are as set forth in Table 6, and the VH CDR1, VH CDR2, and VH CDR3 are as set forth in Table 6. In another specific embodiment, the VL CDR1, VL CDR2, and VL CDR3 are as set forth in Table 8, and the VH CDR1, VH CDR2, and VH CDR3 are as set forth in Table 8. In another specific embodiment, the VL CDR1, VL CDR2, and VL CDR3 are as set forth in Table 10, and the VH CDR1, VH CDR2, and VH CDR3 are as set forth in Table 10. In another specific embodiment, the VL CDR1, VL CDR2, and VL CDR3 are as set forth in Table 12, and the VH CDR1, VH CDR2, and VH CDR3 are as set forth in Table 12.

In a particular embodiment, an antibody described herein, or an antigen-binding fragment thereof, comprises the VL CDR1, VL CDR2, and VL CDR3 of Ab302, and the VL FR1, VL FR2, VL FR3, and VL FR4 of Ab302. In a specific embodiment, the VL CDR1, VL CDR2, and VL CDR3 are as set forth in Table 1, and the VL FR1, VL FR2, VL FR3, and VL FR4 are as set forth in Table 2. In another specific embodiment, the VL CDR1, VL CDR2, and VL CDR3 are as set forth in Table 4, and the VL FR1, VL FR2, VL FR3, and VL FR4 are as set forth in Table 5. In another specific embodiment, the VL CDR1, VL CDR2, and VL CDR3 are as set forth in Table 6, and the VL FR1, VL FR2, VL FR3, and VL FR4 are as set forth in Table 7. In another specific embodiment, the VL CDR1, VL CDR2, and VL CDR3 are as set forth in Table 8, and the VL FR1, VL FR2, VL FR3, and VL FR4 are as set forth in Table 9. In another specific embodiment, the VL CDR1, VL CDR2, and VL CDR3 are as set forth in Table 10, and the VL FR1, VL FR2, VL FR3, and VL FR4 are as set forth in Table 11. In another specific embodiment, the VL CDR1, VL CDR2, and VL CDR3 are as set forth in Table 12, and the VL FR1, VL FR2, VL FR3, and VL FR4 are as set forth in Table 13.

In a particular embodiment, an antibody described herein, or an antigen-binding fragment thereof, comprises the VH CDR1, VH CDR2, and VH CDR3 of Ab302, and the VH FR1, VH FR2, VH FR3, and VH FR4 of Ab302. In a specific embodiment, the VH CDR1, VH CDR2, and VH CDR3 are as set forth in Table 1, and the VH FR1, VH FR2, VH FR3, and VH FR4 are as set forth in Table 2. In another specific embodiment, the VH CDR1, VH CDR2, and VH CDR3 are as set forth in Table 4, and the VH FR1, VH FR2, VH FR3, and VH FR4 are as set forth in Table 5. In another specific embodiment, the VH CDR1, VH CDR2, and VH CDR3 are as set forth in Table 6, and the VH FR1, VH FR2, VH FR3, and VH FR4 are as set forth in Table 7. In another specific embodiment, the VH CDR1, VH CDR2, and VH CDR3 are as set forth in Table 8, and the VH FR1, VH FR2, VH FR3, and VH FR4 are as set forth in Table 9. In another specific embodiment, the VH CDR1, VH CDR2, and VH CDR3 are as set forth in Table 10, and the VH FR1, VH FR2, VH FR3, and VH FR4 are as set forth in Table 11. In another specific embodiment, the VH CDR1, VH CDR2, and VH CDR3 are as set forth in Table 12, and the VH FR1, VH FR2, VH FR3, and VH FR4 are as set forth in Table 13.

In a particular embodiment, an antibody or an antigen-binding fragment described herein, comprises the VL CDR1, VL CDR2, and VL CDR3 of Ab303. In a specific embodiment, the VL CDR1, VL CDR2, and VL CDR3 are as set forth in Table 1. In another specific embodiment, the VL CDR1, VL CDR2, and VL CDR3 are as set forth in Table 4. In another specific embodiment, the VL CDR1, VL CDR2, and VL CDR3 are as set forth in Table 6. In another specific embodiment, the VL CDR1, VL CDR2, and VL CDR3 are as set forth in Table 8. In another specific embodiment, the VL CDR1, VL CDR2, and VL CDR3 are as set forth in Table 10. In another specific embodiment, the VL CDR1, VL CDR2, and VL CDR3 are as set forth in Table 12. In one embodiment, the antibody or antigen-binding fragment further comprises a VH as set forth in Table 3.

In a particular embodiment, an antibody described herein, or an antigen-binding fragment thereof, comprises the VH CDR1, VH CDR2, and VH CDR3 of Ab303. In a specific embodiment, the VH CDR1, VH CDR2, and VH CDR3 are as set forth in Table 1. In another specific embodiment, the VH CDR1, VH CDR2, and VH CDR3 are as set forth in Table 4. In another specific embodiment, the VH CDR1, VH CDR2, and VH CDR3 are as set forth in Table 6. In another specific embodiment, the VH CDR1, VH CDR2, and VH CDR3 are as set forth in Table 8. In another specific embodiment, the VH CDR1, VH CDR2, and VH CDR3 are as set forth in Table 10. In another specific embodiment, the VH CDR1, VH CDR2, and VH CDR3 are as set forth in Table 12. In one embodiment, the antibody or antigen-binding fragment further comprises a VL as set forth in Table 3.

In a particular embodiment, an antibody or an antigen-binding fragment described herein, which specifically binds to human Axl, comprises a VL as set forth in Table 3. In one embodiment, the antibody or antigen-binding fragment further comprises the VH CDR1, VH CDR2 and/or VH CDR3 of Ab303. In a specific embodiment, the VH CDR1, VH CDR2 and/or VH CDR3 of Ab303 are as set forth in Table 1. In another specific embodiment, the VH CDR1, VH CDR2 and/or VH CDR3 of Ab303 are as set forth in Table 4. In another specific embodiment, the VH CDR1, VH CDR2 and/or VH CDR3 of Ab303 are as set forth in Table 6. In another specific embodiment, the VH CDR1, VH CDR2 and/or VH CDR3 of Ab303 are as set forth in Table 8. In another specific embodiment, the VH CDR1, VH CDR2 and/or VH CDR3 of Ab303 are as set forth in Table 10. In another specific embodiment, the VH CDR1, VH CDR2 and/or VH CDR3 of Ab303 are as set forth in Table 12.

In a particular embodiment, an antibody described herein, or an antigen-binding fragment thereof, which specifically binds to human Axl, comprises a VH as set forth in Table 3. In one embodiment, the antibody or antigen-binding fragment further comprises the VL CDR1, VL CDR2 and/or VL CDR3 of Ab303. In a specific embodiment, the VL CDR1, VL CDR2 and/or VL CDR3 of Ab303 are as set forth in Table 1. In another specific embodiment, the VL CDR1, VL CDR2 and/or VL CDR3 of Ab303 are as set forth in Table 4. In another specific embodiment, the VL CDR1, VL CDR2 and/or VL CDR3 of Ab303 are as set forth in Table 6. In another specific embodiment, the VL CDR1, VL CDR2 and/or VL CDR3 of Ab303 are as set forth in Table 8. In another specific embodiment, the VL CDR1, VL CDR2 and/or VL CDR3 of Ab303 are as set forth in Table 10. In another specific embodiment, the VL CDR1, VL CDR2 and/or VL CDR3 of Ab303 are as set forth in Table 12.

In a particular embodiment, an antibody described herein, or an antigen-binding fragment thereof, comprises the VL CDR1, VL CDR2, and VL CDR3 of Ab303, and the VH CDR1, VH CDR2, and VH CDR3 of Ab303. In a specific embodiment, the VL CDR1, VL CDR2, and VL CDR3 are as set forth in Table 1, and the VH CDR1, VH CDR2, and VH CDR3 are as set forth in Table 1. In another specific embodiment, the VL CDR1, VL CDR2, and VL CDR3 are as set forth in Table 4, and the VH CDR1, VH CDR2, and VH CDR3 are as set forth in Table 4. In another specific embodiment, the VL CDR1, VL CDR2, and VL CDR3 are as set forth in Table 6, and the VH CDR1, VH CDR2, and VH CDR3 are as set forth in Table 6. In another specific embodiment, the VL CDR1, VL CDR2, and VL CDR3 are as set forth in Table 8, and the VH CDR1, VH CDR2, and VH CDR3 are as set forth in Table 8. In another specific embodiment, the VL CDR1, VL CDR2, and VL CDR3 are as set forth in Table 10, and the VH CDR1, VH CDR2, and VH CDR3 are as set forth in Table 10. In another specific embodiment, the VL CDR1, VL CDR2, and VL CDR3 are as set forth in Table 12, and the VH CDR1, VH CDR2, and VH CDR3 are as set forth in Table 12.

In a particular embodiment, an antibody described herein, or an antigen-binding fragment thereof, comprises the VL CDR1, VL CDR2, and VL CDR3 of Ab303, and the VL FR1, VL FR2, VL FR3, and VL FR4 of Ab303. In a specific embodiment, the VL CDR1, VL CDR2, and VL CDR3 are as set forth in Table 1, and the VL FR1, VL FR2, VL FR3, and VL FR4 are as set forth in Table 2. In another specific embodiment, the VL CDR1, VL CDR2, and VL CDR3 are as set forth in Table 4, and the VL FR1, VL FR2, VL FR3, and VL FR4 are as set forth in Table 5. In another specific embodiment, the VL CDR1, VL CDR2, and VL CDR3 are as set forth in Table 6, and the VL FR1, VL FR2, VL FR3, and VL FR4 are as set forth in Table 7. In another specific embodiment, the VL CDR1, VL CDR2, and VL CDR3 are as set forth in Table 8, and the VL FR1, VL FR2, VL FR3, and VL FR4 are as set forth in Table 9. In another specific embodiment, the VL CDR1, VL CDR2, and VL CDR3 are as set forth in Table 10, and the VL FR1, VL FR2, VL FR3, and VL FR4 are as set forth in Table 11. In another specific embodiment, the VL CDR1, VL CDR2, and VL CDR3 are as set forth in Table 12, and the VL FR1, VL FR2, VL FR3, and VL FR4 are as set forth in Table 13.

In a particular embodiment, an antibody described herein, or an antigen-binding fragment thereof, comprises the VH CDR1, VH CDR2, and VH CDR3 of Ab303, and the VH FR1, VH FR2, VH FR3, and VH FR4 of Ab303. In a specific embodiment, the VH CDR1, VH CDR2, and VH CDR3 are as set forth in Table 1, and the VH FR1, VH FR2, VH FR3, and VH FR4 are as set forth in Table 2. In another specific embodiment, the VH CDR1, VH CDR2, and VH CDR3 are as set forth in Table 4, and the VH FR1, VH FR2, VH FR3, and VH FR4 are as set forth in Table 5. In another specific embodiment, the VH CDR1, VH CDR2, and VH CDR3 are as set forth in Table 6, and the VH FR1, VH FR2, VH FR3, and VH FR4 are as set forth in Table 7. In another specific embodiment, the VH CDR1, VH CDR2, and VH CDR3 are as set forth in Table 8, and the VH FR1, VH FR2, VH FR3, and VH FR4 are as set forth in Table 9. In another specific embodiment, the VH CDR1, VH CDR2, and VH CDR3 are as set forth in Table 10, and the VH FR1, VH FR2, VH FR3, and VH FR4 are as set forth in Table 11. In another specific embodiment, the VH CDR1, VH CDR2, and VH CDR3 are as set forth in Table 12, and the VH FR1, VH FR2, VH FR3, and VH FR4 are as set forth in Table 13.

In a particular embodiment, an antibody or an antigen-binding fragment described herein, comprises the VL CDR1, VL CDR2, and VL CDR3 of Ab304. In a specific embodiment, the VL CDR1, VL CDR2, and VL CDR3 are as set forth in Table 1. In another specific embodiment, the VL CDR1, VL CDR2, and VL CDR3 are as set forth in Table 4. In another specific embodiment, the VL CDR1, VL CDR2, and VL CDR3 are as set forth in Table 6. In another specific embodiment, the VL CDR1, VL CDR2, and VL CDR3 are as set forth in Table 8. In another specific embodiment, the VL CDR1, VL CDR2, and VL CDR3 are as set forth in Table 10. In another specific embodiment, the VL CDR1, VL CDR2, and VL CDR3 are as set forth in Table 12. In one embodiment, the antibody or antigen-binding fragment further comprises a VH as set forth in Table 3.

In a particular embodiment, an antibody described herein, or an antigen-binding fragment thereof, comprises the VH CDR1, VH CDR2, and VH CDR3 of Ab304. In a specific embodiment, the VH CDR1, VH CDR2, and VH CDR3 are as set forth in Table 1. In another specific embodiment, the VH CDR1, VH CDR2, and VH CDR3 are as set forth in Table 4. In another specific embodiment, the VH CDR1, VH CDR2, and VH CDR3 are as set forth in Table 6. In another specific embodiment, the VH CDR1, VH CDR2, and VH CDR3 are as set forth in Table 8. In another specific embodiment, the VH CDR1, VH CDR2, and VH CDR3 are as set forth in Table 10. In another specific embodiment, the VH CDR1, VH CDR2, and VH CDR3 are as set forth in Table 12. In one embodiment, the antibody or antigen-binding fragment further comprises a VL as set forth in Table 3.

In a particular embodiment, an antibody or an antigen-binding fragment described herein, which specifically binds to human Axl, comprises a VL as set forth in Table 3. In one embodiment, the antibody or antigen-binding fragment further comprises the VH CDR1, VH CDR2 and/or VH CDR3 of Ab304. In a specific embodiment, the VH CDR1, VH CDR2 and/or VH CDR3 of Ab304 are as set forth in Table 1. In another specific embodiment, the VH CDR1, VH CDR2 and/or VH CDR3 of Ab304 are as set forth in Table 4. In another specific embodiment, the VH CDR1, VH CDR2 and/or VH CDR3 of Ab304 are as set forth in Table 6. In another specific embodiment, the VH CDR1, VH CDR2 and/or VH CDR3 of Ab304 are as set forth in Table 8. In another specific embodiment, the VH CDR1, VH CDR2 and/or VH CDR3 of Ab304 are as set forth in Table 10. In another specific embodiment, the VH CDR1, VH CDR2 and/or VH CDR3 of Ab304 are as set forth in Table 12.

In a particular embodiment, an antibody described herein, or an antigen-binding fragment thereof, which specifically binds to human Axl, comprises a VH as set forth in Table 3. In one embodiment, the antibody or antigen-binding fragment further comprises the VL CDR1, VL CDR2 and/or VL CDR3 of Ab304. In a specific embodiment, the VL CDR1, VL CDR2 and/or VL CDR3 of Ab304 are as set forth in Table 1. In another specific embodiment, the VL CDR1, VL CDR2 and/or VL CDR3 of Ab304 are as set forth in Table 4. In another specific embodiment, the VL CDR1, VL CDR2 and/or VL CDR3 of Ab304 are as set forth in Table 6. In another specific embodiment, the VL CDR1, VL CDR2 and/or VL CDR3 of Ab304 are as set forth in Table 8. In another specific embodiment, the VL CDR1, VL CDR2 and/or VL CDR3 of Ab304 are as set forth in Table 10. In another specific embodiment, the VL CDR1, VL CDR2 and/or VL CDR3 of Ab304 are as set forth in Table 12.

In a particular embodiment, an antibody described herein, or an antigen-binding fragment thereof, comprises the VL CDR1, VL CDR2, and VL CDR3 of Ab304, and the VH CDR1, VH CDR2, and VH CDR3 of Ab304. In a specific embodiment, the VL CDR1, VL CDR2, and VL CDR3 are as set forth in Table 1, and the VH CDR1, VH CDR2, and VH CDR3 are as set forth in Table 1. In another specific embodiment, the VL CDR1, VL CDR2, and VL CDR3 are as set forth in Table 4, and the VH CDR1, VH CDR2, and VH CDR3 are as set forth in Table 4. In another specific embodiment, the VL CDR1, VL CDR2, and VL CDR3 are as set forth in Table 6, and the VH CDR1, VH CDR2, and VH CDR3 are as set forth in Table 6. In another specific embodiment, the VL CDR1, VL CDR2, and VL CDR3 are as set forth in Table 8, and the VH CDR1, VH CDR2, and VH CDR3 are as set forth in Table 8. In another specific embodiment, the VL CDR1, VL CDR2, and VL CDR3 are as set forth in Table 10, and the VH CDR1, VH CDR2, and VH CDR3 are as set forth in Table 10. In another specific embodiment, the VL CDR1, VL CDR2, and VL CDR3 are as set forth in Table 12, and the VH CDR1, VH CDR2, and VH CDR3 are as set forth in Table 12.

In a particular embodiment, an antibody described herein, or an antigen-binding fragment thereof, comprises the VL CDR1, VL CDR2, and VL CDR3 of Ab304, and the VL FR1, VL FR2, VL FR3, and VL FR4 of Ab304. In a specific embodiment, the VL CDR1, VL CDR2, and VL CDR3 are as set forth in Table 1, and the VL FR1, VL FR2, VL FR3, and VL FR4 are as set forth in Table 2. In another specific embodiment, the VL CDR1, VL CDR2, and VL CDR3 are as set forth in Table 4, and the VL FR1, VL FR2, VL FR3, and VL FR4 are as set forth in Table 5. In another specific embodiment, the VL CDR1, VL CDR2, and VL CDR3 are as set forth in Table 6, and the VL FR1, VL FR2, VL FR3, and VL FR4 are as set forth in Table 7. In another specific embodiment, the VL CDR1, VL CDR2, and VL CDR3 are as set forth in Table 8, and the VL FR1, VL FR2, VL FR3, and VL FR4 are as set forth in Table 9. In another specific embodiment, the VL CDR1, VL CDR2, and VL CDR3 are as set forth in Table 10, and the VL FR1, VL FR2, VL FR3, and VL FR4 are as set forth in Table 11. In another specific embodiment, the VL CDR1, VL CDR2, and VL CDR3 are as set forth in Table 12, and the VL FR1, VL FR2, VL FR3, and VL FR4 are as set forth in Table 13.

In a particular embodiment, an antibody described herein, or an antigen-binding fragment thereof, comprises the VH CDR1, VH CDR2, and VH CDR3 of Ab304, and the VH FR1, VH FR2, VH FR3, and VH FR4 of Ab304. In a specific embodiment, the VH CDR1, VH CDR2, and VH CDR3 are as set forth in Table 1, and the VH FR1, VH FR2, VH FR3, and VH FR4 are as set forth in Table 2. In another specific embodiment, the VH CDR1, VH CDR2, and VH CDR3 are as set forth in Table 4, and the VH FR1, VH FR2, VH FR3, and VH FR4 are as set forth in Table 5. In another specific embodiment, the VH CDR1, VH CDR2, and VH CDR3 are as set forth in Table 6, and the VH FR1, VH FR2, VH FR3, and VH FR4 are as set forth in Table 7. In another specific embodiment, the VH CDR1, VH CDR2, and VH CDR3 are as set forth in Table 8, and the VH FR1, VH FR2, VH FR3, and VH FR4 are as set forth in Table 9. In another specific embodiment, the VH CDR1, VH CDR2, and VH CDR3 are as set forth in Table 10, and the VH FR1, VH FR2, VH FR3, and VH FR4 are as set forth in Table 11. In another specific embodiment, the VH CDR1, VH CDR2, and VH CDR3 are as set forth in Table 12, and the VH FR1, VH FR2, VH FR3, and VH FR4 are as set forth in Table 13.

In a particular embodiment, an antibody or an antigen-binding fragment described herein, comprises the VL CDR1, VL CDR2, and VL CDR3 of Ab305. In a specific embodiment, the VL CDR1, VL CDR2, and VL CDR3 are as set forth in Table 1. In another specific embodiment, the VL CDR1, VL CDR2, and VL CDR3 are as set forth in Table 4. In another specific embodiment, the VL CDR1, VL CDR2, and VL CDR3 are as set forth in Table 6. In another specific embodiment, the VL CDR1, VL CDR2, and VL CDR3 are as set forth in Table 8. In another specific embodiment, the VL CDR1, VL CDR2, and VL CDR3 are as set forth in Table 10. In another specific embodiment, the VL CDR1, VL CDR2, and VL CDR3 are as set forth in Table 12. In one embodiment, the antibody or antigen-binding fragment further comprises a VH as set forth in Table 3.

In a particular embodiment, an antibody described herein, or an antigen-binding fragment thereof, comprises the VH CDR1, VH CDR2, and VH CDR3 of Ab305. In a specific embodiment, the VH CDR1, VH CDR2, and VH CDR3 are as set forth in Table 1. In another specific embodiment, the VH CDR1, VH CDR2, and VH CDR3 are as set forth in Table 4. In another specific embodiment, the VH CDR1, VH CDR2, and VH CDR3 are as set forth in Table 6. In another specific embodiment, the VH CDR1, VH CDR2, and VH CDR3 are as set forth in Table 8. In another specific embodiment, the VH CDR1, VH CDR2, and VH CDR3 are as set forth in Table 10. In another specific embodiment, the VH CDR1, VH CDR2, and VH CDR3 are as set forth in Table 12. In one embodiment, the antibody or antigen-binding fragment further comprises a VL as set forth in Table 3.

In a particular embodiment, an antibody or an antigen-binding fragment described herein, which specifically binds to human Axl, comprises a VL as set forth in Table 3. In one embodiment, the antibody or antigen-binding fragment further comprises the VH CDR1, VH CDR2 and/or VH CDR3 of Ab305. In a specific embodiment, the VH CDR1, VH CDR2 and/or VH CDR3 of Ab305 are as set forth in Table 1. In another specific embodiment, the VH CDR1, VH CDR2 and/or VH CDR3 of Ab305 are as set forth in Table 4. In another specific embodiment, the VH CDR1, VH CDR2 and/or VH CDR3 of Ab305 are as set forth in Table 6. In another specific embodiment, the VH CDR1, VH CDR2 and/or VH CDR3 of Ab305 are as set forth in Table 8. In another specific embodiment, the VH CDR1, VH CDR2 and/or VH CDR3 of Ab305 are as set forth in Table 10. In another specific embodiment, the VH CDR1, VH CDR2 and/or VH CDR3 of Ab305 are as set forth in Table 12.

In a particular embodiment, an antibody described herein, or an antigen-binding fragment thereof, which specifically binds to human Axl, comprises a VH as set forth in Table 3. In one embodiment, the antibody or antigen-binding fragment further comprises the VL CDR1, VL CDR2 and/or VL CDR3 of Ab305. In a specific embodiment, the VL CDR1, VL CDR2 and/or VL CDR3 of Ab305 are as set forth in Table 1. In another specific embodiment, the VL CDR1, VL CDR2 and/or VL CDR3 of Ab305 are as set forth in Table 4. In another specific embodiment, the VL CDR1, VL CDR2 and/or VL CDR3 of Ab305 are as set forth in Table 6. In another specific embodiment, the VL CDR1, VL CDR2 and/or VL CDR3 of Ab305 are as set forth in Table 8. In another specific embodiment, the VL CDR1, VL CDR2 and/or VL CDR3 of Ab305 are as set forth in Table 10. In another specific embodiment, the VL CDR1, VL CDR2 and/or VL CDR3 of Ab305 are as set forth in Table 12.

In a particular embodiment, an antibody described herein, or an antigen-binding fragment thereof, comprises the VL CDR1, VL CDR2, and VL CDR3 of Ab305, and the VH CDR1, VH CDR2, and VH CDR3 of Ab305. In a specific embodiment, the VL CDR1, VL CDR2, and VL CDR3 are as set forth in Table 1, and the VH CDR1, VH CDR2, and VH CDR3 are as set forth in Table 1. In another specific embodiment, the VL CDR1, VL CDR2, and VL CDR3 are as set forth in Table 4, and the VH CDR1, VH CDR2, and VH CDR3 are as set forth in Table 4. In another specific embodiment, the VL CDR1, VL CDR2, and VL CDR3 are as set forth in Table 6, and the VH CDR1, VH CDR2, and VH CDR3 are as set forth in Table 6. In another specific embodiment, the VL CDR1, VL CDR2, and VL CDR3 are as set forth in Table 8, and the VH CDR1, VH CDR2, and VH CDR3 are as set forth in Table 8. In another specific embodiment, the VL CDR1, VL CDR2, and VL CDR3 are as set forth in Table 10, and the VH CDR1, VH CDR2, and VH CDR3 are as set forth in Table 10. In another specific embodiment, the VL CDR1, VL CDR2, and VL CDR3 are as set forth in Table 12, and the VH CDR1, VH CDR2, and VH CDR3 are as set forth in Table 12.

In a particular embodiment, an antibody described herein, or an antigen-binding fragment thereof, comprises the VL CDR1, VL CDR2, and VL CDR3 of Ab305, and the VL FR1, VL FR2, VL FR3, and VL FR4 of Ab305. In a specific embodiment, the VL CDR1, VL CDR2, and VL CDR3 are as set forth in Table 1, and the VL FR1, VL FR2, VL FR3, and VL FR4 are as set forth in Table 2. In another specific embodiment, the VL CDR1, VL CDR2, and VL CDR3 are as set forth in Table 4, and the VL FR1, VL FR2, VL FR3, and VL FR4 are as set forth in Table 5. In another specific embodiment, the VL CDR1, VL CDR2, and VL CDR3 are as set forth in Table 6, and the VL FR1, VL FR2, VL FR3, and VL FR4 are as set forth in Table 7. In another specific embodiment, the VL CDR1, VL CDR2, and VL CDR3 are as set forth in Table 8, and the VL FR1, VL FR2, VL FR3, and VL FR4 are as set forth in Table 9. In another specific embodiment, the VL CDR1, VL CDR2, and VL CDR3 are as set forth in Table 10, and the VL FR1, VL FR2, VL FR3, and VL FR4 are as set forth in Table 11. In another specific embodiment, the VL CDR1, VL CDR2, and VL CDR3 are as set forth in Table 12, and the VL FR1, VL FR2, VL FR3, and VL FR4 are as set forth in Table 13.

In a particular embodiment, an antibody described herein, or an antigen-binding fragment thereof, comprises the VH CDR1, VH CDR2, and VH CDR3 of Ab305, and the VH FR1, VH FR2, VH FR3, and VH FR4 of Ab305. In a specific embodiment, the VH CDR1, VH CDR2, and VH CDR3 are as set forth in Table 1, and the VH FR1, VH FR2, VH FR3, and VH FR4 are as set forth in Table 2. In another specific embodiment, the VH CDR1, VH CDR2, and VH CDR3 are as set forth in Table 4, and the VH FR1, VH FR2, VH FR3, and VH FR4 are as set forth in Table 5. In another specific embodiment, the VH CDR1, VH CDR2, and VH CDR3 are as set forth in Table 6, and the VH FR1, VH FR2, VH FR3, and VH FR4 are as set forth in Table 7. In another specific embodiment, the VH CDR1, VH CDR2, and VH CDR3 are as set forth in Table 8, and the VH FR1, VH FR2, VH FR3, and VH FR4 are as set forth in Table 9. In another specific embodiment, the VH CDR1, VH CDR2, and VH CDR3 are as set forth in Table 10, and the VH FR1, VH FR2, VH FR3, and VH FR4 are as set forth in Table 11. In another specific embodiment, the VH CDR1, VH CDR2, and VH CDR3 are as set forth in Table 12, and the VH FR1, VH FR2, VH FR3, and VH FR4 are as set forth in Table 13.

In a particular embodiment, an antibody or an antigen-binding fragment described herein, comprises the VL CDR1, VL CDR2, and VL CDR3 of Abcon. In a specific embodiment, the VL CDR1, VL CDR2, and VL CDR3 are as set forth in Table 1. In another specific embodiment, the VL CDR1, VL CDR2, and VL CDR3 are as set forth in Table 4. In another specific embodiment, the VL CDR1, VL CDR2, and VL CDR3 are as set forth in Table 6. In another specific embodiment, the VL CDR1, VL CDR2, and VL CDR3 are as set forth in Table 8. In another specific embodiment, the VL CDR1, VL CDR2, and VL CDR3 are as set forth in Table 10. In another specific embodiment, the VL CDR1, VL CDR2, and VL CDR3 are as set forth in Table 12. In one embodiment, the antibody or antigen-binding fragment further comprises a VH as set forth in Table 3.

In a particular embodiment, an antibody described herein, or an antigen-binding fragment thereof, comprises the VH CDR1, VH CDR2, and VH CDR3 of Abcon. In a specific embodiment, the VH CDR1, VH CDR2, and VH CDR3 are as set forth in Table 1. In another specific embodiment, the VH CDR1, VH CDR2, and VH CDR3 are as set forth in Table 4. In another specific embodiment, the VH CDR1, VH CDR2, and VH CDR3 are as set forth in Table 6. In another specific embodiment, the VH CDR1, VH CDR2, and VH CDR3 are as set forth in Table 8. In another specific embodiment, the VH CDR1, VH CDR2, and VH CDR3 are as set forth in Table 10. In another specific embodiment, the VH CDR1, VH CDR2, and VH CDR3 are as set forth in Table 12. In one embodiment, the antibody or antigen-binding fragment further comprises a VL as set forth in Table 3.

In a particular embodiment, an antibody or an antigen-binding fragment described herein, which specifically binds to human Axl, comprises a VL as set forth in Table 3. In one embodiment, the antibody or antigen-binding fragment further comprises the VH CDR1, VH CDR2 and/or VH CDR3 of Abcon. In a specific embodiment, the VH CDR1, VH CDR2 and/or VH CDR3 of Abcon are as set forth in Table 1. In another specific embodiment, the VH CDR1, VH CDR2 and/or VH CDR3 of Abcon are as set forth in Table 4. In another specific embodiment, the VH CDR1, VH CDR2 and/or VH CDR3 of Abcon are as set forth in Table 6. In another specific embodiment, the VH CDR1, VH CDR2 and/or VH CDR3 of Abcon are as set forth in Table 8. In another specific embodiment, the VH CDR1, VH CDR2 and/or VH CDR3 of Abcon are as set forth in Table 10. In another specific embodiment, the VH CDR1, VH CDR2 and/or VH CDR3 of Abcon are as set forth in Table 12.

In a particular embodiment, an antibody described herein, or an antigen-binding fragment thereof, which specifically binds to human Axl, comprises a VH as set forth in Table 3. In one embodiment, the antibody or antigen-binding fragment further comprises the VL CDR1, VL CDR2 and/or VL CDR3 of Abcon. In a specific embodiment, the VL CDR1, VL CDR2 and/or VL CDR3 of Abcon are as set forth in Table 1. In another specific embodiment, the VL CDR1, VL CDR2 and/or VL CDR3 of Abcon are as set forth in Table 4. In another specific embodiment, the VL CDR1, VL CDR2 and/or VL CDR3 of Abcon are as set forth in Table 6. In another specific embodiment, the VL CDR1, VL CDR2 and/or VL CDR3 of Abcon are as set forth in Table 8. In another specific embodiment, the VL CDR1, VL CDR2 and/or VL CDR3 of Abcon are as set forth in Table 10. In another specific embodiment, the VL CDR1, VL CDR2 and/or VL CDR3 of Abcon are as set forth in Table 12.

In a particular embodiment, an antibody described herein, or an antigen-binding fragment thereof, comprises the VL CDR1, VL CDR2, and VL CDR3 of Abcon, and the VH CDR1, VH CDR2, and VH CDR3 of Abcon. In a specific embodiment, the VL CDR1, VL CDR2, and VL CDR3 are as set forth in Table 1, and the VH CDR1, VH CDR2, and VH CDR3 are as set forth in Table 1. In another specific embodiment, the VL CDR1, VL CDR2, and VL CDR3 are as set forth in Table 4, and the VH CDR1, VH CDR2, and VH CDR3 are as set forth in Table 4. In another specific embodiment, the VL CDR1, VL CDR2, and VL CDR3 are as set forth in Table 6, and the VH CDR1, VH CDR2, and VH CDR3 are as set forth in Table 6. In another specific embodiment, the VL CDR1, VL CDR2, and VL CDR3 are as set forth in Table 8, and the VH CDR1, VH CDR2, and VH CDR3 are as set forth in Table 8. In another specific embodiment, the VL CDR1, VL CDR2, and VL CDR3 are as set forth in Table 10, and the VH CDR1, VH CDR2, and VH CDR3 are as set forth in Table 10. In another specific embodiment, the VL CDR1, VL CDR2, and VL CDR3 are as set forth in Table 12, and the VH CDR1, VH CDR2, and VH CDR3 are as set forth in Table 12.

In a particular embodiment, an antibody described herein, or an antigen-binding fragment thereof, comprises the VL CDR1, VL CDR2, and VL CDR3 of Abcon, and the VL FR1, VL FR2, VL FR3, and VL FR4 of Abcon. In a specific embodiment, the VL CDR1, VL CDR2, and VL CDR3 are as set forth in Table 1, and the VL FR1, VL FR2, VL FR3, and VL FR4 are as set forth in Table 2. In another specific embodiment, the VL CDR1, VL CDR2, and VL CDR3 are as set forth in Table 4, and the VL FR1, VL FR2, VL FR3, and VL FR4 are as set forth in Table 5. In another specific embodiment, the VL CDR1, VL CDR2, and VL CDR3 are as set forth in Table 6, and the VL FR1, VL FR2, VL FR3, and VL FR4 are as set forth in Table 7. In another specific embodiment, the VL CDR1, VL CDR2, and VL CDR3 are as set forth in Table 8, and the VL FR1, VL FR2, VL FR3, and VL FR4 are as set forth in Table 9. In another specific embodiment, the VL CDR1, VL CDR2, and VL CDR3 are as set forth in Table 10, and the VL FR1, VL FR2, VL FR3, and VL FR4 are as set forth in Table 11. In another specific embodiment, the VL CDR1, VL CDR2, and VL CDR3 are as set forth in Table 12, and the VL FR1, VL FR2, VL FR3, and VL FR4 are as set forth in Table 13.

In a particular embodiment, an antibody described herein, or an antigen-binding fragment thereof, comprises the VH CDR1, VH CDR2, and VH CDR3 of Abcon, and the VH FR1, VH FR2, VH FR3, and VH FR4 of Abcon. In a specific embodiment, the VH CDR1, VH CDR2, and VH CDR3 are as set forth in Table 1, and the VH FR1, VH FR2, VH FR3, and VH FR4 are as set forth in Table 2. In another specific embodiment, the VH CDR1, VH CDR2, and VH CDR3 are as set forth in Table 4, and the VH FR1, VH FR2, VH FR3, and VH FR4 are as set forth in Table 5. In another specific embodiment, the VH CDR1, VH CDR2, and VH CDR3 are as set forth in Table 6, and the VH FR1, VH FR2, VH FR3, and VH FR4 are as set forth in Table 7. In another specific embodiment, the VH CDR1, VH CDR2, and VH CDR3 are as set forth in Table 8, and the VH FR1, VH FR2, VH FR3, and VH FR4 are as set forth in Table 9. In another specific embodiment, the VH CDR1, VH CDR2, and VH CDR3 are as set forth in Table 10, and the VH FR1, VH FR2, VH FR3, and VH FR4 are as set forth in Table 11. In another specific embodiment, the VH CDR1, VH CDR2, and VH CDR3 are as set forth in Table 12, and the VH FR1, VH FR2, VH FR3, and VH FR4 are as set forth in Table 13.

In certain aspects, an antibody described herein may be described by its VL alone, or its VH alone, or by its 3 VL CDRs alone, or its 3 VH CDRs alone. See, for example, Rader et al., 1998, Proc. Natl. Acad. Sci. USA, 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also, Clackson et al., 1991, Nature 352:624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also, Kim & Hong, 2007, J. Microbiol. 45:572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In another embodiment, affinity maturation techniques can be used to alter one or more CDR(s), followed by screening of the resultant binding molecules for the desired change in binding. Any affinity maturation techniques known in the art can be used. See also, Holland et al., 2013, J. Immun. Methods 394:55-61, which is incorporated herein by reference in its entirety, describing methods of producing libraries for directed evolution of proteins (e.g., affinity matured antibodies). In certain embodiments, a CDR altered (e.g., by affinity maturation) can result in changes in binding affinity (e.g., on-rate of binding and/or off-rate of binding). In some embodiments, methods known in the art can be used to analyze binding affinity and immunogenicity of affinity matured antibodies. Any method known in the art can be used to ascertain immunospecific binding to Axl (e.g., human Axl ECD), for example, the binding assays and conditions described in the “Examples” section (Section 6) provided herein.

In a specific embodiment, the position of one or more CDRs along the VH (e.g., CDR1, CDR2, or CDR3) and/or VL (e.g., CDR1, CDR2, or CDR3) region of an antibody described herein may vary by one, two, three, four, five, or six amino acid positions as long as immunospecific binding to Axl (e.g., human Axl ECD) is maintained (e.g., substantially maintained, for example, at least 50%, at least 60%, at least 70%, at least 80%, at least 90%, at least 95%). For example, in one embodiment, the position defining a CDR of any of Table 1, Table 4, Table 6, Table 8, Table 10, and Table 12 may vary by shifting the N-terminal and/or C- terminal boundary of the CDR by one, two, three, four, five, or six amino acids, relative to the current CDR position, as long as immunospecific binding to Axl (e.g., human Axl) is maintained (e.g., substantially maintained, for example, at least 50%, at least 60%, at least 70%, at least 80%, at least 90%, at least 95%). In another embodiment, the length of one or more CDRs along the VH (e.g., CDR1, CDR2, or CDR3) and/or VL (e.g., CDR1, CDR2, or CDR3) region of an antibody described herein may vary (e.g., be shorter or longer) by one, two, three, four, five, or more amino acids, so long as immunospecific binding to Axl (e.g., human Axl ECD) is maintained (e.g., substantially maintained, for example, at least 50%, at least 60%, at least 70%, at least 80%, at least 90%, at least 95%). For example, in one embodiment, a VH and/or VL CDR1, CDR2, and/or CDR3 described herein may be one, two, three, four, five or more amino acids shorter than one or more of the CDRs described in any one of Table 1, Table 4, Table 6, Table 8, Table 10, and Table 12, so long as immunospecific binding to Axl (e.g., human Axl ECD) is maintained (e.g., substantially maintained, for example, at least 50%, at least 60%, at least 70%, at least 80%, at least 90%, at least 95%). In another embodiment, a VH and/or VL CDR1, CDR2, and/or CDR3 described herein may be one, two, three, four, five or more amino acids longer than one or more of the CDRs described in any one of Table 1, Table 4, Table 6, Table 8, Table 10, and Table 12, as long as immunospecific binding to Axl (e.g., human Axl ECD) is maintained (e.g., substantially maintained, for example, at least 50%, at least 60%, at least 70%, at least 80%, at least 90%, at least 95%). In another embodiment, the amino terminus of a VH and/or VL CDR1, CDR2, and/or CDR3 described herein may be extended by one, two, three, four, five or more amino acids compared to one or more of the CDRs described in any one of Table 1, Table 4, Table 6, Table 8, Table 10 and Table 12, so long as immunospecific binding to Axl (e.g., human Axl ECD) is maintained (e.g., substantially maintained, for example, at least 50%, at least 60%, at least 70%, at least 80%, at least 90%, at least 95%). In another embodiment, the carboxy terminus of a VH and/or VL CDR1, CDR2, and/or CDR3 described herein may be extended by one, two, three, four, five or more amino acids compared to one or more of the CDRs described in any one of Table 1, Table 4, Table 6, Table 8, Table 10, and Table 12, as long as immunospecific binding to Axl (e.g., human Axl ECD) is maintained (e.g., substantially maintained, for example, at least 50%, at least 60%, at least 70%, at least 80%, at least 90%, at least 95%). In another embodiment, the amino terminus of a VH and/or VL CDR1, CDR2, and/or CDR3 described herein may be shortened by one, two, three, four, five or more amino acids compared to one or more of the CDRs described in any one of Table 1, Table 4, Table 6, Table 8, Table 10, and Table 12, so long as immunospecific binding to Axl (e.g., human Axl) is maintained (e.g., substantially maintained, for example, at least 50%, at least 60%, at least 70%, at least 80%, at least 90%, at least 95%). In one embodiment, the carboxy terminus of a VH and/or VL CDR1, CDR2, and/or CDR3 described herein may be shortened by one, two, three, four, five or more amino acids compared to one or more of the CDRs described in any one of Table 1, Table 4, Table 6, Table 8, Table 10, and Table 12, so long as immunospecific binding to Axl (e.g., human Axl ECD) is maintained (e.g., substantially maintained, for example, at least 50%, at least 60%, at least 70%, at least 80%, at least 90%, at least 95%). Any method known in the art can be used to ascertain whether immunospecific binding to Axl (e.g., human Axl ECD) is maintained, for example, the binding assays and conditions described in the “Examples” section (Section 6) provided herein. For example, Section 6.2 provided herein describes an assay for measuring binding to an ECD of human Axl.

In specific aspects, provided herein is an antibody comprising an antibody light chain and heavy chain, e.g., a separate light chain and heavy chain. With respect to the light chain, in a specific embodiment, the light chain of an antibody described herein is a kappa light chain. In another specific embodiment, the light chain of an antibody described herein is a lambda light chain. In yet another specific embodiment, the light chain of an antibody described herein is a human kappa light chain or a human lambda light chain. In a particular embodiment, an antibody described herein, which specifically binds to an Axl polypeptide (e.g., human Axl) comprises a light chain, wherein the amino acid sequence of the VL chain region comprises any amino acid sequence described herein (e.g., SEQ ID NO: 6 or 12), and wherein the constant region of the light chain comprises the amino acid sequence of a human kappa light chain constant region. In another particular embodiment, an antibody described herein, which specifically binds an Axl polypeptide (e.g., human Axl) comprises a light chain wherein the amino acid sequence of the VL chain region can comprise any amino acid sequence described herein (e.g., SEQ ID NO: 6 or 12), and wherein the constant region of the light chain comprises the amino acid sequence of a human lambda light chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

With respect to the heavy chain, in a specific embodiment, the heavy chain of an antibody described herein can be an alpha (α), delta (δ), epsilon (ε), gamma (γ) or mu (μ) heavy chain. In another specific embodiment, the heavy chain of an antibody described can comprise a human alpha (α), delta (δ), epsilon (ε), gamma (γ) or mu (μ) heavy chain. In a particular embodiment, an antibody described herein, which specifically binds to an Axl polypeptide (e.g., human Axl), comprises a heavy chain wherein the amino acid sequence of the VH chain region can comprise any amino acid sequence described herein (e.g., any of SEQ ID NO: 1), and wherein the constant region of the heavy chain comprises the amino acid sequence of a human gamma (γ) heavy chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

In a specific embodiment, an antibody or antigen-binding fragment described herein, which specifically binds to an Axl polypeptide (e.g., human Axl) comprises a VL chain region and a VH chain region comprising any amino acid sequences described herein, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In another specific embodiment, an antibody or antigen-binding fragment described herein, which specifically binds to an Axl polypeptide (e.g., human Axl) comprises a VL chain region and a VH chain region comprising any amino acid sequences described herein, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. In a particular embodiment, the constant regions comprise the amino acid sequences of the constant regions of a human IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule.

In yet another specific embodiment, an antibody described herein, which specifically binds to an Axl polypeptide (e.g., human Axl), comprises a VL chain region and a VH chain region comprising any amino acid sequences described herein, and wherein the constant regions comprise the amino acid sequences of the constant regions of a human IgG1 (e.g., isotype a, z, or f), human IgG2, or human IgG4. Non-limiting examples of human constant regions are described in the art, e.g., see Kabat et al. (1991) Sequences of Proteins of Immunological Interest, Fifth Edition, U.S. Department of Health and Human Services, NIH Publication No. 91-3242.

In another specific embodiment, an antibody or antigen-binding fragment described herein, which specifically binds to a Axl polypeptide (e.g., human Axl) comprises a VL chain region and a VH chain region comprising any amino acid sequences described herein, and further comprises one or more modifications to the amino acid sequences of the constant regions, wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. In some embodiments, modifications refer to at most 1, 2, 3, 4, 5, or 6 amino acid substitution(s).

In certain embodiments the constant region can be deglycosylated (e.g., via mutation at the glycosylation consensus sequence).

The constant region can mediate several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, and control of immunoglobulin production.

In certain embodiments, the constant region does not have effector function or has a diminished effector function. For example, in certain embodiments, a Axl antibody or antigen-binding fragment thereof described herein can comprise an Fc region in which a portion of the Fc region has been deleted or otherwise altered so as to provide desired biochemical characteristics, such as increased localization in an inflamed tissue, increased tissue penetration, reduced serum half-life, or increased serum half-life, when compared with a fusion protein of approximately the same immunogenicity comprising an unaltered Fc region. In some embodiments, an Axl antibody or antigen-binding fragment thereof described herein comprises an Fc region with altered binding affinity to one or more Fc receptors (FcRs).

In a specific embodiment, an Axl antibody or antigen-binding fragment thereof described herein comprises a wild-type Fc region that is capable of binding one or more Fc receptors (FcRs). In another specific embodiment, an Axl antibody or antigen-binding fragment thereof described herein comprises an Fc region with enhanced binding affinity to one or more Fc receptors (FcRs) relative to a wild-type Fc region. In another specific embodiment, an Axl antibody or antigen-binding fragment thereof described herein comprises an Fc region with reduced binding affinity to one or more Fc receptors (FcRs) relative to a wild-type Fc region. In another specific embodiment, an Axl antibody or antigen-binding fragment thereof described herein comprises an Fc region with no binding affinity to one or more Fc receptors (FcRs). In another specific embodiment, an Axl antibody or antigen-binding fragment thereof described herein comprises an Fc region with no binding affinity to any Fc receptors (FcRs). In another specific embodiments, an Axl antibody or antigen-binding fragment thereof described herein does not comprise an Fc region.

In specific embodiments, an Axl antibody or antigen-binding fragment thereof described herein is capable of eliciting an effector function, e.g., antibody-dependent cell-mediated cytotoxicity (ADCC), antibody-dependent cellular phagocytosis (ADCP) and complement-dependent cytotoxicity (CDC). In a specific embodiment, an Axl antibody or antigen-binding fragment thereof described herein is capable of eliciting ADCC.

Those skilled in the art will appreciate that Fc of an antibody or antigen-binding fragment thereof may interact with both activating and inhibitory Fcγ receptors, which may yield vastly different clinical outcomes. In some embodiments, an Axl antibody or antigen-binding fragment thereof described herein comprises a wild-type Fc region that is capable of binding an activating Fc gamma receptor (FcγR). In some embodiments, an Axl antibody or antigen-binding fragment thereof described herein comprises an Fc region with altered binding affinity to an activating Fc gamma receptor (FcγR). In a specific embodiment, an Axl antibody or antigen-binding fragment thereof described herein comprises an Fc region with enhanced binding affinity to an activating Fc gamma receptor (FcγR) relative to a wild-type Fc region. In another specific embodiment, an Axl antibody or antigen-binding fragment thereof described herein comprises an Fc region with reduced binding affinity to an activating Fc gamma receptor (FcγR) relative to a wild-type Fc region. In another specific embodiment, an Axl antibody or antigen-binding fragment thereof described herein comprises an Fc region with no binding affinity to an activating Fc gamma receptor (FcγR). In some embodiments, an Axl antibody or antigen-binding fragment thereof described herein comprises a wild-type Fc region that is capable of binding an inhibitory Fc gamma receptor (FcγR). In some embodiments, an Axl antibody or antigen-binding fragment thereof described herein comprises an Fc region with altered binding affinity to an inhibitory Fc gamma receptor (FcγR). In a specific embodiment, an Axl antibody or antigen-binding fragment thereof described herein comprises an Fc region with enhanced binding affinity to an inhibitory Fc gamma receptor (FcγR) relative to a wild-type Fc region. In another specific embodiment, an Axl antibody or antigen-binding fragment thereof described herein comprises an Fc region with reduced binding affinity to an inhibitory Fc gamma receptor (FcγR) relative to a wild-type Fc region. In another specific embodiment, an Axl antibody or antigen-binding fragment thereof described herein comprises an Fc region with no binding affinity to an inhibitory Fc gamma receptor (FcγR).

In some embodiments, such a modified antibody may have only a partial deletion of a constant domain or substitution of a few or even a single amino acid. For example, the mutation of 1, 2, 3, 4 or 5 amino acids in selected areas of the CH2 domain may be enough to substantially alter Fc binding. Similarly, it may be desirable to simply delete the part of one or more constant region domain(s) that control a specific effector function to be modulated. Such partial deletions of the constant regions may improve selected characteristics of the antibody while leaving other desirable functions associated with the subject constant region domain intact. Moreover, the constant regions of the disclosed antibodies may be modified through the mutation or substitution of one or more amino acids that enhances the profile of the resulting construct. In this respect it may be possible to alter the activity provided by a conserved binding site (e.g., FcγR binding) while substantially maintaining the configuration and immunogenic profile of the modified antibody. In certain embodiments, the modified antibodies comprise the addition of one or more amino acids to the constant region to enhance desirable characteristics such as decreasing effector function or to eliminate carbohydrate attachment sites.

In certain embodiments, an antibody or antigen-binding fragment thereof disclosed herein can be modified such that it can provide for altered effector functions that, in turn, affect the biological profile of the administered antibody. For example, in some embodiments, the deletion or modification (through point mutations or other means) of a constant region domain may alter Fc receptor binding of the circulating modified antibody. In other embodiments, the constant region modifications increase the serum half-life of the antibody. In other embodiments, the constant region modifications reduce the serum half-life of the antibody. In some embodiments, the constant region is modified to eliminate disulfide linkages or oligosaccharide moieties.

In certain embodiments, an antibody or antigen-binding fragment disclosed herein can be modified such that it does not have one or more effector functions. In some embodiments, the antibody or antigen-binding fragment disclosed herein can be modified such that it has altered ADCC activity, and/or altered complement-dependent cytotoxicity (CDC) activity. In certain embodiments, an antibody or antigen-binding fragment disclosed herein can be modified such that it does not bind an Fc receptor, and/or complement factors. For example, in certain embodiments, an antibody or antigen-binding fragment disclosed herein can be modified such that it preferentially binds to an Fc receptor (e.g., an activating FcγR).

In certain alternative embodiments, an antibody or antigen-binding fragment described herein, which specifically binds to an Axl polypeptide can be bispecific or multi specific and comprise at least two antigen-binding sites with differing specificities. In certain embodiments, the bispecific binding molecule can alter effector function, or enhance or diminish affinity of antibody to FcR. For example, in certain embodiments, an antibody or antigen-binding fragment disclosed herein can be bispecific such that it preferentially binds to an Fc receptor (e.g., an activating FcγR).

In another embodiment, a polypeptide making up an antibody or antigen-binding fragment described herein further comprises a signal sequence, for example, an N-terminal signal sequence. In certain instances, such a polypeptide may contain such a signal sequence when initially produced, e.g., translated, but may have the signal sequence removed prior to or during assembly of the final antibody or antigen-binding fragment. In alternative embodiments, the antibody polypeptide or antigen-binding fragment does not comprise a signal sequence.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Ab301; (ii) the heavy chain comprises a VH chain region comprising a VH CDR1, VH CDR2, and VH CDR3 of Ab301; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Ab302; (ii) the heavy chain comprises a VH chain region comprising a VH CDR1, VH CDR2, and VH CDR3 of Ab302; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Ab303; (ii) the heavy chain comprises a VH chain region comprising a VH CDR1, VH CDR2, and VH CDR3 of Ab303; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Ab304; (ii) the heavy chain comprises a VH chain region comprising a VH CDR1, VH CDR2, and VH CDR3 of Ab304; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Ab305; (ii) the heavy chain comprises a VH chain region comprising a VH CDR1, VH CDR2, and VH CDR3 of Ab305; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Abcon; (ii) the heavy chain comprises a VH chain region comprising a VH CDR1, VH CDR2, and VH CDR3 of Abcon; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Table 1; (ii) the heavy chain comprises a VH chain region comprising a VH CDR1, VH CDR2, and VH CDR3 of Table 1; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Table 4; (ii) the heavy chain comprises a VH chain region comprising a VH CDR1, VH CDR2, and VH CDR3 of Table 4; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Table 6; (ii) the heavy chain comprises a VH chain region comprising a VH CDR1, VH CDR2, and VH CDR3 of Table 6; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Table 8; (ii) the heavy chain comprises a VH chain region comprising a VH CDR1, VH CDR2, and VH CDR3 of Table 8; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Table 10; (ii) the heavy chain comprises a VH chain region comprising a VH CDR1, VH CDR2, and VH CDR3 of Table 10; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising a VL CDR1, VL CDR2, and VL CDR3 of Table 12; (ii) the heavy chain comprises a VH chain region comprising a VH CDR1, VH CDR2, and VH CDR3 of Table 12; (iii) the light chain further comprises a constant light chain domain comprising the amino acid sequence of the constant domain of a human kappa or lambda light chain; and (iv) the heavy chain further comprises a constant heavy chain domain comprising the amino acid sequence of the constant domain of a human IgG, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising the amino acid sequence of SEQ ID NO: 12; (ii) the heavy chain comprises a VH chain region comprising the amino acid sequence of SEQ ID NO: 11; (iii) the light chain further comprises a constant domain comprising the amino acid sequence of the constant domain of a human kappa or lambda light chain; and (iv) the heavy chain further comprises a constant domain comprising the amino acid sequence of the constant domain of a human IgG1, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising the amino acid sequence of SEQ ID NO: 6; (ii) the heavy chain comprises a VH chain region comprising the amino acid sequence of SEQ ID NO: 7; (iii) the light chain further comprises a constant domain comprising the amino acid sequence of the constant domain of a human kappa or lambda light chain; and (iv) the heavy chain further comprises a constant domain comprising the amino acid sequence of the constant domain of a human IgG1, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising the amino acid sequence of SEQ ID NO: 6; (ii) the heavy chain comprises a VH chain region comprising the amino acid sequence of SEQ ID NO: 8; (iii) the light chain further comprises a constant domain comprising the amino acid sequence of the constant domain of a human kappa or lambda light chain; and (iv) the heavy chain further comprises a constant domain comprising the amino acid sequence of the constant domain of a human IgG1, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising the amino acid sequence of SEQ ID NO: 6; (ii) the heavy chain comprises a VH chain region comprising the amino acid sequence of SEQ ID NO: 9; (iii) the light chain further comprises a constant domain comprising the amino acid sequence of the constant domain of a human kappa or lambda light chain; and (iv) the heavy chain further comprises a constant domain comprising the amino acid sequence of the constant domain of a human IgG1, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising the amino acid sequence of SEQ ID NO: 6; (ii) the heavy chain comprises a VH chain region comprising the amino acid sequence of SEQ ID NO: 10; (iii) the light chain further comprises a constant domain comprising the amino acid sequence of the constant domain of a human kappa or lambda light chain; and (iv) the heavy chain further comprises a constant domain comprising the amino acid sequence of the constant domain of a human IgG1, e.g., IgG1, heavy chain.

In another particular embodiment, an antibody described herein, which specifically binds to a Axl polypeptide (e.g., human Axl), comprises a light chain and a heavy chain, wherein (i) the light chain comprises a VL chain region comprising the amino acid sequence of SEQ ID NO: 6; (ii) the heavy chain comprises a VH chain region comprising the amino acid sequence of SEQ ID NO: 5; (iii) the light chain further comprises a constant domain comprising the amino acid sequence of the constant domain of a human kappa or lambda light chain; and (iv) the heavy chain further comprises a constant domain comprising the amino acid sequence of the constant domain of a human IgG1, e.g., IgG1, heavy chain.

In certain embodiments, one or more modifications can be made to the Fc region of an antibody or antigen-binding fragment thereof described herein. Generally, such a modification or modifications can be introduced to alter one or more functional properties of the antibody or antigen-binding fragment, such as serum half-life, complement fixation, Fc receptor binding, and/or antibody-dependent cellular cytotoxicity. Exemplary modifications are described, for example, in International Patent Application Publication No. WO 2008/153926 A2.

In specific embodiments, an antibody described herein, which specifically binds to Axl, e.g., human Axl ECD, comprises framework regions (e.g., framework regions of the VL domain and/or VH domain) that are human framework regions or derived from human framework regions. Non-limiting examples of human framework regions are described in the art, e.g., see Kabat et al. (1991) Sequences of Proteins of Immunological Interest, Fifth Edition, U.S. Department of Health and Human Services, NIH Publication No. 91-3242).

In certain embodiments, an antibody described herein comprises framework regions (e.g., framework regions of the VL domain and/or VH domain) that are primate (e.g., non-human primate) framework regions or derived from primate (e.g., non-human primate) framework regions.

In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD.

In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD, and wherein the antibody comprises VL CDRs that are identical to the VL CDRs of antibody Ab301. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of antibody Ab302. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of antibody Ab303. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of antibody Ab304. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of antibody Ab305. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of antibody Abcon.

In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD, and wherein the antibody comprises VL CDRs that are identical to the VL CDRs of Ab301 as set forth in Table 1. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of Ab302, Ab303, Ab 304, Ab305, or Abcon as set forth in Table 1. In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD, and wherein the antibody comprises VL CDRs that are identical to the VL CDRs of Ab301 as set forth in Table 4. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of Ab302, Ab303, Ab 304, Ab305, or Abcon as set forth in Table 4. In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD, and wherein the antibody comprises VL CDRs that are identical to the VL CDRs of Ab301 as set forth in Table 6. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of Ab302, Ab303, Ab 304, Ab305, or Abcon as set forth in Table 6. In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD, and wherein the antibody comprises VL CDRs that are identical to the VL CDRs of Ab301 as set forth in Table 8. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of Ab302, Ab303, Ab 304, Ab305, or Abcon as set forth in Table 8. In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD, and wherein the antibody comprises VL CDRs that are identical to the VL CDRs of Ab301 as set forth in Table 10. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of Ab302, Ab303, Ab 304, Ab305, or Abcon as set forth in Table 10. In certain embodiments, an antibody described herein comprises a VL domain having at least 80%, at least 85%, at least 90%, at least 95%, or at least 98% sequence identity to the amino acid sequence of SEQ ID NO: 12, wherein the antibody specifically binds to Axl, e.g., human Axl ECD, and wherein the antibody comprises VL CDRs that are identical to the VL CDRs of Ab301 as set forth in Table 12. In certain embodiments, an antibody described herein comprises a VL domain having at least 80%, at least 85%, at least 90%, at least 95%, or at least 98% sequence identity to the amino acid sequence of SEQ ID NO: 6, wherein the antibody specifically binds to Axl, e.g., human Axl ECD, and wherein the antibody comprises VL CDRs that are identical to the VL CDRs of Ab302, Ab303, Ab 304, Ab305, or Abcon as set forth in Table 12.

In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD. In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD. In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD. In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD. In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD.

In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD, and wherein the antibody comprises VH CDRs that are identical to the VH CDRs of antibody Ab301. In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD, and wherein the antibody comprises VH CDRs that are identical to the VH CDRs of antibody Ab302. In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD, and wherein the antibody comprises VH CDRs that are identical to the VH CDRs of antibody Ab303. In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD, and wherein the antibody comprises VH CDRs that are identical to the VH CDRs of antibody Ab304. In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD, and wherein the antibody comprises VH CDRs that are identical to the VH CDRs of antibody Ab305. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of antibody Abcon. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of any one of antibody Ab301, Ab302, Ab303, Ab304 or Ab305.

In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD, and wherein the antibody comprises VH CDRs that are identical to the VH CDRs of Ab301 as set forth in Table 1. In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD, and wherein the antibody comprises VH CDRs that are identical to the VH CDRs of Ab301 as set forth in Table 4. In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD, and wherein the antibody comprises VH CDRs that are identical to the VH CDRs of Ab301 as set forth in Table 6. In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD, and wherein the antibody comprises VH CDRs that are identical to the VH CDRs of Ab301 as set forth in Table 8. In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD, and wherein the antibody comprises VH CDRs that are identical to the VH CDRs of Ab301 as set forth in Table 10. In certain embodiments, an antibody described herein comprises a VH domain having at least 80%, at least 85%, at least 90%, at least 95%, or at least 98% sequence identity to the amino acid sequence of SEQ ID NO: 11, wherein the antibody specifically binds to Axl, e.g., human Axl ECD, and wherein the antibody comprises VH CDRs that are identical to the VH CDRs of Ab301 as set forth in Table 12.

In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD, and wherein the antibody comprises VH CDRs that are identical to the VH CDRs of Ab302 as set forth in Table 1. In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD, and wherein the antibody comprises VH CDRs that are identical to the VH CDRs of Ab302 as set forth in Table 4. In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD, and wherein the antibody comprises VH CDRs that are identical to the VH CDRs of Ab302 as set forth in Table 6. In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD, and wherein the antibody comprises VH CDRs that are identical to the VH CDRs of Ab302 as set forth in Table 8. In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD, and wherein the antibody comprises VH CDRs that are identical to the VH CDRs of Ab302 as set forth in Table 10. In certain embodiments, an antibody described herein comprises a VH domain having at least 80%, at least 85%, at least 90%, at least 95%, or at least 98% sequence identity to the amino acid sequence of SEQ ID NO: 7, wherein the antibody specifically binds to Axl, e.g., human Axl ECD, and wherein the antibody comprises VH CDRs that are identical to the VH CDRs of Ab302 as set forth in Table 12.

In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD, and wherein the antibody comprises VH CDRs that are identical to the VH CDRs of Ab303 as set forth in Table 1. In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD, and wherein the antibody comprises VH CDRs that are identical to the VH CDRs of Ab303 as set forth in Table 4. In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD, and wherein the antibody comprises VH CDRs that are identical to the VH CDRs of Ab303 as set forth in Table 6. In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD, and wherein the antibody comprises VH CDRs that are identical to the VH CDRs of Ab303 as set forth in Table 8. In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD, and wherein the antibody comprises VH CDRs that are identical to the VH CDRs of Ab303 as set forth in Table 10. In certain embodiments, an antibody described herein comprises a VH domain having at least 80%, at least 85%, at least 90%, at least 95%, or at least 98% sequence identity to the amino acid sequence of SEQ ID NO: 8, wherein the antibody specifically binds to Axl, e.g., human Axl ECD, and wherein the antibody comprises VH CDRs that are identical to the VH CDRs of Ab303 as set forth in Table 12.

In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD, and wherein the antibody comprises VH CDRs that are identical to the VH CDRs of Ab304 as set forth in Table 1. In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD, and wherein the antibody comprises VH CDRs that are identical to the VH CDRs of Ab304 as set forth in Table 4. In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD, and wherein the antibody comprises VH CDRs that are identical to the VH CDRs of Ab304 as set forth in Table 6. In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD, and wherein the antibody comprises VH CDRs that are identical to the VH CDRs of Ab304 as set forth in Table 8. In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD, and wherein the antibody comprises VH CDRs that are identical to the VH CDRs of Ab304 as set forth in Table 10. In certain embodiments, an antibody described herein comprises a VH domain having at least 80%, at least 85%, at least 90%, at least 95%, or at least 98% sequence identity to the amino acid sequence of SEQ ID NO: 9, wherein the antibody specifically binds to Axl, e.g., human Axl ECD, and wherein the antibody comprises VH CDRs that are identical to the VH CDRs of Ab304 as set forth in Table 12.

In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD, and wherein the antibody comprises VH CDRs that are identical to the VH CDRs of Ab305 as set forth in Table 1. In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD, and wherein the antibody comprises VH CDRs that are identical to the VH CDRs of Ab305 as set forth in Table 4. In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD, and wherein the antibody comprises VH CDRs that are identical to the VH CDRs of Ab305 as set forth in Table 6. In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD, and wherein the antibody comprises VH CDRs that are identical to the VH CDRs of Ab305 as set forth in Table 8. In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD, and wherein the antibody comprises VH CDRs that are identical to the VH CDRs of Ab305 as set forth in Table 10. In certain embodiments, an antibody described herein comprises a VH domain having at least 80%, at least 85%, at least 90%, at least 95%, or at least 98% sequence identity to the amino acid sequence of SEQ ID NO: 10, wherein the antibody specifically binds to Axl, e.g., human Axl ECD, and wherein the antibody comprises VH CDRs that are identical to the VH CDRs of Ab305 as set forth in Table 12.

In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of Abcon as set forth in Table 1. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of Abcon as set forth in Table 4. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of Abcon as set forth in Table 6. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of Abcon as set forth in Table 8. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of Abcon as set forth in Table 10. In certain embodiments, an antibody described herein comprises a VH domain having at least 80%, at least 85%, at least 90%, at least 95%, or at least 98% sequence identity to the amino acid sequence of SEQ ID NO: 5, wherein the antibody specifically binds to Axl, e.g., human Axl ECD, and wherein the antibody comprises VH CDRs that are identical to the VH CDRs of Abcon as set forth in Table 12.

In other embodiments, the antibodies and antigen binding fragments thereof presented herein that specifically bind to human Axl comprise conservative sequence modifications as described herein. With respect to polypeptides such as antibodies or antigen binding fragment thereof, conservative sequence modifications include conservative amino acid substitutions that include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in a particular embodiment, a predicted nonessential amino acid residue in a human anti-Axl antibody is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In particular embodiments, the conservative sequence modifications described herein modify the amino acid sequences of the Axl antibodies or antigen binding fragments thereof by 50%, or 55%, or 60%, or 65%, or 70%, or 75%, or 80%, or 85%, or 90%, or 95%, or 98%, or 99%. In certain embodiments, the nucleotide and amino acid sequence modifications refer to at most 1, 2, 3, 4, 5, or 6 amino acid substitutions to the CDRs described in any of Table 1, Table 4, Table 6, Table 8, Table 10, and Table 12. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution.

In certain embodiments, an antibody or antigen-binding fragment thereof that specifically binds to Axl, e.g., human Axl ECD described herein comprises: i) a VL domain having at least 80%, at least 85%, at least 90%, at least 95%, or at least 98% sequence identity to the amino acid sequence of SEQ ID NO: 12, ii) a VL domain having at least 80%, at least 85%, at least 90%, at least 95%, or at least 98% sequence identity to the amino acid sequence of SEQ ID NO: 12, and wherein the antibody comprises VL CDRs that are identical to the VL CDRs of antibody Ab301, or iii) a VL domain having at least 80%, at least 85%, at least 90%, at least 95%, or at least 98% sequence identity to the amino acid sequence of SEQ ID NO: 12, and wherein the antibody comprises VL CDRs that are identical to the VL CDRs of Ab301 as set forth in Table 1, Table 4, Table 6, Table 8, Table 10, or Table 12; and iv) a VH domain having at least 80%, at least 85%, at least 90%, at least 95%, or at least 98% sequence identity to the amino acid sequence of SEQ ID NO: 11, v) a VH domain having at least 80%, at least 85%, at least 90%, at least 95%, or at least 98% sequence identity to the amino acid sequence of SEQ ID NO: 11 and wherein the antibody comprises VH CDRs that are identical to the VH CDRs of antibody Ab301, or vi) a VH domain having at least 80%, at least 85%, at least 90%, at least 95%, or at least 98% sequence identity to the amino acid sequence of SEQ ID NO: 11 and wherein the antibody comprises VH CDRs that are identical to the VH CDRs of Ab301 as set forth in Table 1, Table 4, Table 6, Table 8, Table 10, or Table 12.

In other certain embodiments, an antibody or antigen-binding fragment thereof that specifically binds to Axl, e.g., human Axl ECD described herein comprises: i) a VL domain having at least 80%, at least 85%, at least 90%, at least 95%, or at least 98% sequence identity to the amino acid sequence of SEQ ID NO: 6, ii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of antibody Ab302, or iii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of Ab302 as set forth in Table 1, Table 4, Table 6, Table 8, Table 10, or Table 12; and iv) a VH domain having at least 80%, at least 85%, at least 90%, at least 95%, or at least 98% sequence identity to the amino acid sequence of SEQ ID NO: 7, v) a VH domain having at least 80%, at least 85%, at least 90%, at least 95%, or at least 98% sequence identity to the amino acid sequence of SEQ ID NO: 7 and wherein the antibody comprises VH CDRs that are identical to the VH CDRs of antibody Ab302, or vi) a VH domain having at least 80%, at least 85%, at least 90%, at least 95%, or at least 98% sequence identity to the amino acid sequence of SEQ ID NO: 7 and wherein the antibody comprises VH CDRs that are identical to the VH CDRs of Ab302 as set forth in Table 1, Table 4, Table 6, Table 8, Table 10, or Table 12.

In other certain embodiments, an antibody or antigen-binding fragment thereof that specifically binds to Axl, e.g., human Axl ECD described herein comprises: i) a VL domain having at least 80%, at least 85%, at least 90%, at least 95%, or at least 98% sequence identity to the amino acid sequence of SEQ ID NO: 6, ii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of antibody Ab303, or iii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of Ab303 as set forth in Table 1, Table 4, Table 6, Table 8, Table 10, or Table 12; and iv) a VH domain having at least 80%, at least 85%, at least 90%, at least 95%, or at least 98% sequence identity to the amino acid sequence of SEQ ID NO: 8, v) a VH domain having at least 80%, at least 85%, at least 90%, at least 95%, or at least 98% sequence identity to the amino acid sequence of SEQ ID NO: 8 and wherein the antibody comprises VH CDRs that are identical to the VH CDRs of antibody Ab303, or vi) a VH domain having at least 80%, at least 85%, at least 90%, at least 95%, or at least 98% sequence identity to the amino acid sequence of SEQ ID NO: 8 and wherein the antibody comprises VH CDRs that are identical to the VH CDRs of Ab303 as set forth in Table 1, Table 4, Table 6, Table 8, Table 10, or Table 12.

In certain embodiments, an antibody or antigen-binding fragment thereof that specifically binds to Axl, e.g., human Axl ECD described herein comprises: i) a VL domain having at least 80%, at least 85%, at least 90%, at least 95%, or at least 98% sequence identity to the amino acid sequence of SEQ ID NO: 6, ii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of antibody Ab304, or iii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of Ab304 as set forth in Table 1, Table 4, Table 6, Table 8, Table 10, or Table 12; and iv) a VH domain having at least 80%, at least 85%, at least 90%, at least 95%, or at least 98% sequence identity to the amino acid sequence of SEQ ID NO: 9, v) a VH domain having at least 80%, at least 85%, at least 90%, at least 95%, or at least 98% sequence identity to the amino acid sequence of SEQ ID NO: 9 and wherein the antibody comprises VH CDRs that are identical to the VH CDRs of antibody Ab304, or vi) a VH domain having at least 80%, at least 85%, at least 90%, at least 95%, or at least 98% sequence identity to the amino acid sequence of SEQ ID NO: 9 and wherein the antibody comprises VH CDRs that are identical to the VH CDRs of Ab304 as set forth in Table 1, Table 4, Table 6, Table 8, Table 10, or Table 12.

In certain embodiments, an antibody or antigen-binding fragment thereof that specifically binds to Axl, e.g., human Axl ECD described herein comprises: i) a VL domain having at least 80%, at least 85%, at least 90%, at least 95%, or at least 98% sequence identity to the amino acid sequence of SEQ ID NO: 6, ii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of antibody Ab305, or iii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of Ab305 as set forth in Table 1, Table 4, Table 6, Table 8, Table 10, or Table 12; and iv) a VH domain having at least 80%, at least 85%, at least 90%, at least 95%, or at least 98% sequence identity to the amino acid sequence of SEQ ID NO: 10, v) a VH domain having at least 80%, at least 85%, at least 90%, at least 95%, or at least 98% sequence identity to the amino acid sequence of SEQ ID NO: 10 and wherein the antibody comprises VH CDRs that are identical to the VH CDRs of antibody Ab305, or vi) a VH domain having at least 80%, at least 85%, at least 90%, at least 95%, or at least 98% sequence identity to the amino acid sequence of SEQ ID NO: 10 and wherein the antibody comprises VH CDRs that are identical to the VH CDRs of Ab305 as set forth in Table 1, Table 4, Table 6, Table 8, Table 10, or Table 12.

In certain embodiments, an antibody or antigen-binding fragment thereof that specifically binds to Axl, e.g., human Axl ECD described herein comprises: i) a VL domain having at least 80%, at least 85%, at least 90%, at least 95%, or at least 98% sequence identity to the amino acid sequence of SEQ ID NO: 6, ii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of antibody Abcon, or iii) a VL domain having at least 80%, at least 85%, at least 90%, at least 95%, or at least 98% sequence identity to the amino acid sequence of SEQ ID NO: 6, and wherein the antibody comprises VL CDRs that are identical to the VL CDRs of Abcon as set forth in Table 1, Table 4, Table 6, Table 8, Table 10, or Table 12; and iv) a VH domain having at least 80%, at least 85%, at least 90%, at least 95%, or at least 98% sequence identity to the amino acid sequence of SEQ ID NO: 5, v) a VH domain having at least 80%, at least 85%, at least 90%, at least 95%, or at least 98% sequence identity to the amino acid sequence of SEQ ID NO: 5 and wherein the antibody comprises VH CDRs that are identical to the VH CDRs of antibody Abcon, or vi) a VH domain having at least 80%, at least 85%, at least 90%, at least 95%, or at least 98% sequence identity to the amino acid sequence of SEQ ID NO: 5 and wherein the antibody comprises VH CDRs that are identical to the VH CDRs of Abcon as set forth in Table 1, Table 4, Table 6, Table 8, Table 10, or Table 12.

The determination of percent identity between two sequences (e.g., amino acid sequences or nucleic acid sequences) can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the)(BLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

Provided herein are antibodies that specifically bind to Axl and that can modulate Axl activity and/or expression (e.g., inhibit Axl activity and/or expression). In certain embodiments, an Axl antibody or antigen-binding fragment thereof provided herein specifically binds to an Axl polypeptide, e.g., an ECD of human Axl, and that inhibits (e.g., partially inhibits) a Axl activity.

Axl activity can relate to any activity of Axl such as those known or described in the art. Non-limiting examples of Axl activity include: Axl receptor dimerization, Axl receptor phosphorylation (e.g., tyrosine phosphorylation or autophosphorylation in the cytoplasmic domain), signaling downstream of the Axl receptor (e.g., P13K, PLC, GRB2, RAC1, SOCS-1, AKT, STAT1, or MAPK/ERK signaling), Axl ligand (e.g., Gas6) induced enhancement of cell proliferation, or cell survival (e.g., natural killer (NK) cells), modulation of NK cells, dendritic cells or macrophages, phagocytosis, inhibition of proinflammatory cytokine production (e.g., inhibition of TLR-induced production of proinflammatory cytokines (e.g., TNF, IL-6, IL-12 and type I interferons)).

The term “proinflammatory cytokines” or the like, as used in this disclosure, refers to not only inflammatory cytokines, but also includes, for example, growth factors (e.g., VEGF-A), cytokines induced in response to an inflammatory stimulus (e.g., IL-10 and IL-1RA), and chemokines (e.g., MIP-1a and MIP-1B).

In specific embodiments, antibodies and antigen-binding fragments described herein specifically bind to human Axl and block or inhibit (e.g., partially inhibit) binding of Axl ligand (e.g., Gas6) to Axl by at least about 5%, 10%, 15%, 20%, 25%, 30%, 35%, 40%, 45%, 50%, 55%, 60%, 65%, 70%, 75%, 80%, 85%, 90%, 95%, 98%, or 100% as assessed by methods described herein or known to one of skill in the art, e.g., ELISA assay, flow cytometry, or competition assay. In a specific embodiment, antibodies and antigen-binding fragments described herein inhibit binding of Gas6 to Axl (e.g., by at least about 5%, 10%, 15%, 20%, 25%, 30%, 35%, 40%, 45%, 50%, 55%, 60%, 65%, 70%, 75%, 80%, 85%, 90%, 95%, 98%, or 100%). In another specific embodiment, antibodies and antigen-binding fragments described herein do not inhibit binding of Gas6 to Axl.

In certain aspects, inhibition by anti-Axl antibodies described herein (e.g., monoclonal antibody) of Axl ligand (e.g., Gas6) binding to Axl can be characterized by IC₅₀ values, which reflects the concentration of anti-Axl antibodies achieving 50% inhibition of binding of Axl ligand to Axl. Thus, in specific embodiments, an anti-Axl antibody described herein (e.g., antibody Ab301 or an antigen-binding fragment thereof, Ab302 or an antigen-binding fragment thereof, Ab303 or an antigen-binding fragment thereof, Ab304 or an antigen-fragment thereof, Ab305 or an antigen-binding fragment thereof, or Abcon or an antigen-binding fragment thereof, or an antibody comprising CDRs of any of Ab301, Ab302, Ab303, Ab304, Ab305, or Abcon as set forth in Table 1, Table 4, Table 6, Table 8, Table 10, or Table 12, or an antibody comprising a VH and/or VL of Table 3) inhibits binding of Axl ligand to Axl with an IC₅₀ of at most about 10,000 nM, 1,000 nM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 5 nM, 1 nM, 0.75 nM, 0.5 nM, 0.1 nM, 0.05 nM, 0.01 nM, 0.005 nM, or 0.001 nM, as assessed by methods described herein and/or known to one of skill in the art, (e.g., ELISA assay or flow cytometry). In particular embodiments, an anti-Axl antibody described herein inhibits binding of Axl ligand to Axl with an IC₅₀ in the range of about 0.01 nM to 10,000 nM, 0.01 nM to 1,000 nM, 0.05 nM to 500 nM, 0.05 nM to 100 nM, or 0.05 nM to 50 nM, 0.1 nM to 50 nM as assessed by methods described herein and/or known to one of skill in the art, (e.g., ELISA assay or flow cytometry).

In certain embodiments, an anti-Axl antibody described herein can inhibit (e.g., partially inhibit) Axl activity by at least about 10%, 15%, 20%, 25%, 30%, 35%, 40%, 45%, 50%, 55%, 60%, 65%, 70%, 75%, 80%, 85%, 90%, 95%, 98%, 99%, or 100% as assessed by methods described herein and/or known to one of skill in the art, relative to Axl activity in the presence of Axl ligand stimulation without any antibody or with an unrelated antibody (e.g., an antibody that does not specifically bind to Axl). In certain embodiments, an anti-Axl antibody described herein can inhibit (e.g., partially inhibit) Axl activity by at least about 25% to about 65% as assessed by methods described herein and/or known to one of skill in the art, relative to Axl activity in the presence of Axl ligand stimulation without any antibody or with an unrelated antibody (e.g., an antibody that does not specifically bind to Axl). Non-limiting examples of Axl activity can include Axl receptor phosphorylation, Axl receptor signaling, Axl ligand (e.g., Gas6) mediated cell proliferation, and Axl ligand (e.g., Gas6) mediated cell survival (e.g., natural killer (NK) cells), modulation of maturation of NK cells, dendritic cells or macrophages, phagocytosis, inhibition of proinflammatory cytokine production (e.g., inhibition of TLR-induced production of proinflammatory cytokines (e.g., Eotaxin-1, G-CSF, Flt-3L, GM-CSF, Fractalkine, IFNa2, IFNγ, GRO alpha, IL-2, IL-10, MCP-3, IL-12P40, MDC, IL-IRA, IL-1B, IL-4, TNFα, RANTES, MIP-1B, IL-6, IL-8, IP-10, VEGF-A, and/or MIP-1a, IL-12 and type I interferons)).

In one embodiment, an antibody can block (e.g., partially block) or inhibit (e.g., partially inhibit) (e.g., human Axl), specifically tyrosine phosphorylation of one or more residues in the cytoplasmic domain of Axl.

Thus, in specific embodiments, antibodies described herein (e.g., antibody Ab301 or an antigen-binding fragment thereof, Ab302 or an antigen-binding fragment thereof, Ab303 or an antigen-binding fragment thereof, Ab304 or an antigen-fragment thereof, Ab305 or an antigen-binding fragment thereof, or Abcon or an antigen-fragment thereof, or an antibody comprising CDRs of any of Ab301, Ab302, Ab303, Ab304, Ab305, or Abcon as set forth in Table 1, Table 4, Table 6, Table 8, Table 10 or Table 12 or an antibody or antigen-binding fragment comprising a VH and/or VL of Table 3.) specifically bind to human Axl and block, inhibit, or reduce tyrosine (e.g., human Axl) by at least about 5%, 10%, 15%, 20%, 25%, 30%, 35%, 40%, 45%, 50%, 55%, 60%, 65%, 70%, 75%, 80%, 85%, 90%, 95%, 98%, 99%, or 100% as assessed by methods described herein or known to one of skill in the art, e.g., as described in Section 6, relative to phosphorylation in the presence of Axl ligand stimulation without any antibody or with an unrelated antibody (e.g., an antibody that does not specifically bind to Axl). In particular embodiments, antibodies described herein specifically bind to Axl and block or inhibit tyrosine phosphorylation in the cytoplasmic domain of Axl by at least about 25%, optionally to about 65%, 75%, 80% or 85%, as assessed by methods described herein or known to one of skill in the art, e.g., as described in Section 6, below. In certain embodiments, antibodies described herein specifically bind to Axl and block or inhibit tyrosine phosphorylation of Axl by at least about 25% to about 80% as assessed by methods described herein or known to one of skill in the art, e.g., as described in Section 6, below. In certain embodiments, antibodies described herein (e.g., antibody Ab301 or an antigen-binding fragment thereof, Ab302 or an antigen-binding fragment thereof, Ab303 or an antigen-binding fragment thereof, Ab304 or an antigen-fragment thereof, Ab305 or an antigen-binding fragment thereof, or Abcon or an antigen-binding fragment thereof, or an antibody comprising CDRs of any of Ab301, Ab302, Ab303, Ab304, Ab305, or Abcon as set forth in Table 1, Table 4, Table 6, Table 8, Table 10, or Table 12, or an antibody or antigen-binding fragment comprising a VH and/or VL of Table 3) specifically bind to Axl and block or inhibit tyrosine (e.g., human Axl) by at least about 50% to about 100% as assessed by methods described herein or known to one of skill in the art, e.g., as described in Section 6, below.

In specific embodiments, antibodies described herein specifically bind to Axl and block or inhibit tyrosine phosphorylation of Axl with an IC₅₀ of less than about 10 nM, about 1 nM, about 500 pM, about 400 pM, about 200 pM, or about 100 pM, as assessed by methods described herein in Section 6 below or known to one of skill in the art. In specific embodiments, antibodies described herein specifically bind to Axl and block or inhibit tyrosine phosphorylation of Axl with an IC₅₀ in the range of about 10 nM to about 100 pM, about 1 nM to 100 pM, or about 500 pM to about 100 pM. For example, an IC₅₀ for inhibition of tyrosine phosphorylation can be determined by assaying lysates from cells, recombinantly expressing Axl, in ELISA which detects tyrosine phosphorylation, for example, as described in Section 6 below. In certain embodiments, cells, recombinantly expressing Axl, are sorted, e.g., sorted to select for cells highly expressing Axl, prior to use in the phosphorylation inhibition assays. In some embodiments, the cells are not sorted prior to use in the phosphorylation inhibition assays.

In specific embodiments, antibodies described herein (e.g., antibody Ab301 or an antigen-binding fragment thereof, Ab302 or an antigen-binding fragment thereof, Ab303 or an antigen-binding fragment thereof, Ab304 or an antigen-fragment thereof, Ab305 or an antigen-binding fragment thereof, or Abcon or an antigen-binding fragment thereof, or an antibody comprising CDRs of any of Ab301, Ab302, Ab303, Ab304, Ab305, or Abcon as set forth in Table 1, Table 4, Table 6, Table 8, Table 10, or Table 12, or an antibody or antigen-binding fragment comprising a VH and/or VL of Table 3) specifically bind to Axl and block or inhibit phosphorylation of one or more tyrosine residues in the cytoplasmic domain of Axl by at least about 5%, 10%, 15%, 20%, 25%, 30%, 35%, 40%, 45%, 50%, 55%, 60%, 65%, 70%, 75%, 80%, 85%, 90%, 95%, 98%, 99%, or 100% as assessed by methods described herein or known to one of skill in the art, e.g., immunoblotting assay, relative to phosphorylation in the presence of Axl ligand stimulation without any antibody or with an unrelated antibody (e.g., an antibody that does not specifically bind to Axl). In specific embodiments, blocking or inhibition (e.g., partial inhibition) of phosphorylation of Axl by antibodies described herein can be assessed upon Axl ligand stimulation.

Signaling events downstream of Axl receptor phosphorylation can serve as indicators of Axl activity. For example, Axl ligand (e.g., Gas6) binding to its receptor Axl stimulates several distinct signaling pathways, including for example members of phosphatidylinositol (PI) 3-kinases, and mitogen-activated protein kinase (MAPK) (see Korshunov, Clin. Sci. (Lond.), 2012, 122(8):361-368).

Thus, in certain aspects, anti-Axl antibodies described herein which act as inhibitors of Axl activity (e.g., antibody Ab301 or an antigen-binding fragment thereof, Ab302 or an antigen-binding fragment thereof, Ab303 or an antigen-binding fragment thereof, Ab304 or an antigen-fragment thereof, Ab305 or an antigen-binding fragment thereof, or Abcon or an antigen-binding fragment thereof, or an antibody comprising CDRs of any of Ab301, Ab302, Ab303, Ab304, Ab305, or Abcon as set forth in Table 1, Table 4, Table 6, Table 8, Table 10, or Table 12, or an antibody or antigen-binding fragment comprising a VH and/or VL of Table 3) can inhibit signaling of a member of the PI 3-kinases or MAPK. In particular embodiments, anti-Axl antibodies described herein which act as inhibitors of Axl activity can inhibit binding (or inhibit interaction), to the cytoplasmic domain of Axl, of one or more of PI3K, PLC, and Grb2. In certain embodiments, anti-Axl antibodies described herein which act as inhibitors of Axl activity can inhibit activation by Axl of one or more of PI3K, PLC, and Grb2.

In particular embodiments, anti-Axl antibodies described herein which act as inhibitors of Axl activity (e.g., antibody Ab301 or an antigen-binding fragment thereof, Ab302 or an antigen-binding fragment thereof, Ab303 or an antigen-binding fragment thereof, Ab304 or an antigen-fragment thereof, Ab305 or an antigen-binding fragment thereof, or Abcon or an antigen-binding fragment thereof, or an antibody comprising CDRs of any of Ab301, Ab302, Ab303, Ab304, Ab305, or Abcon as set forth in Table 1, Table 4, Table 6, Table 8, Table 10, or Table 12, or an antibody or antigen-binding fragment comprising a VH and/or VL of Table 3) can inhibit downstream signaling such as phosphorylation of MAPK, phosphorylation of P13K, or phosphorylation of AKT. Thus, in certain embodiments, an anti-Axl antibody described herein can inhibit or reduce phosphorylation of MAPK (e.g., Axl ligand (e.g., Gas6) induced phosphorylation of MAPK) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in Section 6 or immunoblotting assay, relative to phosphorylation in the presence of Axl ligand stimulation without any antibody or with an unrelated antibody (e.g., an antibody that does not specifically bind to Axl). In certain embodiments, an anti-Axl antibody described herein can inhibit or reduce phosphorylation of AKT (e.g., Axl ligand (e.g., Gas6) induced phosphorylation of AKT) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., as described in Section 6, relative to phosphorylation in the presence of Axl ligand stimulation without any antibody or with an unrelated antibody (e.g., an antibody that does not specifically bind to Axl).

Cells and cell lines which are appropriate for use in the assays described herein relating to Axl activity are readily available (e.g., ATCC) or can be readily identified using methods known in the art. For example, cells and/or cell lines that express Axl endogenously or that possess Axl signaling or activity are known to one of skill in the art. In certain embodiments, cells or cell lines that are appropriate for use in the assays described herein can express Axl, either endogenously or recombinantly.

Non-limiting examples of cells that can be used in the methods and assays described herein include primary cells, transformed cells, stem cells, mast cells, primordial germ cells, oocytes, spermatocytes, embryonic stem cells, hematopoietic cells, erythroleukemia cells (e.g., F36P and TF-1 cell lines), human monocytic cell lines, e.g., acute monocytic leukemia cell lines, human myeloid leukemia cell lines, such as MOTE cells; gastrointestinal stromal tumor cell lines such as ST-882, GIST430, and GIST882; neuroblastoma cell lines such as SK-N-SH, SK-SY5Y, H-EP1, SK-N-BE(2), SK-N-BE(ZkM17), SK-N-BE(2)C, LA-N-1, or LA-N-1-5s; small cell lung carcinoma cell lines such as H526, ECC12, TMK1, MKN7, GCIY, and HGC27; and melanoma cell lines such as SKMEL3, SKMEL5, G361, MALME-3M, HMCB. In particular embodiments, cells that can be used in the methods and assays described herein include immune cells, such as macrophages, dendritic cells, and natural killer (NK) cells. In particular embodiments, cells that can be used in the methods and assay described herein include THP1 cells.

Alternatively, cells and cell lines that express Axl, e.g., human Axl, can routinely be generated recombinantly. Non-limiting examples of cells that can be engineered to express Axl recombinantly include COS cells, HEK 293 cells, CHO cells, H1299 cells, fibroblasts (e.g., human fibroblasts) such as NIH3T3 cells, and MEFS. In a specific embodiment, cells for use in the methods described herein are HEK 293 cells expressing human Axl ECD.

### 5.2 Antibody Production

Antibodies described herein (or an antigen-binding fragment thereof) that specifically bind to Axl (e.g., ECD of human Axl) can be produced by any method known in the art for the synthesis of antibodies, for example, by chemical synthesis or by recombinant expression techniques. The methods described herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

In a specific embodiment, an antibody described herein is an antibody (e.g., recombinant antibody) prepared, expressed, created or isolated by any means that involves creation, e.g., via synthesis, genetic engineering of DNA sequences. In certain embodiments, such antibody comprise sequences (e.g., DNA sequences or amino acid sequences) that do not naturally exist within the antibody germline repertoire of an animal or mammal (e.g., human) in vivo.

In a certain aspect, provided herein is a method of making an antibody or an antigen-binding fragment thereof that specifically binds to human Axl comprising culturing a cell or host cell described herein. In a certain aspect, provided herein is a method of making an antibody or an antigen-binding fragment thereof that specifically binds to human Axl comprising expressing (e.g., recombinantly expressing) the antibody or antigen-binding fragment thereof using a cell or host cell described herein (e.g., a cell or a host cell comprising polynucleotides encoding an antibody described herein). In a particular embodiment, the cell is an isolated cell. In a particular embodiment, the exogenous polynucleotides have been introduced into the cell. In a particular embodiment, the method further comprises the step of purifying the antibody or antigen-binding fragment thereof obtained from the cell or host cell.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody described herein (e.g., anti-Axl antibody comprising the CDRs of any of Ab301, Ab302, Ab303, Ab304, Ab305, or Abcon as set forth in Table 1, Table 4, Table 6, Table 8, Table 10, or Table 12, or an antibody or antigen-binding fragment comprising a VH and/or VL of Table 3) or a fragment thereof, for example, light chain and/or heavy chain of such antibody.

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., ECD of human Axl) used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). Additionally, a RIMMS (repetitive immunization multiple sites) technique can be used to immunize an animal (Kilptrack et al., 1997 Hybridoma 16:381-9, incorporated by reference in its entirety).

In some embodiments, mice (or other animals, such as rats, monkeys, donkeys, pigs, sheep, hamster, or dogs) can be immunized with an antigen (e.g., Axl, for example human Axl) and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the American Type Culture Collection (ATCC®) (Manassas, Va.), to form hybridomas. Hybridomas are selected and cloned by limited dilution. In certain embodiments, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as NS0 cell line or those derived from MOPC-21 and MPC-11 mouse tumors available from the Salk Institute Cell Distribution Center, San Diego, Calif., USA, and SP-2 or X63-Ag8.653 cells available from the American Type Culture Collection, Rockville, Md., USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against human Axl antigen (e.g., ECD of human Axl). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

In some embodiments, antibody variants having an improved property such as affinity, stability, and/or expression level as compared to a parent antibody may be prepared by affinity maturation. Affinity maturation techniques that are known in the art and/or described herein can be used to alter the CDR(s), followed by screening of the resultant binding molecules for the desired change in binding. Without being bound by theory, libraries of antibodies can be displayed on the surface of an organism (e.g., phage, bacteria, yeast, or mammalian cell) or in association with their encoding mRNA or DNA (e.g., covalently or non-covalently). Affinity selection of the displayed antibodies allows isolation of organisms or complexes carrying the genetic information encoding the antibodies. Two or three rounds of mutation and selection using display methods such as phage display usually results in antibody fragments with improved affinities (e.g., in the low nanomolar or picomolar range). Further methods that can be used to generate antibody libraries and/or antibody affinity maturation are disclosed in Holland et al., 2013, J. of Immunol. Methods 394:55-61, incorporated herein by reference in its entirety.

Antibodies described herein include antibody fragments which recognize specific Axl antigens (e.g., ECD of Axl) and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein or antigen-binding fragments thereof can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

In one aspect, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine immunoglobulin). In particular embodiments, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA and IgE, and any isotype, including IgG1, IgG2, IgG3 and IgG4. Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), which is incorporated by reference herein in its entirety.

Methods for making multispecific (e.g, bispecific antibodies) have been described, see, for example, U.S. Pat. Nos. 7,951,917, 7,183,076, 8,227,577, 5,837,242, 5,989,830, 5,869,620, 6,132,992, and 8,586,713.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301.

Further, antibodies that specifically bind to a Axl antigen can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

Human antibodies which specifically bind to an ECD of human Axl can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741.

In some embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., ECD of human Axl). Such methods are known and are described in the art, see, e.g., Shinmoto et al., Cytotechnology, 2004, 46:19-23; Naganawa et al., Human Antibodies, 2005, 14:27-31.

### 5.2.1 Polynucleotides

In certain aspects, provided herein are polynucleotides comprising a nucleotide sequence encoding an antibody described herein or a fragment thereof (e.g., a variable light chain region and/or variable heavy chain region) that specifically binds to a Axl antigen, and vectors, e.g., vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid molecule(s) encoding an antibody described herein is isolated or purified.

In particular aspects, provided herein are polynucleotides comprising nucleotide sequences encoding antibodies or antigen-binding fragments thereof, which specifically bind to a Axl polypeptide (e.g., human Axl, for example, human Axl) and comprises an amino acid sequence as described herein, as well as antibodies which compete with such antibodies for binding to a Axl polypeptide (e.g., in a dose-dependent manner), or which binds to the same epitope as that of such antibodies.

In certain aspects, provided herein are polynucleotides comprising a nucleotide sequence encoding the light chain or heavy chain of an antibody described herein. The polynucleotides can comprise nucleotide sequences encoding a light chain comprising the VL of antibodies described herein (see, e.g., Table 3). The polynucleotides can comprise nucleotide sequences encoding a heavy chain comprising the VH of antibodies described herein (see, e.g., Table 3). In specific embodiments, a polynucleotide described herein encodes a VL chain region comprising the amino acid sequence of SEQ ID NO: 12. In other specific embodiments, a polynucleotide described herein encodes a VL chain region comprising the amino acid sequence of SEQ ID NO: 6. In specific embodiments, a polynucleotide described herein encodes a VH chain region comprising the amino acid sequence of SEQ ID NO: 11. In specific embodiments, a polynucleotide described herein encodes a VH chain region comprising the amino acid sequence of SEQ ID NO: 7. In specific embodiments, a polynucleotide described herein encodes a VH chain region comprising the amino acid sequence of SEQ ID NO: 8. In specific embodiments, a polynucleotide described herein encodes a VH chain region comprising the amino acid sequence of SEQ ID NO: 9. In specific embodiments, a polynucleotide described herein encodes a VH chain region comprising the amino acid sequence of SEQ ID NO: 10. In some embodiments, a polynucleotide described herein encodes a VH chain region comprising an amino acid sequence described by the consensus sequence of SEQ ID NO: 5.

In particular embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 1. In specific embodiments, provided herein are polynucleotides comprising three VH chain CDRs, e.g., containing VH CDR1, VH CDR2, and VH CDR3 of Ab301 as set forth in Table 1. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 1 and three VH chain CDRs, e.g., containing VH CDR1, VH CDR2, and VH CDR3 of Ab301 as set forth in Table 1.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 1. In specific embodiments, provided herein are polynucleotides comprising three VH chain CDRs, e.g., containing VH CDR1, VH CDR2, and VH CDR3 of Ab302 as set forth in Table 1. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 1 and three VH chain CDRs, e.g., containing VH CDR1, VH CDR2, and VH CDR3 of Ab302 as set forth in Table 1. 1005371 In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 1. In specific embodiments, provided herein are polynucleotides comprising three VH chain CDRs, e.g., containing VH CDR1, VH CDR2, and VH CDR3 of Ab303 as set forth in Table 1. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 1 and three VH chain CDRs, e.g., containing VH CDR1, VH CDR2, and VH CDR3 of Ab303 as set forth in Table 1.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 1. In specific embodiments, provided herein are polynucleotides comprising three VH chain CDRs, e.g., containing VH CDR1, VH CDR2, and VH CDR3 of Ab304 as set forth in Table 1. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 1 and three VH chain CDRs, e.g., containing VH CDR1, VH CDR2, and VH CDR3 of Ab304 as set forth in Table 1.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 1. In specific embodiments, provided herein are polynucleotides comprising three VH chain CDRs, e.g., containing VH CDR1, VH CDR2, and VH CDR3 of Ab305 as set forth in Table 1. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 1 and three VH chain CDRs, e.g., containing VH CDR1, VH CDR2, and VH CDR3 of Ab305 as set forth in Table 1.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 1. In specific embodiments, provided herein are polynucleotides comprising three VH chain CDRs, e.g., containing VH CDR1, VH CDR2, and VH CDR3 of Abcon as set forth in Table 1. In specific embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 1 and three VH chain CDRs, e.g., containing VH CDR1, VH CDR2, and VH CDR3 of Abcon as set forth in Table 1.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 4. In specific embodiments, provided herein are polynucleotides comprising three VH chain CDRs, e.g., containing VH CDR1, VH CDR2, and VH CDR3 of Ab301 as set forth in Table 4. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 4 and three VH chain CDRs, e.g., containing VH CDR1, VH CDR2, and VH CDR3 of Ab301 as set forth in Table 4.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 4. In specific embodiments, provided herein are polynucleotides comprising three VH chain CDRs, e.g., containing VH CDR1, VH CDR2, and VH CDR3 of Ab302 as set forth in Table 4. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 4 and three VH chain CDRs, e.g., containing VH CDR1, VH CDR2, and VH CDR3 of Ab302 as set forth in Table 4.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 4. In specific embodiments, provided herein are polynucleotides comprising three VH chain CDRs, e.g., containing VH CDR1, VH CDR2, and VH CDR3 of Ab303 as set forth in Table 4. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 4 and three VH chain CDRs, e.g., containing VH CDR1, VH CDR2, and VH CDR3 of Ab303 as set forth in Table 4.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 4. In specific embodiments, provided herein are polynucleotides comprising three VH chain CDRs, e.g., containing VH CDR1, VH CDR2, and VH CDR3 of Ab304 as set forth in Table 4. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 4 and three VH chain CDRs, e.g., containing VH CDR1, VH CDR2, and VH CDR3 of Ab304 as set forth in Table 4.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 4. In specific embodiments, provided herein are polynucleotides comprising three VH chain CDRs, e.g., containing VH CDR1, VH CDR2, and VH CDR3 of Ab305 as set forth in Table 4. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 4 and three VH chain CDRs, e.g., containing VH CDR1, VH CDR2, and VH CDR3 of Ab305 as set forth in Table 4.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 4. In specific embodiments, provided herein are polynucleotides comprising three VH chain CDRs, e.g., containing VH CDR1, VH CDR2, and VH CDR3 of Abcon as set forth in Table 4. In specific embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 4 and three VH chain CDRs, e.g., containing VH CDR1, VH CDR2, and VH CDR3 of Abcon as set forth in Table 4.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 6. In specific embodiments, provided herein are polynucleotides comprising three VH chain CDRs, e.g., containing VH CDR1, VH CDR2, and VH CDR3 of Ab301 as set forth in Table 6. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 6 and three VH chain CDRs, e.g., containing VH CDR1, VH CDR2, and VH CDR3 of Ab301 as set forth in Table 6.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 6. In specific embodiments, provided herein are polynucleotides comprising three VH chain CDRs, e.g., containing VH CDR1, VH CDR2, and VH CDR3 of Ab302 as set forth in Table 6. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 6 and three VH chain CDRs, e.g., containing VH CDR1, VH CDR2, and VH CDR3 of Ab302 as set forth in Table 6.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 6. In specific embodiments, provided herein are polynucleotides comprising three VH chain CDRs, e.g., containing VH CDR1, VH CDR2, and VH CDR3 of Ab303 as set forth in Table 6. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 6 and three VH chain CDRs, e.g., containing VH CDR1, VH CDR2, and VH CDR3 of Ab303 as set forth in Table 6.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 6. In specific embodiments, provided herein are polynucleotides comprising three VH chain CDRs, e.g., containing VH CDR1, VH CDR2, and VH CDR3 of Ab304 as set forth in Table 6. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 6 and three VH chain CDRs, e.g., containing VH CDR1, VH CDR2, and VH CDR3 of Ab304 as set forth in Table 6.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 6. In specific embodiments, provided herein are polynucleotides comprising three VH chain CDRs, e.g., containing VH CDR1, VH CDR2, and VH CDR3 of Ab305 as set forth in Table 6. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 6 and three VH chain CDRs, e.g., containing VH CDR1, VH CDR2, and VH CDR3 of Ab305 as set forth in Table 6.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 6. In specific embodiments, provided herein are polynucleotides comprising three VH chain CDRs, e.g., containing VH CDR1, VH CDR2, and VH CDR3 of Abcon as set forth in Table 6. In specific embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 6 and three VH chain CDRs, e.g., containing VH CDR1, VH CDR2, and VH CDR3 of Abcon as set forth in Table 6.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 8. In specific embodiments, provided herein are polynucleotides comprising three VH chain CDRs, e.g., containing VH CDR1, VH CDR2, and VH CDR3 of Ab301 as set forth in Table 8. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 8 and three VH chain CDRs, e.g., containing VH CDR1, VH CDR2, and VH CDR3 of Ab301 as set forth in Table 8.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 8. In specific embodiments, provided herein are polynucleotides comprising three VH chain CDRs, e.g., containing VH CDR1, VH CDR2, and VH CDR3 of Ab302 as set forth in Table 8. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 8 and three VH chain CDRs, e.g., containing VH CDR1, VH CDR2, and VH CDR3 of Ab302 as set forth in Table 8

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 8. In specific embodiments, provided herein are polynucleotides comprising three VH chain CDRs, e.g., containing VH CDR1, VH CDR2, and VH CDR3 of Ab303 as set forth in Table 8. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 8 and three VH chain CDRs, e.g., containing VH CDR1, VH CDR2, and VH CDR3 of Ab303 as set forth in Table 8.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 8. In specific embodiments, provided herein are polynucleotides comprising three VH chain CDRs, e.g., containing VH CDR1, VH CDR2, and VH CDR3 of Ab304 as set forth in Table 8. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 8 and three VH chain CDRs, e.g., containing VH CDR1, VH CDR2, and VH CDR3 of Ab304 as set forth in Table 8.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 8. In specific embodiments, provided herein are polynucleotides comprising three VH chain CDRs, e.g., containing VH CDR1, VH CDR2, and VH CDR3 of Ab305 as set forth in Table 8. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 8 and three VH chain CDRs, e.g., containing VH CDR1, VH CDR2, and VH CDR3 of Ab305 as set forth in Table 8.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 8. In specific embodiments, provided herein are polynucleotides comprising three VH chain CDRs, e.g., containing VH CDR1, VH CDR2, and VH CDR3 of Abcon as set forth in Table 8. In specific embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 8 and three VH chain CDRs, e.g., containing VH CDR1, VH CDR2, and VH CDR3 of Abcon as set forth in Table 8.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 10. In specific embodiments, provided herein are polynucleotides comprising three VH chain CDRs, e.g., containing VH CDR1, VH CDR2, and VH CDR3 of Ab301 as set forth in Table 10. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 10 and three VH chain CDRs, e.g., containing VH CDR1, VH CDR2, and VH CDR3 of Ab301 as set forth in Table 10.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 10. In specific embodiments, provided herein are polynucleotides comprising three VH chain CDRs, e.g., containing VH CDR1, VH CDR2, and VH CDR3 of Ab302 as set forth in Table 10. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 10 and three VH chain CDRs, e.g., containing VH CDR1, VH CDR2, and VH CDR3 of Ab302 as set forth in Table 10.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 10. In specific embodiments, provided herein are polynucleotides comprising three VH chain CDRs, e.g., containing VH CDR1, VH CDR2, and VH CDR3 of Ab303 as set forth in Table 10. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 10 and three VH chain CDRs, e.g., containing VH CDR1, VH CDR2, and VH CDR3 of Ab303 as set forth in Table 10.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 10. In specific embodiments, provided herein are polynucleotides comprising three VH chain CDRs, e.g., containing VH CDR1, VH CDR2, and VH CDR3 of Ab304 as set forth in Table 10. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 10 and three VH chain CDRs, e.g., containing VH CDR1, VH CDR2, and VH CDR3 of Ab304 as set forth in Table 10.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 10. In specific embodiments, provided herein are polynucleotides comprising three VH chain CDRs, e.g., containing VH CDR1, VH CDR2, and VH CDR3 of Ab305 as set forth in Table 10. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 10 and three VH chain CDRs, e.g., containing VH CDR1, VH CDR2, and VH CDR3 of Ab305 as set forth in Table 10.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 10. In specific embodiments, provided herein are polynucleotides comprising three VH chain CDRs, e.g., containing VH CDR1, VH CDR2, and VH CDR3 of Abcon as set forth in Table 10. In specific embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 10 and three VH chain CDRs, e.g., containing VH CDR1, VH CDR2, and VH CDR3 of Abcon as set forth in Table 10.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 12. In specific embodiments, provided herein are polynucleotides comprising three VH chain CDRs, e.g., containing VH CDR1, VH CDR2, and VH CDR3 of Ab301 as set forth in Table 12. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab301 as set forth in Table 12 and three VH chain CDRs, e.g., containing VH CDR1, VH CDR2, and VH CDR3 of Ab301 as set forth in Table 12

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 12. In specific embodiments, provided herein are polynucleotides comprising three VH chain CDRs, e.g., containing VH CDR1, VH CDR2, and VH CDR3 of Ab302 as set forth in Table 12. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab302 as set forth in Table 12 and three VH chain CDRs, e.g., containing VH CDR1, VH CDR2, and VH CDR3 of Ab302 as set forth in Table 12.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 12. In specific embodiments, provided herein are polynucleotides comprising three VH chain CDRs, e.g., containing VH CDR1, VH CDR2, and VH CDR3 of Ab303 as set forth in Table 12. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab303 as set forth in Table 12 and three VH chain CDRs, e.g., containing VH CDR1, VH CDR2, and VH CDR3 of Ab303 as set forth in Table 12.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 12. In specific embodiments, provided herein are polynucleotides comprising three VH chain CDRs, e.g., containing VH CDR1, VH CDR2, and VH CDR3 of Ab304 as set forth in Table 12. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab304 as set forth in Table 12 and three VH chain CDRs, e.g., containing VH CDR1, VH CDR2, and VH CDR3 of Ab304 as set forth in Table 12.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 12. In specific embodiments, provided herein are polynucleotides comprising three VH chain CDRs, e.g., containing VH CDR1, VH CDR2, and VH CDR3 of Ab305 as set forth in Table 12. In specific embodiments, provided herein are polynucleotides comprising a nucleotide sequence encoding an anti-Axl antibody comprising three VL chain CDRs, e.g., containing VL CDR1, VL CDR2, and VL CDR3 of Ab305 as set forth in Table 12 and three VH chain CDRs, e.g., containing VH CDR1, VH CDR2, and VH CDR3 of Ab305 as set forth in Table 12.

In other particular embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 12. In specific embodiments, provided herein are polynucleotides comprising three VH chain CDRs, e.g., containing VH CDR1, VH CDR2, and VH CDR3 of Abcon as set forth in Table 12. In specific embodiments, provided herein are polynucleotides comprising a nucleotide sequence encoding an anti-Axl antibody comprising three VL chain CDRs, e.g., containing VL CDR1, VL CDR2, and VL CDR3 of Abcon as set forth in Table 12 and three VH chain CDRs, e.g., containing VH CDR1, VH CDR2, and VH CDR3 of Abcon as set forth in Table 12.

In certain embodiments, a polynucleotide described herein comprises a nucleotide sequence encoding an antibody provided herein comprising a variable light (VL) chain region comprising an amino acid described herein, wherein the antibody specifically binds to a Axl polypeptide, e.g., a human Axl polypeptide, for example, human Axl (e.g., human Axl).

In certain embodiments, a polynucleotide described herein comprises a nucleotide sequence encoding an antibody provided herein comprising a variable heavy (VH) chain region comprising an amino acid sequence described herein, wherein the antibody specifically binds to a Axl polypeptide, e.g., a human Axl polypeptide, for example, human Axl (e.g., human Axl).

In specific embodiments, a polynucleotide provided herein comprises a nucleotide sequence encoding an antibody described herein comprising: framework regions (e.g., framework regions of the VL domain and VH domain) that are human framework regions, wherein the antibody specifically binds a Axl polypeptide, e.g., a human Axl polypeptide, for example, human Axl (e.g., human Axl).

In a specific embodiment, provided herein is a polynucleotide sequence comprising a nucleotide sequence(s) encoding the VH, VL, or both the VH and VL of an anti-Axl antibody or antigen-binding fragment thereof, for example, as shown in Table 14.

TABLE 14
VH and VL Nucleotide Sequences

| | | Nucleotide Sequence |
|---|---|---|
| Ab301 | VH | gaggtgaagctggaagcctccggcggaggactggtgcagcccggtatgagcgtgaagctgagctgcaccaccagcggcttcac |
| | | cttcagcgactactggatggagtgggtgaggcaagctcccggtaaaggtttagagtgggtggccgaaattcgtaacaaggtgaac |
| | | aactacgccacctactacggcaagtccgtgaagggtcgttttaccatctctcgtgacgacagcaagagcatcgtgtatttacagatg |
| | | aacagcattcgtagcgaggacaccggcatctactactgcaacactttaaggctgttcgcctactggggacaaggtactttagtgacc |
| | | gtgtccgcc |
| | | (SEQ ID NO: 18) |
| | VL | gacattgtgatgacccaagctcctttatccatcagcgtgacacccggtgagagccccagcatgagctgtcgtagcagcaagtcttta |
| | | ctgcatcgtaacggcatcacctacgtgtactggtatttacagaagcccggcaagagcccccagctgctgatctatcgtatgagcaca |
| | | ctggccagcggcgtgcccgatagattctccggcagcggcagcgagaccgacttcactttaaagatcaacaaggtggaggccgag |
| | | gacgtgggcatctactactgcggccagctgctggagaatccctacaccttcggcgccggcacaaggctggagatcaag |
| | | (SEQ ID NO: 19) |
| Ab302 | VH | gaggtgcagctggtggaatccggcggaggtttagtgaagcccggtggctctttaaagctgagctgtgccgcctccggcttcacctt |
| | | ctccgactactggatggagtgggtgcggcaagctcccggtaagggtttagagtgggtggccgagatccggaacaaggtgaacaa |
| | | ctacgccacctactacggcaagtccgtgaagggccggttcaccatctcccgggacgactccaagaacactttatatttacagatgaa |
| | | ctctttaaagaccgaggacaccgccgtgtattactgcaacactttacggctgtttgcctactggggccaaggtactttagtgacagtgt |
| | | cctcc |
| | | (SEQ ID NO: 13) |
| | VL | gacatccagatgacacagagccdtcctctttatccgcttccgtgggagatcgtgtgaccatcacttgtcgttcctccaagtctttactg |
| | | cataggcaaggtatcacctacgtgtactggtaccagcagaagcccggcaaggtgcccaagctgctgatctatcgtatgtccacttta |
| | | gcttccggcgtgccttctcgtttttctggctccggctctggcacagacttcactttaaccatctcctctttacagcccgaggacgtggcc |
| | | acatactactgcggccagctgctggagaatccctacaccttcggccaaggtaccaagctggagatcaag |
| | | (SEQ ID NO: 14) |
| Ab303 | VH | gaggtgcagctggtggagtccggaggaggcgtggtgcaacccggtcgttctttaaggctgtcttgtgctgcttccggcttcaccttct |
| | | ccgactactggatggagtgggtgaggcaagctcccggtaaaggactggagtgggtggccgagatccggaacaaggtgaacaac |
| | | tacgccacctactacggcaagagcgtgaagggtcgtttcaccatctctcgtgacaactccaagaacactttatatttacagatgaact |
| | | ccttacgtgccgaggacaccgccgtgtactactgcaacactttacggctgttcgcttactggggacaaggtactttagtgaccgtgtc |
| | | ctcc |
| | | (SEQ ID NO: 15) |
| | VL | gacatccagatgacacagagccdtcctctttatccgcttccgtgggagatcgtgtgaccatcacttgtcgttcctccaagtctttactg |
| | | cataggcaaggtatcacctacgtgtactggtaccagcagaagcccggcaaggtgcccaagctgctgatctatcgtatgtccacttta |
| | | gcttccggcgtgccttctcgtttttctggctccggctctggcacagacttcactttaaccatctcctctttacagcccgaggacgtggcc |
| | | acatactactgcggccagctgctggagaatccctacaccttcggccaaggtaccaagctggagatcaag |
| | | (SEQ ID NO: 14) |
| Ab304 | VH | gaggtgcagctggtggagtccggaggcggactggtgcaacccggtggatctctgaggctgtcttgtgccgcttccggcttcacctt |
| | | ctccgactactggatggagtgggtgaggcaagctcccggcaaaggactggagtgggtcggcgagatccggaacaaggtgaaca |
| | | actacgccacctactacggcaagtccgtgaagggtcgtttcaccatctctcgtgacgagagcaagaactattatatttacagatgaa |
| | | ctctttaaagaccgaggacaccgccgtgtactactgcaacactttacgtctgttcgcctattggggccaaggtaccacagtgaccgt |
| | | gtcctcc |
| | | (SEQ ID NO: 16) |
| | VL | gacatccagatgacacagagcccttcctctttatccgcttccgtgggagatcgtgtgaccatcacttgtcgttcctccaagtctttactg |
| | | cataggcaaggtatcacctacgtgtactggtaccagcagaagcccggcaaggtgcccaagctgctgatctatcgtatgtccacttta |
| | | gcttccggcgtgccttctcgtttttctggctccggctctggcacagacttcactttaaccatctcctctttacagcccgaggacgtggcc |
| | | acatactactgcggccagctgctggagaatccctacaccttcggccaaggtaccaagctggagatcaag |
| | | (SEQ ID NO: 14) |
| Ab305 | VH | gaggtgcagctggtggaatccggcggaggactggtgcagcccggtcgttctttaaggctgtcttgtacagcctccggcttcaccttc |
| | | agcgactactggatggagtgggtgaggcaagctcccggtaagggactggagtgggtgggcgagatccggaacaaggtgaaca |
| | | actacgccacctactacggcaagagcgtgaagggtcgtttcaccatctctcgtgacgattccaagtccatcgcctatttacagatgaa |
| | | ctctttaaagaccgaggacaccgccgtgtactactgcaacactttacggctgttcgcctattggggccaaggtactttagtgaccgtg |
| | | tccgcc |
| | | (SE ID NO: 17) |
| | VL | gacatccagatgacacagagcccttcctctttatccgcttccgtgggagatcgtgtgaccatcacttgtcgttcctccaagtctttactg |
| | | cataggcaaggtatcacctacgtgtactggtaccagcagaagcccggcaaggtgcccaagctgctgatctatcgtatgtccacttta |
| | | gcttccggcgtgccttctcgtttttctggctccggctctggcacagacttcactttaaccatctcctctttacagcccgaggacgtggcc |
| | | acatactactgcggccagctgctggagaatccctacaccttcggccaaggtaccaagctggagatcaag |
| | | (SEQ ID NO: 14) |

In specific aspects, provided herein is a polynucleotide comprising a nucleotide sequence encoding an antibody comprising a light chain and a heavy chain, e.g., a separate light chain and heavy chain. With respect to the light chain, in a specific embodiment, a polynucleotide provided herein comprises a nucleotide sequence encoding a kappa light chain. In another specific embodiment, a polynucleotide provided herein comprises a nucleotide sequence encoding a lambda light chain. In yet another specific embodiment, a polynucleotide provided herein comprises a nucleotide sequence encoding an antibody described herein comprising a human kappa light chain or a human lambda light chain. In a particular embodiment, a polynucleotide provided herein comprises a nucleotide sequence encoding an antibody described herein, which specifically binds to an Axl polypeptide, e.g., a human Axl polypeptide, for example, human Axl (e.g., human Axl), wherein the antibody comprises a light chain, and wherein the amino acid sequence of the VL chain region can comprise any amino acid sequence described herein (e.g., SEQ ID NO: 12 or 6), and wherein the constant region of the light chain comprises the amino acid sequence of a human kappa light chain constant region. In another particular embodiment, a polynucleotide provided herein comprises a nucleotide sequence encoding an antibody described herein, which specifically binds to an Axl polypeptide, e.g., a human Axl polypeptide, for example, human Axl (e.g., human Axl), and comprises a light chain, wherein the amino acid sequence of the VL chain region can comprises any amino acid sequence described herein (e.g., SEQ ID NO: 12 or 6), and wherein the constant region of the light chain comprises the amino acid sequence of a human lambda light chain constant region. For example, human constant region sequences can be those described in U.S. Pat. No. 5,693,780.

In a particular embodiment, a polynucleotide provided herein comprises a nucleotide sequence encoding an antibody described herein, which specifically binds to an Axl polypeptide, e.g., a human Axl polypeptide, for example, human Axl (e.g., human Axl), wherein the antibody comprises a heavy chain, wherein the amino acid sequence of the VH chain region can comprise any amino acid sequence described herein (e.g., SEQ ID NO: 11, 7, 8, 9, or 10), and wherein the constant region of the heavy chain comprises the amino acid sequence of a human gamma (γ) heavy chain constant region.

In yet another specific embodiment, a polynucleotide provided herein comprises a nucleotide sequence encoding an antibody described herein (or an antigen-binding fragment thereof), which specifically binds an Axl polypeptide, e.g., a human Axl polypeptide, for example, human Axl (e.g., human Axl), wherein the antibody comprises a VL chain region and a VH chain region comprising any amino acid sequences described herein, and wherein the constant regions comprise the amino acid sequences of the constant regions of a human IgG1 (e.g., isotype a, z, or f), human IgG2, or human IgG4.

In a specific embodiment, provided herein are polynucleotides comprising a nucleotide sequence encoding an anti-Axl antibody, or an antigen-binding fragment or domain thereof, designated herein.

As a non-limiting example, in some embodiments, the VL of an antibody or antigen-binding fragment described herein, which specifically binds to a Axl polypeptide (e.g., human Axl), can be encoded by a nucleotide that comprises nucleotide sequence of SEQ ID NO: 19. In other embodiments, the VL of an antibody or antigen-binding fragment described herein, which specifically binds to a Axl polypeptide (e.g., human Axl), can be encoded by a nucleotide that comprises nucleotide sequence of SEQ ID NO: 14.

In other embodiments, the VH of antibody or antigen-binding fragment described herein, which specifically binds to a Axl polypeptide (e.g., human Axl), can be encoded by a nucleotide that comprises nucleotide sequence of SEQ ID NO: 18. In other embodiments, the VH of an antibody or antigen-binding fragment described herein, which specifically binds to a Axl polypeptide (e.g., human Axl), can be encoded by a nucleotide that comprises nucleotide sequence of SEQ ID NO: 13. In other embodiments, the VH of an antibody or antigen-binding fragment described herein, which specifically binds to a Axl polypeptide (e.g., human Axl), can be encoded by a nucleotide that comprises nucleotide sequence of SEQ ID NO: 15. In other embodiments, the VH of an antibody or antigen-binding fragment described herein, which specifically binds to a Axl polypeptide (e.g., human Axl), can be encoded by a nucleotide that comprises nucleotide sequence of SEQ ID NO: 16. In other embodiments, the VH of an antibody or antigen-binding fragment described herein, which specifically binds to a Axl polypeptide (e.g., human Axl), can be encoded by a nucleotide that comprises nucleotide sequence of SEQ ID NO: 17.

In certain embodiments, an antibody or antigen-binding fragment described herein, which specifically binds to a Axl polypeptide (e.g., human Axl), can be encoded by a nucleotide that further comprises a signal sequence.

In some embodiments, the VH of an antibody or antigen-binding fragment described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 18. In some embodiments, the VL of an antibody or antigen-binding fragment described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 19. In certain embodiments, an antibody described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 18 and a nucleotide that comprising a nucleotide sequence having at least 80%, at least 85%, at least 90%, at least 95%, or more sequence identity to SEQ ID NO: 19.

In some embodiments, the VL of an antibody or antigen-binding fragment described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 14.

In some embodiments, the VH of an antibody or antigen-binding fragment described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 13. In certain embodiments, an antibody described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 14 and a nucleotide that comprising a nucleotide sequence having at least 80%, at least 85%, at least 90%, at least 95%, or more sequence identity to SEQ ID NO: 13.

In some embodiments, the VH of an antibody or antigen-binding fragment described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 15. In certain embodiments, an antibody described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 14 and a nucleotide that comprising a nucleotide sequence having at least 80%, at least 85%, at least 90%, at least 95%, or more sequence identity to SEQ ID NO: 15.

In some embodiments, the VH of an antibody or antigen-binding fragment described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 16. In certain embodiments, an antibody described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 14 and a nucleotide that comprising a nucleotide sequence having at least 80%, at least 85%, at least 90%, at least 95%, or more sequence identity to SEQ ID NO: 16.

In some embodiments, the VH of an antibody or antigen-binding fragment described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 17. In certain embodiments, an antibody described herein, which specifically binds to a Axl polypeptide (e.g., human Axl), can be encoded by a nucleotide that comprising a nucleotide sequence having at least 80%, at least 85%, at least 90%, at least 95%, or more sequence identity to SEQ ID NO: 14 and a nucleotide that comprising a nucleotide sequence having at least 80%, at least 85%, at least 90%, at least 95%, or more sequence identity to SEQ ID NO: 17.

Also provided herein are polynucleotides encoding an anti-Axl antibody or a fragment thereof that are optimized, e.g., by codon/RNA optimization, replacement with heterologous signal sequences, and elimination of mRNA instability elements. Methods to generate optimized nucleic acids encoding an anti-Axl antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid. Such methods can increase expression of an anti-Axl antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-Axl antibody encoded by polynucleotides that have not been optimized.

In certain embodiments, an optimized polynucleotide sequence encoding an anti-Axl antibody described herein or a fragment thereof (e.g., VL domain and/or VH domain) can hybridize to an antisense (e.g., complementary) polynucleotide of an unoptimized polynucleotide sequence encoding an anti-Axl antibody described herein or a fragment thereof (e.g., VL domain and/or VH domain). In specific embodiments, an optimized nucleotide sequence encoding an anti-Axl antibody described herein or a fragment hybridizes under high stringency conditions to antisense polynucleotide of an unoptimized polynucleotide sequence encoding an anti-Axl antibody described herein or a fragment thereof. In a specific embodiment, an optimized nucleotide sequence encoding an anti-Axl antibody described herein or a fragment thereof hybridizes under high stringency, intermediate or lower stringency hybridization conditions to an antisense polynucleotide of an unoptimized nucleotide sequence encoding an anti-Axl antibody described herein or a fragment thereof. Information regarding hybridization conditions have been described, see, e.g., U.S. Patent Application Publication No. US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies described herein and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, for example, to generate chimeric and humanized antibodies.

If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library or a cDNA library generated from, or nucleic acid, preferably poly A+RNA, isolated from any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-Axl antibodies described herein can be readily isolated and sequenced using conventional procedures (e.g., by using oligonucleotide probes that are capable of binding specifically to genes encoding the heavy and light chains of the anti-Axl antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-Axl antibodies in the recombinant host cells.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant domains in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH chain region and/or VL chain region provided herein.

Hybridization conditions have been described in the art and are known to one of skill in the art. For example, hybridization under stringent conditions can involve hybridization to filter-bound DNA in 6×sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel, F.M. et al., eds., 1989, Current Protocols in Molecular Biology, Vol. I, Green Publishing Associates, Inc. and John Wiley & Sons, Inc., New York at pages 6.3.1-6.3.6 and 2.10.3.

### 5.2.2 Cells and Vectors

In certain aspects, provided herein are cells (e.g., host cells) expressing (e.g., recombinantly) antibodies described herein (or an antigen-binding fragment thereof) which specifically bind to an ECD of human Axl and related polynucleotides and expression vectors. Provided herein are vectors (e.g., expression vectors) comprising polynucleotides comprising nucleotide sequences encoding anti-Axl antibodies or a fragment for recombinant expression in host cells, preferably in mammalian cells. Also provided herein are host cells comprising such vectors for recombinantly expressing anti-Axl antibodies described herein (e.g., human or humanized antibody). In a particular aspect, provided herein are methods for producing an antibody described herein, comprising expressing such antibody from a host cell.

Recombinant expression of an antibody described herein (e.g., a full-length antibody, heavy and/or light chain of an antibody, or a single chain antibody described herein) that specifically binds to human Axl involves construction of an expression vector containing a polynucleotide that encodes the antibody. Once a polynucleotide encoding an antibody molecule, heavy and/or light chain of an antibody, or a fragment thereof (e.g., heavy and/or light chain variable domains) described herein has been obtained, the vector for the production of the antibody molecule can be produced by recombinant DNA technology using techniques well-known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or a fragment thereof. Thus, provided herein are host cells containing a polynucleotide encoding an antibody described herein or fragments thereof, or a heavy or light chain thereof, or fragment thereof, or a single chain antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3), operably linked to a promoter for expression of such sequences in the host cell. In certain embodiments, for the expression of double-chained antibodies, vectors encoding both the heavy and light chains, individually, can be co-expressed in the host cell for expression of the entire immunoglobulin molecule, as detailed below. In certain embodiments, a host cell contains a vector comprising a polynucleotide encoding both the heavy chain and light chain of an antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3), or a fragment thereof. In specific embodiments, a host cell contains two different vectors, a first vector comprising a polynucleotide encoding a heavy chain or a heavy chain variable region of an antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3), or a fragment thereof, and a second vector comprising a polynucleotide encoding a light chain or a light chain variable region of an antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3), or a fragment thereof. In other embodiments, a first host cell comprises a first vector comprising a polynucleotide encoding a heavy chain or a heavy chain variable region of an antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3), or a fragment thereof, and a second host cell comprises a second vector comprising a polynucleotide encoding a light chain or a light chain variable region of an antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3). In specific embodiments, a heavy chain/heavy chain variable region expressed by a first cell associated with a light chain/light chain variable region of a second cell to form an anti-Axl antibody described herein (e.g., antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or an antigen-binding fragment thereof. In certain embodiments, provided herein is a population of host cells comprising such first host cell and such second host cell.

In a particular embodiment, provided herein is a population of vectors comprising a first vector comprising a polynucleotide encoding a light chain/light chain variable region of an anti-Axl antibody described herein (e.g., antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3), and a second vector comprising a polynucleotide encoding a heavy chain/heavy chain variable region of an anti-Axl antibody described herein (e.g., antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3).

A variety of host-expression vector systems can be utilized to express antibody molecules described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, MDCK, HEK 293, NS0, PER.C6, VERO, CRL7O3O, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or an antigen-binding fragment thereof are CHO cells, for example CHO cells from the CHO GS System™ (Lonza). In a specific embodiment, a mammalian expression vector is pOptiVEC™ or pcDNA3.3. In a particular embodiment, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In certain embodiments, antibodies described herein are produced by CHO cells or NS0 cells. In a specific embodiment, the expression of nucleotide sequences encoding antibodies described herein which specifically bind to human Axl is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The antibody coding sequence can be cloned individually into non-essential regions (for example, the polyhedrin gene) of the virus and placed under control of an AcNPV promoter (for example, the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, COS, MDCK, HEK 293, NIH 3T3, W138, BT483, Hs578T, HTB2, BT2O and T47D, NS0 (a murine myeloma cell line that does not endogenously produce any immunoglobulin chains), CRL7O3O and HsS78Bst cells. In certain embodiments, anti-Axl antibodies described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) are produced in mammalian cells, such as CHO cells.

For long-term, high-yield production of recombinant proteins, stable expression cells can be generated. For example, cell lines which stably express an anti-Axl antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or an antigen-binding fragment thereof can be engineered. In specific embodiments, a cell provided herein stably expresses a light chain/light chain variable domain and a heavy chain/heavy chain variable domain which associate to form an antibody described herein (e.g., an antibody comprising the CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or an antigen-binding fragment thereof.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-Axl antibody described herein or a fragment thereof. Such engineered cell lines can be particularly useful in screening and evaluation of compositions that interact directly or indirectly with the antibody molecule.

A number of selection systems can be used, including, but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology can be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In a specific embodiment, antibodies described herein are isolated or purified.

### 5.3 Pharmaceutical Compositions and Kits

Provided herein are compositions, pharmaceutical compositions, and kits comprising one or more antibodies (e.g., anti-Axl antibodies comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) described herein, or antigen-binding fragments thereof, or conjugates thereof. In particular aspects, compositions (e.g., pharmaceutical compositions) described herein can be for in vitro, in vivo, or ex vivo uses. Non-limiting examples of uses include uses to modulate (e.g., inhibit) Axl activity and uses to manage or treat a disorder, for example, cancer. In specific embodiments, provided herein is a pharmaceutical composition comprising an antibody (e.g., a humanized antibody) described herein (or an antigen-binding fragment thereof) and a pharmaceutically acceptable carrier or excipient.

As used herein, the term “pharmaceutically acceptable” means being approved by a regulatory agency of the Federal or a state government, or listed in the U.S. Pharmacopeia, European Pharmacopeia or other generally recognized Pharmacopeia for use in animals, and, more particularly in humans.

Therapeutic formulations containing one or more antibodies provided herein (e.g., antibodies comprising CDRs of any of Ab301, Ab302, Ab303, Ab304, Ab305 and Abcon as set forth in Table 1, Table 4, Table 6, Table 8, Table 10 or Table 12, or an antibody or antigen- binding fragment comprising a VH and/or VL of Table 3) or an antigen-binding fragment thereof can be prepared for storage by mixing the antibody having the desired degree of purity with optional physiologically acceptable carriers, excipients or stabilizers (Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.), in the form of lyophilized formulations or aqueous solutions. Acceptable carriers, excipients, or stabilizers are nontoxic to recipients at the dosages and concentrations employed, and include buffers such as phosphate, citrate, and other organic acids; and/or non-ionic surfactants such as TWEEN™, PLURONICS™ or polyethylene glycol (PEG).

Formulations, such as those described herein, can also contain more than one active compounds (for example, molecules, e.g., antibody or antibodies described herein) as necessary for the particular indication being treated. In certain embodiments, formulations comprise an antibody provided herein and one or more active compounds with complementary activities that do not adversely affect each other. Such molecules are suitably present in combination in amounts that are effective for the purpose intended. For example, an antibody described herein can be combined with one or more other therapeutic agents (e.g., a tyrosine kinase inhibitor such as imatinib mesylated or sunitinib, an immune checkpoint inhibitor (for example, an immune checkpoint blockade that inhibits the activity of PD-1, PD-L1, PD-L2, CTLA-4, TIM-3, or LAG-3, such as PD-1 or PD-L1 antibody), or a histone deacetylase inhibitor such as vorinostat). Such combination therapy can be administered to the patient serially or simultaneously or in sequence.

The formulations to be used for in vivo administration can be sterile. This is readily accomplished by filtration through, e.g., sterile filtration membranes.

In specific aspects, the pharmaceutical compositions provided herein contain therapeutically effective amounts of one or more of the antibodies provided herein, and optionally one or more additional prophylactic of therapeutic agents, in a pharmaceutically acceptable carrier. Such pharmaceutical compositions are useful in the prevention, treatment, management or amelioration of a condition or disorder described herein or one or more symptoms thereof.

Pharmaceutical carriers suitable for administration of the antibodies provided herein include any such carriers known to those skilled in the art to be suitable for the particular mode of administration.

In addition, the antibodies described herein can be formulated as the sole pharmaceutically active ingredient in the composition or can be combined with other active ingredients (such as one or more other prophylactic or therapeutic agents).

Compositions provided herein can contain one or more antibodies provided herein (e.g., antibodies comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or an antigen-binding fragment thereof. In one embodiment, the antibodies are formulated into suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, one or more antibodies described herein is (are) mixed with a suitable pharmaceutical carrier. The concentrations of the antibody or antibodies in the compositions can, for example, be effective for delivery of an amount, upon administration, that treats, prevents, or ameliorates a condition or disorder described herein or a symptom thereof.

In one embodiment, compositions provided herein are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

In certain aspects, an antibody provided herein (e.g., antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) is included in the pharmaceutically acceptable carrier in an effective amount sufficient to exert a therapeutically useful effect in the absence of, or with minimal or negligible, undesirable side effects on the patient treated.

Concentrations of anti-Axl antibody in a pharmaceutical composition provided herein will depend on, e.g., the physicochemical characteristics of the antibody, the dosage schedule, and amount administered as well as other factors known to those of skill in the art. 1006281 The pharmaceutical compositions, in another embodiment, provide a dosage of about 50 mg of antibody per kilogram of body weight for administration over a period of time, e.g., every day or few days, every week, every 2 weeks, or every 3 weeks. Pharmaceutical dosage unit forms can be prepared to provide from about 500 mg.

Pharmaceutical compositions described herein are provided for administration to humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, such as tablets, capsules, pills, powders, granules, and oral or nasal solutions or suspensions, and oil-water emulsions containing suitable quantities of an anti-Axl antibody or pharmaceutically acceptable derivatives thereof. The antibody is, in one embodiment, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of an anti-Axl antibody sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles. Hence, in specific aspects, multiple dose form is a multiple of unit-doses which are not segregated in packaging.

In certain embodiments, one or more anti-Axl antibodies described herein (e.g., antibodies comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or an antigen-binding fragment thereof are in a liquid pharmaceutical formulation. Liquid pharmaceutically administrable compositions can, for example, be prepared by dissolving, dispersing, or otherwise mixing an antibody and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington's Pharmaceutical Sciences (1990) Mack Publishing Co., Easton, Pa.; Remington: The Science and Practice of Pharmacy, 21st ed. (2006) Lippincott Williams & Wilkins, Baltimore, MD. Dosage forms or compositions containing antibody in the range of 0.005% to 100% with the balance made up from non-toxic carrier can be prepared.

Parenteral administration, in one embodiment, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing an anti-Axl antibody described herein is an effective mode of administration. Another embodiment is a sterile aqueous or oily solution or suspension containing an anti-Axl antibody described herein injected as necessary to produce the desired pharmacological effect.

In specific embodiments, an anti-Axl antibody described herein can be suspended in micronized or other suitable form. The form of the resulting mixture depends upon a number of factors, including the intended mode of administration and the solubility of the compound in the selected carrier or vehicle.

In other embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving an anti-Axl antibody provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides an example of a formulation. In one embodiment, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain aspects, anti-Axl antibodies provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

Anti-Axl antibodies and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In some embodiments, anti-Axl antibodies described herein are targeted (or otherwise administered) to the visual organs, bone marrow, gastrointestinal tract, lungs, brain, or joints. In specific embodiments, an anti-Axl antibody described herein is capable of crossing the blood-brain barrier.

Provided herein is a pharmaceutical pack or kit comprising one or more containers filled with one or more of the ingredients of the pharmaceutical compositions described herein, such as one or more anti-Axl antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided herein are kits that can be used in the above methods. In one embodiment, a kit comprises an antibody described herein, preferably a purified antibody, in one or more containers. In a specific embodiment, kits described herein contain a substantially isolated Axl antigen (e.g., ECD of human Axl) as a control. In another specific embodiment, the kits described herein further comprise a control antibody which does not react with a Axl antigen. In another specific embodiment, kits described herein contain one or more elements for detecting the binding of a modified antibody to a Axl antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized Axl antigen. The Axl antigen provided in the kit can also be attached to a solid support. In a more specific embodiment, the detecting means of the above described kit includes a solid support to which a Axl antigen is attached. Such a kit can also include a non-attached reporter-labeled anti-human antibody or anti-mouse/rat antibody. In this embodiment, binding of the antibody to the Axl antigen can be detected by binding of the said reporter-labeled antibody.

### 5.4 Uses and Methods

In particular aspects, provided herein are methods of modulating Axl activity with an anti-Axl antibody (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) described herein or an antigen-binding fragment thereof. In certain embodiments, the anti-Axl antibody or antigen-binding fragment may have Axl antagonist activity.

In specific embodiments, provided herein are methods of inhibiting (e.g., partially inhibiting) Axl activity with an anti-Axl antibody described herein which may have an Axl antagonist activity.

In certain embodiments, provided herein are methods of managing or treating a condition or disorder using an anti-Axl antibody described herein which may have Axl antagonist activity.

Provided herein are methods of managing or treating a condition by inhibiting an activity of Axl with an anti-Axl antibody (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) described herein. In certain embodiments, an antibody or antigen-binding fragment disclosed herein may have Axl antagonist activity. Non-limiting examples of a condition which can be treated or managed with a Axl antibody or antigen-binding fragment thereof disclosed herein include cancer, sepsis, and infection, described in more detail below.

In certain embodiments, a condition which can be treated or managed with an anti-Axl antibody is one which can be managed or treated by enhancing an immune response. In certain embodiments, an antibody or antigen-binding fragment disclosed herein may have an Axl antagonist activity.

An anti-Axl antibody described herein or an antigen-binding fragment thereof, which may have an Axl antagonist activity, for use in the methods provided herein is capable of inhibiting (e.g., partially inhibiting) or decreasing/reducing Axl expression and/or a Axl activity. Activities of Axl are known in the art. In specific embodiments, an anti-Axl antibody described herein inhibits (e.g., partially inhibits) one or more of the following Axl activities: phosphorylation (e.g., autophosphorylation) of Axl receptor (e.g., cytoplasmic domain of Axl, such as cytoplasmic kinase domain of Axl), promotion of phagocytosis, modulation of maturation of NK cells, dendritic cells or macrophages, inhibition of toll-like receptor (TLR) activation of dendritic cells, inhibition or decrease in TLR-induced proinflammatory cytokine (e.g., TNF, IL-6, IL-12 and type I interferon production, increase in SOCS1 expression, association with interleukin-15 (IL-15) to protect cells against TNF-induced cell death (e.g., fibroblasts and DCs), and activation of Stat1 in dendritic cells. Methods for measuring these activities are known in the art. In certain embodiments, an antibody or antigen-binding fragment disclosed herein may have an Axl antagonist activity.

In specific embodiments, an anti-Axl antibody described herein which may have an Axl antagonist activity inhibits (e.g., partially inhibits), by at least about 10%, 20%, 30%, 40%, 50%, 60%, 70%, 80%, 90%, 97%, 98%, 99%, or 100%, one or more such Axl activities. In certain embodiments, an antibody or antigen-binding fragment disclosed herein may have an Axl antagonist activity

In particular aspects, provided herein is a method of enhancing an immune response in a subject comprising administering to a subject in need thereof an effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits Axl.

In specific embodiments, a subject (e.g., human subject) in need of enhancing an immune response include an immunocompromised subject, for example a subject suffering from an infection (e.g., bacterial, viral, fungal, or protozoan). In certain embodiments, an immunocompromised subject has cancer and is undergoing, or had undergone treatment with, anti-cancer therapy, such as, chemotherapy. Other non-limiting examples of immunocompromised subjects include, but are not limited to, subjects who are HIV positive, subjects with AIDS or SCID, subjects with diabetes, and subjects who have undergone transplants and are taking immunosuppressants. In certain embodiments, an immunocompromised subject is taking immunosuppressants (e.g., steroids) to manage or treat asthma, arthritis (e.g., rheumatoid arthritis), or allergy or an allergic condition.

In a particular embodiment, provided herein is a method of enhancing an immune response to a vaccine in a subject, comprising administering to a subject in need thereof, who is or has been administered the vaccine, an effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits Axl. In specific embodiments, the antibody is administered before, concurrently, or after administration of the vaccine.

An example of vaccines in the methods provides herein include cancer/tumor vaccines, such as vaccines comprising tumor cells or tumor cell lysates and vaccines comprising dendritic cells or dendritic cell lysates activated against a tumor.

Provided herein is a method of managing, preventing, protecting against, or treating cancer in a subject, comprising administering to a subject in need thereof a therapeutically effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity or inhibits or reduces Axl expression. In particular embodiments, such a method involves inhibition of tumor growth, cancer cell proliferation, cancer cell migration, inhibition of tumor-associated macrophage activity (e.g., tumor-promoting activity such as production of cytokines), and modulating stromal cells such as macrophages or dendritic cells

Non-limiting examples of cancers to be managed, prevented, protected against, or treated in the methods provided herein include breast cancer, pancreatic cancer, leukemia, lung cancer such as non-small cell lung cancer, glioblastoma, melanoma, prostate cancer, colon cancer, gastric cancer, pituitary adenomas, ovarian cancer, renal cancer, bladder cancer, and sarcomas, including rhabdomyosarcomas.

In some embodiments, an antibody or antigen-binding fragment described herein, which specifically binds to a Axl polypeptide described herein is capable of inhibiting tumor growth at a site of local administration. In some embodiments, an antibody or antigen-binding fragment described herein, which specifically binds to a Axl polypeptide described herein is capable of inhibiting tumor growth in vivo (e.g., in a mouse model and/or in a human having cancer) at a site of local administration. In some embodiments, an antibody or antigen-binding fragment described herein, which specifically binds to a Axl polypeptide described herein administered to a patient using the methods described herein has one or more of the following non-limiting effects at a site of local administration: inhibit proliferation of tumor cells, inhibit tumor growth, reduce the tumorigenicity of a tumor, trigger cell death of tumor cells, increase cell contact-dependent growth inhibition, increase tumor cell apoptosis, decrease survival of tumor cells, reduce the number of tumor cells, decrease tumorigenic frequency, reduce the number or frequency of cancer stem cells, reduce the tumor size, reduce the cancer cell population; inhibit or stop cancer cell infiltration into peripheral organs including, inhibit metastasis, inhibit tumor growth.

In some embodiments, an antibody or antigen-binding fragment described herein relieves one or more of the symptoms associated with cancer, reduces morbidity and mortality, and/or improves quality of life.

In a specific embodiment, provided herein is a method of treating cancer in a subject comprising administering to a subject in need thereof an antibody described herein (e.g., antibody Ab301, Ab302, Ab303, Ab304, Ab305 or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits Axl, in an effective amount to enhancing an immune response to the cancer.

In one aspect, provided herein is a method of managing, preventing, protecting against, or treating metastasis in a subject, comprising administering to a subject in need thereof a therapeutically effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity or inhibits or reduces Axl expression.

In a certain aspect, provided herein is a method of inhibiting Axl expression and/or activity of a tumor-associated macrophage comprising contacting a tumor-associated macrophage with an effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity or inhibits or reduces Axl expression. In a specific embodiment, such a method disrupts tumor-macrophage cross talk and inhibits the ability of tumor-associated macrophages to promote cancer cell proliferation.

In a certain aspect, provided herein is a method of modulating (e.g., inhibiting) stromal cell (e.g., macrophage or dendritic cell) activity, comprising contacting a stromal cell with an effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity or inhibits or reduces Axl expression in the stromal cell. In a specific embodiment, such method is for modulating stromal cell activity in a subject with cancer.

In a certain aspect, provided herein is a method of modulating (e.g., inhibiting) natural killer (NK) cell activity, comprising contacting an NK cell with an effective amount of an antibody described herein (e.g., antibody Ab301, Ab302, Ab303, Ab304 or Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity or inhibits or reduces Axl expression in the NK cell. In a specific embodiment, such method is for modulating NK cell activity in a subject with cancer.

In another aspect, provided herein is a method of modulating (e.g., inhibiting) T cell activity, comprising contacting a T cell with an effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity or inhibits or reduces Axl expression in the T cell. In a specific embodiment, such method is for modulating stromal cell activity in a subject with cancer.

In a particular embodiment, provided herein is a method of inhibiting or reducing tumor growth or cancer cell proliferation in a subject, comprising administering to a subject in need thereof a therapeutically effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity or inhibits or reduces Axl expression.

In a specific embodiment, provided herein is a method of treating cancer in a subject comprising administering (e.g., administering concurrently or sequentially) to a subject in need thereof (i) an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits Axl, and (ii) another anti-cancer agent.

In one embodiment, provided herein is a method of managing, preventing, protecting against, or treating an infection in a subject, comprising administering to a subject in need thereof a therapeutically effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity.

In a specific embodiment, the infection is an infection caused by bacteria (gram-negative bacteria or gram-positive bacteria), fungi, viruses, or parasites.

In a particular embodiment, provided herein is a method of managing or treating sepsis in a subject, comprising administering to a subject in need thereof a therapeutically effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity.

Sepsis (a systemic inflammatory response syndrome or SIRS) is an immunological condition characterized by whole-body inflammation caused by infection, such as infection by bacteria (gram-negative bacteria or gram-positive bacteria), fungi, viruses, or parasites. Stages of sepsis include, but are not limited to, onset of sepsis, severe sepsis, and septic shock. Severe sepsis can be accompanied by organ dysfunction.

In certain embodiments, the sepsis is advanced sepsis. In a particular embodiment, the sepsis is characterized with increased expression of SOCS, for example during the stage of severe sepsis. In a particular embodiment, the sepsis is characterized with elevated levels of circulating Gas6 in a subject with sepsis. Levels or concentrations of circulating Gas6 can be measured from blood samples using conventional methods, such as ELISAs. Levels or concentrations of Gas6 can be compared with a reference value to determine whether Gas6 levels or concentrations are elevated in a subject with sepsis. For example, the reference values can be a set standard range of levels and concentrations of Gas6 accepted in the art as correlating with those of a population of healthy subjects not suffering from sepsis. As another example, the reference values can be levels of concentrations of Gas6 in a subject prior to the onset of sepsis or at the early stages of sepsis. Symptoms of sepsis include, but are not limited to, high fevers, hot, flushed skin, elevated heart rate, hyperventilation, altered mental status, swelling, and low blood pressure.

In a particular embodiment, provided herein is a method of managing, alleviating or treating one or more symptoms of sepsis in a subject, comprising administering to a subject in need thereof a therapeutically effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity.

In certain embodiments, the method of managing or treating sepsis provided herein further comprises the step of administering an antibiotic to a subject in need thereof, for example, prior to, concurrently with, or subsequent to administering an anti-Axl antibody described herein.

In certain aspects, provided herein are methods for managing, treating, preventing, or protecting against infection in a subject, comprising administering to a subject in need thereof an effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity.

In certain embodiments, the infection is caused by microbes, e.g., bacteria, viruses or parasites. In certain embodiments, the viral infection is HIV, herpes, shingles, influenza, common cold, and encephalitis.

In certain embodiments, provided herein are methods for preventing or inhibiting entry of a viruses by contacting a cell to an effective amount of an antibody described herein (e.g., antibody Ab301, Ab302, Ab303, Ab304, Ab305, or Abcon, or an antibody comprising CDRs of any of Ab301, Ab302, Ab303, Ab304, Ab305 and Abcon as set forth in Table 1, Table 4, Table 6, Table 8, Table 10 or Table 12, or an antibody or antigen-binding fragment comprising a VH and/or VL of Table 3) or antigen-binding fragment thereof that specifically binds to human Axl and inhibits a Axl activity. In particular embodiments, the method provided herein prevents or inhibits entry of lymphocytic choriomeningitis virus, HIV, herpes virus (e.g., herpes simplex virus or herpes zoster), influenza virus, or common cold virus.

In certain embodiments, the anti-Axl antibody or antigen-binding fragment thereof for use in the methods provided herein is an antibody (e.g., monoclonal antibody, such as a humanized monoclonal antibody) comprising CDRs of Table 1, Table 4, Table 6, Table 8, Table 10 or Table 12. In particular embodiments, the anti-Axl antibody inhibits a Axl activity.

In other aspects, provided herein are methods of enhancing a type I interferon (IFN) response against a pathogen in a subject, comprising administering to a subject in need thereof an effective amount of an anti-Axl antibody described herein. In certain embodiments, an antibody or antigen-binding fragment disclosed herein may have an Axl antagonist activity.

As used herein, “administer” or “administration” refers to the act of injecting or otherwise physically delivering a substance (e.g., a humanized anti-Axl antibody provided herein or an antigen-binding fragment thereof) to a subject or a patient (e.g., human), such as by mucosal, topical, intradermal, parenteral, intravenous, intramuscular delivery and/or any other method of physical delivery described herein or known in the art.

As used herein, the terms “effective amount” or “therapeutically effective amount” refer to an amount of a therapy (e.g., an antibody or pharmaceutical composition provided herein) which is sufficient to reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy (e.g., a therapy other than an anti-Axl antibody provided herein). In some embodiments, “effective amount” as used herein also refers to the amount of an antibody described herein to achieve a specified result.

As used herein, the term “in combination” in the context of the administration of other therapies refers to the use of more than one therapy. The use of the term “in combination” does not restrict the order in which therapies are administered. The therapies may be administered, e.g., serially, sequentially, concurrently, or concomitantly.

As used herein, the terms “manage,” “managing,” and “management” refer to the beneficial effects that a subject derives from a therapy (e.g., a prophylactic or therapeutic agent), which does not result in a cure of a condition associated with Axl. In certain embodiments, a subject is administered one or more therapies (e.g., prophylactic or therapeutic agents, such as an antibody described herein) to “manage” a condition or disorder described herein, one or more symptoms thereof, so as to prevent the progression or worsening of the condition or disorder.

As used herein, the terms “impede” or “impeding” in the context of a condition or disorder provided herein refer to the total or partial inhibition (e.g., less than 100%, 95%, 90%, 80%, 70%, 60%, 50%, 40%, 30%, 20%, 10%, or 5%) or blockage of the development, recurrence, onset or spread of a condition or disorder provided herein and/or symptom related thereto, resulting from the administration of a therapy or combination of therapies provided herein (e.g., a combination of prophylactic or therapeutic agents, such as an antibody described herein).

As used herein, the term “prophylactic agent” refers to any agent that can totally or partially inhibit the development, recurrence, onset or spread of a condition or disorder provided herein, and/or symptom related thereto in a subject. In certain embodiments, the term “prophylactic agent” refers to an antibody described herein. In certain other embodiments, the term “prophylactic agent” refers to an agent other than an antibody described herein. Generally, a prophylactic agent is an agent which is known to be useful to or has been or is currently being used to prevent a condition or disorder provided herein, and/or a symptom related thereto or impede the onset, development, progression and/or severity of a condition or disorder provided herein, and/or a symptom related thereto. In specific embodiments, the prophylactic agent is an anti-Axl antibody, as described herein.

As used herein, the terms “subject” and “patient” are used interchangeably. As used herein, a subject is a mammal such as a non-primate (e.g., cows, pigs, horses, cats, dogs, goats, rabbits, rats, mice, etc.) or a primate (e.g., monkey and human), for example a human. In one embodiment, the subject is a mammal, e.g., a human, diagnosed with a condition or disorder provided herein. In another embodiment, the subject is a mammal, e.g., a human, at risk of developing a condition or disorder provided herein. In a preferred embodiment, the subject is human.

As used herein, the terms “therapies” and “therapy” can refer to any protocol(s), method(s), compositions, formulations, and/or agent(s) that can be used in the prevention, treatment, management, or amelioration of a condition or disorder or symptom thereof (e.g., a condition or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a condition or disorder or one or more symptoms thereof. In certain embodiments, the term “therapy” refers to a therapy other than an anti-Axl antibody described herein or pharmaceutical composition thereof. In specific embodiments, an “additional therapy” and “additional therapies” refer to a therapy other than a treatment using an anti-Axl antibody described herein or pharmaceutical composition thereof. In a specific embodiment, a therapy includes the use of an anti-Axl antibody described herein as an adjuvant therapy. For example, using an anti-Axl antibody described herein in conjunction with a drug therapy, biological therapy, surgery, and/or supportive therapy.

As used herein, the term “therapeutic agent” refers to any agent that can be used in the treatment, management or amelioration of a condition or disorder or symptom thereof. In certain embodiments, the term “therapeutic agent” refers to an anti-Axl antibody described herein or an antigen-binding fragment thereof. In certain other embodiments, the term “therapeutic agent” refers to an agent other than an antibody described herein. In specific embodiments, a therapeutic agent is an agent which is known to be useful for, or has been or is currently being used for the treatment, management or amelioration of a condition or disorder provided herein or one or more symptoms or condition associated therewith or one or more symptoms related thereto.

In one embodiment, for example, an antibody or antigen-binding fragment thereof that specifically binds to human Axl as described herein can be administered in combination with another therapeutic agent for addressing one or more of the indications described herein. In a specific embodiment, such a therapeutic agent can be a stimulator of T cell responses.

In some embodiments, an antibody or antigen-binding fragment thereof that specifically binds to human Axl as described herein can be administered in combination with another therapeutic agent (for example, a monoclonal antibody), wherein the therapeutic agent is an immune checkpoint modulator (preferably, an immune checkpoint blocker). As a non-limiting example, the immune checkpoint modulator can be a modulator of (preferably, an inhibitor of) Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), OX40, CD27, CD28, CD40, CD137 (4-1BB), CGEN-15001T, CGEN-15022, CGEN-15027, CGEN-15049, CGEN-15052, CGEN-15092, Lymphocyte activation gene-3 (LAG-3), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, or Herpes Virus Entry Mediator (HVEM). In a specific embodiment, the immune checkpoint modulator inhibits the activity of PD-1. In another specific embodiment, the immune checkpoint modulator inhibits the activity of PD-L1.

In certain embodiments, a method for treating a disease in a patient disclosed herein comprises administering to the patient a therapeutically effective amount of an antibody or antigen-binding fragment described herein, which specifically binds to a Axl polypeptide (e.g., human Axl) and one or more other therapeutic agents, wherein a therapeutic agent is a checkpoint inhibitor (e.g., PD1 inhibitor or PD-L1 inhibitor). In some embodiments, provided herein are methods for treating a neoplastic disease in a subject by administering in combination with an antibody or antigen-binding fragment described herein, which specifically binds to a Axl polypeptide, an immune checkpoint inhibitor that inhibits, decreases or interferes with the activity of a negative checkpoint regulator. In some embodiments, the negative checkpoint regulator is selected from the group consisting of PD-1, PD-L1, and PD-L2.

In specific embodiments, a therapeutic agent can be an immune checkpoint inhibitor, e.g., a PD-1, PD-L1, or CTLA-4 inhibitor. Such exemplary agents can include, e.g., Yervo™ (ipilimumab) or tremelimumab (to CTLA-4), BMS-936558/nivolumab (to PD-1), MK-3475/pembrolizumab (to PD-1). In additional embodiments, such a therapeutic agent can be, for example, CDX-1127 (to CD27), CP-870893 (to CD40), lucatumumab (to CD40), or dacetuzumab (to CD40).

In some embodiments, a method of treating cancer in a subject comprises administering to the subject a therapeutically effective amount of an antibody or antigen-binding fragment described herein, which specifically binds to a Axl polypeptide. In some embodiments, a method of treating cancer in a subject comprises administering to the subject a therapeutically effective amount of antibody or antigen-binding fragment described herein, which specifically binds to a Axl polypeptide in combination with a checkpoint inhibitor. In some embodiments, the checkpoint inhibitor is an anti-PD-1 antibody (e.g., MEDI0680, REGN2810, BGB-A317, or PDR001). In some embodiments, the checkpoint inhibitor is an anti-PD-1 antibody (e.g., nivolumab (OPDIVO), pembrolizumab (KEYTRUDA), or pidilzumab). In some embodiments, the checkpoint inhibitor is an anti-PD-L1 antibody (e.g., BMS935559 (MDX-1105), atezolizumab (TECENTRIQ), durvalumab (MEDI4736; IMFINZI), avelumab (MSB0010718C), or LY3300054).

It will be appreciated that the combination of an antibody or antigen-binding fragment described herein, which specifically binds to a Axl polypeptide and one or more additional therapeutic agent(s) can be administered in any order or concurrently.

Additional therapeutic agents which may be used in combination with the anti-Axl antibodies and antigen-binding fragments of the present invention include without limitation: chemotherapeutic agents, radiotherapy, adjuvants and immunostimulatory agents such as CD40 ligand, Flt3 ligand including soluble Flt3 ligand (CDX-301), cytokines, colony-stimulating factors (such as G-CSF or GM-CSF), STING agents, CSFR inhibitors, anti-41BB antibodies, anti-0X40 antibodies, anti-CD40 antibodies (such as CDX-1140), anti-CD27 antibodies (such as varlilumab), LPS (endotoxin), ssRNA, dsRNA or Toll-like Receptor (TLR) agonists (e.g., TLR3 agonist such as Poly IC or PolyICLC, a TLR4 agonist, a TLR5 agonist, a TLR7 agonist, a TLR8 agonist, or a TLR 9 agonist).

Tumor antigens can also be used as additional therapeutic agents in combination with the anti-Tyro3 antibodies and antigen-binding fragments of the present invention.Tumor antigens which may be used in combination with the anti-Axl antibodies and antigen-binding fragments of the present invention include any antigen or antigenic determinant which is present on (or associated with) a tumor cell and not typically on normal cells, or an antigen or antigenic determinant which is present on or associated with tumor cells in greater amounts than on normal (non-tumor) cells, or an antigen or antigenic determinant which is present on tumor cells in a different form than that found on normal (non-tumor) cells. Such antigens include tumor-specific antigens, including tumor-specific membrane antigens, tumor-associated antigens, including tumor-associated membrane antigens, embryonic antigens on tumors, growth factor receptors, growth factor ligands, and any other type of antigen that is associated with cancer. A tumor antigen may be, for example, an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. The tumor antigen may be, for example, bhCG, gp100 or Pmel17, CEA, gp100, TRP-2, NY-BR-1, NY-CO-58, MN (gp250), idiotype, Tyrosinase, Telomerase, SSX2, MUC-1, MAGE-A3, high molecular weight-melanoma associated antigen (HMW-MAA) MART1, melan-A, EGFRvIII, NY-ESO-1, MAGE-1, MAGE-3, WT1, Her2, or mesothelin. Other antigens, including vaccine antigens from infectious disease pathogens, such as viruses, bacteria, parasites and fungi, may also be used as additional therapeutic agents in combination with the anti-Tyro3 antibodies and antigen-binding fragments of the present invention.

In some embodiments, combined administration can include co-administration, either in a single pharmaceutical formulation or using separate formulations, or consecutive administration in either order but generally within a time period such that all active agents can exert their biological activities simultaneously. In some embodiments, an antibody or antigen-binding fragment described herein, which specifically binds to a Axl polypeptide and at least one additional therapeutic agent can be administered using the same methods or can be administered using different methods known in the art.

### 5.4.1 Diagnostic Uses

In one aspect, anti-Axl antibodies described herein and antigen-binding fragments thereof, which specifically bind to an ECD of human Axl can be used for diagnostic purposes to detect, diagnose, or monitor a condition described herein (e.g., a condition involving Axl and/or abnormal Axl signaling and/or abnormal Axl expression). In specific embodiments, anti-Axl antibodies described herein or an antigen-binding fragment thereof for use in diagnostic purposes are labeled.

In certain embodiments, provided herein are methods for the detection of a condition described herein comprising: (a) assaying the expression of Axl in cells or a tissue sample of a subject using one or more antibodies described herein or an antigen-binding fragment thereof; and (b) comparing the level of Axl expression with a control level, e.g., levels in normal tissue samples (e.g., from a patient not having a condition described herein, or from the same patient before onset of the condition), whereby an increase or decrease in the assayed level of Axl expression compared to the control level of Axl expression is indicative of a condition described herein.

Antibodies described herein can be used to assay Axl levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (1251, 1211), carbon (14C), sulfur (35S), tritium (3H), indium (1211n), and technetium (99Tc); luminescent labels, such as luminol; and fluorescent labels, such as fluorescein and rhodamine, and biotin.

In one embodiment, monitoring of a condition described herein is carried out by repeating the method for diagnosing for a period of time after initial diagnosis.

Presence of the labeled molecule can be detected in the subject using methods known in the art for in vivo scanning. Skilled artisans will be able to determine the appropriate method for detecting a particular label. Methods and devices that may be used in the diagnostic methods include, but are not limited to, computed tomography (CT), whole body scan such as position emission tomography (PET), magnetic resonance imaging (MRI), and sonography. In a preferred embodiment, the subject is human.

### 6. EXAMPLES

The examples in this section (i.e., Section 6) are offered by way of illustration, and not by way of limitation.

### 6.1 Example 1: Generation of Anti-Human Axl Monoclonal Antibodies

Ab301 was obtained by repeated immunization of female rats with purified human Axl extracellular domain emulsified in Freund's Complete Adjuvant. Antibody serum titers were determined after immunization by ELISA, and cell fusion was performed to generate hybridoma cells. For cell fusion, lymphocytes were obtained from the lymph nodes and spleens of immunized rats. Hybridoma cells were generated by fusing the lymphocytes with NS0 myeloma fusion partner cells. Conditioned media from hybridoma cells were then screened and later re-screened for binding to human Axl by standard ELISA methods. Hybridoma clones that secreted anti-Axl antibodies were subcloned by limiting dilution. The VH and VL regions of antibodies from clones of interest were sequenced and sub-cloned into a construct containing human IgG1 constant regions to generate chimeric antibodies.

### 6.1.1 Humanization of Anti-Axl Antibodies

Using antibody numbering systems from IMGT and Kabat, the CDRs were identified. A combined IMGT/Kabat CDR sequence was used for optimal retention of CDR-loop conformation.

Online databases of human IgG sequences were searched for comparison to the murine VH domain using BLAST search algorithms, and candidate human variable domains were selected from the top 200 BLAST results. These were reduced to four candidates based on a combination of framework homology, maintaining key framework residues and canonical loop structure. The closest human germline was chosen as a 5th framework. CDRs of the rat VH were grafted into these 5 acceptor frameworks to become the humanized VH variants. These humanized variants were checked to determine whether they had been humanized in accordance with WHO's definition of humanized antibodies: The variable domain of a humanized chain has a V region amino acid sequence which, when analyzed as a whole, is closer to human than to other species (assessed using the Immunogenetics Information System® (IMGT®) DomainGapAlign tool).

Likewise, online databases of human IgK sequences were searched for comparison to the rat VL domain using BLAST search algorithms, and candidate human variable domains selected from the top 200 BLAST results. These were reduced to four candidates based on a combination of framework homology, maintaining key framework residues and canonical loop structure. The closest human germline was chosen as a 5th framework. CDRs of the rat VH were grafted into these 5 acceptor frameworks to become the humanized VL variants. When the humanized variants were checked to determine whether they had been humanized in accordance with WHO's definition of humanized antibodies, it was determined that all of the light chains remained rat, although very close to humanized as the light chain sequence is very close to the rat germline, with only two differences in the CDR. A 6th framework was then designed using a germline sequence that was less similar to the parental antibody but allowed for the sequence to be humanized in accordance with WHO's definition while retaining the original CDRs.

Each of the 5 humanized VH domains were synthesized in-frame with a human IgG1 isotype constant domain sequence. A single (6^{th}) humanized VL domain was synthesized in-frame with a human IgK isotype constant domain sequence.

### 6.2 Example 2: Binding to Human Axl

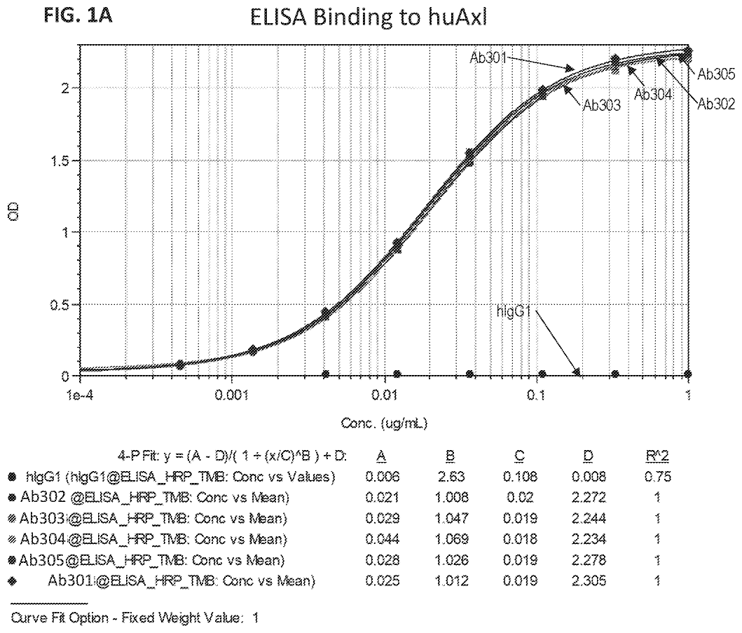

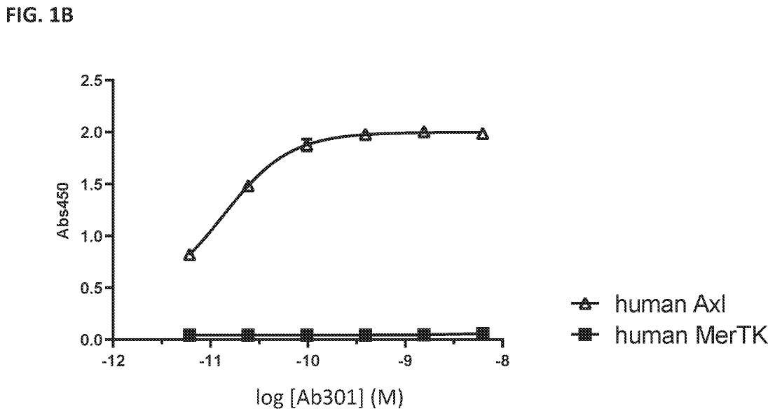

Binding of Ab301 and four humanized antibody subclones to human Axl was determined by ELISA using the following protocol: Microtiter plates were coated with recombinant human Axl-msFc, in phosphate-buffered saline (PBS), and then blocked with 5% bovine serum albumin (BSA) in PBS. Protein A purified human monoclonal antibodies and an isotype control were added at various concentrations and incubated at 37° C. The plates were washed with PBS/Tween and then incubated with a goat-anti-human IgG Fc-specific polyclonal reagent conjugated to horseradish peroxidase (HRP) at 37° C. After washing, the plates were developed with HRP substrate, and analyzed at OD 450-650 using a microtiter plate reader. Data is shown in FIG. 1A.

Binding of Ab301 to human Axl extracellular domain and its lack of binding to human MerTK extracellular domain was determined using the following protocol: Nunc MaxiSorb™ plates (Thermo Fisher Scientific) were coated with 100 ng of the purified extracellular domain of human Axl or human MerTK in 10 nM borate buffer, overnight at 4° C. Plates were washed and blocked for 1 hour at room temperature with Tris-buffered saline (TBS) with 1% BSA. A titration of Ab301 was added to the coated plates for 1 hour at room temperature, followed by washing and incubation with an HRP-conjugated goat anti-human antibody (Jackson Immunoresearch) for 1 hour at room temperature. Washed plates were incubated with 3,3′,5,5′-Tetramethylbenzidine (TMB) substrate (Thermo Fisher Scientific) and read using a Biotek® plate reader. Data are plotted as the optical density at 450 nm (Abs450) as a function of the log-transformed molar concentration of Ab301, and fit to a 4-parameter binding curve using GraphPad Prism™. Data is shown in FIG. 1B.

### 6.3 Example 3: Binding to Axl Expressing Cells

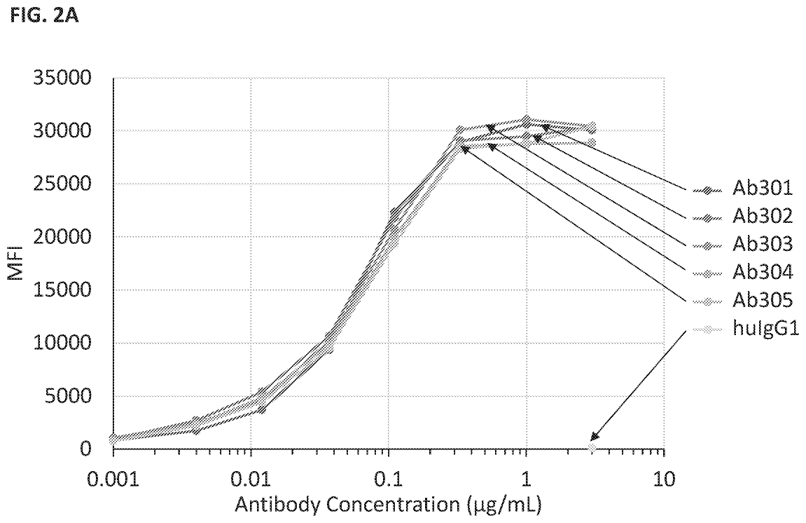

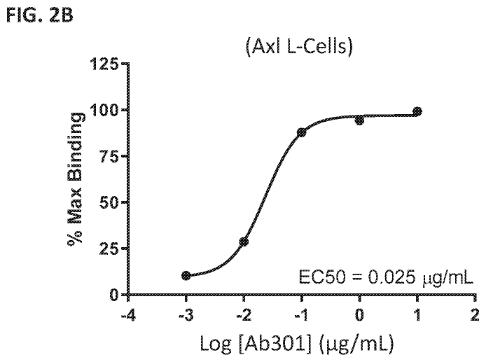

The ability of anti-Axl human antibodies to bind to Axl on cells expressing human Axl on their surface was investigated by flow cytometry as follows: Purified antibodies were incubated with either L cells expressing human Axl on their surface or H1299 cells at room temperature on a plate shaker. After 20 minutes, the cells were washed with PBS containing 0.1% BSA and 0.05% NaN₃ (PBA), and the bound antibodies were detected by incubating the cells with a phycoerythrin (PE)-labeled goat anti-human IgG Fc-specific probe. The excess probe was washed from the cells with PBA and the cell-associated fluorescence was determined by analysis using a FACSCanto II™ instrument (BD Biosciences, NJ, USA) according to the manufacturer's directions. Data is shown in FIG. 2A and FIG. 2B.

### 6.4 Example 4: Determination of Affinity and Rate Constants by Bio-Layer Interferometry (BLI)

Binding affinity and binding kinetics of the various anti-Axl antibodies were examined by bio-layer interferometry (BLI) using an Octet™ QK^{e} instrument (Pall ForteBio, Menlo Park, Calif.) according to the manufacturer's guidelines, using the following protocol: Purified antibodies were captured on Anti-Human Fc Capture (AHC) biosensors (Fortebio Product No. 18-5060). Each antibody was prepared in dilution buffer (10 mM PO₄, 150 mM NaCl, 1 mg/mL BSA and 0.05% Tween® 20, pH 7.2) to 1 μg/mL, and loaded onto freshly hydrated and conditioned AHC biosensors for 120 seconds at 30° C. and 1000 rpm plate shake speed to achieve a target response of 1.0 nm. For one assay, eight biosensors were loaded with the same antibody.

Binding was determined by exposing the antibody loaded biosensors to the analyte, i.e., soluble human biotinylated Axl-CD4-HIS (Celldex, 80 kD by SDS-PAGE). Affinity measurements were determined using 2-fold serial dilutions of analyte ranging from 50 nM to 0.78 nM in dilution buffer at 30° C. and 1000 rpm plate shake speed. Association of the antibody loaded biosensors in analyte wells was carried out for 300 seconds, and the biosensors were then moved to dilution buffer wells for 600 seconds for dissociation measurements.

Corresponding controls were conducted in each case by keeping the two remaining biosensors with captured antibody in dilution buffer wells for association and dissociation steps. The data for the control biosensors was used to subtract background and account for biosensor drift and antibody dissociation from the biosensors.

Fortebio's Data Analysis Software version 11.0.0.4 (Pall ForteBio, Menlo Park, Calif.) was used in each case to derive kinetic parameters from the concentration series of analyte in dilution buffer binding to captured antibody. The association and dissociation curves were fitted to a 1:1 binding model using the data analysis software according to the manufacturer's guidelines.

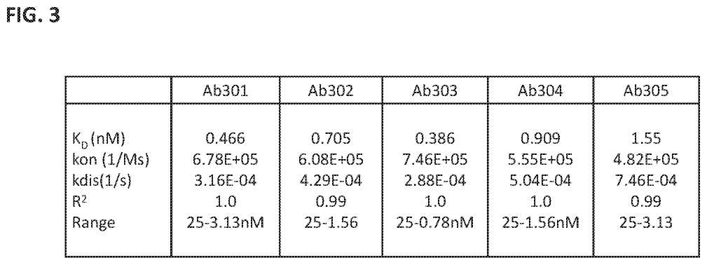

The affinity and kinetic parameters (with background subtracted) as determined are shown in FIG. 3, where kon=rate of association, kdis=rate of dissociation, and K_{D}=affinity constant, determined by the ratio kdis/kon.

### 6.5 Example 5: Domain Binding Analysis

An ELISA-based method was utilized to determine to which of the four domains of the Axl extracellular domain Ab301 binds.

Table 15, utilizing standard one letter amino acid codes, shows the amino acid sequences of each of the four domains that comprise the human Axl extracellular domain.

TABLE 15
Amino acid sequences of each of the Ig-like domains that comprise the human
Axl extracellular domain

| hAxl ECDs | Amino Acid Sequence |
|---|---|
| Domain 1 (Ig-like | ESPFVGNPGNITGARGLTGTLRCQLQVQGEPPEVHWLRDGQILELADSTQTQV |
| domain 1) or D1 | PLGEDEQDDWIVVSQLRITSLQLSDTGQYQCLVFLGHQTFVSQPGYVGLEG |
| (SEQ ID NO: 1) | |
| Domain 2 (Ig-like | LPYFLEEPEDRTVAANTPFNLSCQAQGPPEPVDLLWLQDAVPLATAPGHGPQR |
| domain 2) or D2 | SLHVPGLNKTSSFSCEAHNAKGVTTSRTATITVLPQQ |
| (SEQ ID NO: 2) | |
| Domain 3 (FnIII- | PRNLHLVSRQPTELEVAWTPGLSGIYPLTHCTLQAVLSDDGMGIQAGEPDPPE |
| like domain 1) or | EPLTSQASVPPHQLRLGSLHPHTPYHIRVACTSSQGPSSWTHTWLPVETPEG |
| D3 | |
| (SEQ ID NO: 3) | |
| Domain 4 (FnIII- | VPLGPPENISATRNGSQAFVHWQEPRAPLQGTLLGYRLAYQGQDTPEVLMDIG |
| like domain 2) or | LRQEVTLELQGDGSVSNLTVCVAAYTAAGDGPWSLPVPLEAWRPGQAQPVHQL |
| D4 | VKEPS |
| (SEQ ID NO: 4) | |

Human Axl domains (D1-D4) of the Axl extracellular domain (Axl-ECD) were expressed in Exi293 cells, purified by nickel affinity chromatography and buffer exchanged to PBS. 100 ng of each domain was coated onto 96 well high binding ELISA plates (Greiner Bio-One) in borate buffer (Pierce Biotechnology) for 2 hours at room temperature, and subsequently blocked with TBS containing 1% BSA for 1 hour at room temperature. Ab301 was added at 5 μg/mL for 1 hour at room temperature, washed, and subsequently incubated with an HRP- conjugated goat anti-human antibody (Jackson Immunoresearch). Plates were washed and incubated with TMB (Pierce Biotechnology).

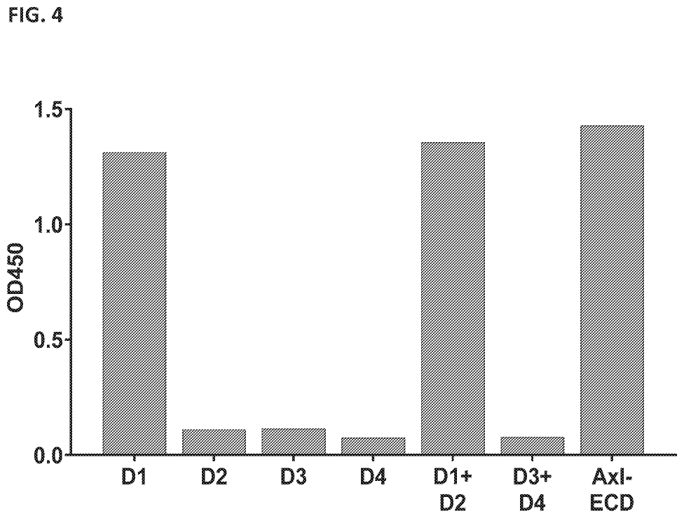

Data is shown in FIG. 4. Results show that Ab301 binds to recombinantly purified Domain 1 of the Axl-ECD alone, Domain 1 and Domain 2 of Axl-ECD, and the intact Axl extracellular domain (ECD).

### 6.6 Example 6: Ligand Blocking Assay

Blocking of Gas6 Binding to Axl (ELISA)

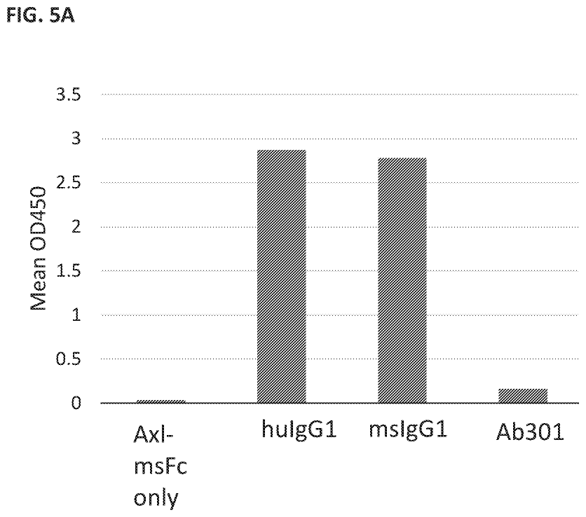

The ability of Ab301 to prevent Gas6 from binding to Axl was assessed by an ELISA assay. Gas6-biotin was captured to streptavidin microtiter plates. Protein A purified human Ab301 or an isotype control (human or mouse) were mixed with soluble human Axl-msFcand incubated at 37° C. The plates were washed with PBS/Tween and then incubated with a goat-anti-mouse IgG Fc-specific polyclonal reagent conjugated to horseradish peroxidase at 37° C. After washing, the plates were developed with HRP substrate, and analyzed at OD 450-650 using a microtiter plate reader. Data is shown in FIG. 5A, which shows that Ab301 prevented Gas6 from binding to human Axl.

Blocking of Gas6 Binding to H1299 Cells

Gas6 was fluorescently labeled with Alexa-647 (Gas6-647) according to manufacturer's instructions (Thermo Fisher Scientific). H1299 cells were incubated with FACS Buffer (PBS with 2% newborn calf serum) for 1 hour at 4° C., washed and incubated with (or without) 100 nM Ab301 for 2 hours on ice. Cells were then centrifuged and resuspended in FACS buffer containing 50 nM Gas6-647 for 1 hour, subsequently washed, and resuspended in buffer containing 7-Aminoactinomycin D (7-AAD). Fluorescence on live cells was analyzed by flow cytometry using an Accuri™ C6 instrument (BD Biosciences). Fluorescent data are represented by mean fluorescent intensity (MFI). Data is shown in FIG. 5B, which shows that Ab301 was able to block Gas6 binding to Axl expressed on the surface of H1299 cells.

### 6.7 Example 7: Axl, AKT and ERK Phosphorylation Assay

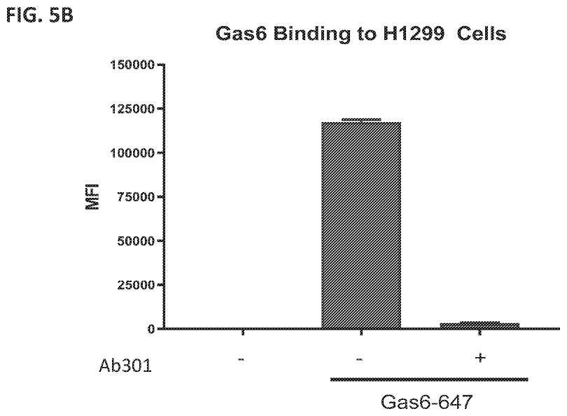

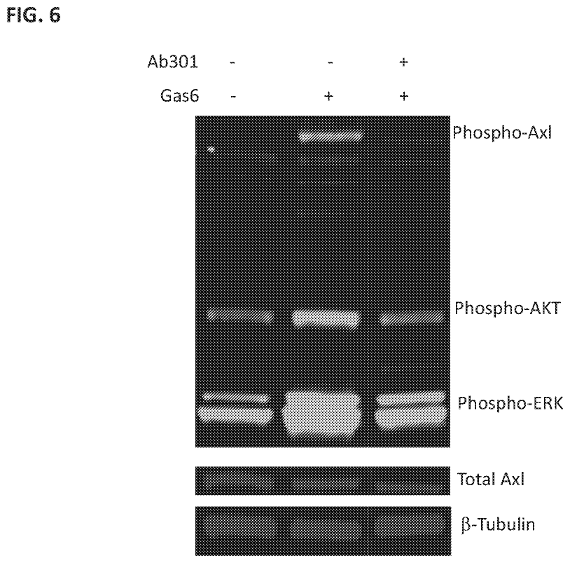

Inhibition of Gas6-dependent phosphorylation of Axl, AKT and ERK by Ab301 was assessed using the following protocol: Serum-deprived H1299 cells (ATTC®) were treated with 100 nM Ab301 for one hour at 37° C., followed by incubation with 100 nM Gas6 for 10 minutes. Cell lysates were subjected to sodium dodecyl sulfate polyacrylamide gel electrophoresis (SDS-PAGE) and immunoblotting with antibodies raised against phospho-Axl, total Axl, phospho-AKT, phospho-ERK and β-tubulin. Antibodies were obtained from Cell Signaling, with the exception of β-tubulin, which was obtained from Odyssey. Images were obtained with a LI-COR® Odyssey instrument. Data is shown in FIG. 6, which shows that Ab301 inhibited Gas6-dependent phosphorylation of Axl, AKT and ERK in a H1299 lung cancer cell line.

### 6.8 Example 8: Cytokine and Chemokine Production in Human Dendritic Cells

The impact of Ab301 on pro-inflammatory cytokine/chemokine production by human dendritic cells was evaluated using the following method: Dendritic cells were derived from human monocytes as follows: peripheral blood mononucleocytes (PBMCs) were added to a T175 cm² flasks, and monocytes were allowed to adhere for approximately 2 hours at 37° C., 6% CO₂. The non-adherent cells were removed and the monocytes were cultured for 7 days in Roswell Park Memorial Institute media (RPMI) containing 10% fetal bovine serum (FBS), 10 ng/mL IL-4 (R&D Systems) and 100 ng/mL granulocyte-macrophage colony-stimulating factor (GM-CSF, R&D Systems). The cells were harvested and frozen for future use.

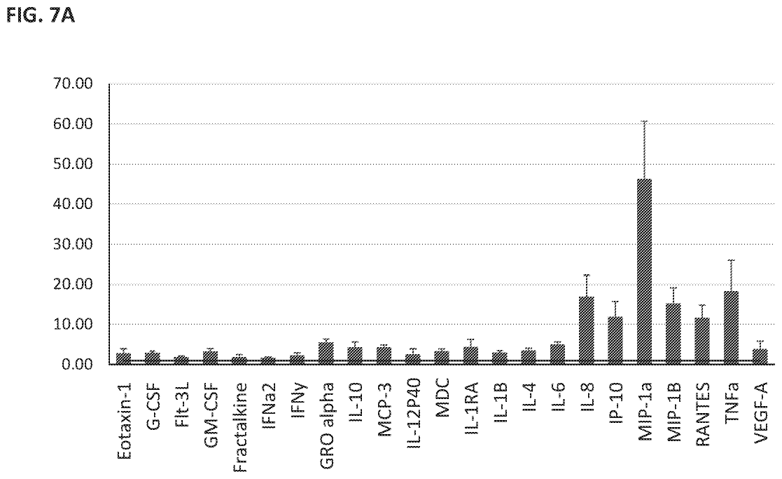

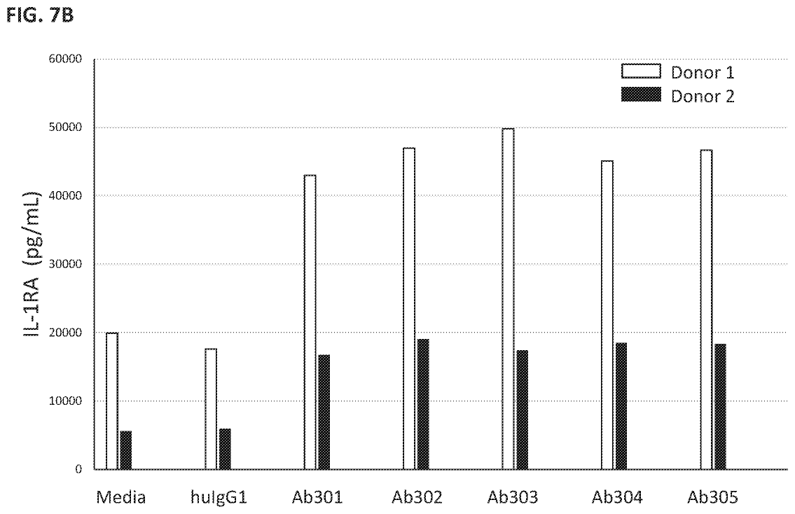

On the day of the experiment, the cells were thawed, washed twice in RPMI and counted. The cells were then incubated in the presence of anti-Axl antibody or appropriate isotype control overnight at 37° C., 6% CO₂. All reagents were prepared in RPMI (no serum). The supernatant was collected, clarified and either used immediately or stored frozen for cytokine analysis. Production of IL-IRA and TNF-α was evaluated using an ELISA kit (R&D Systems). Multiplex analysis was performed by Eve Technologies (Alberta, Calif.) using a Luminex® assay. Data is shown in FIG. 7A, which shows the induction of pro-inflammatory cytokines and chemokines in human dendritic cells by Ab301 compared to isotype control.

In a separate experiment, the impact of Ab301-Ab305 on IL-IRA production by human dendritic cells was evaluated using the same method described in this example. Data is shown in FIG. 7B, which shows the induction of IL-IRA in human dendritic cells from two donors by Ab301-Ab305 compared to isotype control.

### 6.9 Example 9: IL-1RA Production in Human PBMCs

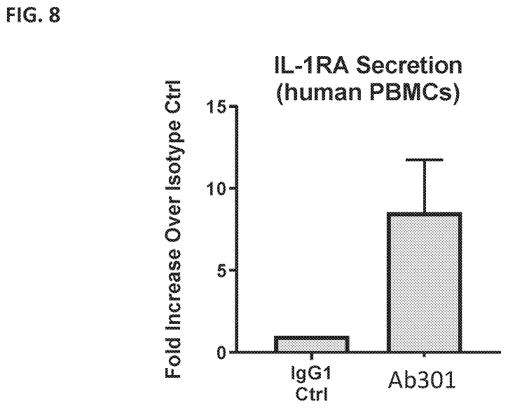

The effect of Ab301 on the production of IL-IRA by human PBMCs was assessed using the following method: Human PBMCs were treated with 100 nM of Ab301 for 24 hours in the absence of serum. IL-1RA was measured from conditioned media by using an ELISA kit (R&D Biosystems), following manufacturer's recommendations. Cytokine data were normalized to human IgG1 control-treated sample. Data is shown in FIG. 8, which shows the elicitation of IL-1RA secretion from human PBMCs by Ab301.

### 6.10 Example 10: Blocking of Gas6 Binding to Axl-Expressing Cells

Blocking of Gas6 binding to Axl-expressing cells by Ab302 was measured using the following methods:

Mouse L cells (from the American Type Culture Collection (ATCC) transfected to stably express human Axl (huAxl L cells) were incubated with increasing concentrations of Ab302 on ice for 1 hour, followed by incubation with 7.5 nM of Alexa-647-labeled Gas6 (Gas6-647) on ice. Cells were washed, fixed in 4% paraformaldehyde (PFA) in PBS (Electron Microscopy Sciences), and mean fluorescence intensity (MFI) was quantified in an Accuri™ C6 flow cytometer.

huAxl L cells were incubated with 7.5 nM Gas6-647 on ice for 1 hour, followed by incubation with 250 nM Ab302 for 5 hours on ice. Cells were fixed in 4% PFA, and MFI values were quantified in an Accuri™ C6 flow cytometer. An anti-Axl monoclonal antibody that does not block Gas6 binding (non-blocking Axl mAb) was used as a control.

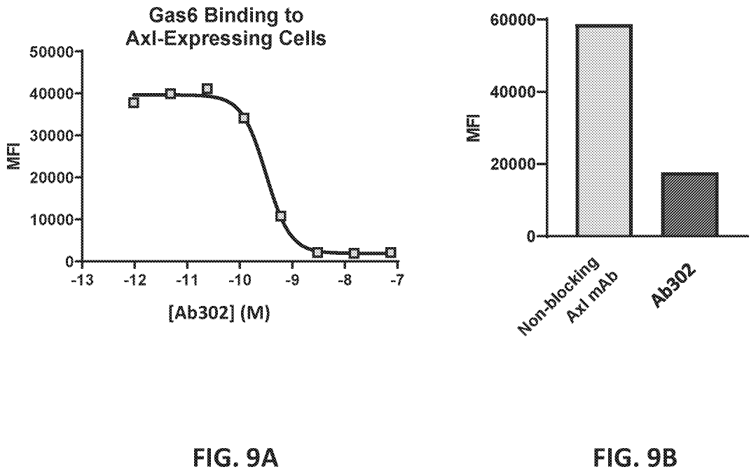

As shown in FIG. 9A, Ab302 blocked Gas6 binding to Axl-expressing cells in a concentration-dependent manner. Further, as shown in FIG. 9B, Ab302 displaced Axl-bound Gas6.

### 6.11 Example 11: Activation of Signaling Pathways that Lead to Immune Activation in Human Macrophages

Activation by Ab302 of signaling pathways that lead to immune activation was measured using the following method: Human macrophages were differentiated by treating peripheral blood mononuclear cells (PBMC) from 2 donors with 100 ng/mL M-CSF for 7 days. 300 μl of 100 nM Ab302 was wet-coated onto 24 well plates after which 1×10⁶ macrophages were added for 1 hour at 37° C. Lipopolysaccharides (LPS) was used as a positive control and added at a final concentration of 100 ng/mL to plated macrophages for 1 hour. After incubation, cells were lysed and subjected to quantification with a Li-Cor Odyssey® CLx.

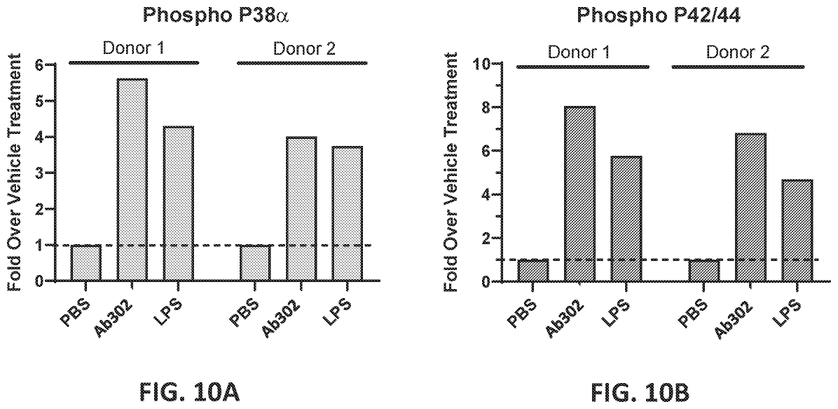

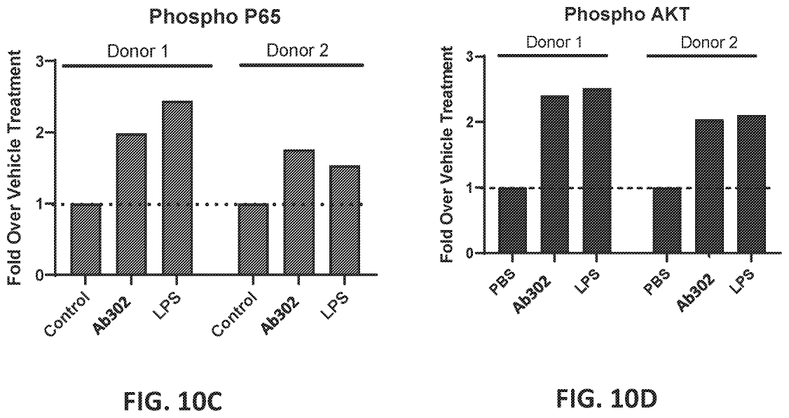

The experiments described herein demonstrate that Ab302 induced phosphorylation of p38α, p42/44, p65 and AKT, as presented in FIGS. 10A-10D, respectively. These data demonstrate that Ab302 treatment induced activation of signaling pathways that lead to immune activation in human macrophages.

### 6.12 Example 12: Antibody-Dependent Cell-Mediated Cytotoxicity (ADCC) in Axl-Expressing SKOV3 Cells

Induction of ADCC by Ab302 was measured using the following methods:

SKOV3 cells (from ATCC) were incubated with PE-labeled isotype control or Ab302 in FACS staining buffer (R&D systems) for 30 minutes on ice. Cells were washed three times and stained with 7-Aminoactinomycin D (7-AAD) for 10 minutes, washed and fixed. Fluorescence was measured using a BD Accuri™ C6 flow cytometer.

Axl-expressing target SKOV3 cells (from ATCC) were lifted, counted and resuspended in low IgG serum containing RPMI (Promega) to a concentration of 3.0×10⁶ cells/mL. They were added to appropriate wells of a white, 96-well plate. Human IgG1 isotype control and monoclonal antibodies (Ab302 or a mutant version of Ab302 that has a mutated Fc domain to reduce or prevent binding to Fc receptors) were each diluted in ADCC media (Promega) to appropriate concentrations and added to target cells. ADCC Reporter Bioassay Effector Cells (Promega) were thawed in a 37° C. water bath for 2 minutes, diluted in ADCC media, and added at a 1:1 ratio to target cells. The cell mixtures were incubated for 6 hours at 37° C. in 5% CO₂ followed by addition of Bio-Glo™ (Promega) for 10 minutes at room temperature. Fluorescence was measured in a BioTek Synergy™ HT plate reader.

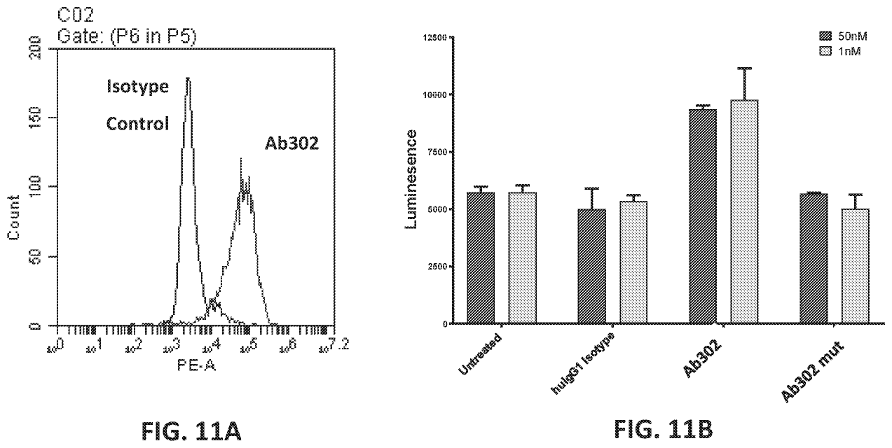

The experiments described herein show that Ab302 induced ADCC in Axl-expressing SKOV3 cells (FIG. 11A). Further, AB302, but not the human IgG1 control or the Ab302 mutant control, induced ADCC in Axl-expressing SKOV3 cells (FIG. 11B).

### 6.13 Example 13: Mixed Lymphocyte Reaction

The mixed lymphocyte reaction to Ab302 was measured using the following method: CD4⁺ T cells were incubated with allogeneic dendritic cells (DCs) and Ab302 or the human IgG1 isotype control for 72 hours. The supernatant was collected, clarified and either used immediately or stored frozen for cytokine analysis. Production of IL-2 was evaluated by ELISA (R&D Systems).

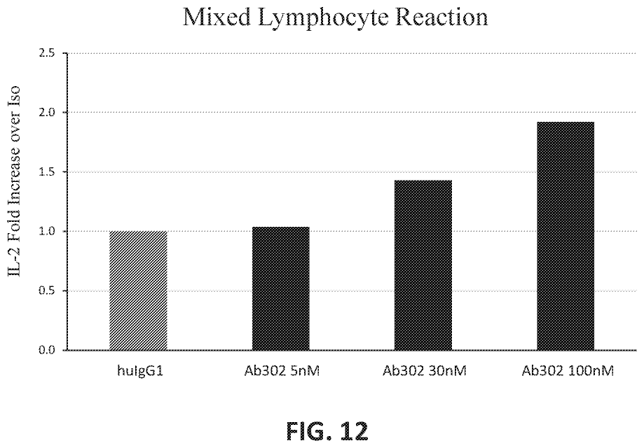

The data presented in FIG. 12 demonstrates that IL-2 production increased in response to Ab302 in a concentration-dependent manner.

What is claimed:

1. An isolated antibody, or an antigen-binding fragment thereof, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

2. The antibody or antigen-binding fragment of claim 1, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

3. The antibody or antigen-binding fragment of claim 1, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

4. The antibody or antigen-binding fragment of claim 1, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 28 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

5. The antibody or antigen-binding fragment of any one of claims 1-4, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively, or conservative sequence modifications thereof.

6. The antibody or antigen-binding fragment of any one of claims 1-4, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively, or conservative sequence modifications thereof.

7. The antibody or antigen-binding fragment of any one of claims 1-4, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively.

8. The antibody or antigen-binding fragment of any one of claims 1-4, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 40, 41, 42 and 43, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 45, 46 and 43, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 41, 47 and 43, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 48, 49, 50 and 51, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 44, 125, 126 and 43, respectively.

9. An isolated antibody, or an antigen-binding fragment thereof, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

10. The antibody or antigen-binding fragment of embodiment 9, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

11. The antibody or antigen-binding fragment of claim 9, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

12. The antibody or antigen-binding fragment of claim 9, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

13. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively, or conservative sequence modifications thereof.

14. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively, or conservative sequence modifications thereof.

15. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively.

16. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 60, 61, 62 and 63, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 65, 66 and 63, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 67, 68 and 63, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 69, 49, 70 and 78, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 64, 131, 132 and 63, respectively.

17. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively, or conservative sequence modifications thereof.

18. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively, or conservative sequence modifications thereof.

19. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively.

20. The antibody or antigen-binding fragment of any one of claims 9-12, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 71, 61, 72 and 63, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 65, 74 and 63, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 67, 75 and 63, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 76, 49, 77 and 78, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 73, 131, 133 and 63, respectively.

21. An isolated antibody, or an antigen-binding fragment thereof, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

22. The antibody or antigen-binding fragment of claim 21, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

23. The antibody or antigen-binding fragment of claim 21, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

24. The antibody or antigen-binding fragment of claim 21, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 30, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 33, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 37 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 123, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

25. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively, or conservative sequence modifications thereof.

26. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively, or conservative sequence modifications thereof.

27. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively.

28. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 79, 80, 81 and 82, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 84, 85 and 82, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 86, 87 and 82, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 88, 49, 89 and 90, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 83, 134, 135 and 82, respectively.

29. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively, or conservative sequence modifications thereof.

30. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively, or conservative sequence modifications thereof.

31. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively.

32. The antibody or antigen-binding fragment of any one of claims 21-24, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 91, 80, 92 and 93, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 84, 95 and 93, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 52, 53, 54 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 86, 96 and 93, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 56, 57, 58 and 59, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 97, 49, 98 and 99, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 127, 128, 129 and 55, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 94, 134, 115 and 93, respectively.

33. An isolated antibody, or an antigen-binding fragment thereof, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively, or conservative sequence modifications thereof; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

34. The antibody or antigen-binding fragment of claim 33, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively, or conservative sequence modifications thereof;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively, or conservative sequence modifications thereof;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively, or conservative sequence modifications thereof;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively, or conservative sequence modifications thereof; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively, or conservative sequence modifications thereof; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively, or conservative sequence modifications thereof.

35. The antibody or antigen-binding fragment of claim 33, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively; and/or
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

36. The antibody or antigen-binding fragment of claim 33, which specifically binds to human Axl, comprising:
(A) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 20, 21 and 22, respectively;
(B) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 23, 24 and 22, respectively;
(C) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 38, 31 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 25, 26 and 22, respectively;
(D) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 39, 34 and 35, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 27, 36 and 29, respectively; or
(E) (i) a light chain variable region (VL) comprising VL complementarity determining region 1 (CDR1), VL CDR2, and VL CDR3, wherein the VL CDR1, VL CDR2, and VL CDR3 comprise the amino acid sequences of SEQ ID NOS: 130, 124 and 32, respectively; and
(ii) a heavy chain variable region (VH) comprising VH complementarity determining region 1 (CDR1), VH CDR2, and VH CDR3, wherein the VH CDR1, VH CDR2, and VH CDR3 comprise the amino acid sequences of SEQ ID NOS: 120, 121 and 122, respectively.

37. The antibody or antigen-binding fragment of any one of claims 33-36, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively, or conservative sequence modifications thereof; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively, or conservative sequence modifications thereof.

38. The antibody or antigen-binding fragment of any one of claims 33-36, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively, or conservative sequence modifications thereof;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively, or conservative sequence modifications thereof;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively, or conservative sequence modifications thereof;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively, or conservative sequence modifications thereof; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively, or conservative sequence modifications thereof; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively, or conservative sequence modifications thereof.

39. The antibody or antigen-binding fragment of any one of claims 33-36, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively; and/or
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively.

40. The antibody or antigen-binding fragment of any one of claims 33-36, wherein:
(A) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 100, 61, 101 and 93, respectively;
(B) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 65, 103 and 93, respectively;
(C) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 107, 108, 109 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 67, 104 and 93, respectively;
(D) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 111, 112, 113 and 114, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 105, 49, 106 and 99, respectively; or
(E) (i) the light chain variable region (VL) further comprises VL framework region 1 (FR1), VL FR2, VL FR3, and VL FR4, wherein the VL FR1, VL FR2, VL FR3 and VL FR4 comprise the amino acid sequences of SEQ ID NOS: 117, 118, 119 and 110, respectively; and
(ii) the heavy chain variable region (VH) further comprises VH framework region 1 (FR1), VH FR2, VH FR3, and VH FR4, wherein the VH FR1, VH FR2, VH FR3, and VH FR4 comprise the amino acid sequences of SEQ ID NOS: 102, 131, 116 and 93, respectively.

41. The antibody or antigen-binding fragment of any one of claims 1-36, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 5.

42. The antibody or an antigen-binding fragment of claim 41, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 5.

43. The antibody or antigen-binding fragment of any one of claims 1-8, wherein:
(i) the light chain variable region (VL) comprises an amino acid sequence of SEQ ID NO: 6; and/or
(ii) the heavy chain variable region (VH) comprises an amino acid sequence of SEQ ID NO: 5.

44. The antibody or an antigen-binding fragment of claim 43, wherein:
(i) the light chain variable region (VL) comprises an amino acid sequence of SEQ ID NO: 6; and
(ii) the heavy chain variable region (VH) comprises an amino acid sequence of SEQ ID NO: 5.

45. The antibody or antigen-binding fragment of any one of claims 9-16, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 7.

46. The antibody or an antigen-binding fragment of claim 45, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 7.

47. The antibody or antigen-binding fragment of any one of claims 9-16, wherein:
(i) the light chain variable region (VL) comprises an amino acid sequence of SEQ ID NO: 6; and/or
(ii) the heavy chain variable region (VH) comprises an amino acid sequence of SEQ ID NO: 7.

48. The antibody or an antigen-binding fragment of claim 47, wherein:
(i) the light chain variable region (VL) comprises an amino acid sequence of SEQ ID NO: 6; and
(ii) the heavy chain variable region (VH) comprises an amino acid sequence of SEQ ID NO: 7.

49. The antibody or antigen-binding fragment of any one of claims 9-12 and 17-20, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 8.

50. The antibody or an antigen-binding fragment of claim 49, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 8.

51. The antibody or antigen-binding fragment of any one of claims 9-12 and 17-20, wherein:
(i) the light chain variable region (VL) comprises an amino acid sequence of SEQ ID NO: 6; and/or
(ii) the heavy chain variable region (VH) comprises an amino acid sequence of SEQ ID NO: 8.

52. The antibody or an antigen-binding fragment of claim 51, wherein:
(i) the light chain variable region (VL) comprises an amino acid sequence of SEQ ID NO: 6; and
(ii) the heavy chain variable region (VH) comprises an amino acid sequence of SEQ ID NO: 8.

53. The antibody or antigen-binding fragment of any one of claims 21-28, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 9.

54. The antibody or an antigen-binding fragment of claim 53, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 9.

55. The antibody or antigen-binding fragment of any one of claims 21-28, wherein:
(i) the light chain variable region (VL) comprises an amino acid sequence of SEQ ID NO: 6; and/or
(ii) the heavy chain variable region (VH) comprises an amino acid sequence of SEQ ID NO: 9.

56. The antibody or an antigen-binding fragment of claim 55, wherein:
(i) the light chain variable region (VL) comprises an amino acid sequence of SEQ ID NO: 6; and
(ii) the heavy chain variable region (VH) comprises an amino acid sequence of SEQ ID NO: 9.

57. The antibody or antigen-binding fragment of any one of claims 21-24 and 29-32, wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 10.

58. The antibody or an antigen-binding fragment of claim 49, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 10.

59. The antibody or antigen-binding fragment of any one of claims 21-24 and 29-32, wherein:
(i) the light chain variable region (VL) comprises an amino acid sequence of SEQ ID NO: 6; and/or
(ii) the heavy chain variable region (VH) comprises an amino acid sequence of SEQ ID NO: 10.

60. The antibody or an antigen-binding fragment of claim 51, wherein:
(i) the light chain variable region (VL) comprises an amino acid sequence of SEQ ID NO: 6; and
(ii) the heavy chain variable region (VH) comprises an amino acid sequence of SEQ ID NO: 10.

61. The antibody or antigen-binding fragment of any one of claims 33-40, wherein:
(i) the light chain variable region (VL) comprises an amino acid sequence having at least 80%, at least 85%, at least 90%, at least 95%, at least 98%, or 100% sequence identity to SEQ ID NO: 12; and/or
(ii) the heavy chain variable region (VH) comprises an amino acid sequence having at least 80%, at least 85%, at least 90%, at least 95%, at least 98%, or 100% sequence identity to SEQ ID NO: 11.

62. The antibody or an antigen-binding fragment of claim 61, wherein:
(i) the light chain variable region (VL) comprises an amino acid sequence having at least 80%, at least 85%, at least 90%, at least 95%, at least 98%, or 100% sequence identity to SEQ ID NO: 12; and
(ii) the heavy chain variable region (VH) comprises an amino acid sequence having at least 80%, at least 85%, at least 90%, at least 95%, at least 98%, or 100% sequence identity to SEQ ID NO: 11.

63. The antibody or antigen-binding fragment of any one of claims 33-40, wherein:
(i) the light chain variable region (VL) comprises an amino acid sequence of SEQ ID NO: 12; and/or
(ii) the heavy chain variable region (VH) comprises an amino acid sequence of SEQ ID NO: 11.

64. The antibody or an antigen-binding fragment of claim 63, wherein:
(i) the light chain variable region (VL) comprises an amino acid sequence of SEQ ID NO: 12; and
(ii) the heavy chain variable region (VH) comprises an amino acid sequence of SEQ ID NO: 11.

65. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 5.

66. The antibody or an antigen-binding fragment of claim 65, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 5.

67. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 5.

68. The antibody or an antigen-binding fragment of claim 67, wherein:
(i) the light chain variable region (VL) comprises an amino acid sequence of SEQ ID NO: 6; and
(ii) the heavy chain variable region (VH) comprises an amino acid sequence of SEQ ID NO: 5.

69. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 7.

70. The antibody or an antigen-binding fragment of claim 69, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 7.

71. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 7.

72. The antibody or an antigen-binding fragment of claim 71, wherein:
(i) the light chain variable region (VL) comprises an amino acid sequence of SEQ ID NO: 6; and
(ii) the heavy chain variable region (VH) comprises an amino acid sequence of SEQ ID NO: 7.

73. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 8.

74. The antibody or an antigen-binding fragment of claim 73, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 8.

75. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 8.

76. The antibody or an antigen-binding fragment of claim 75, wherein:
(i) the light chain variable region (VL) comprises an amino acid sequence of SEQ ID NO: 6; and
(ii) the heavy chain variable region (VH) comprises an amino acid sequence of SEQ ID NO: 8.

77. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 9.

78. The antibody or an antigen-binding fragment of claim 77, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 9.

79. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 9.

80. The antibody or an antigen-binding fragment of claim 79, wherein:
(i) the light chain variable region (VL) comprises an amino acid sequence of SEQ ID NO: 6; and
(ii) the heavy chain variable region (VH) comprises an amino acid sequence of SEQ ID NO: 9.

81. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 6; and/or
(ii) the heavy chain variable region (VH) comprises an amino acid sequence having at least 80%, at least 85%, at least 90%, at least 95%, at least 98%, or 100% sequence identity to SEQ ID NO: 10.

82. The antibody or an antigen-binding fragment of claim 81, wherein:
(i) the light chain variable region (VL) comprises an amino acid sequence having at least 80%, at least 85%, at least 90%, at least 95%, at least 98%, or 100% sequence identity to SEQ ID NO: 6; and
(ii) the heavy chain variable region (VH) comprises an amino acid sequence having at least 80%, at least 85%, at least 90%, at least 95%, at least 98%, or 100% sequence identity to SEQ ID NO: 10.

83. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 6; and/or
(ii) the heavy chain variable region (VH) comprises an amino acid sequence of SEQ ID NO: 10.

84. The antibody or an antigen-binding fragment of claim 83, wherein:
(i) the light chain variable region (VL) comprises an amino acid sequence of SEQ ID NO: 6; and
(ii) the heavy chain variable region (VH) comprises an amino acid sequence of SEQ ID NO: 10.

85. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence having at least 80%, at least 85%, at least 90%, at least 95%, at least 98%, or 100% sequence identity to SEQ ID NO: 12; and/or
(ii) the heavy chain variable region (VH) comprises an amino acid sequence having at least 80%, at least 85%, at least 90%, at least 95%, at least 98%, or 100% sequence identity to SEQ ID NO: 11.

86. The antibody or an antigen-binding fragment of claim 85, wherein:
(i) the light chain variable region (VL) comprises an amino acid sequence having at least 80%, at least 85%, at least 90%, at least 95%, at least 98%, or 100% sequence identity to SEQ ID NO: 12; and
(ii) the heavy chain variable region (VH) comprises an amino acid sequence having at least 80%, at least 85%, at least 90%, at least 95%, at least 98%, or 100% sequence identity to SEQ ID NO: 11.

87. An isolated antibody, or an antigen-binding fragment thereof, which specifically binds to human Axl, comprising a light chain variable region (VL) and a heavy chain variable region (VH), wherein:
(i) the light chain variable region (VL) comprises an amino acid sequence of SEQ ID NO: 12; and/or
(ii) the heavy chain variable region (VH) comprises an amino acid sequence of SEQ ID NO: 11.

88. The antibody or an antigen-binding fragment of claim 87, wherein:
(i) the light chain variable region (VL) comprises an amino acid sequence of SEQ ID NO: 12; and
(ii) the heavy chain variable region (VH) comprises an amino acid sequence of SEQ ID NO: 11.

89. The antibody or antigen-binding fragment of any one of claims 1 to 63 and 65 to 87, which is a humanized antibody.

90. The antibody or antigen-binding fragment of any one of claims 1-89, which specifically binds to Ig-like domain 1 in the extracellular domain of human Axl.

91. The antibody or antigen-binding fragment of any one of claims 1-90, which does not specifically bind to the extracellular domain of MerTK.

92. An isolated antibody, or an antigen-binding fragment thereof, which specifically binds to Ig-like domain 1 in the extracellular domain of human Axl.

93. The antibody or antigen-binding fragment of claim 92, which does not specifically bind to the extracellular domain of MerTK.

94. An isolated antibody, or an antigen-binding fragment thereof, which binds to the same epitope of human Axl as the antibody of any of claims 1 to 93.

95. An isolated antibody, or an antigen-binding fragment thereof, which competes for binding to human Axl with the antibody of any of claims 1 to 93.

96. An isolated antibody, or an antigen-binding fragment thereof, which binds to Ig-like domain 1 in the extracellular domain of human Axl and inhibits binding of Gas6 to Axl and/or, inhibits Axl phosphorylation.

97. The antibody or antigen-binding fragment of claim 96, which does not specifically bind to the extracellular domain of MerTK.

98. The antibody or antigen-binding fragment of any one of claims 1 to 97, wherein the antibody comprises a heavy chain constant region or a light chain constant region.

99. The antibody or antigen-binding fragment of claim 98, wherein the antibody comprises a heavy chain constant region and a light chain constant region.

100. The antibody or antigen-binding fragment of claim 98, wherein the antibody comprises a human heavy chain constant region or a human light chain constant region.

101. The antibody or antigen-binding fragment of claim 100 wherein the antibody comprises a human heavy chain constant region and a human light chain constant region.

102. The antibody or antigen-binding fragment of any one of claims 98 to 101, wherein the antibody or antigen-binding fragment is an IgG antibody or antigen-binding fragment thereof.

103. The antibody or antigen-binding fragment of claim 102, wherein the antibody or antigen-binding fragment is an IgG1 antibody or antigen-binding fragment thereof.

104. The antibody or antigen-binding fragment of any one of claims 98 to 103, wherein the antibody comprises a kappa light chain constant region or a lambda light chain constant region.

105. The antibody or antigen-binding fragment of any one of claims 98 to 103, wherein the antibody comprises a human kappa light chain constant region or a human lambda light chain constant region.

106. The antibody or antigen-binding fragment of any one of claims 1 to 105, which is a monoclonal antibody.

107. The antibody or antigen-binding fragment of any one of claims 1 to 106, which is a bispecific antibody.

108. The antibody or antigen-binding fragment of any one of claims 1 to 107, which is fused to a heterologous polypeptide.

109. The antibody or antigen-binding fragment of any one of claims 1 to 108, which is conjugated to an agent.

110. The antibody or antigen-binding fragment of claim 109, wherein the agent is a toxin.

111. The antibody or antigen-binding fragment of any one of claims 1-110, which inhibits binding of Gas6 to Axl.

112. The antibody or antigen-binding fragment of any one of claims 1-111, which comprises an Fc region or domain that is capable of binding Fc receptors.

113. The antibody or antigen-binding fragment of any one of claims 1-112, which is capable of eliciting an effector function.

114. The antibody or antigen-binding fragment of any one of claims 1-113, which is capable of eliciting antibody-dependent cell-mediated cytotoxicity.

115. A human or humanized monoclonal antibody, or an antigen-binding fragment thereof, that preferably binds to Ig-like domain 1 in the extracellular domain of human Axl and which exhibits one or more of the following properties:
i) binds to human Axl with an affinity constant (equilibrium dissociation constant) KD of 10 nM or less, preferably 5 nM or less or preferably 1 nM or less as determined by bio-layer interferometry;
ii) binds to human Axl in cells with an EC50 of 1 μg/mL or less, or preferably 0.1 μg/mL or less, as determined by flow cytometry;
iii) inhibits Gas6 binding to human Axl;
iv) inhibits Gas6-dependent phosphorylation of Axl, AKT and ERK;
v) when tested in vitro increases the secretion of Eotaxin-1, G-CSF, Flt-3L, GM-CSF, Fractalkine, IFNa2, IFNγ, GRO alpha, IL-10, MCP-3, IL-12P40, MDC, IL-1RA, IL-1B, IL-4, TNFα, RANTES, MIP-1B, IL-6, IL-8, IP-10, VEGF-A, and/or MIP-1a from human monocyte-derived dendritic cells by at least 100% as determined by a Luminex assay when compared to isotype control;
vi) when tested in vitro increases IL-1RA secretion from human PBMCs by at least 5-fold as determined by ELISA when compared to isotype control;
vii) when tested in vitro increases IL-1RA secretion from human dendritic cells by at least 2-fold as determined by ELISA when compared to isotype control; and
viii) when tested in vitro increases IL-2 secretion from a co-culture of CD4⁺ T cells and dendritic cells by at least 40% as determined by ELISA when compared to isotype control.

116. A composition comprising a therapeutically effective amount of the antibody or antigen-binding fragment of any one of claims 1 to 115.

117. A pharmaceutical composition comprising the antibody or antigen-binding fragment of any one of claims 1 to 115 and a pharmaceutically acceptable carrier.

118. A polynucleotide comprising one or more nucleotide sequences encoding a VH chain region, a VL chain region, or both a VL chain region and a VH chain region, of an antibody or antigen-binding fragment of any one of claims 1 to 115.

119. A polynucleotide comprising one or more nucleotide sequences encoding a heavy chain, a light chain, or both heavy chain and a light chain of an antibody or antigen-binding fragment of any one of claims 1 to 115.

120. A population of polynucleotides comprising (i) a first polynucleotide comprising a nucleotide sequence encoding a VH or a heavy chain of the antibody or antigen-binding fragment of any one of claims 1 to 115 and (ii) a second polypeptide comprising a nucleotide sequence encoding a VL or a light chain of the antibody or antigen-binding fragment.

121. A vector comprising the polynucleotide of claim 118 or 119.

122. A population of vectors comprising (i) a first vector comprising a nucleotide sequence encoding a VH or a heavy chain of the antibody or antigen-binding fragment of any one of claims 1 to 115, and (ii) a second vector comprising a nucleotide sequence encoding a VL or a light chain of the antibody or antigen-binding fragment.

123. An isolated cell comprising the polynucleotide of claim 118 or 119.

124. An isolated cell comprising the population of polynucleotides of claim 120.

125. A population of cells comprising (i) a first host cell comprising a polynucleotide comprising a nucleotide sequence encoding a VH or a heavy chain of the antibody or antigen-binding fragment of any one of claims 1 to 115, and (ii) a second host cell comprising a polynucleotide comprising a nucleotide sequence encoding a VL or a light chain of the antibody or antigen-binding fragment.

126. An isolated cell producing the antibody or antigen-binding fragment of any one of claims 1 to 115.

127. A kit comprising the antibody or antigen-binding fragment of any one of claims 1 to 115.

128. A method of making an antibody or an antigen-binding fragment thereof that specifically binds to human Axl, comprising culturing the cell or population of cells of any one of claims 123 to 126 to express the antibody or antigen-binding fragment.

129. A method of making an antibody or an antigen-binding fragment thereof that specifically binds to human Axl, comprising expressing the polynucleotide or population of polynucleotides of any one of claims 118 to 120.

130. A method of managing, protecting against, or treating cancer in a subject, comprising administering to a subject in need thereof a therapeutically effective amount of the antibody or antigen-binding fragment of any one of claims 1 to 115.

131. A method of enhancing an immune response in a subject comprising administering to a subject in need thereof an effective amount of the antibody or antigen-binding fragment of any one of claims 1 to 115.

132. The method of claim 131, wherein the subject is an immunocompromised subject.

133. The method of claim 132, wherein the immunocompromised subject is suffering from an infection, has cancer, is undergoing, or has had undergone treatment with, an anti-cancer therapy, is HIV positive, or has AIDS or SCID, or diabetes, or has had a transplant and is taking immunosuppressants.

134. The method of claim 133, wherein the subject has been treated with an immunosuppressant.

135. A method of enhancing an immune response to a vaccine in a subject, comprising administering to a subject in need thereof, who is or has been administered the vaccine, an effective amount of the antibody or antigen-binding fragment of any one of claims 1 to 115.

136. The method of claim 135, wherein the vaccine is a cancer or tumor vaccine.

137. A method of managing, preventing, protecting against, or treating metastasis in a subject, comprising administering to a subject in need thereof a therapeutically effective amount of the antibody or antigen-binding fragment of any one of claims 1 to 115.

138. A method of managing or treating sepsis in a subject, comprising administering to a subject in need thereof a therapeutically effective amount of the antibody or antigen-binding fragment of any one of claims 1 to 115.

139. A method for activating or enhancing an innate immune response in a subject, comprising administering to a subject in need thereof an effective amount of the antibody or antigen-binding fragment of any one of claims 1 to 115.

140. The method of claim 137, wherein the subject has cancer, or is being treated for cancer with an anti-cancer therapeutic agent, or the subject has an infection.

141. A method of increasing proinflammatory cytokine production in a subject in need thereof, comprising administering to the subject an antibody or antigen-binding fragment thereof that specifically binds to human Axl such that the production of at least one proinflammatory cytokine is increased.

142. The method of claim 141, wherein the at least one proinflammatory cytokine is Eotaxin-1, G-CSF, Flt-3L, GM-CSF, Fractalkine, IFNa2, IFNγ, GRO alpha, IL-2, IL-10, MCP-3, IL-12P40, MDC, IL-1RA, IL-1B, IL-4, TNFα, RANTES, MIP-1B, IL-6, IL-8, IP-10, VEGF-A, and/or MIP-1a.

143. A method of increasing proinflammatory cytokine production in a subject in need thereof, comprising administering to the subject an antibody or an antigen-binding fragment thereof of any one of claims 1 to 115 such that the production of at least one proinflammatory cytokine is increased.

144. The method of claim 143, wherein the at least one proinflammatory cytokine is Eotaxin-1, G-CSF, Flt-3L, GM-CSF, Fractalkine, IFNa2, IFNγ, GRO alpha, IL-2, IL-10, MCP-3, IL-12P40, MDC, IL-1RA, IL-1B, IL-4, TNFα, RANTES, MIP-1B, IL-6, IL-8, IP-10, VEGF-A, and/or MIP-1a.

145. The method of claim 143, wherein the at least one proinflammatory cytokine is TNFα.

146. The method of claim 143, wherein the at least one proinflammatory cytokine is RANTES.

147. The method of claim 143, wherein the at least one proinflammatory cytokine is MIP-1B.

148. The method of claim 143, wherein the at least one proinflammatory cytokine is IL-8

149. The method of claim 143, wherein the at least one proinflammatory cytokine is MIP-1a.

150. The method of claim 143, wherein the at least one proinflammatory cytokine is IP-10.

151. The method of claim 143, wherein the at least one proinflammatory cytokine is IL-1RA.

152. The method of claim 143, wherein the at least one proinflammatory cytokine is IL-2.

153. A method of increasing proinflammatory secretory factor production in a subject in need thereof, comprising administering to the subject an antibody or an antigen-binding fragment thereof of any one of claims 1 to 115 such that the production of at least one proinflammatory secretory factor is increased.

154. The method of claim 153, wherein the production of at least one proinflammatory secretory factor is TNFα, IL-1RA, fibroblast growth factor 2 (FGF-2), eotaxin-1 (CCL11), transforming growth factor alpha (TGF-a) granulocyte-colony stimulating factor (G-CSF), Fms-related tyrosine kinase 3 ligand (Flt-3L), granulocyte macrophage-colony stimulating factor (GM-CSF), fractalkine (CX3CL1), interferon alpha-2 (IFN-a2), interferon-gamma (IFN-γ), growth-regulated oncogene alpha (GRO alpha), interleukin-2 (IL-2), interleukin-10 (IL-10), monocyte chemotactic protein 3 (MCP-3), interleukin-12 p40 (IL-12P40), macrophage-derived chemokine (MDC), platelet-derived growth factor AA homodimer (PDGF-AA), interleukin-13 (IL-13), platelet-derived growth factor BB homodimer (PDGF-BB), soluble CD40 ligand (sCD40L), interleukin-1B (IL-1B), interleukin-4 (IL-4), interleukin-6 (IL-6), interleukin-8 (IL-8), interferon γ-induced protein 10 (IP-10), macrophage inflammatory protein (MIP)-1a, MIP-10, Regulated on Activation Normal T cell Expressed and Secreted (RANTES), vascular endothelial growth factor A (VEGF-A) and/or IL-18.

155. The method of any one of claims 130 to 154, wherein the antibody or antigen-binding fragment thereof does not substantially induce phosphorylation of Axl.

156. The method of any one of claims 130 to 155, further comprising administering to the subject a second therapeutic agent.

157. The method of claim 156, wherein the second therapeutic agent is an immune checkpoint blockade.

158. The method of claim 157, wherein the immune checkpoint blockade inhibits the activity of PD-1, PD-L1, PD-L2, CTLA-4, TIM-3, or LAG-3.

159. The method of claim 157, wherein the immune checkpoint blockade inhibits the activity of PD-1.

160. The method of claim 157, wherein the immune checkpoint blockade inhibits the activity of PD-L1.

161. The method of any one of claims 156 to 160, wherein the second therapeutic agent is a monoclonal antibody.
